SEQUENCE LISTING (62 sequences)

SEQ ID NO: 1 - DNA, 2558 bp, Bacillus anthracis
atggataagccggatgtagatacgattgaaggcttatctccagcgatttcaatcgatcaaaaaacgacgagtcgtaatccgcgttcaactgttggaacggtaacggagatttatgattacttacgtttattatttgcgcgaattggtacgccaatttgtccgaatcatggcattgaaattacatcgcaaacagtagagcaaatggtagaccgtgtacttgagtaccctgaacgtacgaaattacaagtgttagctcctatcgtttctgggcgtaaaggtgcacatgtaaaagtacttgaagatattaagaagcaaggttatgttcgtgtacgtgttgatggtgaaatgctcgatgtgtctgaagatattgcgttagataaaaataagaagcattctattgaagttgtaattgaccgtattgttgtaaaagaaggaatcgcaagccgtcttgctgattctcttgaaagtgcattaaagcttggcgggggacgagtgttaatcgatgttatgggagaagaggagcttctatttagtgaacatcatgcttgtccgcattgtggtttttcaattggagaattagagccgcgtatgttctcattcaatagtccgttcggtgcatgtccttcttgtgatgggcttggctcaaagttagaggtagatttagaacttgttattccgaactgggatttatcattaaatgagcatgcgattgcgccttgggaaccgacaagttcacaatattacccacagcttttacaatctgtatgtaatcattatggcgtggatatggatgtgcctgtaaaagatataccgaaagatttatttgataaagtgttgtacggaagcggtgaagagaaagtttatttccgctatgtaaatgaatttggtcaagtaaaggaaaatgagattttatttgaaggtgttattccaaatattgaacgtcgttatcgtgagacgagttcggattacattcgtgagcaaatggaaaagtatatggcagaacaagcttgtccgaagtgtaaaggcggacgcttaaagcctgaaagtttagctgttttcgttggcggaaaaacgattgctgatgtaacgaagtattctgttcaagaagtacaggaattcttctcaaatgtggagctaacagagaaacaacaaaaaattgcccatttaattttaagagaaattcaagagcgcgttgggttcttagtaaacgttggtttagattatttaacgttaagtcgtgccgcaggaactttatctggtggtgaggcgcaacgtattcgtttagcaacgcaaattggttcgcgtcttactggggtgctttacattcttgatgagccttctatcggtttgcatcagcgcgataacgatcgtcttattcgtacattgcaagaaatgcgtgatttaggtaatacgttaattgttgttgagcatgatgaagatacgatgatggcagctgattatttactggatatcgggcctggcgcaggtattcacggtggacaagttgtatcagcgggtacaccagctgaagtgatgcaagatgagaattcactaacaggtaagtatttaagcggtaaagagtttattccagttccacttgaaagacgtaaaggtgatggacgtaaagtggagattgtcggtgcaaaagagaataacttaaagaacgcgaagatgtcattcccgcttggtacgtttgtagcggtaacgggtgtatctggatcaggtaaaagtacgatgattaatgaagtactatataaatcgttagcgcaaaagttatataaagcgaaagcgaagccaggtactcataaagaaattaaaggtcttgagcatttagataaagttatcgatattgatcaatcgccaatcggtcgtacaccacgttctaatccagcaacctatacaggtgtgttcgatgatattcgtgatgtgtttgcgcaaacgaatgaagcgaaagtgcgcggatatcaaaaagggcgtttcagctttaacgtaaaaggtggacgttgtgaagcgtgccgtggtgatggaattattaaaattgagatgcacttcttaccagacgtatacgttccgtgtgaagtttgtcacggtaaacgttacaaccgtgaaacgttagaagtgaaatataaagataaaaacatttctgaagtgttagggatgacgattgaagacggagtagagttcttcgctaatatcccaaaaattaaacgtaaacttcaaacgcttgtagacgttgggcttggttatatgaaattagggcaaccagccacgactttatctggtggtgaagcacagcgtgtgaaattagcttctgaattacaccgtcgttctacaggacgtacactatacattttagacgagccaacgactggtttacatgcgcatgatatcgcccgtcttctagaagtgctgcaacgtcttgttgagagcggtgagacggtacttgtcattgaacataatttagatgtaattaaaacagcggattatatcgttgaccttggaccagaaggcggagacaaaggtggacaaatcgttgcttccggaacgccagagcaagt

SEQ ID NO: 2 - DNA, 1728 bp, Bacillus anthracis
atggctcacaataaaacgttagcaggacagttatatagtgagttgaaagactttttcccgaataatgcagttgaatattttgttagttattacgattattatcagccagaagcgtatgtgccacaaacagatacgtttattgaaaaagacgcgcagattaatgatgaaatcgataaattgcgtcactcagcaacgtccgcattatttgaacgggatgatgtaattattgttgcgagtgtttcgtgtatatatggtttaggttctccagaagaataccgcgagttagttgtttcacttcgagttggtatggaaaaggaccgcaatcaattgcttcgtgaacttgttgatgtgcagtatggacgtaatgatattgatttcaagcgtggtacattccgcgtgcgcggagatgtagttgaaatcttcccggcatcacttgacgagcattgcattcgaattgagttttttggcgatgaaattgatcgtattcgcgaagtaaatgctttaacgggagaagtattagcagaacgtgatcatgtagcaatcttcccagcatctcacttcgttacacgtgaagaaaagatgaaggtcgctattgaaaatatcgaaaaagaattagaagagcgtttaaaggaattaaatgataacggtaagttgttagaagcgcagcgtatagaacagcgtacacgttatgatttagaaatgatgcgcgagatgggcttttgttcagggattgaaaactattcccgtcatttaacacttcgtccagcgggtgcaacgccgtatacgttattagactatttcccgaaagatttcttaatcgttatggatgagtcccacgtatcagtgccgcaagtaagagcgatgtataacggggaccaagcgcgtaaacaagtgcttgtggatcatggattccgtctgccatcagctttagataatagaccgctcacatttgatgagtttgaagagaaaacgaatcaagttatttacgtttcagcaacgccaggaccgtatgaattagagcagtcgccagaagtaatagaacaaattattcgtccaacagggcttttagatccgccaattgatatacgaccaattgaagggcagattgacgatctattaggagagattcaagatcgcattgcaaaaaatgaacgtgtattaattacaactttaacgaagaagatgtcagaggatttaacagactacttaaaagatgtaggaattaaggtgaattatctgcattctgaagtgaaaacgttagaacgtattgaaattatacgagatcttcgccttggtaagtttgatgttctcgttggtattaacttattgcgagaaggattagatattccagaagtatcccttgtagctattttagatgccgataaggaaggattcttgcgttcagagcgttcgttaattcaaacaattggccgtgcagcacgtaatgaaaacggtcgcgttattatgtacgcagatcgtataacgagatcgatggggattgcgattgaagagacgaagcgtcgtcgtagtatacaagaagcttacaatgaagagcatggtattacgccgaaaacgattcaaaaaggtgtgcgtgatgtaatccgtgcaacgacagctgctgaagagccggaaacatatgaagcgacgccagctaagaagatgacgaaaaaagagcgtgaaaagacaattgcgaagatggaagcagaaatgaaagaagcagcaaaagca

SEQ ID NO: 3 - RNA, 16 nt, Escherichia coli
  misc_feature 1, 9, 10, 11, 12, 13: n = A,U,C or G
naggaggunnnnnaug

SEQ ID NO: 4 - protein, 31 aa, Bacillus anthracis
MKKRKVLIPLMALSTILVSSTGNLEVIQAEV

SEQ ID NO: 5 - protein, 5 aa, Bacillus anthracis
IQAEV

SEQ ID NO: 6 - DNA, 25 bp, Artificial Sequence
  Primer
ctgtgctttgcgaatggaaagaagc

SEQ ID NO: 7 - DNA, 43 bp, Artificial Sequence
  Primer
gttttcattcatacacttagacaagcgttggcttttgcacttc

SEQ ID NO: 8 - DNA, 24 bp, Artificial Sequence
  Primer
gacaagcgttggcttttgcacttc

SEQ ID NO: 9 - DNA, 44 bp, Artificial Sequence
  Primer
caaaagccaacgcttgtctaagtgtatgaatgaaaaccgagtgg

SEQ ID NO: 10 - DNA, 25 bp, Artificial Sequence
  Primer
aagtgtatgaatgaaaaccgagtgg

SEQ ID NO: 11 - DNA, 28 bp, Artificial Sequence
  Primer
catataaaggttccacaattgccttttc

SEQ ID NO: 12 - DNA, 28 bp, Artificial Sequence
  Primer
gaagcagaaatgaagccaatactcaatc

SEQ ID NO: 13 - DNA, 27 bp, Artificial Sequence
  Primer
ggttccacaattgccttttcaataatc

SEQ ID NO: 14 - protein, 6 aa, Escherichia coli
KVVKNK

SEQ ID NO: 15 - DNA, 12 bp, Bacillus subtilis
  misc_feature 5, 6, 7, 8: n = A,T,C or G
gaacnnnngttc

SEQ ID NO: 16 - protein, 792 aa, Bacillus anthracis
MKFEQVFFRWGFIIVVIGFLLGRAYILTNILPFALPFFAAVYVMKRDKMPLAFLALMGGALSVSIDNLFFTFASIFTFFIYNIFFSRFTRKTVGLVPFQVFISALTAHLVVVYFAQQTVTMYDLLVSTIEAGLSFVLTMIFLQSVPLLVERKGKQQALETEEIVCLIILLASVLTGTTDWFVYDASIQHIFTRYLVLVFAFIAGAATGSTVGVVTGLILSLANVSSLSQLSLLAFSGLLGGLLKEGKRIGVSLGLLIGTSLITLYVDKQTNIVTTLIESGVAIAFFLLTPKLVMDRIAKFMPGTQEHSQDQQQYLRRMRDVTANKINQFANVFAALSNSFSVYGYVEEEDKETEADLFLSTITAKTCQTCFKKDQCWVVNFDKTYDYMKQIMSETEEGTLQHNRKLVREWDKHCVRGKKVTDLVAGELDHFYEGQKLRKQMKENRRIVAEQLLGVSKVMEDFAKEIQRERENHQVQEEQILQAFRDFGVEIEHVDIYCLDRGSIDIEMLIPVASNEHGECEKLVAPMLSDILKENIVVKHEEKSSYPNGHSLISFGSAKTYSLDTGLATAAKGGGFVSGDSYAMMDLSVGKYALAISDGMGNGQRAHMESKETVKLLQKILQSGIDEEIAIKSINSILSLRTTEEMFTTLDLAMVDLRDASAKFLKIGSTPSFVKRANNILKIEASNLPMGIIEDVEVDVVGEQLKTGDILIMMSDGIFEGAQHVENHELWMKRKIKELQTEDPQEIADIIMEEVIRSGDGYINDDMTIVVAKVKKNMPKWATIPIVGMQAQ

SEQ ID NO: 17 - DNA, 2379 bp, Bacillus anthracis
atgaagtttgagcaagttttctttagatggggatttattattgttgttattggttttcttttaggacgagcatatatattaacaaacattttaccgtttgcactgccgttttttgctgctgtttatgttatgaagcgggataaaatgccgcttgcattcttggccctaatggggggcgcactgtcagtttcgatagataatttattctttacctttgcatctatttttactttcttcatttataatatcttctttagtcggtttacacgtaaaactgttggacttgttccattccaagtatttatctccgcattaaccgcacatttagttgtcgtatattttgcgcaacaaacagtcaccatgtacgatttacttgttagtacaattgaggccgggcttagcttcgtattaactatgatatttttacaaagtgtcccgcttttagtagaaagaaaagggaaacaacaagccttagagacagaagaaattgtttgtttaattatattactagcatctgttttaacaggtacaacagattggttcgtatatgacgcttctattcaacatatttttactaggtatttagtgcttgtatttgcgtttatcgcaggagcggctacaggatctacagtaggggtcgtcactgggttaatattaagcctggcaaatgtctccagtttgtcccaacttagcctacttgctttttctggattgcttggtggtttgttaaaagaagggaagcgcataggtgttagtttaggtttattaattgggacaagcctgattacgctatatgtagacaagcaaacaaacattgtgacaactttaatcgaatctggtgtggcgattgctttcttcttattaacaccgaaacttgttatggatcgtattgctaaatttatgccaggcacacaggagcattcgcaagatcaacaacagtatttaagaaggatgcgtgatgttacagcgaataaaattaatcaatttgctaatgtgtttgctgctttatctaatagcttttctgtatatggatacgtggaggaagaagataaagagacggaggcagatctgttcttaagtacaattactgcaaaaacatgtcaaacatgctttaaaaaggaccaatgctgggtagttaatttcgataaaacatacgattatatgaaacaaataatgagtgaaacagaagaggggacgttacagcataatcggaagttagttcgtgaatgggataagcattgtgtgagaggaaagaaagtgacggatttagtggcaggcgaattagatcacttctatgagggacaaaaattaagaaaacaaatgaaagaaaatcgtagaatagtagcggagcaactattgggtgtatcaaaagttatggaggattttgctaaggagatacaaagagagcgagaaaaccatcaagtacaggaagaacagattctgcaagcgtttcgtgattttggtgtagaaatagagcatgttgatatttattgtttagatagaggaagtattgatattgaaatgttgattccagttgcatctaatgaacacggagagtgtgaaaaattagttgcaccgatgctttctgatattctaaaggaaaatatcgttgttaagcatgaagaaaaatcttcttatccgaatggccacagcttaatatcatttggttcagcaaaaacgtattctcttgatacgggcttagccacagctgcaaaaggcggtgggtttgtttcaggtgattcttatgcgatgatggatttaagtgttggtaaatatgctcttgcgattagtgatggtatgggaaatgggcaaagagctcatatggagagtaaagaaacagtgaaattattacaaaaaatacttcaatcaggcattgatgaagaaatagcgattaagtctattaactctattctttctttaagaacaacagaagagatgtttactacgttagatttagctatggtagatttgcgggatgcgagtgcgaagtttttaaagattggatcgacgccgagttttgttaaacgcgcaaacaatattttgaaaattgaagcaagtaatttgccgatgggaataattgaggatgttgaagttgatgtagtgggtgagcaattaaaaacaggcgatatccttattatgatgagcgatgggatttttgagggagcgcaacatgtggagaatcatgaattatggatgaagcgtaaaattaaagagttgcaaactgaagatccgcaagaaatcgctgatatcatcatggaagaggtaattcgctctggtgatggttatataaatgatgatatgactattgtagtggcaaaagtgaagaaaaatatgccgaagtgggctaccattccaattgtgggaatgcaggcacaataa

SEQ ID NO: 18 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 19 - DNA, 2295 bp, Bacillus anthracis
atgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtcaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataa

SEQ ID NO: 20 - protein, 809 aa, Bacillus anthracis
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLARYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 21 - DNA, 2430 bp, Bacillus anthracis
atgaatataaaaaaagaatttataaaagtaattagtatgtcatgtttagtaacagcaattactttgagtggtcccgtctttatcccccttgtacagggggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggatgctggcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaaattgatattcgtgattctttatctgaagaagaaaaagagcttttaaatagaatacaggtggatagtagtaatcctttatctgaaaaagaaaaagagtttttaaaaaagctgaaacttgatattcaaccatatgatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtgccaaagagtaaaatagatacaaaaattcaagaagcacagttaaatataaatcaggaatggaataaagcattagggttaccaaaatatacaaagcttattacattcaacgtgcataatagatatgcatccaatattgtagaaagtgcttatttaatattgaatgaatggaaaaataatattcaaagtgatcttataaaaaaggtaacaaattacttagttgatggtaatggaagatttgtttttaccgatattactctccctaatatagctgaacaatatacacatcaagatgagatatatgagcaagttcattcaaaagggttatatgttccagaatcccgttctatattactccatggaccttcaaaaggtgtagaattaaggaatgatagtgagggttttatacacgaatttggacatgctgtggatgattatgctggatatctattagataagaaccaatctgatttagttacaaattctaaaaaattcattgatatttttaaggaagaagggagtaatttaacttcgtatgggagaacaaatgaagcggaattttttgcagaagcctttaggttaatgcattctacggaccatgctgaacgtttaaaagttcaaaaaaatgctccgaaaactttccaatttattaacgatcagattaagttcattattaactcataa

SEQ ID NO: 22 - protein, 800 aa, Bacillus anthracis
MTRNKFIPNKFSIISFSVLLFAISSSQAIEVNAMNEHYTESDIKRNHKTEKNKTEKEKFKDSINNLVKTEFTNETLDKIQQTQDLLKKIPKDVLEIYSELGGEIYFTDIDLVEHKELQDLSEEEKNSMNSRGEKVPFASRFVFEKKRETPKLIINIKDYAINSEQSKEVYYEIGKGISLDIISKDKSLDPEFLNLIKSLSDDSDSSDLLFSQKFKEKLELNNKSIDINFIKENLTEFQHAFSLAFSYYFAPDHRTVLELYAPDMFEYMNKLEKGGFEKISESLKKEGVEKDRIDVLKGEKALKASGLVPEHADAFKKIARELNTYILFRPVNKLATNLIKSGVATKGLNVHGKSSDWGPVAGYIPFDQDLSKKHGQQLAVEKGNLENKKSITEHEGEIGKIPLKLDHLRIEELKENGIILKGKKEIDNGKKYYLLESNNQVYEFRISDENNEVQYKTKEGKITVLGEKFNWRNIEVMAKNVEGVLKPLTADYDLFALAPSLTEIKKQIPQKEWDKVVNTPNSLEKQKGVTNLLIKYGIERKPDSTKGTLSNWQKQMLDRLNEAVKYTGYTGGDVVNHGTEQDNEEFPEKDNEIFIINPEGEFILTKNWEMTGRFIEKNITGKDYLYYFNRSYNKIAPGNKAYIEWTDPITKAKINTIPTSAEFIKNLSSIRRSSNVGVYKDSGDKDEFAKKESVKKIAGYLSDYYNSANHIFSQEKKRKISIFRGIQAYNEIENVLKSKQIAPEYKNYFQYLKERITNQVQLLLTHQKSNIEFKLLYKQLNFTENETDNFEVFQKIIDEK

SEQ ID NO: 23 - DNA, 2403 bp, Bacillus anthracis
atgactagaaataaatttatacctaataagtttagtattatatccttttcagtattactatttgctatatcctcctcacaggctatagaagtaaatgctatgaatgaacattacactgagagtgatattaaaagaaaccataaaactgaaaaaaataaaactgaaaaagaaaaatttaaagacagtattaataacttagttaaaacagaatttaccaatgaaactttagataaaatacagcagacacaagacttattaaaaaagatacctaaggatgtacttgaaatttatagtgaattaggaggagaaatctattttacagatatagatttagtagaacataaggagttacaagatttaagtgaagaagagaaaaatagtatgaatagtagaggtgaaaaagttccgtttgcatcccgttttgtatttgaaaagaaaagggaaacacctaaattaattataaatatcaaagattatgcaattaatagtgaacaaagtaaagaagtatattatgaaattggaaaggggatttctcttgatattataagtaaggataaatctctagatccagagtttttaaatttaattaagagtttaagcgatgatagtgatagtagcgaccttttatttagtcaaaaatttaaagagaagctagaattgaataataaaagtatagatataaattttataaaagaaaatttaactgaatttcagcatgcgttttctttagcgttttcttattattttgcacctgaccatagaacggtattagagttatatgcccccgacatgtttgagtatatgaataagttagaaaaagggggatttgagaaaataagtgaaagtttgaagaaagaaggtgtggaaaaagataggattgatgtgctgaaaggagaaaaagcacttaaagcttcaggtttagtaccagaacatgcagatgcttttaaaaaaattgctagagaattaaatacatatattctttttaggcctgttaataagttagctacaaaccttattaaaagtggtgtggctacaaagggattgaatgttcatggaaagagttcggattggggccctgtagctggatacataccatttgatcaagatttatctaagaagcatggtcaacaattagctgtcgagaaaggaaatttagaaaataaaaaatcaattacagagcatgaaggtgaaataggtaaaataccattaaagttagaccatttaagaatagaagagttaaaggaaaatgggataattttgaagggtaaaaaagaaattgataatggtaaaaaatattatttgttagaatcgaataatcaggtatatgaatttagaattagcgatgaaaacaacgaagtacaatacaagacaaaagaaggtaaaattactgttttaggggaaaaattcaattggagaaatatagaagtgatggctaaaaatgtagaaggggtcttgaagccgttaacagctgactatgatttatttgcacttgccccaagtttaacagaaataaaaaaacaaataccacaaaaagaatgggataaagtagttaacaccccaaattcattagaaaagcaaaaaggtgttactaatttattgattaaatatggaattgagaggaaaccggattcaactaagggaactttatcaaattggcaaaaacaaatgcttgatcgtttgaatgaagcagtcaaatatacaggatatacagggggggatgtggttaaccatggcacagagcaagataatgaagagtttcctgaaaaagataacgaaatttttataattaatccagaaggtgaatttatattaactaaaaattgggagatgacaggtagatttatagaaaaaaacattacgggaaaagattatttatattattttaaccgttcttataataaaatagctcctggtaataaagcttatattgagtggactgatccgattacaaaagccaaaataaataccatccctacgtcagcagagtttataaaaaacttatccagtatcagaagatcttcaaatgtaggagtttataaagatagtggcgacaaagacgaatttgcaaaaaaagaaagcgtgaaaaaaattgcaggatatttgtcagactattacaattcagcaaatcatattttttctcaggaaaaaaagcgtaaaatatcaatatttcgtggaatccaagcctataatgaaattgaaaatgttctaaaatctaaacaaatagcaccagaatacaaaaattattttcaatatttaaaggaaaggattaccaatcaagttcaattgcttctaacacatcaaaaatctaatattgaatttaaattattgtataaacaattaaactttacagaaaatgaaacggataattttgaggtcttccaaaaaattattgatgaaaaataa

SEQ ID NO: 24 - protein, 206 aa, Bacillus anthracis
MEKLTKRQQDILDFIKLKVQEKGYPPSVREIGQAVGLASSSTVHGHLSRLEEKGYIRRDPTKPRAIEILGEDRMDTETQSVIQVPIVGKVTAGLPITAVESVEEHFPLPASIVAGADQVFMLRISGDSMIEAGIFDGDLVVVRQQQSAYNGEIVVALTEDNEATVKRFYKEKDHFRLQPENSSLEPIILKQVSVIGKVIGVYRDLH

SEQ ID NO: 25 - DNA, 621 bp, Bacillus anthracis
atggaaaagttaacgaaacgccagcaagacattctcgactttattaagctaaaagtacaagaaaaaggatatccaccctccgtacgtgaaatcggtcaagcagtcggcctcgcttctagttctacagtgcacggacatttatcaagattagaagaaaaaggatacattcgacgcgatccaacaaaaccacgtgcaattgaaattttaggtgaagaccgaatggatacagaaacacaatctgttattcaagttccaattgtcggaaaagttactgccggtttaccaattacagcggtcgaaagcgtagaggagcacttccctcttccagctagcattgtcgcaggagcagatcaagtgtttatgttacgtatttccggggatagtatgattgaggctggcattttcgatggagatttagttgttgttcgccaacaacagtctgcatataatggtgaaattgtagtcgctttaacagaagataatgaagcaactgttaaacgtttctataaagaaaaagaccatttccgtctacaaccggaaaactcttcattagaacctatcattttaaagcaagtgtcagttatcggtaaagtaattggcgtatatcgtgatttacattaa

SEQ ID NO: 26 - protein, 343 aa, Bacillus anthracis
MSDRQAALDMALKQIEKQFGKGSIMKLGEQAERKVSTVSSGSLALDVALGVGGYPRGRIIEIYGPESSGKTTVSLHAIAEVQRQGGQAAFIDAEHAMDPVYAQKLGVNIDELLLSQPDTGEQGLEIAEALVRSGAVDIIVIDSVAALVPKAEIEGDMGDSHVGLQARLMSQALRKLSGAINKSKTIAIFINQIREKVGVMFGNPETTPGGRALKFYSTVRLEVRRAEQLKQGNDIVGNKTKVKVVKNKVAPPFRVAEVDIMYGEGISREGEILDMASELDIVQKSGAWYSYNEERLGQGRENSKQFLKENTDLREEIAFFIREHHGISEDSGAEGMEDPNLLD

SEQ ID NO: 27 - DNA, 1032 bp, Bacillus anthracis
atgagtgatcgtcaagcagcgttagatatggcgttaaaacaaatagagaagcaattcggtaaaggttcaattatgaaattaggagaacaagcagagcgcaaagtttctacggtttctagtggttctttagcacttgatgtggcattaggggtaggcggatacccacgcggccgtattatcgaaatttacggacctgaaagttcaggtaaaacaacagtttcattacacgcaattgcagaagtacagcgtcaaggtggacaagcagcgttcattgatgctgagcatgcaatggatcctgtatatgcacaaaaactaggtgttaacatcgatgaattactattatcacaacctgatacaggggagcaaggtttagaaatcgcagaagcacttgtacgaagtggtgcggttgatattatcgtaattgactctgtagcagctcttgtaccgaaagctgaaattgaaggagacatgggtgactcacacgtaggtttacaagctcgtctaatgtctcaagcacttcgtaaactttcaggtgcaatcaataaatcaaaaacaatcgcaatctttattaaccaaattcgtgaaaaagttggggttatgttcggaaatcaactacaagttgcttaatcgtaaagattaaggagtttaccatgggtttcccttgctagtgatagcatgttaaaaaaccttgtgaaccttattgcctaagggtgtaactattcatatagttgctaacggtgaagcccaccattattcagggtaataccgtgccaagctccatagtgatatggggaaggtgtagagactaccgaaaaggacttttagttaactgagtagggtacggcagatgataggctactgctggaagtgcaaggctctctgtaaagagatgaagagatagtccggactatagagatggaaaatctatagatagtgccagaaacaactccaggtggtcgtgcgttgaaattctattcaactgttcgtcttgaagtgcgtcgtgcggagcaattaaaacaaggtaacgacatc

SEQ ID NO: 28 - DNA, 1722 bp, Bacillus anthracis
atgaaagataggcaaggaacggttatatatgtcggaaaggcaaaagtgcttaaaaatcgtgtgcgctcgtactttactggttcgcatgacgggaaaacacttcggttagtaggagaaattgtagattttgaatatattgtaacctcctcaaatctagaggcgctcattttggagttaaacttaataaaaaaacatgacccaaaatataatattcaattaaaagatgataaaacatatccttttattaaaattacagctgagaaacaaccgcgcttacttattacgcgaaatgtaaaaaaggataaaggaaagtattttggcccttatccgaatgcacaatcagctcatgaaacgaaaaaactgctggatcgtatgtatccgcttcgtaagtgctcaaatatgccggataaagtttgtttatattatcatatgggtcaatgtttagcaccttgtgtgaaagaagtgacggaagaacaaaataaagaaattgtagatgagattattaagtttttaaatggtgggcataaagaagttcgttcagaattagaaacaaaaatgtatgaagcttcagagaaactagagtttgaacgtgcaaaagagttacgtgatcaaatcgctcatatcgatgcgattatggaaaaacaaaagatgattatgagtgatttagtggaccgtgatgtgtttggctatgcagttgataaagggtggatgtgtgttcaagttttcttcgttcggaaaggaaagttaattgaacgtgatgtttctatgtttccaatatatgatgaaccagaagagggattcttaacgtttatcggtcaattttatgaaaacagcagtcattttaagccgaaagaaatagttgttccaggaagtatagactcagaattagtagaacgctttttagaagtggaagcgacacagccgaaacgcggtaagaaaaaagatcttgtagaactggcaaataaaaatgcgaagattgccctggaagagaaattctatttaattgaacgtgatgaagagcgaacgattaaagctgtagagaatttagggaagcagctcggaattgaaacgccttatcgtattgaagcatttgataactcaaatattcaagggacaaatcctgtttctgcaatgattgcttttattgatgggaaaccagctaagaaagaatacaggaaatataaaattaaaacagttcaaggaccagatgattatgagtctatgagagaagttgtgagacgccgttatacaagggcgctgaaagagggtttacctttaccagatttaatcattattgatggcggaaaaggtcatctggcggctgcaagtgatgttctagaaaatgagctcggtttatatattccgatggcaggtcttgtaaaagatgacaaacataaaacatctcatttaattattggagatccacctgaacctgtgatgctggagagaaatagccaagaattttatttattgcagcgtgttcaagatgaagtgcatcgatttgcaattacatttcatcgtcaattacacgggaaatctgtcattcaatcagcactggatgatattcctggaatcggtgataaacggaaaaaggtattgttaaaacattttggttcattaaagaagatgaaagaagcttctatagaggaatttgtcgaagcaggtatgccgaaaaatgtcgcagagacgatttatacttatttaacagataagaagacgttg

SEQ ID NO: 29 - DNA, 14 bp, Bacillus subtilis
  misc_feature 6, 8: r = G or A
  misc_feature 9, 13: y = C or T
  misc_feature 7: n = A,T,C or G
cgaacrnrygttyc

SEQ ID NO: 30 - DNA, 20 bp, Artificial Sequence
  Primer
taacgaccgcgctccaaaag

SEQ ID NO: 31 - DNA, 47 bp, Artificial Sequence
  Primer
ggcatatttttcttcacttttgcccactttactcctccaagctgacc

SEQ ID NO: 32 - DNA, 48 bp, Artificial Sequence
  Primer
ggtcagcttggaggagtaaagtgggcaaaagtgaagaaaaatatgccg

SEQ ID NO: 33 - DNA, 20 bp, Artificial Sequence
  Primer
tgcattcatcccgtattgct

SEQ ID NO: 34 - DNA, 25 bp, Artificial Sequence
  Primer
agataaaatacagcagacacaagac

SEQ ID NO: 35 - DNA, 20 bp, Artificial Sequence
  Primer
tagttgaatccggtttcctc

SEQ ID NO: 36 - DNA, 31 bp, Artificial Sequence
  Primer
ggtgtggctacacagggattgaatgttcatg

SEQ ID NO: 37 - DNA, 31 bp, Artificial Sequence
  Primer
catgaacattcaatccctgtgtagccacacc

SEQ ID NO: 38 - DNA, 33 bp, Artificial Sequence
  Primer
gattgaatgttcatggacagagttcggattggg

SEQ ID NO: 39 - DNA, 33 bp, Artificial Sequence
  Primer
cccaatccgaactctgtccatgaacattcaatc

SEQ ID NO: 40 - DNA, 31 bp, Artificial Sequence
  Primer
acagggattgaatgtccatggacagagttcg

SEQ ID NO: 41 - DNA, 31 bp, Artificial Sequence
  Primer
cgaactctgtccatggacattcaatccctgt

SEQ ID NO: 42 - DNA, 25 bp, Artificial Sequence
  Primer
caggagggttaattgatagtccgtc

SEQ ID NO: 43 - DNA, 24 bp, Artificial Sequence
  Primer
tttgcgtcttatttggctttaacg

SEQ ID NO: 44 - DNA, 36 bp, Artificial Sequence
  Primer
agtgagggttttatagctgagtttggacatgctgtg

SEQ ID NO: 45 - DNA, 36 bp, Artificial Sequence
  Primer
cacagcatgtccaaactcagctataaaaccctcact

SEQ ID NO: 46 - DNA, 14 bp, Bacillus anthracis
atgttttttcacat

SEQ ID NO: 47 - DNA, 14 bp, Bacillus anthracis
  misc_feature 5, 6, 7, 8, 9, 10: n = A,T,C or G
atgtnnnnnnacat

SEQ ID NO: 48 - DNA, 15 bp, Bacillus anthracis
aaatgttctccattt

SEQ ID NO: 49 - DNA, 15 bp, Bacillus anthracis
  misc_feature 6, 7, 8, 9, 10: n = A,T,C or G
aaatgnnnnncattt

SEQ ID NO: 50 - DNA, 1062 bp, Escherichia coli
atggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgacaacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaa

SEQ ID NO: 51 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 52 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKMVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 53 - DNA, 1032 bp, Bacillus anthracis
atgagtgatcgtcaagcagcgttagatatggcgttaaaacaaatagagaagcaattcggtaaaggttcaattatgaaattaggagaacaagcagagcgcaaagtttctacggtttctagtggttctttagcacttgatgtggcattaggggtaggcggatacccacgcggccgtattatcgaaatttacggacctgaaagttcaggtaaaacaacagtttcattacacgcaattgcagaagtacagcgtcaaggtggacaagcagcgttcattgatgctgagcatgcaatggatcctgtatatgcacaaaaactaggtgttaacatcgatgaattactattatcacaacctgatacaggggagcaaggtttagaaatcgcagaagcacttgtacgaagtggtgcggttgatattatcgtaattgactctgtagcagctcttgtaccgaaagctgaaattgaaggagacatgggtgactcacacgtaggtttacaagctcgtctaatgtctcaagcacttcgtaaactttcaggtgcaatcaataaatcaaaaacaatcgcaatctttattaaccaaattcgtgaaaaagttggggttatgttcggaaatccagaaacaactccaggtggtcgtgcgttgaaattctattcaactgttcgtcttgaagtgcgtcgtgcggagcaattaaaacaaggtaacgacatcgttggtaataaaacaaaagtaaaagtagttaaaaataaagtggcaccaccattccgtgttgcggaagttgatattatgtacggagaaggtatttcaagagaaggtgaaatcttagatatggcctctgaacttgatatcgttcaaaagagcggtgcttggtactcttataatgaagaacgcttaggacaaggtcgtgaaaattcgaagcagttcttaaaagaaaatacggatttaagagaagaaattgccttctttattcgtgagcatcacggaattagcgaagattctggtgctgaaggtatggaagatccaaatcttcttgattaa

SEQ ID NO: 54 - protein, 343 aa, Bacillus anthracis
MSDRQAALDMALKQIEKQFGKGSIMKLGEQAERKVSTVSSGSLALDVALGVGGYPRGRIIEIYGPESSGKTTVSLHAIAEVQRQGGQAAFIDAEHAMDPVYAQKLGVNIDELLLSQPDTGEQGLEIAEALVRSGAVDIIVIDSVAALVPKAEIEGDMGDSHVGLQARLMSQALRKLSGAINKSKTIAIFINQIREKVGVMFGNPETTPGGRALKFYSTVRLEVRRAEQLKQGNDIVGNKTKVKMVKNKVAPPFRVAEVDIMYGEGISREGEILDMASELDIVQKSGAWYSYNEERLGQGRENSKQFLKENTDLREEIAFFIREHHGISEDSGAEGMEDPNLLD

SEQ ID NO: 55 - DNA, 5520 bp, Bacillus anthracis
tcatccatacttaataaactcgcatcttttattagtagtacatcatcacagtgttgatagaagatgtagcaatcctttttcacttcattatgaaacgcgttctccgcttcttctccctctaaacacttctttctctcataactaaatatgtgccatagatacccacatgcatgcttatctccataaaggaaaatatcttctttttcctcatcacttacatgatttgcaaaatgatcctcccatcgcttccgaaaatatacaccccacttttgaaactctcttaccttcatatttttctttcgcaacatatctaaaaactccatcttctccccctatacaataaaaaccgagcgtcacttccactcggttttcattcatacacttattgtgataactctacttcttttattttctctttcattctcgctttatcacgatttaaaatctcttttaaatacttacctgtatacgagcgctcttctttcactacttgctctggcgttccggaagcaacgatttgtccacctttgtctccgccttctggtccaaggtcaacgatataatccgctgttttaattacatctaaattatgttcaatgacaagtaccgtctcaccgctctcaacaagacgttgcagcacttctagaagacgggcgatatcatgcgcatgtaaaccagtcgttggctcgtctaaaatgtatagtgtacgtcctgtagaacgacggtgtaattcagaagctaatttcacacgctgtgcttcaccaccagataaagtcgtggctggttgccctaatttcatataaccaagcccaacgtctacaagcgtttgaagtttacgtttaatttttgggatattagcgaagaactctactccgtcttcaatcgtcatccctaacacttcagaaatgtttttatctttatatttcacttctaacgtttcacggttgtaacgtttaccgtgacaaacttcacacggaacgtatacgtctggtaagaagtgcatctcaattttaataattccatcaccacggcacgcttcacaacgtccaccttttacgttaaagctgaaacgccctttttgatatccgcgcactttcgcttcattcgtttgcgcaaacacatcacgaatatcatcgaacacacctgtataggttgctggattagaacgtggtgtacgaccgattggcgattgatcaatatcgataactttatctaaatgctcaagacctttaatttctttatgagtacctggcttcgctttcgctttatataacttttgcgctaacgatttatatagtacttcattaatcatcgtacttttacctgatccagatacacccgttaccgctacaaacgtaccaagcgggaatgacatcttcgcgttctttaagttattctcttttgcaccgacaatctccactttacgtccatcacctttacgtctttcaagtggaactggaataaactctttaccgcttaaatacttacctgttagtgaattctcatcttgcatcacttcagctggtgtacccgctgatacaacttgtccaccgtgaatacctgcgccaggcccgatatccagtaaataatcagctgccatcatcgtatcttcatcatgctcaacaacaattaacgtattacctaaatcacgcatttcttgcaatgtacgaataagacgatcgttatcgcgctgatgcaaaccgatagaaggctcatcaagaatgtaaagcaccccagtaagacgcgaaccaatttgcgttgctaaacgaatacgttgcgcctcaccaccagataaagttcctgcggcacgacttaacgttaaataatctaaaccaacgtttactaagaacccaacgcgctcttgaatttctcttaaaattaaatgggcaattttttgttgtttctctgttagctccacatttgagaagaattcctgtacttcttgaacagaatacttcgttacatcagcaatcgtttttccgccaacgaaaacagctaaactttcaggctttaagcgtccgcctttacacttcggacaagcttgttctgccatatacttttccatttgctcacgaatgtaatccgaactcgtctcacgataacgacgttcaatatttggaataacaccttcaaataaaatctcattttcctttacttgaccaaattcatttacatagcggaaataaactttctcttcaccgcttccgtacaacactttatcaaataaatctttcggtatatcttttacaggcacatccatatccacgccataatgattacatacagattgtaaaagctgtgggtaatattgtgaacttgtcggttcccaaggcgcaatcgcatgctcatttaatgataaatcccagttcggaataacaagttctaaatctacctctaactttgagccaagcccatcacaagaaggacatgcaccgaacggactattgaatgagaacatacgcggctctaattctccaattgaaaaaccacaatgcggacaagcatgatgttcactaaatagaagctcctcttctcccataacatcgattaacactcgtcccccgccaagctttaatgcactttcaagagaatcagcaagacggcttgcgattccttcttttacaacaatacggtcaattacaacttcaatagaatgcttcttatttttatctaacgcaatatcttcagacacatcgagcatttcaccatcaacacgtacacgaacataaccttgcttcttaatatcttcaagtacttttacatgtgcacctttacgcccagaaacgataggagctaacacttgtaatttcgtacgttcagggtactcaagtacacggtctaccatttgctctactgtttgcgatgtaatttcaatgccatgattcggacaaattggcgtaccaattcgcgcaaataataaacgtaagtaatcataaatctccgttaccgttccaacagttgaacgcggattacgactcgtcgttttttgatcgattgaaatcgctggagataagccttcaatcgtatctacatccggcttatccatttgccctaaaaactggcgtgcatacgcagataacgattctacgtatctgcgctgcccttctgcataaatcgtatcaaatgctaatgaggatttccctgaaccagacaatcctgttacaacgacaagttgatttctcggaatggttacatcaatattttttaagttatgtgctctagcaccttttacaacgataaaatccttgctcttactcactcttttcacccttccgcttttaattctaatagtaaatctcttaattcagctgcacgctcgaagtctaatgcttttgctgcttctttcatttctgcttccatcttcgcaattgtcttttcacgctcttttttcgtcatcttcttagctggcgtcgcttcatatgtttccggctcttcagcagctgtcgttgcacggattacatcacgcacacctttttgaatcgttttcggcgtaataccatgctcttcattgtaagcttcttgtatactacgacgacgcttcgtctcttcaatcgcaatccccatcgatctcgttatacgatctgcgtacataataacgcgaccgttttcattacgtgctgcacggccaattgtttgaattaacgaacgctctgaacgcaagaatccttccttatcggcatctaaaatagctacaagggatacttctggaatatctaatccttctcgcaataagttaataccaacgagaacatcaaacttaccaaggcgaagatctcgtataatttcaatacgttctaacgttttcacttcagaatgcagataattcaccttaattcctacatcttttaagtagtctgttaaatcctctgacatcttcttcgttaaagttgtaattaatacacgttcattttttgcaatgcgatcttgaatctctcctaatagatcgtcaatctgcccttcaattggtcgtatatcaattggcggatctaaaagccctgttggacgaataatttgttctattacttctggcgactgctctaattcatacggtcctggcgttgctgaaacgtaaataacttgattcgttttctcttcaaactcatcaaatgtgagcggtctattatctaaagctgatggcagacggaatccatgatccacaagcacttgtttacgcgcttggtccccgttatacatcgctcttacttgcggcactgatacgtgggactcatccataacgattaagaaatctttcgggaaatagtctaataacgtatacggcgttgcacccgctggacgaagtgttaaatgacgggaatagttttcaatccctgaacaaaagcccatctcgcgcatcatttctaaatcataacgtgtacgctgttctatacgctgcgcttctaacaacttaccgttatcatttaattcctttaaacgctcttctaattctttttcgatattttcaatagcgaccttcatcttttcttcacgtgtaacgaagtgagatgctgggaagattgctacatgatcacgttctgctaatacttctcccgttaaagcatttacttcgcgaatacgatcaatttcatcgccaaaaaactcaattcgaatgcaatgctcgtcaagtgatgccgggaagatttcaactacatctccgcgcacgcggaatgtaccacgcttgaaatcaatatcattacgtccatactgcacatcaacaagttcacgaagcaattgattgcggtccttttccataccaactcgaagtgaaacaactaactcgcggtattcttctggagaacctaaaccatatatacacgaaacactcgcaacaataattacatcatcccgttcaaataatgcggacgttgctgagtgacgcaatttatcgatttcatcattaatctgcgcgtctttttcaataaacgtatctgtttgtggcacatacgcttctggctgataataatcgtaataactaacaaaatattcaactgcattattcgggaaaaagtctttcaactcactatataactgtcctgctaacgttttattgtgagccatgacaagcgttggcttttgcacttctttaatgacatttgaaatcgtaaatgtcttacccgttcctgtcgccccaagcaacacttgctttttctttccactattaattccctctacaagcttctctatagctaccggctgatcaccttgcggggaatacgctgagacaatttcaaattgacgttccaagaaaatccgacctgctacaaaaaatatagacaaatgtatattcttagagtgttcctgcttctacgtgtcctgccttgccaaccactcgctactacagtttgatctaacactccacaggcgtaaattccgatgtagcccaccgtacatatatacaatctatttacgttagtattgtatatttttttgcttgtaaaaggtttattgacgcgttcaaatctctatccattacatttccacagttacatgtatatactcggtcggatagctttaagtctactt

SEQ ID NO: 56 - protein, 7 aa, Escherichia coli
VKVVKNK

SEQ ID NO: 57 - protein, 6 aa, Escherichia coli
KMVKNK

SEQ ID NO: 58 - RNA, 17 nt, Escherichia coli
  misc_feature 1, 9, 10, 11, 12, 13, 14: n = A,U,C or G
naggaggunnnnnnaug

SEQ ID NO: 59 - RNA, 18 nt, Escherichia coli
  misc_feature 1, 9, 10, 11, 12, 13, 14, 15: n = A,U,C or G
naggaggunnnnnnnaug

SEQ ID NO: 60 - RNA, 19 nt, Escherichia coli
  misc_feature 1, 9, 10, 11, 12, 13, 14, 15, 16: n = A,U,C or G
naggaggunnnnnnnnaug

SEQ ID NO: 61 - RNA, 20 nt, Escherichia coli
  misc_feature 1, 9, 10, 11, 12, 13, 14, 15, 16, 17: n = A,U,C or G
naggaggunnnnnnnnnaug

SEQ ID NO: 62 - RNA, 21 nt, Escherichia coli
  misc_feature 1, 9, 10, 11, 12, 13, 14, 15, 16, 17, 18: n = A,U,C or G
naggaggunnnnnnnnnnaug

(19) United States
(12) Patent Application Publication
(10) Pub. No.: US 2007/0031457 A1
(43) Pub. Date: Feb. 8, 2007

(54) Modified Bacillus anthracis, vaccine compositions and methods of use thereof

(76) Inventors: Thomas W. Dubensky JR., Piedmont, CA (US); Daniel A. Portnoy, Albany, CA (US); Richard L. Calendar, Berkeley, CA (US); John E. Hearst, Berkeley, CA (US); David N. Cook, Lafayette, CA (US)

Correspondence Address: MORRISON & FOERSTER LLP, 755 PAGE MILL RD, PALO ALTO, CA, 94304-1018, US

(21) Appl. No.: 11/173,770

(22) Filed: Jun. 30, 2005

Related U.S. Application Data
(63) Continuation-in-part of application No. PCT/US05/02987, filed on Feb. 2, 2005.
(63) Continuation-in-part of application No. PCT/US04/23881, filed on Jul. 23, 2004.
Application No. PCT/US04/23881 is a continuation-in-part of application No. 10/883,599, filed on Jun. 30, 2004.
Application No. 10/883,599 is a continuation-in-part of application No. 10/773,618, filed on Feb. 6, 2004.
Application No. PCT/US04/23881 is a continuation-in-part of application No. 10/773,618, filed on Feb. 6, 2004.
Application No. PCT/US04/23881 is a continuation-in-part of application No. 10/883,599, filed on Jun. 30, 2004.
Application No. 10/883,599 is a continuation-in-part of application No. 10/773,618, filed on Feb. 6, 2004.
Application No. PCT/US05/02987 is a continuation-in-part of application No. 10/773,618, filed on Feb. 6, 2004.
(63) Continuation-in-part of application No. PCT/US04/23881, filed on Jul. 23, 2004.
Application No. PCT/US04/23881 is a continuation-in-part of application No. 10/883,599, filed on Jun. 30, 2004.
Application No. 10/883,599 is a continuation-in-part of application No. 10/773,618, filed on Feb. 6, 2004.
Application No. PCT/US04/23881 is a continuation-in-part of application No. 10/773,618, filed on Feb. 6, 2004.
(63) Continuation-in-part of application No. 10/883,599, filed on Jun. 30, 2004.
Application No. 10/883,599 is a continuation-in-part of application No. 10/773,618, filed on Feb. 6, 2004.
(63) Continuation-in-part of application No. 10/773,618, filed on Feb. 6, 2004.
(60) Provisional application No. 60/649,510, filed on Feb. 2, 2005.
(60) Provisional application No. 60/599,522, filed on Aug. 5, 2004.
(60) Provisional application No. 60/599,522, filed on Aug. 5, 2004.
(60) Provisional application No. 60/584,886, filed on Jun. 30, 2004.
(60) Provisional application No. 60/584,886, filed on Jun. 30, 2004.

(51) Int. Cl. A61K 39/07; C12N 1/21

(52) U.S. Cl. 424/246.1; 435/252.31

(57) ABSTRACT

A variety of modified Bacillus anthracis bacteria useful in vaccines are provided. For instance, asporogenic strains of Bacillus anthracis are provided. In addition, Bacillus anthracis strains attenuated in their ability to repair their nucleic acid, such as in their nucleic acid excision repair ability or recombination repair ability, are provided. Strains expressing an antigen, such as protective antigen, under the control of a heterologous promoter and/or an inducible promoter are also provided. Bacillus anthracis bacteria comprising mutations in toxin genes are further provided. Vaccine compositions comprising the bacteria, methods of making the modified strains, and methods of using the vaccines are also provided.

### CROSS-REFERENCE TO RELATED APPLICATIONS

This application claims the priority benefit of each of the following applications, the disclosures of each of which are hereby incorporated by reference herein in their entirety: U.S. Provisional Application Ser. No. 60/649,510, filed Feb. 2, 2005; International Application No. PCT/US2005/002987, filed Feb. 2, 2005 (Attorney Docket No. 282172004340); U.S. Provisional Application No. 60/599,522, filed Aug. 5, 2004; International Application No. PCT/US2004/023881, filed Jul. 23, 2004; U.S. Provisional Application Ser. No. 60/584,886, filed Jun. 30, 2004; U.S. patent application Ser. No. 10/883,599, filed Jun. 30, 2004; and U.S. patent application Ser. No. 10/773,618, filed Feb. 6, 2004. This application is a continuation-in-part of International Application No. PCT/US2005/002987, filed Feb. 2, 2005, which is a continuation-in-part of International Application No. PCT/US2004/023881, filed Jul. 23, 2004, which is a continuation-in-part of U.S. patent application Ser. No. 10/883,599, filed Jun. 30, 2004, which is a continuation-in-part of U.S. patent application Ser. No. 10/773,618, filed Feb. 6, 2004.

### STATEMENT REGARDING FEDERALLY SPONSORED RESEARCH OR DEVELOPMENT

This invention was made, in part, with government support under grant number 1U01AI061199-01, awarded by the National Institutes of Health. The government may have certain rights in the invention.

### FIELD OF THE INVENTION

The present invention relates generally to vaccine compositions and immunotherapy. In particular, the present invention relates to vaccine compositions comprising modified Bacillus anthracis bacteria.

### BACKGROUND OF THE INVENTION

A variety of vaccines have been developed for clinical use, mostly targeting the prevention of infectious diseases caused by viruses, bacteria and parasites. Vaccines can be prepared from live attenuated microbes, inactivated (killed) microbes, or components of the microbes themselves. Live attenuated microbes contain genetic alterations, such as deletion or modification of virulence factors, resulting in a less virulent microbe. For inactivated vaccines, a microbe may be chemically or physically inactivated. Ideally, such vaccines cannot cause an infection but are still able to stimulate a desired immune response. Examples of inactivated vaccines include polio and influenza viruses, and bacterial vaccines against cholera and pertussis, although live attenuated vaccines are an option for polio, influenza, and cholera as well. In order to elicit the desired immune response, it is important that the inactivated microbe comprises the appropriate antigens prior to inactivation. It has been observed in some cases that inactivating the microbe results in a significantly reduced immune response because de novo gene expression by an infecting microbe is required to stimulate an optimal immune response. Methods that have been used to inactivate bacteria include the use of acetone, alcohol, formalin, glutaraldehyde, paraformaldehyde, or phenol, heating, or ultraviolet irradiation (Pace et al., Vaccine 16:1563-1574 (1998)).

Efforts to develop a safe, effective vaccine against one deadly agent, Bacillus anthracis, using traditional technologies have been largely unsuccessful. The dormant and extremely durable spore form of Bacillus anthracis, the causative agent of anthrax, is an ideal biological weapon (World Health Organization. Health aspects of chemical and biological weapons: a report of a WHO group of consultants. Geneva Switzerland: World Health Organization; Mock, M. and A. Fouet, Annu. Rev. Microbiol. 55:647-671(2001)). Inhaled spores are transported by alveolar macrophages to the lymph nodes surrounding the lungs, where they germinate, multiply, and produce high levels of cytotoxins, killing up to 99% of immunologically naive victims who don't receive antibitiotic therapy. The only licensed human anthrax vaccine, anthrax vaccine absorbed (AVA), was developed in the late 1950s and is poorly immunogenic (Report, Case Report: Use of Anthrax Vaccine in the United States: Recommendations of the Advisory Committee on Immunization Practices, in Clinical Toxicology. 2001. p. 85-100). The prolonged 18-month vaccination regimen and required annual boosters are problematic for immunization of military personnel both in terms of safety and in terms of practicality. AVA is based on processed B. anthracis culture supernatants containing various amounts of lethal factor (LF) (encoded by the lef gene), edema factor (EF) (encoded by the cya gene), and protective antigen (PA) (encoded by the pagA gene) formulated with an adjuvant. In addition to being weakly immunogenic, AVA represents only a small fraction of the expressed bacterial proteins and does not stimulate immunity against other known B. anthracis virulence determinants, for example capsule. Thus, the possibility of new strains strategically engineered to subvert the present vaccine constitutes a genuine threat. Given the development and use around the world of B. anthracis spores as a biological weapon, there is a clear need for a safe, effective anthrax vaccine that combines safety with potency to elicit broad and durable protective immune responses in vaccinated individuals.

### SUMMARY OF THE INVENTION

The invention provides a variety of modified Bacillus anthracis bacteria suitable for use in vaccines. In some embodiments, the modified Bacillus anthracis bacteria comprise mutations that attenuate the ability of the bacteria to repair its nucleic acid. In some embodiments, the modified Bacillus anthracis are asporogenic or sporulation-deficient. Modified Bacillus anthracis bacteria expressing an antigen, such as protective antigen, under the control of a heterologous promoter and/or an inducible promoter are also provided. Modified Bacillus anthracis bacteria comprising mutations in toxin genes are further provided. In some embodiments, the modified bacteria have decreased toxicity, and/or have increased immunogenicity. In some embodiments, the ability of the modified Bacillus anthracis to proliferate is attenuated (preferably while sufficient gene expression is maintained). The invention also provides methods for modifying Bacillus anthracis for use in vaccines. The invention further provides immunogenic compositions and vaccine compositions comprising the modified Bacillus anthracis. Methods of using the modified bacteria and compositions comprising the bacteria for the induction of immune responses in a host and/or for the prevention of disease are also provided.

In one aspect, the present invention provides modified Bacillus anthracis bacteria, and compositions thereof (e.g., vaccine compositions), which are capable of generating an immune response upon administration to a host. In some embodiments, the immune response is a response which protects the host from a disease related to infection by Bacillus anthracis, such as anthrax. In some embodiments, the immune response comprises a CD4+ immune response, a CD8+ immune response, or both a CD4+ and CD8+ immune response. In some embodiments, the immune response comprises an immune response specific to lethal factor (LF), edema factor (EF), protective antigen (PA), capsule, and/or whole bacteria. In some embodiments, the bacteria are defective with respect to at least one nucleic acid repair enzyme. In some embodiments, the nucleic acid of the bacteria has been modified so that the bacteria are attenuated for proliferation. Methods of using the bacteria and compositions to generate an immune response in a host to Bacillus anthracis and/or protect a host from disease comprising administering an effective amount of the bacteria and/or compositions to the host are further provided.

In another aspect, the present invention provides a Bacillus anthracis strain that is attenuated for nucleic acid repair. In some embodiments, the strain is defective with respect to at least one DNA repair enzyme. In some embodiments, the DNA repair enzyme is UvrA, UvrB, UvrC and/or RecA. In some embodiments, the nucleic acid of the bacteria of the strain has been modified so that the bacteria are attenuated for proliferation. In some embodiments, the nucleic acid of the bacteria has been modified by reaction with a nucleic acid targeting compound that reacts directly with nucleic acid, so that the bacteria are attenuated for proliferation. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In another aspect, the present invention provides a Bacillus anthracis bacterium, wherein the nucleic acid of the bacterium has been modified so that the bacterium is attenuated for proliferation. In some embodiments, the nucleic acid of the bacterium has been modified by reaction with a nucleic acid targeting compound that reacts directly with nucleic acid, so that the bacterium is attenuated for proliferation. Compositions, such as immunogenic compositions and vaccine compositions, comprising the bacterium are provided. Methods of inducing an immune response in a host comprising administering an effective amount of a composition comprising the modified bacterium are also provided, as are methods of protecting a host from a disease, comprising administering to the host an effective amount of a composition comprising the modified bacteria.

In another aspect, the present invention provides a Bacillus anthracis bacterium comprising a mutation in the lef gene, the cya gene, or both genes that decreases the toxicity of the bacterium (relative to the bacterium without the mutation). Compositions, such as vaccine compositions, comprising the bacteria and methods of using the bacteria are also provided.

In another aspect, the present invention provides a Bacillus anthracis bacterium comprising a mutation in pagA that decreases the toxicity of the bacterium (relative to the bacterium without the mutation). Compositions, such as vaccine compositions, comprising the bacteria and methods of using the bacteria are also provided.

In another aspect, the present invention provides a Bacillus anthracis bacterium comprising an additional copy of a gene encoding a Bacillus anthracis antigen. Compositions, such as vaccine compositions, comprising the bacteria and methods of using the bacteria are also provided.

In another aspect, the present invention provides an asporogenic Bacillus anthracis strain that is attenuated for nucleotide excision repair (NER). In some embodiments, the strain comprises an inhibiting mutation, or a modification that attenuates expression of, at least one sporulation gene. In some embodiments, the strain is defective with respect to SpoIIE. In some embodiments, the strain comprises a mutation in the spoIIE gene. Moreover, in some embodiments, the strain is defective with respect to one or more enzymes selected from the group consisting of UvrA, UvrB, and UvrC. In some embodiments, the strain is defective with respect to SpoIIE, UvrA, and UvrB. In some embodiments, the strain comprises a mutation in at least one DNA nucleotide excision repair gene. For instance, in some embodiments, the strain comprises a mutation in one or more genes selected from the group consisting of uvrA gene, uvrB gene, and uvrC gene. In some embodiments, the strain further comprises a mutation in, or a modification that attenuates expression of, a recombinational repair gene, such as recA. In some embodiments, the mutation or modification comprises a conditional mutation or a transcriptional or translational activator or repressor. In some embodiments, the mutation comprises a conditional mutation. In some embodiments, the modification comprises a nucleic acid molecule encoding a RecA protein, wherein expression of the RecA protein is under the control of a transcriptional or translational activator or repressor (e.g., a transcriptional repressor such as the lac repressor). In some embodiments, the strain is defective with respect to RecA (e.g., comprises a mutant recA gene which encodes a temperature-sensitive RecA or an inducible or repressible recA mutant). In some embodiments, the strain further comprises an inhibiting mutation, or a modification that attenuates expression of, at least one toxin gene. In some embodiments, the at least one toxin gene comprises the lef gene, cya gene, or lef and cya genes. In some embodiments, the strain comprises one or more mutations in the lef gene, the cya gene, or both genes, that decreases the toxicity of the strain. In some embodiments, the strain further comprises a nucleic acid encoding at least one antigen, wherein the nucleic acid encoding the at least one antigen is operably linked to a heterologous promoter. In some embodiments, the heterologous promoter is inducible. For instance, the promoter is, in some embodiments, inducible by ultraviolet light, a nucleic acid targeted compound, a nucleic acid cross-linking compound, and/or an SOS regulatory pathway. In some embodiments, the antigen is protective antigen and the nucleic acid encoding the protective antigen is operably linked to an SOS regulatory sequence. In some embodiments, the nucleic acid encoding the at least one antigen is, or is derived from, a gene selected from the group consisting of the following: pagA gene; lef gene; cya gene; pagA and lef genes; pagA and cya genes; cya and lef genes; and pagA, lef, and cya genes. In some embodiments, the strain comprises a poly-D-glutamate capsule, while in some alternative embodiments the strain lacks a poly-D-glutamate capsule. In some embodiments, the nucleic acid of the bacteria of the strain has been modified so that the bacteria are attenuated for proliferation. By way of non-limiting example, the nucleic acid may have been modified by reaction with a nucleic acid targeting compound that reacts directly with the nucleic acid (e.g., the nucleic acid has been treated with a psoralen and UVA radiation). A bacterium of the strain is also provided which further comprises at least one covalently linked nucleic acid cross-linking compound (e.g., a psoralen), linked to the genomic DNA of the bacterium. A vaccine or composition comprising the Bacillus anthracis strain is also provided, as well as a method of inducing an immune response in a host to Bacillus anthracis comprising administering to the host an effective amount of a composition comprising a bacterium of the strain.

In yet another aspect, the invention provides a sporulation-deficient Bacillus anthracis strain that is attenuated for nucleotide excision repair (NER). In some embodiments, the strain comprises an inhibiting mutation, or a modification that attenuates expression of, at least one sporulation gene. In some embodiments, the strain is defective with respect to SpoIIE. In some embodiments, the strain comprises a mutation in the spoIIE gene. Moreover, in some embodiments, the strain is defective with respect to one or more enzymes selected from the group consisting of UvrA, UvrB, and UvrC. In some embodiments, the strain comprises a mutation in at least one DNA nucleotide excision repair gene. For instance, in some embodiments, the strain comprises a mutation in one or more genes selected from the group consisting of uvrA gene, uvrB gene, and uvrC gene. In some embodiments, the strain further comprises a mutation in, or a modification that attenuates expression of, a recombinational repair gene, such as recA. In some embodiments, the mutation or modification comprises a conditional mutation or a transcriptional or translational activator or repressor. In some embodiments, the mutation comprises a conditional mutation. In some embodiments, the modification comprises a nucleic acid molecule that encodes a RecA protein, wherein expression of the RecA protein is under the control of a transcriptional or translational activator or repressor (e.g., the lac repressor). In some embodiments, the strain further comprises an inhibiting mutation, or a modification that attenuates expression of, at least one toxin gene. In some embodiments, the at least one toxin gene comprises the lef gene, cya gene, or lef and cya genes. In some embodiments, the strain comprises a nucleic acid encoding at least one antigen, wherein the nucleic acid encoding the at least one antigen is operably linked to a heterologous promoter. In some embodiments, the heterologous promoter is inducible. For instance, the promoter is, in some embodiments, inducible by ultraviolet light, a nucleic acid targeted compound, a nucleic acid cross-linking compound, and/or an SOS regulatory pathway. In some embodiments, the nucleic acid encoding the at least one antigen is or is derived from a gene selected from the group consisting of the following: pagA gene; lef gene; cya gene; pagA and lef genes; pagA and cya genes; cya and lef genes; and pagA, lef, and cya genes. In some embodiments, the strain comprises a poly-D-glutamate capsule, while in some alternative embodiments the strain lacks a poly-D-glutamate capsule. A bacterium of the strain is also provided which further comprises at least one covalently linked nucleic acid cross-linking compound (e.g., a psoralen), linked to the genomic DNA of the bacterium. A vaccine or composition comprising the Bacillus anthracis strain is also provided, as well as a method of inducing an immune response in a host to Bacillus anthracis comprising administering to the host an effective amount of a composition comprising a bacterium of the strain.

15 In another aspect, the invention provides a Bacillus anthracis strain comprising a heterologous nucleic acid encoding an antigen, such as protective antigen.

161 In another aspect, the invention provides a Bacillus anthracis strain comprising a nucleic acid encoding an antigen, wherein the nucleic acid encoding the antigen is operably linked to a heterologous promoter. In some embodiments, the nucleic acid encoding the antigen is, or is derived from a gene selected from, the group consisting of the following: pagA gene; lef gene; cya gene; pagA and lef genes; pagA and cya genes; cya and lef genes; and pagA, lef, and cya genes. In some embodiments, the heterologous promoter is inducible. In some embodiments, the heterologous promoter is inducible by ultraviolet light, a nucleic acid cross-linking compound, ultraviolet light and a nucleic acid cross-linking compound, an SOS regulatory pathway, and/or a change or shift in temperature. The invention further provides a vaccine or composition comprising a bacterium of the Bacillus anthracis strain. The present invention also provides a method of inducing an immune response in a host to Bacillus anthracis comprising administering to a host an effective amount of a composition comprising a bacterium of the strain.

In an additional aspect, the invention provides a Bacillus anthracis strain comprising at least one inhibiting mutation in each of uvrA gene, uvrB gene, lef gene, cya gene, and spoIIE gene. Also provided is a bacterium of the Bacillus anthracis strain, wherein the bacterium further comprises at least one covalently linked nucleic acid cross-linking compound linked to the genomic DNA of the bacterium. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In still another aspect, the invention provides an asporogenic Bacillus anthracis strain that is attenuated for nucleic acid repair. In one embodiment, the strain is defective with respect to SpoIIE. In some embodiments the strain is also defective with respect to UvrA and/or UvrB (e.g., comprises a mutation in the uvrAB genes). In some embodiments, the strain is defective with respect to UvrC (e.g., comprises a mutation in the uvrC gene). In some embodiments the strain is also defective with respect to RecA (e.g., wholly defective, partially defective, or conditionally defective with respect to RecA). In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. In some embodiments, the Bacillus anthracis strain is a pXO1+ and pXO2+ strain. A composition, such as a vaccine composition, comprising a bacterium of the strain is also provided. Methods of inducing an immune response in a host to Bacillus anthracis comprising administering to the host an effective amount of the composition are also provided. In addition, methods of protecting a host from disease (i.e., a disease caused by infection with B. anthracis), comprising administering to the host an effective amount of a composition comprising a bacterium of the strain are also provided.

In another aspect, the invention provides an asporogenic Bacillus anthracis strain that is attenuated for nucleic acid repair, wherein the strain is defective with respect to at least one DNA repair enzyme selected from the group consisting of UvrA, UvrB, UvrC, and RecA. In some embodiments, the bacterium of the Bacillus anthracis strain has been modified so that the bacteria are attenuated for proliferation. In some embodiments, the nucleic acid has been modified with a nucleic acid targeting compound that reacts directly with the nucleic acid (e.g., a psoralen compound activated by UVA irradiation), so that the bacterium is attenuated for proliferation. In some embodiments, the bacterium is defective with respect to SpoIIE. For example, in some embodiments the bacterium comprises a mutation in the spoIIE gene which renders the bacterium defective with respect to SpoIIE. In some embodiments, the strain is defective with respect to UvrA, UvrB, or both UvrA and UvrB. For instance, in some embodiments, the strain comprises a mutation in the uvrA gene, the uvrB gene, or both the uvrA and uvrB gene. In some embodiments, the strain is defective with respect to SpoIIE, UvrA, and UvrB. In some embodiments, the strain is defective with respect to UvrC (e.g., the strain comprises a mutation in the uvrC gene). In some embodiments, the strain is defective with respect to RecA. In some embodiments, the strain comprises a mutation in the recA gene. In other embodiments, the strain is a repressible recA mutant. In some embodiments, the strain comprises a recA gene that is under the control of the lac repressor. In further embodiments, the strain comprises a temperature sensitive recA gene. In some additional embodiments, the strain expresses protective antigen under the control of an SOS regulatory sequence. In some embodiments, the strain comprises one or more mutations in the lef gene, the cya gene, or both genes that decreases the toxicity of the strain. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In another aspect, the invention provides a Bacillus anthracis strain comprising a heterologous expression cassette comprising a sequence encoding protective antigen (PA), or an antigen derived therefrom, wherein the sequence encoding the protective antigen, or antigen derived therefrom, is operably linked to an inducible promoter. In some embodiments, the expression of the protective antigen is induced by treatment of the strain with a psoralen and UVA radiation. For instance, in some embodiments, the nucleic acid encoding protective antigen is operably linked to an SOS regulatory sequence, or any promoter that is induced in response to photochemical treatment or other modification of the DNA of the bacterium. In some embodiments, the strain comprises an inhibiting mutation in, or a modification that attenuates expression of, at least one nucleic acid repair gene (e.g., a nucleotide excision repair (NER) gene, a recombinational repair gene, or an NER gene and a recombinational repair gene). In some embodiments, the strain comprises an inhibiting mutation in, or a modification that attenuates expression of, a nucleic acid repair gene that is selected from the group consisting of uvrA; uvrB; uvrC; uvrA and uvrB; and uvrA, uvrB, and uvrC. In some embodiments, the strain comprises an inhibiting mutation in, or a modification that attenuates expression of, recA. In some embodiments, the mutation or modification comprises a conditional mutation or a transcriptional or translational activator or repressor. In some embodiments, the mutation comprises a conditional mutation. In some embodiments, the modification comprises a nucleic acid molecule that encodes a RecA protein, wherein expression of the RecA protein is under the control of a transcriptional or translational activator or repressor (e.g., a transcriptional repressor, such as the lac repressor). In some embodiments, the strain comprises a mutation that attenuates the ability of the strain to repair its nucleic acid. For instance, in some embodiments, the strain is defective with respect to at least one DNA repair enzyme, such as UvrA, UvrB, or both UvrA and UvrB. In some embodiments, the strain comprises a mutation in the uvrA gene, the uvrB gene, or both the uvrA and uvrB gene. In some embodiments, the strain is defective with respect to UvrC (e.g., comprises a mutation in the uvrC gene). In some embodiments, the strain is defective with respect to RecA. A strain that is defective with respect to RecA can, by way of example, comprise a mutation in its recA gene, comprise a recA gene under control of a repressible promoter, or comprise a temperature sensitive recA gene. In some embodiments, the strain is a repressible recA mutant. In some embodiments, the strain comprises a recA gene that is under the control of the lac repressor. In some embodiments, the strain comprises one or more mutations in the lef gene, the cya gene, or both genes that decreases the toxicity of the strain. In some embodiments, the nucleic acid of the bacteria of the strain has been modified so that the bacteria are attenuated for proliferation. In some embodiments, the nucleic acid of the bacteria has been modified by reaction with a nucleic acid targeting compound that reacts directly with the nucleic acid (e.g., a psoralen compound activated by UVA irradiation). In some embodiments, the bacteria of the strain further comprise at least one covalently linked nucleic acid cross-linking compound (e.g., a psoralen), linked to the genomic DNA of the bacteria. In addition, the strain is, in some embodiments, asporogenic (e.g., is defective with respect to SpoIIE, such as a spoIIE mutant). In some embodiments, the strain comprises an inhibiting mutation in, or a modification that attenuates expression of, at least one sporulation gene or toxin gene (e.g., spoIIE gene; lef gene; cya gene; spoIIE and lef genes; spoIIE and cya genes; lef and cya genes; spoIIE, lef, and cya genes). In some embodiments, the strain comprises a poly-D-glutamate capsule, whereas in other embodiments, the strain lacks a poly-D-glutamate capsule. Vaccines and compositions comprising bacteria of the strain are also provided. A method of inducing an immune response in a host to Bacillus anthracis comprising administering to the host an effective amount of a composition comprising a bacterium of the strain is also provided. A method of protecting a host from a disease, comprising administering to the host an effective amount of a composition (e.g., a vaccine) comprising a bacterium from the strain is also provided.

In another aspect, the invention provides a Bacillus anthracis strain comprising a heterologous expression cassette that expresses protective antigen, or an antigen derived therefrom, under the control of an SOS regulatory sequence. In some embodiments, the Bacillus anthracis strain is attenuated for nucleic acid repair (e.g., is defective for UvrA, UvrB, UvrC, and/or RecA). In some embodiments, the strain is asporogenic. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. A composition, such as a vaccine composition, comprising a bacterium of the strain is also provided. Methods of inducing an immune response in a host to Bacillus anthracis comprising administering to the host an effective amount of the composition are also provided. In addition, methods of protecting a host from a disease, comprising administering to the host an effective amount of a composition comprising a bacterium of the strain are also provided.

In yet another aspect, the invention provides a Bacillus anthracis strain that is defective with respect to recA. In some embodiments, the strain is a conditional recA mutant. In some embodiments, the strain is a temperature sensitive recA mutant. In some embodiments, the strain is a repressible recA mutant. In some embodiments, the strain comprises an expression cassette which expresses RecA antisense RNA. In some embodiments, the strain is an inducible recA mutant. In some embodiments, the recA gene is under control of a lac repressor. In some embodiments, the strain comprises a recA gene that is operably linked to a repressible promoter. In some embodiments, the strain comprises a heterologous expression cassette encoding protective antigen, wherein the protective antigen is operably linked to an SOS regulatory sequence. In some embodiments, the strain is defective with respect to at least one additional DNA repair enzyme such as UvrA, UvrB, or both UvrA and UvrB. (For instance, the strain optionally comprises a mutation in the uvrA gene, the uvrB gene, or both the uvrA and uvrB gene.) In some embodiments, the strain is defective with respect to UvrC (e.g., comprises a mutation in the uvrC gene). In some embodiments, the modified strain comprises one or more mutations in the lef gene, the cya gene, or both genes that decreases the toxicity of the strain. In some embodiments, the nucleic acid of the bacteria of the strain has been modified (e.g., by a nucleic acid targeting compound that reacts directly with the nucleic acid, such as a psoralen compound activated by UVA irradiation), so that the bacteria are attenuated for proliferation. In some embodiments, the Bacillus anthracis strain is asporogenic (e.g., defective with respect to SpoIIE, such as spoIIE mutant). Compositions, such as vaccines, comprising a bacterium from the Bacillus anthracis strain are also provided. A method of inducing an immune response in a host to Bacillus anthracis comprising administering to the host an effective amount of a composition comprising a bacterium of the strain is also provided. A method of protecting a host from a disease, comprising administering to the host an effective amount of a composition comprising a bacterium from the strain is further provided.

In another aspect, the invention provides a Bacillus anthracis strain comprising a temperature-sensitive recA gene. In some embodiments, the strain comprises a recA gene which comprises a mutation analogous to the V246M mutation of the recA44 temperature-sensitive recA mutant of E. coli. For instance, in some embodiments, the bacteria of the strain comprise a mutation in a Bacillus anthracis recA gene, wherein the recA gene encodes a temperature-sensitive RecA protein comprising a V244M mutation. In some embodiments, the bacteria of the strain comprise a recA gene derived from a foreign bacterium, such as E. coli, wherein the recA gene encodes a temperature-sensitive RecA protein. In some embodiments, the bacteria comprise a recA gene derived from E. coli, wherein the recA gene comprises a mutation analogous to the V246M mutation of the E. coli recA44 temperature-sensitive recA mutant. In some embodiments, the strain comprises a recA gene that comprises the recA44(ts) allele of E. Coli. In some embodiments, the strain is defective with respect to UvrA and/or UvrB (e.g., comprises a mutation in the uvrA and/or uvrB gene). In some embodiments, the strain is defective with respect to UvrC (e.g., comprises a mutation in the uvrC gene). In some embodiments, the strain is asporogenic. In some embodiments, the strain comprises a heterologous expression cassette that expresses protective antigen under the control of an SOS regulatory sequence. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. A composition, such as a vaccine composition, comprising a bacterium of the strain is also provided. Methods of inducing an immune response in a host to Bacillus anthracis comprising administering to the host an effective amount of the composition are also provided. In addition, methods of protecting a host from a disease, comprising administering to the host an effective amount of a composition comprising a bacterium of the strain are also provided.

In an additional aspect, the invention provides a Bacillus anthracis bacterium which is a repressible or inducible recA mutant. In some embodiments, the bacterium is a repressible recA mutant. In other embodiments, the bacterium is an inducible recA mutant. In some embodiments, the bacterium comprises a nucleic acid encoding a RecA protein, wherein expression of the RecA protein is under the control of a heterologous transcriptional repressor or activator. In some embodiments, the bacterium comprises a recA gene, wherein expression of the recA gene is under control of a lac repressor. In some embodiments, the bacterium comprises a nucleic acid encoding a RecA protein, wherein the nucleic acid is operably linked to a heterologous operator which binds a repressor or activator. In some embodiments, the bacterium comprises a nucleic acid that produces a RecA antisense RNA upon transcription, wherein the nucleic acid is operably linked to an operator which binds a repressor or activator. Compositions, such as immunogenic compositions and vaccine compositions, comprising the bacteria are provided. Methods of inducing an immune response in a host comprising administering an effective amount of a composition comprising the bacteria are also provided, as are methods of protecting a host from a disease, comprising administering to the host an effective amount of a composition comprising the bacteria. Methods of protecting a host from a disease comprising administering an effective amount of a composition comprising the bacteria are also provided.

In another aspect, the invention provides a Bacillus anthracis strain attenuated for nucleic acid repair, wherein the strain is defective with respect to RecA and at least one additional DNA repair enzyme selected from the group consisting of UvrA, UvrB, and UvrC. Compositions, such as vaccine compositions, comprising the bacterium of the strain are also provided. Methods of using compositions comprising the strain are also provided.

In another aspect, the invention provides an asporogenic Bacillus anthracis strain attenuated for nucleic acid repair, wherein the strain is defective with respect to SpoIIE and at least one additional DNA repair enzyme selected from the group consisting of UvrA, UvrB, and UvrC. Compositions, such as vaccine compositions, comprising the bacterium of the strain are also provided. Methods of using compositions comprising the strain are also provided.

In still another aspect, the invention provides an asporogenic Bacillus anthracis strain that is attenuated for nucleic acid repair, comprising a temperature sensitive recA gene and a mutation in uvrA, uvrB, or uvrC (e.g., a deletion of uvrA, uvrB, or uvrC). Compositions, such as vaccine compositions, comprising the bacterium of the strain are also provided. Methods of using compositions comprising the strain are also provided.

In yet another aspect, the invention provides an asporogenic Bacillus anthracis strain that is attenuated for nucleic acid repair, comprising a repressible recA gene and a mutation in uvrA, uvrB, or uvrC (e.g., a deletion of uvrA, uvrB, or uvrC). Compositions, such as vaccine compositions, comprising the bacterium of the strain are also provided. Methods of using compositions comprising the strain are also provided.

In another aspect, the invention provides an asporogenic Bacillus anthracis bacterium, wherein the nucleic acid of the bacterium has been modified so that the bacterium is attenuated for proliferation. In some embodiments, the bacterium has been modified by reaction with a nucleic acid targeting compound that reacts directly with the nucleic acid, so that the bacterium is attenuated for proliferation. In some embodiments, the nucleic-acid targeted compound is a nucleic acid alkylator. In some embodiments, the nucleic acid targeted compound is a psoralen compound (e.g., a psoralen compound activated by UVA irradiation). In some embodiments, the bacterium comprises at least one covalently linked nucleic acid cross-linking compound (e.g., a psoralen), linked to the genomic DNA of the bacterium. In some embodiments, the bacterium is defective with respect to SpoIIE. In some embodiments, the bacterium comprises a mutation in a sporulation gene, such as, but not limited to, spoIIE. In some embodiments, the bacterium is attenuated for nucleic acid repair. For instance, in some embodiments, the bacterium is defective with respect to at least one DNA repair enzyme (e.g., UvrA, UvrB, and/or UvrC). In some embodiments, the bacterium comprises a mutation in one or more genes selected from the group consisting of uvrA, uvrB, and uvrC. In some embodiments, the bacterium is defective with respect to SpoIIE, UvrA, and UvrB. In some embodiments, the bacterium is attenuated for recombinational repair. For instance, in some embodiments, the bacterium is defective with respect to RecA. In some embodiments, the bacterium comprises a mutation in the recA gene. In some embodiments, the bacterium comprises a recA gene that is under the control of the lac repressor. For instance, in some embodiments, the bacterium comprises a mutant recA gene which encodes a temperature-sensitive RecA. In some embodiments, the bacterium is a repressible or inducible recA mutant. In some embodiments, the bacterium expresses protective antigen (PA) under the control of an SOS regulatory sequence. In some embodiments, the bacterium comprises one or more mutations in the lef gene, the cya gene, or both genes that decreases the toxicity of the strain. Compositions, such as immunogenic compositions and vaccine compositions, comprising the bacterium are provided. Methods of inducing an immune response in a host comprising administering an effective amount of a composition comprising the bacterium are also provided, as are methods of protecting a host from a disease, comprising administering to the host an effective amount of a composition comprising the bacterium.

In another aspect, the invention provides an asporogenic Bacillus anthracis bacterium that is attenuated for nucleic acid repair, wherein the nucleic acid of the bacterium has been modified so that the bacterium is attenuated for proliferation. In some embodiments, the bacterium has been modified with a nucleic acid targeting compound that reacts directly with the nucleic acid, so that the bacterium is attenuated for proliferation. In some embodiments, the nucleic-acid targeted compound is a nucleic acid alkylator. In some embodiments, the nucleic acid targeted compound is a psoralen compound (e.g., a psoralen compound activated by UVA irradiation). In some embodiments, the bacterium is defective with respect to SpoIIE. In some embodiments, the bacterium comprises a mutation in a sporulation gene, such as, but not limited to, SpoIIE. In some embodiments, the bacterium is defective with respect to UvrA, UvrB, or both UvrA and UvrB. For instance, in some embodiments, the bacterium comprises a mutation in the uvrA gene, the uvrB gene, or both the uvrA and uvrB gene. In some embodiments, the bacterium is defective with respect to SpoIIE, UvrA, and UvrB. In some embodiments, the bacterium is defective with respect to UvrC (e.g., the strain comprises a mutation in the uvrC gene). For instance, in some embodiments, the bacterium is defective with respect to RecA. In some embodiments, the bacterium comprises a mutation in the recA gene (e.g., comprises a temperature sensitive recA gene). In some embodiments, the bacterium comprises a recA gene that is under the control of the lac repressor. In some embodiments, the bacterium is a repressible recA mutant. In some embodiments, the bacterium expresses protective antigen (PA) under the control of an SOS regulatory sequence. In some embodiments, the bacterium comprises one or more mutations in the lef gene, the cya gene, or both genes that decreases the toxicity of the strain. Compositions, such as immunogenic compositions and vaccine compositions, comprising the bacterium are provided. Methods of inducing an immune response in a host comprising administering an effective amount of a composition comprising the bacterium are also provided, as are methods of protecting a host from a disease, comprising administering to the host an effective amount of a composition comprising the bacterium.

In another aspect, the invention provides a sporulation-deficient Bacillus anthracis bacterium, wherein the nucleic acid of the bacterium has been modified so that the bacterium is attenuated for proliferation. In some embodiments, the bacterium has been modified with a nucleic acid targeted compound that reacts directly with the nucleic acid. In some embodiments, the nucleic-acid targeted compound is a nucleic acid alkylator. In some embodiments, the nucleic acid targeted compound is a psoralen compound (e.g., a psoralen compound activated by UVA irradiation). In some embodiments, the bacterium comprises at least one covalently linked nucleic acid cross-linking compound (e.g., a psoralen), linked to the genomic DNA of the bacterium. In some embodiments, the bacterium is defective with respect to SpoIIE. In some embodiments, the bacterium comprises a mutation in a sporulation gene, such as, but not limited to, spoIIE. In some embodiments, the bacterium is attenuated for nucleic acid repair. For instance, in some embodiments, the bacterium is defective with respect to at least one DNA repair enzyme (e.g., UvrA, UvrB, and/or UvrC). In some embodiments, the bacterium comprises a mutation in one or more genes selected from the group consisting of uvrA, uvrB, and uvrC. In some embodiments, the bacterium is attenuated for recombinational repair. For instance, in some embodiments, the bacterium is defective with respect to RecA. In some embodiments, the bacterium comprises a mutation in the recA gene. For instance, in some embodiments, the bacterium comprises a mutant recA gene which encodes a temperature-sensitive RecA. In some embodiments, the bacterium is a repressible or inducible recA mutant. In some embodiments, the bacterium expresses protective antigen (PA) under the control of an SOS regulatory sequence. In some embodiments, the bacterium comprises one or more mutations in the lef gene, the cya gene, or both genes that decreases the toxicity of the strain. The invention further provides a vaccine or composition comprising the bacterium. In addition, the invention provides a method of inducing an immune response in a host to Bacillus anthracis comprising administering to the host an effective amount of a composition comprising the bacterium.

In still another aspect, the invention provides a Bacillus anthracis strain which is defective with respect to SpoIIE. In some embodiments, the strain is sporulation-deficient. In other embodiments, the strain is asporogenic. In some embodiments, the strain comprises a mutation in SpoIIE. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In a further aspect, the invention provides a sporulation-deficient Bacillus anthracis strain that is attenuated for nucleic acid repair. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In another aspect, the invention provides an asporogenic Bacillus anthracis strain that is attenuated for nucleic acid repair. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In some embodiments of each of the aforementioned aspects, as well as other aspects described herein, the strain and/or bacterium comprises a mutation in one or more gene selected from the group consisting of: cya, lef and/orpagA (e.g., a mutation that decreases the toxicity of the strain and/or bacterium relative to the same strain and/or bacterium without the mutation).

In some embodiments of each of the aforementioned aspects, as well as other aspects described herein, the strain and/or bacterium comprises a polynucleotide encoding an antigen derived from edema factor, lethal factor, or protective antigen (e.g., an antigen comprising a mutation that decreases the toxicity of Bacillus anthracis).

In some embodiments of each of the aforementioned aspects, as well as other aspects described herein, the strain and/or bacterium is isolated.

Methods of making and using the aforementioned strains and modified bacteria, as well as the other strains described herein are also provided.

Polynucleotides, expression cassettes, and vectors are provided as are host cells comprising the polynucleotides, expression cassettes, and vectors.

### DRAWINGS

FIG. 1 shows the attenuation of Bacillus anthracis Sterne strain with and without deletion of uvrAB. The log titer is plotted vs. nM concentration of psoralen S-59 present during growth and UVA irradiation (6 J/cm²).

FIG. 2 shows inactivation of various B. anthracis strains versus concentration of psoralen (S-59). Inactivation was measured by colony forming units (CFU). The S-59 concentrations were 0-2000 nM. The indicated B. anthracis strains were exposed to the indicated concentration of S-59, then treated with ultraviolet light (6.5 J/cm²), followed by dilution and plating. The exposure time to the UV light was limited to about one minute. The B. anthracis strains were Sterne (- [symbol] -)(open diamond); new Sterne (-□-)(open large square); spoIIE (-Δ-)(open triangle); uvrAB (- [symbol] -)(open small square); and uvrAB/SpoIIE (-ο-) (open circle). “New Sterne” was derived from the same bacterial stock as “Sterne,” that is, the bacterium of “new Sterne” is identical to the bacterium of “Sterne,” but they were derived from the same stock at different times.

FIG. 3 shows colony forming units of B. anthracis before and after heating. The following strains of B. anthracis were tested: Sterne; uvrAB mutant; spoIIE mutant, spoIIE/uvrAB/cyaA mutant, and spoIIE/uvrAB mutant.

FIG. 4 shows photographs of B. anthracis Sterne in log phase in BHI culture (FIG. 4A), B. anthracis Sterne in static phase in PA medium (FIG. 4B), B. anthracis Sterne ΔspoIIE mutant in log phase in BHI culture (FIG. 4C), and B. anthracis Strene ΔspoIIE mutant in static phase in PA medium (FIG. 4D).

FIG. 5 shows colony-forming units of various B. anthracis strains following psoralen/UVA treatment at different psoralen concentrations.

FIG. 6 shows microtiter plates showing the results of MTT assays assessing the metabolic activity of various B. anthracis strains that were previously treated with psoralen and UVA light or were untreated.

FIG. 7 shows a graph of the quantitative data obtained from MTT assays assessing the metabolic activity of various B. anthracis strains that were previously treated with psoralen and UVA light or were untreated.

FIG. 8 shows the results of a polypeptide expression assay in which the metabolic activity of a variety of B. anthracis strains following psoralen/UVA-treatment or no treatment has been assessed.

FIG. 9 shows a western blot showing the levels of expression of LF, EF, and PA by B. anthracis Sterne ΔuvrAB or B. anthracis Sterne ΔspoIIE ΔuvrAB cyaK346Q/K353Q (Sterne 3) bacteria that were either treated with psoralen and UVA light or untreated.

FIG. 10 shows a gel showing levels of expression of LF, EF, and PA by B. anthracis Sterne ΔuvrAB or B. anthracis Sterme ΔspoIIE ΔuvrAB cyaK346Q/K353Q (Sterne 3) bacteria that were either treated with psoralen and UVA light or untreated.

FIG. 11 shows a western blot showing the levels of expression of LF, EF, and PA by B. anthracis Sterne or B. anthracis Sterne ΔspoIIE ΔuvrAB cyaK346Q/K353Q (Sterne 3) bacteria that were either treated with psoralen and UVA light or untreated.

### DETAILED DESCRIPTION OF THE INVENTION

The present invention involves modified Bacillus anthracis strains and the use of the modified Bacillus anthracis in compositions, especially vaccine compositions. Modified Bacillus anthracis strains are provided which are attenuated for nucleic acid repair. Modified strains are also provided which are asporogenic (or sporulation-deficient), less toxic, and/or more immunogenic than the non-modified strains. In addition, modified Bacillus anthracis strains are provided which are modified so that proliferation of the strain is attenuated. Methods of both making and using the vaccine compositions are also provided.

The terms “modified” or “modification” as used herein with respect to strains and bacteria are intended to encompass such modification as chemical, physical, and genetic modification. For instance, in some embodiments, the bacteria are modified by a genetic mutation which attenuates the bacteria's ability to repair its nucleic acid. In some embodiments, a bacterium is modified by cross-linking of its genomic DNA with a psoralen activated by UVA radiation.

Attenuated strains of Bacillus anthracis which are particularly sensitive to inactivation by psoralens, a group of compounds that form irreversible cross-links in the genomes of bacteria after illumination with ultraviolet A (UVA) light, so that they are non-viable, are described (see, e.g., Examples 2, 3, 14, and 17 below). In addition, the inventors describe the construction of an asporogenic strain of B. anthracis (see Examples 4 and 16, below) and a temperature sensitive recA mutant of B. anthracis (see Example 5, below). The introduction of mutations into the B. anthracis antigens Lef and Cya for decreased toxicity is also described (Example 6). The construction of a modified B. anthracis strain that expresses protective antigen under the control of SOS regulatory sequences is also provided (Example 7). This modified strain of Bacillus anthracis is designed to express protective antigen at high levels in the presence of DNA modification induced by an agent such as psoralen for enhanced immunogenicity. (Psoralen is also optionally used to attenuate the B. anthracis for proliferation.)

In one aspect, the present invention provides modified Bacillus anthracis bacteria, and compositions thereof (e.g., vaccine compositions), which are capable of generating an immune response upon administration to a host. In some embodiments, the immune response is a response which protects the host from a disease related to infection by Bacillus anthracis, such as anthrax. In some embodiments, the immune response comprises a CD4+ immune response, a CD8+ immune response, or both a CD4+ and CD8+ immune response. In some embodiments, the immune response comprises an immune response specific to lethal factor (LF), edema factor (EF), protective antigen (PA), capsule, and/or whole bacteria. In some embodiments, the bacteria are defective with respect to at least one nucleic acid repair enzyme. In some embodiments, the nucleic acid of the bacteria has been modified so that the bacteria are attenuated for proliferation. Methods of using the bacteria and compositions to generate an immune response in a host to Bacillus anthracis and/or protect a host from disease comprising administering an effective amount of the bacteria and/or compositions to the host are further provided.

In another aspect, the present invention provides a Bacillus anthracis strain that is attenuated for nucleic acid repair. In some embodiments, the strain is defective with respect to at least one DNA repair enzyme. In some embodiments, the DNA repair enzyme is UvrA, UvrB, UvrC and/or RecA. In some embodiments, the nucleic acid of the bacteria of the strain has been modified so that the bacteria are attenuated for proliferation. In some embodiments, the nucleic acid of the bacteria has been modified by reaction with a nucleic acid targeting compound that reacts directly with nucleic acid, so that the bacteria are attenuated for proliferation. Compositions, such as immunogenic compositions and vaccine compositions, comprising the bacteria of the strain are provided. Methods of inducing an immune response in a host comprising administering an effective amount of a composition comprising a bacterium from the strain are also provided, as are methods of protecting a host from a disease, comprising administering to the host an effective amount of a composition comprising a bacterium from the strain.

In another aspect, the present invention provides a Bacillus anthracis bacterium, wherein the nucleic acid of the bacterium has been modified so that the bacterium is attenuated for proliferation. In some embodiments, the nucleic acid of the bacterium has been modified by reaction with a nucleic acid targeting compound that reacts directly with nucleic acid, so that the bacterium is attenuated for proliferation. Compositions, such as immunogenic compositions and vaccine compositions, comprising the bacterium are provided. Methods of inducing an immune response in a host comprising administering an effective amount of a composition comprising the modified bacterium are also provided, as are methods of protecting a host from a disease, comprising administering to the host an effective amount of a composition comprising the modified bacteria.

In another aspect, the present invention provides a Bacillus anthracis bacterium comprising a mutation in the lef gene, the cya gene, or both genes that decreases the toxicity of the bacterium (relative to the bacterium without the mutation).

In another aspect, the present invention provides a Bacillus anthracis bacterium comprising a mutation in pagA that decreases the toxicity of the bacterium (relative to the bacterium without the mutation).

In another aspect, the present invention provides a Bacillus anthracis bacterium comprising an additional copy of a gene encoding a Bacillus anthracis antigen.

In another aspect, the present invention provides an asporogenic Bacillus anthracis strain that is attenuated for nucleotide excision repair (NER). In some embodiments, the strain comprises strain comprises an inhibiting mutation in, or a modification that attenuates expression of, at least one sporulation gene. In some embodiments, the strain is defective with respect to SpoIIE. In some embodiments, the strain comprises a mutation in the spoIIE gene. Moreover, in some embodiments, the strain is defective with respect to one or more enzymes selected from the group consisting of UvrA, UvrB, and UvrC. In some embodiments, the strain comprises a mutation in at least one DNA nucleotide excision repair gene. For instance, in some embodiments, the strain comprises a mutation in one or more genes selected from the group consisting of uvrA gene, uvrB gene, and uvrC gene. In some embodiments, the strain further comprises an inhibiting mutation in, or a modification that attenuates expression of, a recombinational repair gene, such as recA. In some embodiments, the mutation or modification comprises a conditional mutation or a transcriptional or translational activator or repressor. In some embodiments, the mutation comprises a conditional mutation. In some embodiments, the modification comprises a nucleic acid molecule encoding a RecA protein under the control of a transcriptional or translational activator or repressor (e.g., the lac repressor). In some embodiments, the strain comprises strain further comprising an inhibiting mutation, or a modification that attenuates expression of, at least one toxin gene. In some embodiments, the at least one toxin gene comprises the lef gene, cya gene, or lef and cya genes. In some embodiments, the strain further comprises a nucleic acid encoding at least one antigen, wherein the nucleic acid encoding the at least one antigen is operably linked to a heterologous promoter. In some embodiments, the heterologous promoter is inducible. For instance, the promoter is, in some embodiments, inducible by ultraviolet light, a nucleic acid targeted compound, a nucleic acid cross-linking compound, and/or an SOS regulatory pathway. In some embodiments, the nucleic acid encoding the at least one antigen is or is derived from: pagA gene; lefgene; cya gene; pagA and lef genes; pagA and cya genes; cya and lef genes; orpagA, lef, and cya genes. In some embodiments, the strain comprises a poly-D-glutamate capsule, while in some alternative embodiments the strain lacks a poly-D-glutamate capsule. A bacterium of the strain is also provided which further comprises at least one covalently linked nucleic acid cross-linking compound (e.g., a psoralen), linked to the genomic DNA of the bacterium. A vaccine or composition comprising the Bacillus anthracis strain is also provided, as well as a method of inducing an immune response in a host to Bacillus anthracis comprising administering to a host an effective amount of a composition comprising a bacterium of the strain.

In another aspect, the invention provides a Bacillus anthracis strain comprising a nucleic acid encoding an antigen, wherein the nucleic acid encoding the antigen is operably linked to a heterologous promoter. In some embodiments, the nucleic acid encoding the antigen is, or is derived from: pagA gene; lef gene; cya gene; pagA and lef genes; pagA and cya genes; cya and lef genes; orpagA, lef, and cya genes. In some embodiments, the heterologous promoter is inducible. In some embodiments, the heterologous promoter is inducible by: ultraviolet light, a nucleic acid cross-linking compound, ultraviolet light and a nucleic acid cross-linking compound, an SOS regulatory pathway, and/or a change or shift in temperature. The invention further provides a vaccine or composition comprising a bacterium of the Bacillus anthracis strain. The present invention also provides a method of inducing an immune response in a host to Bacillus anthracis comprising administering to a host an effective amount of a composition comprising a bacterium of the strain.

In an additional aspect, the invention provides a Bacillus anthracis strain comprising at least one inhibiting mutation in each of uvrA gene, uvrB gene, lef gene, cya gene, and spoIIE gene. Also provided is a bacterium of the Bacillus anthracis strain, wherein the bacterium further comprises at least one covalently linked nucleic acid cross-linking compound linked to the genomic DNA of the bacterium.

In another aspect, the invention provides an asporogenic Bacillus anthracis strain that is attenuated for nucleic acid repair. In one embodiment, the strain is defective with respect to SpoIIE. In some embodiments the strain is also defective with respect to UvrA, UvrB, and/or UvrC (e.g., comprises a mutation in the uvrAB gene). In some embodiments the strain is also defective with respect to RecA. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. A composition, such as a vaccine composition, comprising a bacterium of the strain is also provided. Methods of inducing an immune response in a host to Bacillus anthracis comprising administering to the host an effective amount of the composition are also provided. In addition, methods of protecting a host from a disease, comprising administering to the host an effective amount of a composition comprising a bacterium of the strain are also provided.

In another aspect, the invention provides a Bacillus anthracis strain comprising a heterologous expression cassette comprising a nucleic acid sequence encoding protective antigen, wherein the nucleic acid sequence encoding protective antigen is operably linked to an inducible promoter. In some embodiments, the expression of the protective antigen is induced by treatment of the strain with a psoralen and UVA radiation. For instance, in some embodiments, the protective antigen is operably linked to an SOS regulatory sequence, or any promoter that is induced in response to photochemical treatment or other modification of the DNA of the bacterium. In some embodiments, the strain comprises an inhibiting mutation in, or a modification that attenuates expression of, at least one nucleic acid repair gene (e.g., a nucleotide excision repair (NER) gene, a recombinational repair gene, or an NER gene and a recombinational repair gene). In some embodiments, the strain comprises an inhibiting mutation in, or a modification that attenuates expression of, the nucleic acid repair gene is selected from the group consisting of uvrA; uvrB; uvrC; uvrA and uvrB; and uvrA, uvrB, and uvrC. In some embodiments, the strain comprises an inhibiting mutation in, or a modification that attenuates expression of, recA. In some embodiments, the mutation or modification comprises a conditional mutation or a transcriptional or translational activator or repressor. In some embodiments, the mutation comprises a conditional mutation. In some embodiments, the modification comprises a nucleic acid molecule encoding a RecA protein under the control of a transcriptional or translational activator or repressor. In some embodiments, the strain comprises a mutation that attenuates the ability of the strain to repair its nucleic acid. For instance, in some embodiments, the strain is defective with respect to at least one DNA repair enzyme, such as UvrA, UvrB, or both UvrA and UvrB. In some embodiments, the strain comprises a mutation in the uvrA gene, the uvrB gene, or both the uvrA and uvrB gene. In some embodiments, the strain is defective with respect to UvrC (e.g., comprises a mutation in the uvrC gene). In some embodiments, the strain is defective with respect to RecA. A strain that is defective with respect to RecA can, by way of example, comprise a mutation in the recA gene, comprise a recA gene under control of a repressible promoter, or comprise a temperature sensitive recA gene. In some embodiments, the strain comprises one or more mutations in the lef gene, the cya gene, or both genes that decreases the toxicity of the strain. In some embodiments, the nucleic acid of the bacteria of the strain has been modified so that the bacteria are attenuated for proliferation. In some embodiments, the bacteria of the strain further comprise at least one covalently linked nucleic acid cross-linking compound (e.g., a psoralen), linked to the genomic DNA of the bacteria. In addition, the strain is, in some embodiments, asporogenic (e.g., a spoIIE mutant). In some embodiments, the strain comprises an inhibiting mutation in, or a modification that attenuates expression of, at least one sporulation gene or toxin gene (e.g., spoIIE gene; lef gene; cya gene; spoIIE and lefgenes; spoIIE and cya genes; lef and cya genes; spoIIE, lef, and cya genes). In some embodiments, the strain comprises a poly-D-glutamate capsule, whereas in other embodiments, the strain lacks a poly-D-glutamate capsule. Vaccines and compositions comprising the strain are also provided. A method of inducing an immune response in a host to Bacillus anthracis comprising administering to the host an effective amount of a composition comprising a bacterium of the strain is also provided. A method of protecting a host from a disease, comprising administering to the host an effective amount of a composition (e.g., a vaccine) comprising a bacterium from the strain is also provided.

The invention also provides a Bacillus anthracis strain comprising a heterologous expression cassette that expresses protective antigen under the control of an SOS regulatory sequence. In some embodiments, the Bacillus anthracis strain is attenuated for nucleic acid repair (e.g., is defective for UvrA, UvrB, UvrC, and/or RecA). In some embodiments, the strain is asporogenic. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. A composition, such as a vaccine composition, comprising a bacterium of the strain is also provided. Methods of inducing an immune response in a host to Bacilllus anthracis comprising administering to the host an effective amount of the composition also provided. In addition, methods of protecting a host from a disease, comprising administering to the host an effective amount of a composition comprising a bacterium of the strain are also provided.

In yet another aspect, the invention provides a Bacillus anthracis strain that is defective with respect to RecA. In some embodiments, the strain is a conditional recA mutant. In some embodiments, the strain is a temperature sensitive recA mutant. In some embodiments, the strain is a repressible recA mutant. In some embodiments, the strain comprises a heterologous expression cassette encoding protective antigen, wherein the protective antigen is operably linked to an SOS regulatory sequence. In some embodiments is defective with respect to at least one additional DNA repair enzyme such as UvrA, UvrB, or both UvrA and UvrB. (For instance, the strain optionally comprises a mutation in the uvrA gene, the uvrB gene, or both the uvrA and uvrB gene.) In some embodiments, the strain is defective with respect to UvrC (e.g., comprises a mutation in the uvrC gene). In some embodiments, the modified strain comprises one or more mutations in the lef gene, the cya gene, or both genes that decreases the toxicity of the strain. In some embodiments, the nucleic acid of the bacteria of the strain has been modified so that the bacteria are attenuated for proliferation. In some embodiments, the Bacillus anthracis strain is asporogenic (e.g., defective with respect to SpoIIE). Vaccine compositions comprising a bacterium from the Bacillus anthracis strain are also provided. A method of inducing an immune response in a host to Bacillus anthracis comprising administering to the host an effective amount of a composition comprising a bacterium from the strain is also provided. A method of protecting a host from a disease, comprising administering to the host an effective amount of a composition comprising a bacterium from the strain is further provided.

The invention further provides a Bacillus anthracis strain comprising a temperature sensitive recA gene. In some embodiments, the strain is defective with respect to UvrA and/or UvrB. In some embodiments, the strain is defective with respect to UvrC. In some embodiments, the strain is asporogenic. In some embodiments, the strain comprises a heterologous expression cassette that expresses protective antigen under the control of an SOS regulatory sequence. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. A composition, such as a vaccine composition, comprising a bacterium of the strain is also provided. Methods of inducing an immune response to Bacillus anthracis in a host comprising administering to the host an effective amount of the composition are also provided. In addition, methods of protecting a host from a disease, comprising administering to the host an effective amount of a composition comprising a bacterium of the strain are also provided.

In another aspect, the invention provides a Bacillus anthracis bacterium comprising a mutation in its recA gene, wherein the recA gene encodes a temperature-sensitive RecA protein comprising a V244M mutation. Vaccines and compositions comprising the bacterium are provided as are methods of inducing an immune response in a host to Bacillus anthracis comprising administering to the host an effective amount of a composition comprising the bacterium.

In a further aspect, the invention provides a Bacillus anthracis bacterium comprising a recA gene derived from a foreign bacterium, wherein the recA gene encodes a temperature-sensitive RecA protein. In some embodiments, the recA gene is derived from E. coli, wherein the recA gene encodes a temperature-sensitive RecA protein comprising a V246M mutation. Vaccines and compositions comprising the bacterium are provided as are methods of inducing an immune response in a host to Bacillus anthracis comprising administering to the host an effective amount of a composition comprising the bacterium.

In another aspect, the invention provides a Bacillus anthracis bacterium which is a repressible or inducible recA mutant. In some embodiments, the recA gene is under control of a lac repressor. Vaccines and compositions comprising the bacterium are provided as are methods of inducing an immune response in a host to Bacillus anthracis comprising administering to the host an effective amount of a composition comprising the bacterium.

In another aspect, the invention provides an Bacillus anthracis strain attenuated for nucleic acid repair, wherein the strain is defective with respect to RecA and at least one additional DNA repair enzyme selected from the group consisting of UvrA, UvrB, and UvrC. In some embodiments, the strain defective with respect to RecA is also defective with respect to both UvrA and UvrB. Compositions, such as vaccine compositions, comprising the bacterium of the strain are also provided. Methods of using compositions comprising the strain are also provided.

In another aspect, the invention provides an asporogenic Bacillus anthracis strain attenuated for nucleic acid repair, wherein the strain is defective with respect to SpoIIE and at least one additional DNA repair enzyme selected from the group consisting of UvrA, UvrB, and UvrC. In some embodiments, the asporogenic strain defective with respect to SpoIIE is also defective with respect to both UvrA and UvrB. Compositions, such as vaccine compositions, comprising the bacterium of the strain is also provided. Methods of using compositions comprising the strain are also provided.

In still another aspect, the invention provides an asporogenic Bacillus anthracis strain that is attenuated for nucleic acid repair, comprising a temperature sensitive recA gene and a mutation in the uvrAB gene. Compositions, such as vaccine compositions, comprising the bacterium of the strain is also provided. Methods of using compositions comprising the strain are also provided.

In another aspect, the invention provides an asporogenic Bacillus anthracis bacterium, wherein the nucleic acid of the bacterium has been modified so that the bacterium is attenuated for proliferation. In some embodiments, the bacterium has been modified with a nucleic acid targeted compound that reacts directly with the nucleic acid. In some embodiments, the nucleic-acid targeted compound is a nucleic acid alkylator. In some embodiments, the nucleic acid targeted compound is a psoralen compound (e.g., a psoralen compound activated by UVA irradiation). In some embodiments, the bacterium comprises at least one covalently linked nucleic acid cross-linking compound (e.g., a psoralen), linked to the genomic DNA of the bacterium. In some embodiments, the bacterium is defective with respect to SpoIIE. In some embodiments, the bacterium comprises a mutation in a sporulation gene, such as, but not limited to, spoIIE. In some embodiments, the bacterium is attenuated for nucleic acid repair. For instance, in some embodiments, the bacterium is defective with respect to at least one DNA repair enzyme (e.g., UvrA, UvrB, and/or UvrC). In some embodiments, the bacterium comprises a mutation in one or more genes selected from the group consisting of uvrA, uvrB, and uvrC. In some embodiments, the bacterium is attenuated for recombinational repair. For instance, in some embodiments, the bacterium is defective with respect to RecA. In some embodiments, the bacterium comprises a mutation in the recA gene. For instance, in some embodiments, the bacterium comprises a mutant recA gene which encodes a temperature-sensitive RecA. In some embodiments, the bacterium is a repressible or inducible recA mutant. In some embodiments, the bacterium expresses protective antigen (PA) under the control of an SOS regulatory sequence. In some embodiments, the bacterium comprises one or more mutations in the lef gene, the cya gene, or both genes that decreases the toxicity of the strain. The invention further provides a vaccine or composition comprising the bacterium. In addition, the invention provides a method of inducing an immune response in a host to Bacillus anthracis comprising administering to the host an effective amount of a composition comprising the bacterium.

Methods of making the bacteria and vaccine compositions described herein are also provided.

As used herein, including the appended claims, the singular forms of words such as “a,” “an,” and “the” include their corresponding plural references unless the context clearly dictates otherwise.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

In some embodiments, the present invention provides an attenuated B. anthracis, where limited cross-linking of the genome, of the nucleic acid excision repair mutant vaccine strain, prevents replication and prevents colony formation, but where the minimal nature of the cross-linking allows the B. anthracis to substantially retain the biological properties of the parental live bacterium. In some embodiments, these retained biological properties include the ability to stimulate innate and adaptive immune responses, including bacterial biosynthesis of antigen, and secretion of the antigen, thus stimulating presentation of the antigen by MHC class I and/or MHC class II pathways, cytotoxic. T cell response, and/or antibody production in the vaccinated host.

I. Modified Bacillus Anthracis Bacteria

In some embodiments, the modified Bacillus anthracis strain is derived (indirectly or directly) from a strain selected from the group consisting of Ames, Vollum, A1.a/10, A1.b/23, A2/29, A3.a/34, A3.b/57, A4/69, B/80, Δsterne, VN41Δ1, Dames, NNR1Δ1, and DNH1. In some embodiments, the Bacillus anthracis strain is derived from the Ames strain or the Sterne strain. For instance, in some embodiments, the modified strain of Bacillus anthracis is derived from the Sterne strain. In other embodiments, the modified strain of Bacillus anthracis is derived from the Ames strain. In some embodiments, the Bacillus anthracis bacteria are derived from ANR 1.

In some embodiments, the modified strains are isolated. A strain, bacterium, or composition which is “isolated” is a strain, bacterium, or composition, which is in a form not found in nature. Isolated strains, bacteria, or compositions include those which have been purified to a degree that they are no longer in a form in which they are found in nature. In some, but not all, embodiments, a strain, bacterium, or composition which is isolated is substantially pure.

In some embodiments, the Bacillus anthracis comprises one or more virulence plasmids. In some embodiments, the virulence plasmid comprises the native sequence. In other embodiments, the virulence plasmid is a recombinant plasmid. In some embodiments, the Bacillus anthracis strain comprises a pXO1 plasmid. In some other embodiments, the Bacillus anthracis strain comprises a pXO2 plasmid. In some embodiments, the Bacillus anthracis strain comprises both a pXO1 plasmid and a pXO2 plasmid (i.e., pXO1+ and pXO2+). In other embodiments, the strain lacks a pXO1 plasmid and/or the pXO2 plasmid. In some other embodiments, the strain is pXO1− and pXO2+. In some embodiments, the strain is pXO1+ and pXO2−. In still other embodiments, the strain is pXO1− and pXO2−.

B. anthracis can include a capsule, where the capsule comprises poly-gamma-D-glutamate (also referred to as a poly-D-glutamate capsule). The capsule has an anti-phagocytic role, promotes virulence, and has immunological properties. Intracellular host conditions stimulate biosynthesis of the capsule. The poly-gamma-D-glutamate is encoded by a large plasmid (pXO2) residing in the bacterium (see, e.g., Mesnage, et al. (1998) J. Bacteriol. 180:52-58; Fouet and Mesnage (2002) Curr. Top. Microbiol. Immunol. 271:87-113; Green, et al. (1985) Infection Immunity 49:291-297).

In some embodiments, the modified strain comprises a capsule encoding plasmid, such as a pXO2 plasmid which encodes a poly-D-glutamate capsule. In other embodiments, the strain does not comprise a capsule-encoding plasmid or is otherwise defective in production of the poly-D-glutamate capsule.

A. Bacillus Anthracis Bacteria Modified by Mutations and/or Heterologous Polynucleotides

In some embodiments, the modified Bacillus anthracis strain of the invention has been modified by mutation. The mutation may be a mutation of any type. For instance, the mutation may constitute a point mutation, a frame-shift mutation, an insertion, a deletion of part or all of a gene. In addition, in some embodiments of the modified strains, a portion of the B. anthracis genome has been replaced with a heterologous polynucleotide. In some embodiments, the modification comprises insertion of a heterologous polynucleotide into the genomic DNA of B. anthracis. In some embodiments, the mutations are naturally-occurring. In other embodiments, the mutations are the results of artificial mutation pressure. In still other embodiments, the mutations in the B. anthracis genome are the result of genetic engineering. For safety reasons, in some embodiments, it may be preferred that the mutation be a double mutation or a deletion mutant rather than a point mutation. A mutant in which a gene “xyz” has been deleted is alternatively referred to herein as Δxyz, deltaxyz, xyz⁻, or an xyz deletion mutant. For instance, a bacterial strain in which the uvrA gene has been deleted is alternatively referred to herein as uvrA mutant, ΔuvrA, or uvrA⁻. In addition, it will be understood by one of ordinary skill in the art that a reference to a particular mutant or strain as an “xyz” mutant or “xyz” strain will sometimes refer to a mutant or strain in which the xyz gene has been deleted. In some embodiments, the Bacillus anthracis strains have been modified by introduction of a heterologous polynucleotide, such as a vector, plasmid, and/or expression cassette into the bacteria. Although in some embodiments, the heterologous polynucleotide may be integrated into the genome, in other embodiments, the bacteria comprise a heterologous polynucleotide which is not integrated.

In some embodiments, a modified strain is attenuated for nucleic acid repair; attenuated for sporulation; less toxic; and/or more immunogenic (relative to wild-type or the same strain without the modification).

In some embodiments, the modified bacteria are defective with respect to one or more nucleic acid repair enzymes (e.g., a nucleotide excision repair enzyme or a recombinational repair enzyme), one or more sporulation proteins, or one or more toxins (relative to wild-type or the same bacteria without the modification). For instance, in some embodiments, the modified bacteria are defective with respect to one or more of the following proteins: UvrA, UvrB, UvrC, RecA, SpoIIE, lethal factor, and edema factor. In some embodiments, the modified strains are defective with respect to a protein due to a mutation in the gene encoding the protein or a modification that attenuates expression of the gene encoding the protein (for instance, due to the introduction of heterologous nucleic acid molecules into the Bacillus anthracis, such as antisense expression cassettes).

In some embodiments, the modified bacteria comprise a mutation in one or more genes selected from the group consisting of nucleic acid repair genes (e.g., nucleotide excision repair genes and recombinational repair genes), sporulation genes, and toxin genes. For instance, in some embodiments, the modified bacteria comprise a mutation in one or more genes selected from the group consisting of uvrA, uvrB, uvrC, recA, spoIIE, lef, and cya.

In some embodiments, the mutations in the Bacillus anthracis strains described herein are inhibiting mutations.

An “inhibiting mutation” of a nucleic acid encompasses, e.g., deletion mutations, frameshift mutations, point mutations, and/or insertion mutations, where the functional result of the mutation comprises an inhibition of one or more functions of the gene. For example, an inhibiting mutation in a gene encoding a nucleotide excision repair gene can be one that results in a reduction in rate of formation of the repair complex, in a reduction in rate of binding of the repair complex to DNA, in a reduction in rate of DNA incision efficiency, in an increase in frequency of damaged DNA lesions in the genome, in an increase in stable mutations arising from said increase in frequency, and/or an increase in cell death.

In some embodiments, expression of one or more genes in the modified bacteria are attenuated (relative to wild-type or the same bacteria without the modification). In some embodiments, the one or more genes whose expression is attenuated are selected from the group consisting of nucleic acid repair genes (e.g., nucleotide excision repair genes and recombinational repair genes), sporulation genes, and toxin genes. For instance, in some embodiments, the modified bacteria are attenuated in expression of one or more genes selected from the group consisting of uvrA, uvrB, uvrC, recA, spoIIE, lef and cya.

A “modification that attenuates expression of a gene” or a “modification attenuating expression of a gene” in a bacterium encompasses a mutation or other modification, such as introduction of a heterologous nucleic acid molecule into the bacterium, wherein the mutation or other modification in the bacterium results in a decrease in the expression of the specified gene. The decrease in the expression of the gene can be, but is not limited to, a decrease in the transcription of the gene and/or a decrease in the translation of the gene. Thus, a modification that attenuates expression of a gene embraces reagents and methods wherein translation of the gene is reduced by an antisense nucleic acid.

The present invention contemplates reagents and methods using antisense nucleic acid technology, e.g., antisense RNA or antisense DNA. This includes, but is not limited to, the use of a genomic polynucleotide or plasmid-based polynucleotide encoding the antisense nucleic acid, wherein the antisense nucleic acid inhibits translation of a B. anthracis nucleic acid (see, e.g., Wagner and Simons (1994) Ann. Rev. Microbiol. 48:713-742; Johansson and Cossart (2003) Trends Microbiol. 11:280-285; Wang and Kuramitsu (2003) FEMS Microbiol. Lett. 220:171-176; Lipman (1997) Nucleic Acids Res. 25:3580-3583; Asano, et al. (1998) J. Biol. Chem. 273:11826-11838; Mao, et al. (1995) J. Biol. Chem. 270:19684-19687). In some embodiments, the modification that attenuates expression of a gene in the Bacillus anthracis is a heterologous antisense expression cassette encoding an RNA complementary to a portion of an RNA product (e.g., mRNA) of the gene and able to hybridize to the RNA product under cellular conditions.

In some embodiments, the present invention embraces B. anthracis strains and methods wherein expression of a sporulation gene such as the spoIIE gene, a toxin gene such as the lef gene or cya gene, and/or a nucleic acid repair gene such as a nucleotide excision repair (NER) gene or a recombinational repair gene, and/or activity of the polypeptide expressed from said gene or genes, is reduced or inhibited by at least about 10%, by at least about 20%, by at least about 30%, by at least about 40%, by at least about 50%, by at least about 60%, by at least about 70%, by at least about 80%, by at least about 90%, by at least about 95%, by at least about 99%, or by at least about 99.9% (relative to wild-type or the non-modified strain).

In some embodiments, the B. anthracis strain is attenuated for nucleic acid repair. In some embodiments, the B. anthracis contains a mutation (i.e., a genetic mutation) that reduces (preferably, significantly) the ability of the bacteria to repair modifications to their nucleic acid, including, but not limited to, adducts occurring on pyrimidine residues within the bacterial genome resulting from modification by nucleic acid modifying agents, such as, for example, psoralens, 4-nitroquinoline oxide, cisplatin, mitomycin C, or benzo[a]pyrene. Such a mutation (also referred to herein as a “genetic mutation”) could be in any of a variety of genes that are involved in the DNA repair mechanisms of the bacteria (Aravind et al., Nucleic Acids Research 27(5):1223-1242 (1999)). Bacteria that are deficient in their ability to repair damage to their nucleic acid provide an added level of safety and efficacy to the use of the bacteria of the present invention. In particular, using the appropriate repair deficient mutants, the bacteria are exquisitely sensitive to nucleic acid modification (see Section I.B, below). The nucleic acid of the bacteria may be modified to a lesser degree than for the non-mutant bacteria, yet still ensure the desired amount of attenuation of proliferation. This provides a larger window of efficacy in which to operate so that the expression of the bacterial nucleic acid is sufficient to generate the desired antigenic proteins and/or other factors required to induce a desired immune response de novo in an animal immunized with designated vaccine compositions. The larger window of efficacy results from a random distribution of infrequent DNA modifications in the mutant bacteria compared to the non-mutant bacteria, and as a result, the expression of desired antigenic proteins and/or other necessary factors, within a B. anthracis population comprising a vaccine dose is not affected or is substantially less affected. It also provides an added level of safety as the level of attenuation of proliferation achieved cannot be compromised by repair of the modified nucleic acid. In another embodiment, the genetic mutation alters the susceptibility of the bacteria to treatment with a nucleic acid targeted compound, for example by altering the permeability of the bacteria to the compound or by altering the ability of the compound to access and bind the bacterial nucleic acid. Such mutations may also impact the efficacy of the process of attenuating proliferation while leaving bacterial gene expression substantially unaffected.

In some embodiments, the modified Bacillus anthracis strain or bacterium is attenuated for nucleic acid repair (e.g., nucleotide excision repair and/or recombinational repair), relative to wild-type or the non-modified strain or bacterium. In some embodiments, the ability of the modified strain or bacterium to repair its nucleic acid by one or more nucleic acid repair pathways is at least about 10%, at least about 25%, at least about 50%, at least about 75%, at least about 90%, at least about 95%, or at least about 98%, reduced relative to wild-type or a non-modified strain or bacterium. In some embodiments, the ability of the modified strain or bacterium to repair a modification made to its nucleic acid in order to attenuate the strain or bacterium for proliferation, by one or more nucleic acid repair pathways relevant to the modification, is at least about 10%, at least about 25%, at least about 50%, at least about 75%, at least about 90%, at least about 95%, or at least about 98%, reduced relative to wild-type or a non-modified strain or bacterium. In some embodiments, the bacterium or strain is attenuated for nucleic acid repair due to a mutation. In some other embodiments, the bacterium or strain is attenuated because expression of one or more genes is reduced. In some embodiments, the bacterium or strain is attenuated with respect to nucleic acid repair (relative to wild-type or a non-modified bacterium) only under certain conditions (e.g., a certain temperature or pH or the presence or absence of certain agents, such as IPTG, which induce or repress expression).

In some embodiments, the modified strain or bacterium is defective with respect to at least one DNA repair enzyme (relative to wild-type or the non-modified strain or bacterium). In some embodiments, the activity level of a DNA repair enzyme in a Bacillus anthracis strain or bacterium which is defective with respect to that DNA repair enzyme is decreased by at least about 10%, at least about 25%, at least about 50%, at least about 75%, at least about 90%, at least about 95%, or at least about 98%, relative to wild-type or a non-modified strain or bacterium. In some embodiments, the bacterium or strain is defective with respect to a DNA repair enzyme because the bacterium or strain does not express the DNA repair enzyme or shows decreased expression (or conditional expression) of the DNA repair enzyme. In some other embodiments, the bacterium or strain is defective with respect to a DNA repair enzyme because the bacterium or strain expresses a mutant form of the DNA repair enzyme, that is less active or is conditionally active. In some embodiments, the bacterium or strain that is defective with respect to a DNA repair enzyme because the bacterium or strain is conditionally defective with respect the enzyme. For instance, in some embodiments, the bacterium or strain may only be defective with respect to the DNA repair enzyme (relative to wild-type or a non-modified bacterium) under certain conditions (e.g., a certain temperature or pH or the presence or absence of certain agents, such as IPTG).

To illustrate the advantages of using a repair deficient mutant, one can consider the mechanism of the attenuation of bacterial proliferation (see Section I.B, below). The bacterial nucleic acid is modified either by strand breakage or pyrimidine dimers, or by chemical modifications such as monoadducts or crosslinks. If the mechanisms for repair of these modifications are intact, a certain number of modifications will be required in order to overcome the bacterial DNA repair mechanisms, and achieve sufficient attenuation of proliferation. The greater the modification of nucleic acid, the greater the reduction in protein expression, and the greater the reduction of protein expression of any given gene, within a bacterial population comprising a vaccine dose. Even though the levels of modification required to attenuate proliferation are much lower than the levels required to stop protein expression, protein expression will still be reduced to some extent, possibly to a level unacceptable to generate a desired immune response in a vaccinated individual. The use of DNA repair deficient mutants significantly reduces the levels of nucleic acid modification needed to attenuate proliferation such that a lower modification level will result in adequate attenuation of proliferation. Since the nucleic acid modification is much lower, the expression of proteins will be less affected, providing for a higher level of expression of the protein(s) of interest. Such repair deficient mutants are particularly useful in the preparation of vaccines, where the safety of the vaccine can be increased by a slight modification of the nucleic acid, leaving a sufficiently high level of protein expression, in particular of the antigen to which the immune response is targeted.

In one embodiment the repair deficient mutant is unable to repair interstand crosslinks. Such mutants include, but are not limited to, mutations in uvr genes, i.e. uvrA, uvrB, uvrC, and uvrD genes as well as recA genes. The mutations may be in one or more of these genes. These mutations result in attenuation in the activity of the corresponding enzymes UvrA (an ATPase), UvrB (a helicase), UvrC (a nuclease), UvrD (a helicase II) and RecA (a recombinase). These mutants would most typically be used in conjunction with a crosslinking compound, such as a psoralen. (See, e.g., Section I.B, below.) Since the bacterial nucleic acid is crosslinked in some locations, and these crosslinks cannot be repaired, the bacteria are unable to replicate as the original strands of nucleic acid can not be separated. Since they cannot be repaired, very few crosslinks are needed, the bacterial nucleic acid is for the most part accessible for transcription, and protein expression is not altered significantly. In some embodiments, a population of repair deficient bacterial mutants that are unable to repair interstrand crosslinks are suitably crosslinked such that essentially every bacterium in the population contains at least one crosslink, such that attenuation of replication is essentially complete, wherein the bacterial gene expression of the population is sufficiently active.

In some embodiments, the B. anthracis strain comprises an inhibiting mutation in or a modification that attenuates expression of at least one nucleic acid repair gene. The nucleic acid repair gene is, e.g., a nucleic acid excision repair gene or a recombinational repair gene. Nucleic acid repair, mediated by a number of individual enzymes and protein complexes, includes repair by nucleotide excision repair (NER) and recombinational repair. Nucleic acid repair enzymes, protein complexes, and pathways have been characterized in B. anthracis, B. subtilis, E. coli, and in other organisms. In E. coli, the NER pathway begins with the uvrABC endonuclease which comprises a complex of UvrA, UvrB, and UvrC proteins. The role of proteins such as RecA, RecF, and RecR, in repairing DNA damage has been described. Expression of the excision repair genes uvrA, uvrB, and uvrD, but not uvrC, are upregulated as part of the SOS response. In some embodiments, the strain further comprises an inhibiting mutation in or a modification that attenuates expression of another gene, e.g., spoIIE gene, lef gene, cya gene, or any combination thereof.

In some embodiments, the modified Bacillus anthracis strain is attenuated for nucleotide excision repair. For instance, in some embodiments, the strain is defective with respect to a nucleotide excision repair enzyme. In some embodiments, the strain comprises a inhibiting mutation in, or a modification that attenuates expression of one or more nucleotide excision repair genes (e.g., UvrA, UvrB, UvrC, and/or UvrD). In some embodiments the strain comprises a mutation in a nucleotide excision repair gene.

In some embodiments, the modified Bacillus anthracis strain is attenuated for recombinational repair. For instance, in some embodiments, the bacteria are defective with respect to a recombination repair enzyme. In some embodiments, the bacteria of the strain comprise an inhibiting mutation in, or a modification that attenuates expression of, a recombination repair enzyme, such as, but not limited to, RecA. The inhibited, mutated, or attenuated recombinational repair genes of the present invention encompass, but are not limited to, recA, recD, recF, recG,or ruvABC (Munakata, et al. (1991) Photochem. Photobiol. 54:761-768; Carrasco, et al. (2002) Mol. Genet. Genomics 266:899-906; Alonso and Luder (1991) Biochimie 73:277-280; Kuzminov (1999) Microbiol. Mol. Biol. Rev. 63:751-813). In some embodiments, the recombinational repair gene is recN. In other embodiments, the bacteria or strain that is defective with respect to a DNA repair enzyme and/or a recombination repair enzyme is not defective with respect to RecN. (The terms “inhibited,” “mutated,” and “attenuated” are not necessarily exclusive of each other, and can coexist, when describing, e.g., a reagent, molecule, nucleic acid, gene, enzyme, protein, or bacterium.)

In some embodiments, the modification that attenuates expression of a recombination repair enzyme (e.g., recA) places expression of the recombination repair enzyme under the control of a transcriptional activator or repressor. In some embodiments, the modification that attenuates expression of a recombination repair enzyme places expression of the recombination repair enzyme under the control of a translational activator or repressor. In some embodiments, the transcriptional repressor is the lac repressor.

In one embodiment the modified Bacillus anthracis strain is defective with respect to RecA. In some embodiments, the defective strain comprises a mutation in the recA gene. In some embodiments, the defective strain is a conditional recA mutant, such as a temperature sensitive recA mutant. In some embodiments, the defective strain is a repressible recA mutant. In some embodiments, the strain defective in RecA is an inducible recA mutant. In some embodiments, the strain that is defective with respect to RecA comprises a recA gene under the control of a transcriptional repressor. For instance, in some embodiments, a recA gene may be operably linked to a promoter and a regulatory sequence which binds a transcriptional repressor and the transcriptional repressor is encoded by an expression cassette introduced within the B. anthracis. In some alternative embodiments, two expression cassettes are introduced to the bacteria. One expression cassette comprises a polynucleotide encoding an anti-recA antisense RNA operably linked to a promoter and a regulatory sequence which binds the transcriptional repressor. The other expression cassette encodes the transcriptional repressor. In some embodiments, the transcriptional repressor is the lac repressor.

In some embodiments, a mutation (e.g., an inhibiting mutation) in the recA gene is a conditional mutation. In such a mutation, the mutation in the recA gene results in the attenuation in the activity of recA only under certain conditions (i.e. non-permissive conditions), such as a suitable pH or temperature of the bacterial population. Bacteria comprising a conditional recA mutation can be cultured under permissive conditions in order to grow sufficient levels of the bacteria and then placed under non-permissive conditions for treatment to modify the nucleic acid, then optionally stored under non-permissive conditions such that the nucleic acid damage is not adequately repaired. As an example of this, a recA temperature sensitive mutant is grown at 30 ° C., where it grows well, and is treated to modify the nucleic acid at 42 ° C., which is non-permissive for recA such that it is very sensitive to treatment, such as psoralen crosslinking.

The present invention provides regulatory sequences sensitive to, e.g., temperature, pH, osmotic changes, or alterations in the concentrations of oxygen, ions, or metabolites for use in the nucleic acids, bacteria and methods of the present invention (see, e.g., Repoila and Gottesman (2003) J. Bacteriol. 185:6609-6614; Schofield, et al. (2003) Appl. Environ. Microbiol. 69:3385-3392; Hanna, et al. (2001) J. Bact. 183:5964-5973; Deuerling, et al. (1995) J. Bacteriol. 177:4105-4112). One or more of these regulatory sequences can be used to allow expression of recA during growth of B. anthracis, but to prevent or inhibit expression of recA before, or shortly before, or during exposure of the B. anthracis to reagents that inflict DNA damage. In the case of a temperature sensitive mutant, the location of the mutation need not necessarily be in a regulatory region; the mutation can be in the coding region of the gene. For example, a temperature sensitive recA mutant was produced by a mutation in the coding region of the recA gene (Kawashima et al. Mol. Gen. Genet. 193:288-92 (1984)).

In some embodiments, the Bacillus anthracis bacterium which is a temperature-sensitive recA mutant comprises a temperature-sensitive recA mutant gene. For instance, in some embodiments, the strain comprises a mutant recA gene which encodes a temperature-sensitive RecA protein. In some embodiments, the temperature-sensitive RecA is functional at temperatures suitable for growing the bacteria, but non-functional or less functional at higher temperatures which can be used during treatment with nucleic-acid targeted compounds and/or ultraviolet light when it is desirable to attenuate the ability of the bacteria for recombinational repair. In some embodiments, the temperature-sensitive recA mutant gene comprises a mutation analogous to that of the recA44 temperature sensitive allele of E. coli (V246M; Kawashima et al., Mol. Gen. Genet. 193:288-92 (1984); Hall et al., J. Bacteriol. 121:892-900 (1975)). In some embodiments, the bacterium or strain comprises a recA gene which encodes a temperature-sensitive RecA protein comprising a mutation analogous to the V246M mutation of the recA44 temperature sensitive recA mutant of E. coli. For instance, in some embodiments, the modified Bacillus anthracis bacterium comprises a mutant Bacillus anthracis recA gene which encodes a temperature-sensitive mutant RecA protein comprising the mutation V244M (or a functional fragment or variant of such a protein). In some embodiments, the Bacillus anthracis bacterium comprises a polynucleotide comprising a polynucleotide encoding SEQ ID NO:54 (see sequence below), or a functional fragment or variant thereof.

In some alternative embodiments, the modified B. anthracis bacterium comprises a recA gene derived from a foreign bacterium, wherein the recA gene encodes a temperature-sensitive RecA protein. In some embodiments, the mutant recA gene is derived from E. coli. For instance, in some embodiments, the modified Bacillus anthracis bacterium comprises an E. coli recA gene which encodes a temperature-sensitive RecA protein, or a functional fragment thereof. For instance, in some embodiments, the modified B. anthracis bacterium (or strain) comprises a recA gene that comprises the recA44(ts) allele of E. coli. Alternatively, the modified B. anthracis comprises the coding sequence of the recA44(ts) allele operably linked to a promoter functional in Bacillus anthracis. In some embodiments, the Bacillus anthracis bacterium comprises a polynucleotide comprising a polynucleotide encoding SEQ ID NO:54 (see sequence below), or a functional fragment thereof. In some embodiments, the bacterium comprises an E. coli recA gene, wherein the E. coli recA gene encodes a temperature-sensitive RecA protein comprising a V246M mutation within the sequence 245KVVKNK250 (SEQ ID NO:14) or a V247M mutation within the sequence 245VKVVKNK251 (SEQ ID NO:56). In some embodiments, the Bacillus anthracis bacterium or strain comprises a nucleic acid encoding a temperature-sensitive RecA protein in which the sequence KVVKNK (SEQ ID NO:14) has been mutated to KMVKNK (SEQ ID NO:57).

In some embodiments, the bacteria comprise a nucleic acid molecule encoding a RecA protein, or functional fragment or variant thereof, which is a temperature-sensitive mutant.

It is possible that upon vaccination, the conditions may permit expression of recA, resulting in some repair and presenting a safety issue. In some embodiments, it may be preferable to use a mutation of the recA gene which is a double mutation or a deletion mutant for safety reasons.

The invention further provides modified B. anthracis strains and bacteria which are repressible or inducible recA mutants. In some embodiments, the bacteria are repressible mutants. In some embodiments, the bacteria are inducible mutants.

In some embodiments, the invention provides a Bacillus anthracis bacterium (and strain) that comprises a nucleic acid molecule encoding a RecA protein, wherein expression of the RecA protein is under the control of a transcriptional or translational activator or repressor (e.g., a transcriptional repressor, such as the lac repressor). In some embodiments, a RecA protein is expressed in the bacterium under the control of a heterologous transcriptional repressor or activator. In some embodiments, expression of the recA gene is under control of a lac repressor.

In some embodiments, the invention provides a Bacillus anthracis bacterium (and strain) that comprises a nucleic acid encoding a RecA protein, wherein the nucleic acid is operably linked to a heterologous operator which binds a repressor or activator. (Typically a promoter is also operably linked to the nucleic acid encoding the RecA protein and the operator.) In some embodiments, the operator binds a repressor. In some embodiments, the operator binds the lac repressor.

In some embodiments, the invention provides a Bacillus anthracis bacterium (and strain) that comprises a nucleic acid that produces a RecA antisense RNA upon transcription, wherein the nucleic acid is operably linked to an operator which binds a repressor or activator. (Typically a promoter is also operably linked to the nucleic acid encoding the RecA protein and the operator.) In some embodiments, the operator binds a repressor. In some embodiments, the operator binds the lac repressor.

The invention further provides a polynucleotide comprising a nucleic acid that produces a RecA antisense RNA upon transcription, wherein the nucleic acid is operably linked to an operator that binds a lac repressor. For instance, the invention provides a polynucleotide comprising a nucleic acid sequence complementary to a recA RNA transcript, or a portion thereof, which will hybridize thereto under cellular conditions, wherein the nucleic acid sequence is operably linked to an operator that binds a lac repressor (or is operably linked to both the operator and a promoter suitable for transcription of the nucleic acid sequence in the bacteria). An expression cassette, vector, and host cell comprising this polynucleotide is also provided.

In some embodiments, the modified B. anthracis bacterium or strain comprises an expression cassette that expresses a RecA antisense RNA.

In some embodiments, the inducible recA mutant comprises a recA gene which is operably linked to an inducible promoter or a repressible promoter. For instance, in some embodiments, the recA gene is operably linked to a regulatory sequence (such as a lac operator) which binds a transcriptional repressor (e.g., the lac repressor). In some embodiments, addition of an inducer molecule such as IPTG induces transcription of recA.

In some embodiments, the modification made to the B. anthracis strain or bacterium comprises a nucleic acid molecule encoding a RecA protein, wherein expression of the RecA protein is under the control of a transcriptional or translational activator or repressor (i.e., expression of the RecA protein is controlled (or modulated) by the activator or repressor). The control may be direct or indirect. In some embodiments, the expression is inducible. In some embodiments, the expression is repressible. In some embodiments, expression of the RecA protein is under the control of a transcriptional repressor (e.g., the lac repressor). In some embodiments, the nucleic acid encoding the RecA protein is operably linked to an operator which binds the repressor or activator. For instance, the nucleic acid encoding the RecA protein may be operably linked both to a promoter and to an operator which binds a repressor or activator such as the lac repressor.

In some another embodiments, the present invention provides a recA gene under the control of a transcriptional or translational activator or repressor. The transcriptional activator can be activated during conditions where the B. anthracis is grown, and where the transcriptional activator can be deactivated at a later time, that is, when psoralen is introduced. Similarly, the transcriptional repressor can be engineered so that it is inactive when the B. anthracis is grown, and so that it is active when the B. anthracis is treated with psoralen. The recA gene of the present invention can be functionally (or operably) linked with a nucleic acid encoding anti-sense nucleic acid, in a way that allows expression of RecA during growth of the B. anthracis, but inhibits or prevents expression of RecA during and after treatment with psoralen.

The present invention provides a transcriptional activator, an transcriptional repressor, an translational activator, and an translatable repressor, e.g., for regulating RecA expression. The present invention also provides a nucleic acid encoding an antisense polynucleotide, where the antisense polynucleotide can inhibit translation of RecA.

Provided in some embodiments is a transcriptional or translational activator or repressor that supports expression of RecA during growth or preparation of a culture of B. anthracis cells, where the modulator (i.e., effector) of the repressor (or activator) is withdrawn (or terminated) shortly prior to administrating a DNA-modifying agent. Removal or termination of the modulator of the repressor (or activator), shortly before administering the DNA-modifying agent, serves to reduce RecA-mediated repair of the DNA, thus allowing DNA modification at relatively low concentrations of the DNA-modifying agent. In some embodiments, the repressor is a transcriptional repressor.

Also provided is a transcriptional or translational repressor (or activator), where the modulator of the repressor (or activator) can be added after growth or preparation of a culture of B. anthracis cells. In some embodiments, the modulator is introduced shortly before administering a DNA-modifying agent. Here, the repressor (or activator) provokes a reduction in RecA expression in the B. anthracis cell, thus allowing DNA modification at relatively low concentrations of a DNA modifying agent. In some embodiments, a transcriptional repressor is utilized.

As indicated above, it is also possible to construct the Bacillus anthracis strain such that the recA is under the control of the lac repressor, such that expression of recA can be induced by isopropyl-beta-D-thiogalactopyranoside (IPTG) when desired, but not during photochemical inactivation or post-immunization. The possibility of recA expression can then be eliminated for the inactivation and/or immunization steps by withholding further IPTG from the strain and/or eliminating IPTG from the strain's environment.

In some embodiments, to generate a Bacillus anthracis bacterium which is a repressible RecA mutant for use in the vaccines of the invention, two expression cassettes are introduced into the genome of the bacterium (e.g., Bacillus anthracis ΔuvrAB). The first expression cassette encodes the lac I^{q} protein under the control of a highly-active constitutive promoter, for example, but not limited to the B. anthracis promoters Pamy, Pntr, or the pagA promoter. The second cassette expresses RecA anti-sense RNA, also under the control of a selected promoter, for example, but not limited to, a B. anthracis promoter such as the Pamy, Pntr, or pagA promoter, but, importantly, the lacOip operator is placed at the 3′ end of the selected promoter. Thus, expression of the RecA anti-sense RNA is prevented in the presence of the lac I^{q} protein. In this configuration, functional RecA protein is produced under normal growth conditions, due to constitutive expression of the lac I^{q} protein which prevents expression of the RecA anti-sense RNA. Addition of IPTG to the culture will result in binding of this inducer molecule to the lac I^{q} repressor, and prevent it from binding to the lacOip operator. This results in the high-level synthesis of RecA anti-sense RNA and, in turn, inhibition of translation of RecA protein, mediated through complementary binding to RecA message. The expression cassettes can be assembled onto the pKSV7 integration vector and introduced into Bacillus anthracis ΔuvrAB at any desired location, but desirably within intergenic regions of the bacterial chromosome. In this setting, the expression of recA is shut-off by addition of IPTG to the fermentation culture prior to illumination with UVA light.

In some embodiments, the B. anthracis strain is an inducible recA mutant which is under control of the lac repressor. In some embodiments, the recA promoter of Bacillus anthracis (or a promoter operably linked to sequence encoding RecA or a derivative thereof) is engineered to contain a downstream lacOip site. An expression cassette is introduced into Bacillus anthracis encoding lacl^{q} under the control of a highly expressed constitutive promoter, for example, but not limited to the promoters for the B. anthracis genes agA, ntr, or amy. In this configuration, functional RecA protein is produced only in the presence of IPTG or other inducer molecule. Upon removing the inducer, recA transcription is blocked and the amount of RecA protein will decrease depending on its half-life in the bacterium. In this configuration, IPTG is present when it is desirable to culture the cells normally, and it is removed when it is desired to introduce DNA modifications and abrogate DNA repair. The expression cassette encoding lacOip can be assembled onto the pKSV7 integration vector and introduced into Bacillus anthracis at any desired location, but desirably within intergenic regions of the bacterial chromosome.

The present invention provides an engineered E. coli lac repressor and operator, for use in B. anthracis. See, e.g., Yansura and Henner (1984) Proc. Natl. Acad. Sci. USA 81:439-443, disclosing the E. coli lac repressor and operator in B. subtilis. However, a variety of other regulatory elements useful as part of inducible and/or repressible expression systems in bacteria are known in the art. For instance, heat-inducible expression systems for use in B. anthracis are available (Baillie, et al. (1998) FEMS Microbiol. Lett. 163:43-47). A number of response elements are disclosed for B. subtilis. These elements respond to, e.g., heat shock, cold-induction, salt stress, ethanol, oxygen starvation, reactive oxygen (e.g., peroxide), nutrient starvation, nutrient addition (e.g., aminobutyrate, glucose, maltose, or arabinose), nitrogen limitation, alkali shift (sudden shift to pH 8.9). For example, a cold response element in B. subtilis is induced with a shift down in temperature from 37 degrees to 18 degrees (see, e.g., Hecker, et al. (1996) Mol. Microbiol. 19:417-428; Yasbin, et al. (1992) Mol. Microbiol. 6:1263-1270; Mongkolsuk and Helmann (2002) Mol. Microbiol. 45:9-15; Sakamoto and Murata (2002) Curr. Opin. Microbiol. 5:208-210; Fisher (1999) Mol. Microbiol. 32:223-232; Nickel, et al. (2004) Mol. Genet. Genomics 272:98-107; Chan and Lim (2003) J. Mol. Biol. 333:21-31; Atalla and Schumann (2003) J. Bacteriol. 185:5019-5022; Kawai, et al. (2003) Mol. Microbiol. 47:1113-1122; Kaan, et al. (2002) Microbiol. 148:3441-3455; Belitsky and Sonenshein (2002) Mol. Microbiol. 45:569-583; Fuangthong, et al. (2002) J. Bacteriol. 184:3276-3286; Stulke, et al. (1997) Mol. Microbiol. 25:65-78; Mota, et al. (2001) J. Bacteriol. 183:4190-4201; Noone, et al. (2000) J. Bacteriol. 182:1592-1599; Schonert, et al. (1999) Res. Microbiol. 150:167-177).

In some embodiments, the repair deficient mutant lacks the ability to make a photolyase which repairs pyrimidine dimers. For example, the mutation may be in a gene equivalent to a phrB gene. Such a mutant could be used in conjuction with ultraviolet irradiation (e.g. UVB, UVC) of the bacteria to produce pyrimidine dimers in the bacterial nucleic acid.

In one embodiment, the bacteria comprises at least one mutation that reduces (preferably, significantly reduces) the ability of the bacteria to repair modifications to their nucleic acid in combination with at least one mutation not related to repair mechanisms.

In some embodiments, the invention provides a Bacillus anthracis strain which is defective with respect to at least one DNA repair enzyme (relative to wild type). In some embodiments, the strain that is defective with respect to at least one DNA repair enzyme is attenuated for DNA repair relative to wild type. In some embodiments, the capacity of the bacteria for DNA repair by at least one DNA repair pathway is reduced by at least about 10%, at least about 25%, at least about 50%, at least about 75%, or at least about 90% relative to wild type. In some embodiments, the capacity of the bacteria for DNA repair by at least one DNA repair pathway is reduced by at least about 10% relative to wild type. In some embodiments, the capacity of the bacteria for DNA repair by at least one DNA repair pathway is reduced by at least about 25% relative to wild type. In some embodiments, the capacity of the bacteria for DNA repair by at least one DNA repair pathway is reduced by at least about 50% relative to wild type. In some embodiments, the capacity of the bacteria for DNA repair by at least one DNA repair pathway is reduced by at least about 75% relative to wild type. Methods for assessing the ability of a strain to effect DNA repair are well known to those of ordinary skill in the art. In some embodiments, the strain is defective with respect to one or more of the following enzymes: PhrB, UvrA, UvrB, UvrC, UvrD, and RecA. In some embodiments, the strain is defective with respect to UvrA, UvrB, or both enzymes. In some embodiments, the strain is defective with respect to UvrC. In some embodiments, the strain is defective with respect to RecA. In some embodiments, the bacteria comprise a genetic mutation in one or more gene selected from the group consisting of phrB, uvrA, uvrB, uvrC, uvrD and recA. In some embodiments, the bacteria comprise genetic mutations in both uvrA and uvrB. In some embodiments, the bacteria comprise a genetic mutation in uvrC. In some embodiments, the Bacillus anthracis is a uvrAB deletion mutant. In some embodiments, the bacteria comprise a genetic mutation in recA.

Thus, the invention provides an isolated Bacillus anthracis strain, comprising a genetic mutation that attenuates its ability to repair its nucleic acid (i.e., is attenuated for DNA repair). In some embodiments, the mutant strain is defective with respect to at least one DNA repair enzyme (such asUvrA and/or UvrB, or UvrC). In some embodiments, the mutant strain comprises a genetic mutation in the uvrA gene and/or the uvrB gene. In some embodiments, the uvrA gene, the uvrB gene , or both genes are deleted. (The coding sequence of the uvrA gene is shown as SEQ ID NO:1, below, and the coding sequence of the uvrB gene is shown as SEQ ID NO:2, below.) In some embodiments, the modified Bacillus anthracis strain is the Bacillus anthracis Sterne ΔuvrAB strain deposited with the American Type Culture Collection (ATCC),

University Blvd., Manassas, Va., 20110-2209, United States of America, on Feb. 20, 2004, under the provisions of the Budapest Treaty on the International Recognition of the. Deposit of Microorganisms for the Purposes of Patent Procedure, and identified by accession number PTA-5825, or a mutant of the deposited strain which is defective with respect to UvrA and UvrB. In some embodiments, the modified strain is the Bacillus anthracis Sterne ΔuvrAB strain deposited with the American Type Culture Collection (ATCC), 10801 University Blvd., Manassas, Va., 20110-2209, United States of America, on Feb. 20, 2004, under the provisions of the Budapest Treaty on the International Recognition of the Deposit of Microorganisms for the Purposes of Patent Procedure, and identified by accession number PTA-5825. In some embodiments, the mutant strain comprises a genetic mutation in the uvrC gene.

In some embodiments, the mutant strain is attenuated with respect to RecA. In some embodiments, the mutant strain comprises a genetic mutation in the recA gene. In some embodiments the mutant strain comprises a conditional recA gene. In some embodiments, the mutant strain comprises a mutation in the recA gene that makes expression of the recA protein temperature sensitive. In some alternative embodiments, a mutant strain of B. anthracis is constructed which comprises a repressible recA gene (e.g, the recA gene is under control of the lac repressor (inducible by IPTG), permitting expression of recA during growth, but not during inactivation (such as with S-59/UVA) and/or post-immunization). In some embodiments, the mutant strain comprises one or more mutations in the lef gene, cya gene, or both genes, that decreases the toxicity of the strain relative to the non-mutated strain. In some embodiments, the modified strain is also asporogenic.

The invention further provides an isolated Bacillus anthracis strain comprising a temperature sensitive recA gene. In some embodiments, the strain is defective with respect to UvrA and/or UvrB. In some embodiments, the strain is defective with respect to UvrC. In some embodiments, the strain is asporogenic. In some embodiments, the strain comprises a heterologous expression cassette that expresses protective antigen under the control of an SOS regulatory sequence. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased.

Methods of altering the genome of Bacillus anthracis are known to those skilled in the art. One method of generating mutations in Bacillus anthracis is by allelic exchange using an allelic exchange vector known to those in the art. An exemplary allelic exchange plasmid is pKSV7 described in Camilli et al., Molecular Microbiology, 8:143-147 (1993). As a first step in generating a mutant Bacillus anthracis, the region of the genome to be deleted or otherwise mutated and approximately 1000 bps both upstream and downstream of the B. anthracis genome is PCR-amplified and then cloned into the pKSV7 plasmid vector (or an analogous vector). (A Bacillus genera-specific or B. anthracis-specific temperature (ts) replicon may be substituted for the Listeria ts replicon present in the pKSV7 allelic exchange plasmid vector.) Restriction endonuclease recognition sites in the region to be deleted or mutated may be used to delete the desired portion of the targeted gene in the region. Alternatively, a portion of the targeted gene within the region may be removed and replaced with sequences containing the desired mutation or other alteration. The region of the B. anthracis genome that is amplified can be altered, for instance, using restriction enzymes or a combination of restriction enzymes and synthetic gene sequences, before or after cloning into the allelic exchange plasmid. In some embodiments, the sequence may be altered as a PCR amplicon and then cloned into pKSV7. In alternative embodiments, the amplicon is first inserted into another plasmid first and then altered, excised, and inserted into pKSV7. Alternatively, the PCR amplicon is inserted directly into the pKSV7 plasmid and then altered, for instance, using convenient restriction enzymes. The pKSV7 plasmid containing the altered sequence is then introduced into B. anthracis. This can be done via electroporation. The bacteria are then selected on media at a permissive temperature in the presence of chloramphenicol. This is followed by selection for single cross-over integration into the bacterial chromosome by passaging for multiple generations at a non-permissive temperature in the presence of chloramphenicol. Lastly, colonies are passaged for multiple generations at the permissive temperature in media not containing the antibiotic. Some additional details regarding this method as applied to the deletion of uvrAB from Bacillus anthracis are provided, by way of non-limiting example, in Example 1 and Example 2, below. Example 4, below, also provides an example of the use of pKSV7 to delete spoIIE.

One of ordinary skill in the art will be able to readily adapt the pKSV7 allelic exchange method (or common methods known in the art) to introduce any of the mutations described herein into Bacillus anthracis, including, but not limited, to the recA mutations described herein.

Confirmation of the presence of a desired mutation in a bacterium can be determined by PCR and/or sequencing using standard methods known in the art. For the PCR or sequencing, primers are used which are complementary to those regions flanking the area of interest on the chromosome where the deletion or other mutation was intended. Alternatively, the presence of the desired mutation can be determined by functional assay. For instance, the presence of nucleotide excision repair mutation such as deletion of uvrAB can be assessed using an assay which tests the ability of the bacteria to repair its nucleic acid using the nucleotide excision repair (NER) machinery and comparing that ability against wild-type Bacillus anthracis. Such functional assays are known in the art. For instance, cyclobutane dimer excision or the excision of UV-induced (6-4) products can be measured to determine a deficiency in an NER enzyme in the mutant (see, e.g., Franklin et al., Proc. Natl. Acad. Sci. USA, 81: 3821-3824 (1984)). Alternatively, survival measurements can be made to assess a deficiency in nucleic acid repair. For instance, the bacteria can be subjected to psoralen/UVA treatment and then assessed for its ability to proliferate and/or survive in comparison to wild-type.

Methods for assessing activity and expression, as well as for measuring the stimulation, inhibition, or inactivation, of nucleic acid repair proteins and nucleic acid repair pathways are known in the art (see, e.g., Lin and Sancar (1990) J. Biol. Chem. 265:21337-21341; Smith, et al. (2002) J. Bacteriol. 184:488-493; Ramaswamy and Yeung (1994) J. Biol. Chem. 269:485-492; Crowley and Hanawalt (2001) Mutat. Res. 485:319-329; Bierne, et al. (1997) Mol. Microbiol. 26:557-567; Courcelle and Hanawalt (2001) Proc. Natl. Acad. Sci. USA 98:8196-8202). Expression and/or activity can be assessed by standard assays, e.g., Northern blots, antibody-based assays, and in vitro or in vitro functional assays using UV-light or chemically damaged plasmid substrates (see, e.g., Mu and Sancar (1997) Prog. Nucleic Acid Res. Mol. Biol. 56:63-81; Sancar (1996) Annu. Rev. Biochem. 65:43-81; Clark (1991) Biochemie 73:523-532).

Additional information regarding the mutation of microbes such as Bacillus anthracis to render them defective with respect to nucleic acid repair is provided in U.S. Provisional Application No. 60/446,051, filed Feb. 6, 2003; U.S. Provisional Application No. 60/449,153, filed Feb. 21, 2003; U.S. Provisional Application No. 60/490,089, filed Jul. 24, 2003; U.S. Provisional Application No. 60/511,869, filed Oct. 15, 2003; U.S. patent application Ser. No. 10/773,618, filed Feb. 6, 2004 (U.S. Patent Publication No. 2004/0197343 Al); and the U.S. patent application Ser. No., 10/883,599, filed Jun. 30, 2004, the disclosures of each of which are incorporated by reference herein in their entirety.

In some preferred embodiments, the nucleic acid of the strain that is defective with respect to nucleic acid repair has been modified so that the strain is also attenuated for proliferation. Methods of modifying the nucleic acid of a strain so that the strain is attenuate for proliferation are described herein.

Either as an alternative to or in addition to one or more of the nucleic acid repair mutations described above (e.g., recA, uvrA, uvrB, uvrC, and uvrAB), in some embodiments the modified B. anthracis strains comprise other mutations.

In some embodiments, the B. anthracis bacteria are defective with respect to the formation of spores (i.e., are sporulation-deficient). For instance, in some embodiments, the B. anthracis strains are asporogenic. In some embodiments, the bacteria are defective with respect to a sporulation protein. In some embodiments, the B. anthracis bacteria comprise an inhibiting mutation in, or a modification that attenuates expression of, at least one sporulation gene, a gene required or used for sporulation, e.g., spoIIE, spoOA, spoIVB, spoIIA, and spoIIG (see, e.g., Liu, et al. (2003) Nucleic Acids Res. 31:6891-6903; Guidi-Rontani, et al. (1999) Mol. Microbiol. 33:407-414; Brown, et al. (1994) Mol. Microbiol. 14:411-426; Worsham et al. (1999) Can. J. Microbiol., 45:1-8). In some embodiments, the B. anthracis strain comprises a mutation in spoIIE. In some embodiments, the asporogenic strains are defective with respect to SpoIIE, a protein phosphatase that causes release of a sigma factor called sigma F (Hilbert, et al., J Bacteriol., 185: 1590-8 (2003)). In some embodiments, the strains are rendered asporogenic by mutation of the spoIIE gene. In some embodiments, part or all of the spoIIE gene is deleted. In some embodiments, the spoIIE gene is deleted. In some embodiments, the apsorogenic or sporulation-deficient bacteria and/or strain does not comprise a mutation in either spoOA or spoIVB. In some embodiments the bacteria and/or strain are not defective with respect to SpoOA or SpoIVB.

In some embodiments, expression and/or activity of a sporulation gene or production of a B. anthracis spore or spore component is reduced in the sporulation-deficient mutant by at least about 25%, at least about 50%, at least about 75%, at least about 90%, at least about 97%, or at least about 100%.

Expression and/or activity of a sporulation gene or production of aB. anthracis spore or spore component, can be assessed by, methods known in the art. For instance, expression or activity can be assessed, by e.g., a Congo red agar assay; PCR assays; a spore germination assay; Anthrax Bio-Threat Alerts test strips (Tetracore, Gaithersberg, Md.); antibody-based assays; DNA binding assays; dipicolinic acid assays; and mouse assays (see, e.g., Worsham and Sowers.(l999) Can. J. Microbiol. 45:1-8; Drago, et al. (2002) J. Clin. Microbiol. 40:4399; King, et al. (2003) J. Clin. Microbiol. 41:3454-3455; Hartley and Baeumner (2003) Analyt. Bioanal. Chem. 376:319-327; Carl, et al. (1992) J. Infect. Dis. 165:1145-1148; Zhou, et al. (2002) Proc. Natl. Acad. Sci. USA 99:5241-5246; Bruno and Kiel (1999) Biosens. Bioelectron. 14:457-464; Beverly, et al. (1996) Rapid Commun. Mass Spectrom. 10:455-458; Book, et al. (2001) J. Med. Microbiol. 50:702-711).

Accordingly, in some embodiments, the invention provides an isolated, asporogenic Bacillus anthracis strain that is attenuated for nucleic acid repair. In one embodiment, the strain is defective with respect to SpoIIE. In some embodiments, the strain is also defective with respect to UvrA and/or UvrB (e.g., comprises a mutation in the uvrAB gene). In some embodiments, the strain is defective with respect to UvrC (e.g., comprises a mutation in the uvrC gene). In some embodiments the strain is also defective with respect to RecA. In some embodiments, the nucleic acid of the bacteria of the strain has been modified so that the bacteria are attenuated for proliferation. In some embodiments, the strain comprises one or more mutations in the lef gene and/or the cya gene so that the toxicity of the strain is decreased. A composition, such as a vaccine composition, comprising a bacterium of the strain is also provided. Methods of inducing an immune response in a host comprising administering to the host an effective amount of the composition are also provided. In addition, methods of protecting a host from a disease, comprising administering to the host an effective amount of a composition comprising a bacterium of the strain are also provided.

In some embodiments, the Bacillus anthracis bacteria are defective with respect to one or more toxins, such as lethal factor (LF) or edema factor (EF). In some embodiments, the bacteria comprise a mutation in the lef gene, or the cya gene, or both. In some embodiments, the mutation in the lef gene or the cya gene (or both) reduces the toxicity of the strain. In some embodiments, the toxicity of the strain is reduced by at least about 5%, at least about 10%, at least about 20%, at least about 30%, at least about 50%, or at least about 75%. The present invention also encompasses, without limitation, embodiments of a B. anthracis strain comprising an inhibiting mutation in, or a modification that attenuates expression of, at least one toxin gene, such as the lef gene, the cya gene, or both the lef gene and the cya gene. A “toxin gene” encompasses, but is not limited to, a gene that mediates, e.g., edema, shock, damage to a host cell, pathogenesis, or death to a host cell or organism (see, e.g., Brossier, et al. (2000) Infection Immunity 68:1781-1876). B. anthracis toxin genes include, e.g., the lef gene and cya gene. In some embodiments, the mutation is a point mutation. In other embodiments, the mutation is a deletion. In some embodiments, the cya mutation is K346Q, K353Q, or both K346Q and K353Q. In some embodiments, the lef mutation is H686A. In some embodiments, the K346 codon, K353 codon, or both the K346 and K353 codons are deleted from the cya gene. In some embodiments, the lef mutation comprises a deletion of the H686 codon.

In some embodiments, the B. anthracis bacteria comprise a mutation in the pagA gene. In some embodiments, the mutation in the pagA gene decreases the toxicity of the B. anthracis bacteria (relative to an equivalent B. anthracis without the mutation). In some embodiments, the toxicity of the strain is reduced by at least about 5%, at least about 10%, at least about 20%, at least about 30%, at least about 50%, or at least about 75%. In some embodiments, the mutated pagA gene encodes a dominant-negative form of protective antigen (PA). In some embodiments, the mutation comprises one or more of the following: K397D, D425K, F427A, and a 2beta2-2beta3 loop deletion (residues 302-325). In some embodiments, the mutated pagA gene encodes a mutant protective antigen protein comprising both K397D and D425K mutations.

Toxicity of a modified strain of B. anthracis relative to wild-type and/or relative to the strain without the modification can be assessed using methods known in the art, such as determination of LD₅₀ in an animal model. See, e.g., Section I.B, below.

The present invention encompasses, without limitation, embodiments of a B. anthracis strain comprising an inhibiting mutation in, or a modification that attenuates expression of, at least one sporulation gene or toxin gene, such as the SpoIIE gene; the lef gene; the cya gene; in both the spoIIE gene and the lef gene; in both the spoIIE gene and the cya gene; in both the lef gene and the cya gene; or in all three of the spoIIE gene, lef gene, and cya gene.

In some embodiments, the strain and/or bacterium comprises a polynucleotide encoding an antigen derived from edema factor, lethal factor, or protective antigen (e.g., an antigen comprising a mutation that reduces the toxicity of a Bacillus anthracis strain). In some embodiments, the antigen is derived from protective antigen. For instance, in some embodiments, the antigen is a dominant negative form of protective antigen. In some embodiments, the antigen is a mutant form of edema factor or lethal factor.

In some embodiments, an antigen that is derived from another antigen (or other polypeptide) is a fragment or variant of the antigen (or other polypeptide). In some embodiments, the fragment is at least about 8 amino acids, at least about 12 amino acids in length, at least about 20 amino acids, at least about 50 amino acids, at least about 100 amino acids, or at least about 200 amino acids in length. In some embodiments, the variant comprises a mutation such as, but not limited to, a point mutation or deletion mutant. In some embodiments, the variant is at least about 80% identical, at least about 90% identical, at least about 95% identical, or at least about 98% identical to the antigen (or other polypeptide), or a fragment thereof. In some embodiments, the fragments or variants are antigenic. In some embodiments, the antigenic fragment or variant comprises at least one epitope from the original antigen. In some embodiments, the fragments or variants are less toxic (to a host) when expressed in a Bacillus anthracis than the native protein. A variety of non-limiting examples of mutant antigens that are derived from the antigen lethal factor, edema factor, or protective antigen are described herein.

A variety of algorithms and software packages useful for predicting antigenic regions (including epitopes) within proteins are available to those skilled in the art. For instance, algorthims that can be used to select epitopes that bind to MHC class I and class II molecules are publicly available. For instance, the publicly available “SYFPEITHI” algorithm can be used to predict MHC-binding peptides (Rammensee et al. (1999) Immunogenetics 50:213-9). For other examples of publicly available algorithms, see the following references: Parker et al. (1994) J. Immunol 152:163-75; Singh and Raghava (2001) Bioinformatics 17:1236-1237; Singh and Raghava (2003) Bioinformatics 19:1009-1014; Mallios (2001) Bioinformatics 17:942-8; Nielsen et al. (2004) Bioinformatics 20:1388-97; Donnes et al. (2002) BMC Bioinformatics 3:25; Bhasin, et al. (2004) Vaccine 22:3195-204; Guan et al. (2003) Nucleic Acids Res 31:3621-4; Reche et al. (2002) Hum. Immunol. 63:701-9; Schirle et al. (2001) J. Immunol Methods 257:1-16; Nussbaum et al. (2001) Immunogenetics (2001) 53:87-94; Lu et al. (2000) Cancer Res. 60:5223-7. See also, e.g., Vector NTIS Suite (Informax, Inc, Bethesda, Md.), GCG Wisconsin Package (Accelrys, Inc., San Diego, Ca.), Welling, et al. (1985) FEBS Lett. 188:215-218, Parker, et al. (1986) Biochemistry 25:5425-5432, Van Regenmortel and Pellequer (1994) Pept. Res. 7:224-228, Hopp and Woods (1981) PNAS 78:3824-3828, and Hopp (1993) Pept. Res. 6:183-190. Some of the algorthims or software packages discussed in the references listed above in this paragraph are directed to the prediction of MHC class I and/or class II binding peptides or epitopes, others to identification of proteasomal cleavage sites, and still others to prediction of antigenicity based on hydrophilicity.

Once a candidate antigenic fragment is believed to have been identified, the polynucleotide sequence encoding that sequence can be incorporated into an expression cassette and introduced into B. anthracis. The immunogenicity of the antigenic fragment can then be confirmed by assessing the immune response generated by the bacteria expressing the fragments.

In some embodiments where the bacteria comprise a mutation in, or a modification that attenuates expression of, a toxin gene, the bacteria further comprise a nucleic acid encoding an antigen, wherein the nucleic acid is operably linked to a heterologous promoter. In some embodiments, the antigen is endogenous to Bacillus anthracis or is derived from an antigen endogenous to Bacillus anthracis. In some embodiments, the antigen is, or is derived from, a polypeptide encoded by, e.g., pagA gene; lef gene; cya gene; pagA gene and lef gene; pagA gene and cya gene; lef gene and cya gene; or all three of pagA gene, lef gene, and cya gene. Where the heterologous promoter is operably linked to a nucleic acid encoding an antigen, the heterologous promoter is, in some embodiments, an inducible heterologous promoter.

In some embodiments, the modified Bacillus anthracis bacteria comprise at least one heterologous nucleic acid sequence (i.e., heterologous polynucleotide). In some embodiments, the bacteria comprise more than one heterologous nucleic acid sequence. The heterologous nucleic acid sequences are, in some embodiments, expression cassettes or expression vectors. Optionally, the heterologous nucleic acid sequences have been integrated into the B. anthracis chromosome.

In some embodiments, the modified Bacillus anthracis bacteria comprise an additional copy of a Bacillus anthracis gene encoding an antigen, such as protective antigen. For instance, in some embodiments, an additional copy of the pagA gene is incorporated into the B. anthracis genome. In some embodiments where the bacteria are asporogenic, the antigen encoded by the heterologous nucleic acid is a protein specific to spore formation.

In some embodiments, the modified Bacillus anthracis bacteria comprise a heterologous nucleic acid encoding an antigen derived from a Bacillus anthracis antigen (e.g. lethal factor, edema factor, or protective antigen).

In some embodiments, the modified B. anthracis bacteria comprise a heterologous nucleic acid encoding a mutant protective antigen protein. In some embodiments, the mutant protective antigen protein is a dominant-negative form of protective antigen. In some embodiments, the mutant protective antigen protein comprises one or more of the following mutations: K397D, D425K, F427A, and 2beta2-2beta3 loop deletion (residues 302-325). In some embodiments, the mutant protective antigen protein comprises both K397D and D425K.

Expression cassettes suitable for use in the B. anthracis are known to those of ordinary skill in the art. For instance, it is known that an expression cassette suitable for use in the bacteria typically comprises a polynucleotide encoding a polypeptide (e.g., a heterologous protein) and a promoter operably linked to the protein-encoding polynucleotide. The expression cassette optionally further comprises a polynucleotide encoding a signal peptide sequence, so that the expression cassette comprises a promoter, polynucleotide encoding a signal peptide sequence, and a coding sequence, all operably linked, so that the expression cassette encodes a fusion protein comprising both the signal peptide sequence and the desired polypeptide sequence. In addition, an expression cassette optionally comprises the following elements: (1) prokaryotic promoter; (2) Shine-Dalgarno sequence; (3) a polynucleotide encoding a signal peptide; and, (4) a polynucleotide encoding a polypeptide (such as a heterologous protein).

In some embodiments, the expression cassette may also contain a transcription termination sequence inserted downstream from the C-terminus of the translational stop codon related to the heterologous polypeptide. For instance, a transcription termination sequence may be used in constructs designed for stable integration within the bacterial chromosome. While not required, inclusion of a transcription termination sequence as the final ordered element in a heterologous gene expression cassette may prevent polar effects on the regulation of expression of adjacent genes, due to read-through transcription. Appropriate sequence elements known to those who are skilled in the art that promote either rho-dependent or rho-independent transcription termination can be placed in the heterologous protein expression cassette.

In some embodiments, the promoter used in an expression cassette described herein is a constitutive promoter. In other embodiments, the promoter used in an expression cassette described herein is an inducible promoter. The inducible promoter can be induced by a molecule (e.g., a protein) endogenous to the bacteria in which the expression cassette is to be used. Alternatively, the inducible promoter can be induced by a molecule (e.g. a small molecule or protein) heterologous to the bacteria in which the expression cassette is to be used. A variety of inducible promoters are well-known to those of ordinary skill in the art.

Optionally, at the 3′-end of the promoter is a poly-purine Shine-Dalgarno sequence, the element required for engagement of the 30S ribosomal subunit (via 16S rRNA) to the heterologous gene RNA transcript and initiation of translation. The Shine-Dalgarno sequence has typically the following consensus sequence: 5′-NAGGAGGU-N₅₋₁₀-AUG (start codon)-3′(SEQ ID NO:3). There are variations of the poly-purine Shine-Dalgarno sequence.

Suitable signal sequences are also known to those of ordinary skill in art. For instance, a sequence encoding a secA1 signal peptide can be used in the expression cassette and/or expression vector. One example of a secA1 signal peptide suitable for use in Bacillus anthracis comes from the Pag (Protective Antigen) gene from Bacillus anthracis (MKKRKVLIPLMALSTILVSSTGNLEVIQAEV (SEQ ID NO:4); signal peptidase cleavage site represented by′: IQA′EV (SEQ ID NO:5)).

The expression cassette is optionally contained within an expression vector, such as, but not limited to, a plasmid. In some embodiments, the vector is an integration vector. (In some embodiments, the strain comprises an integrated expression cassette.)

For instance, expression vectors suitable for use in B. anthracis are known to those of ordinary skill in the art. There are a variety of vectors suitable for use as a plasmid construct backbone for assembly of the expression cassettes. A particular plasmid construct backbone is selected based on whether expression of the heterologous gene from the bacterial chromosome or from an extra-chromosomal episome is desired.

Incorporation of the heterologous gene expression cassette into the bacterial chromosome of B. anthracis can be accomplished with an integration vector that contains an expression cassette for a phage integrase that catalyzes sequence-specific integration of the vector into the B. anthracis chromosome. The integrase and attachment site of a B. anthracis phage can be used to derive an integration vector to incorporate desired antigen expression cassettes into the vaccine composition. For instance, the integrase and attachment site from the B. anthracis temperate phage w-beta can be used to derive a B. anthracis specific integration vector (see, e.g., Ivanovics, G. (1962) J. Gen. Microbiol. 28:87-101, incorporated by reference herein in its entirety). Another, less lysogenic phage, w-alpha, has also been reported in the literature (see, e.g., McCloy, E.W. (1951) Studies on a lysogenic Bacillus strain. I. A bacteriophage specific for Bacillus anthracis. J. Hyg. 49:114-125, incorporated by reference herein in its entirety).

Alternatively, incorporation of an expression cassette into the B. anthracis chromosome can be accomplished through allelic exchange methods, known to those skilled in the art. In particular, compositions in which it is desired to not incorporate a gene encoding an antibiotic resistance protein as part of the construct containing the heterologous gene expression cassette, methods of allelic exchange are desirable. For example, the pKSV7 vector (Camilli et. al. Mol. Microbiol. 1993 8,143-157), contains a temperature-sensitive Listeria-derived Gram positive replication origin which is exploited to select for recombinant clones at the non-permissive temperature that represent the pKSV7 plasmid recombined into the Listeria chromosome. The pKSV7 allelic exchange plasmid vector contains a multiple cloning site sequence in order to facilitate construction of plasmids containing the heterologous protein expression cassette, and also a chloramphenicol resistance gene. For insertion into the Bacillus anthracis chromosome, the heterologous antigen expression cassette construct may be flanked by approximately 1 kb of chromosomal DNA sequence that corresponds to the precise location of desired integration. The pKSV7-heterologous protein expression cassette plasmid may be introduced into a desired bacterial strain by electroporation, according to standard methods for electroporation of Gram positive bacteria. A non-limiting example of a method of effecting allelic exchange in B. anthracis using the pKSV7 vector is provided in Example 2 below. This result demonstrates that the pKSV7 vector-based technique of allelic exchange can be used to effect genetic modification in Bacillus species, which, like Listeria, are low G+C content organisms. In particular, allelic exchange using the pKSV7 vector can be used in strains of B. anthracis to delete or modify DNA repair genes, such as UvrAB, or to add a desired antigen expression cassette at any desired location within the bacterial chromosome.

An isolated Bacillus anthracis strain comprising a heterologous expression cassette comprising a nucleic acid sequence encoding protective antigen (pagA) is provided, wherein the nucleic acid sequence encoding the protective antigen is operably linked to an inducible promoter. In some embodiments, the nucleic acid sequence encoding protective antigen is operably linked to an SOS regulatory sequence, such as an SOS box. In some embodiments, the expression of the protective antigen is induced by treatment of the strain with a nucleic acid targeting compound that modifies the nucleic acid of the strain (see below for further information regarding nucleic acid targeting compounds). For instance, in some embodiments, expression is induced by treatment with a psoralen, such as S-59, and ultraviolet (UVA) light. In some embodiments, the expression cassette is on a plasmid or vector. In some embodiments, the expression cassette is integrated in the genomic DNA. In some embodiments, the expression cassette is produced in the genomic DNA (for instance by insertion of appropriate regulatory sequences so that they are operably linked to paga).

In some embodiments, the protein that is expressed by the nucleic acid sequence on the heterologous expression cassette is a fragment or variant of protective antigen. In some embodiments, the functional fragment or variant is an immunogenic fragment or variant of protective antigen.

Accordingly, an isolated Bacillus anthracis strain comprising a heterologous expression cassette comprising a nucleic acid sequence encoding a mutant protective antigen protein (e.g., a dominant-negative mutant) is also provided, wherein the nucleic acid sequence encoding the protective antigen is operably linked to an inducible promoter.

In some embodiments, the nucleic acid sequence encoding protective antigen (or a mutant protective antigen protein) may be operably linked to an SOS regulatory sequence. In some embodiments, expression of protective antigen is under control of an SOS regulatory sequence. In some embodiments, the strain comprises a mutation that attenuates the ability of the strain to repair its nucleic acid. For instance, in some embodiments, the strain is defective with respect to at least one DNA repair enzyme, such as UvrA, UvrB, or both UvrA and UvrB. In some embodiments, the strain comprises a mutation in the uvrA gene, the uvrB gene, or both the uvrA and uvrB gene. In some embodiments, the strain is defective with respect to UvrC (e.g., comprises a mutation in the uvrC gene). In some embodiments, the strain is defective with respect to RecA. A strain that is defective with respect to RecA can, by way of example, comprise a mutation in the recA gene, comprise a recA gene under control of a repressible promoter, or comprise a temperature sensitive recA gene. In some embodiments, the strain comprises one or more mutations in the lef gene, the cya gene, or both genes that decreases the toxicity of the strain. In some embodiments, the nucleic acid of the bacteria of the strain has been modified so that the bacteria are attenuated for proliferation. In addition, the strain is, in some embodiments, asporogenic (e.g., a spoIIE mutant).

Accordingly, the invention also provides an isolated Bacillus anthracis strain comprising a heterologous expression cassette that expresses protective antigen under the control of an SOS regulatory sequence. In some embodiments, the heterologous expression cassette is integrated into the B. anthracis chromosome. A Lex A repressor site exists upstream of the promoter for the B. anthracis recA and uvrAB genes, which are part of the SOS regulon. The regulatory sequences from these B. anthracis genes or from the corresponding genes in related bacteria such as B. subtilis can be placed on the heterologous expression cassette upstream of the protective antigen coding sequence and a suitable promoter, including, as non-limiting examples, B. anthracis promoters such as Pntr, Pamy, or the PagA promoter. A description of the construction of an exemplary Bacillus anthracis strain that expresses protective antigen under control of the SOS regulatory sequence is provided in the specific example, Example 7, below.

LexA binds to a specific DNA sequence (the “SOS box”) upstream of the genes it regulates, and represses transcription. RecA is one of the genes regulated in this manner. Exposure to DNA damage, e.g., UV light and/or psoralen, activates RecA coprotease activity which then triggers the cleavage of LexA. Once LexA is cleaved, LexA is released from its binding sites, where this release results in increased expression of the regulated genes. These regulated genes include recA, as mentioned above, as well as LexA, and 20-40 other genes. In other words, the SOS genes are regulated by two proteins, LexA and RecA, which themselves are products of SOS-regulated genes. A number of SOS boxes have been identified, where these SOS boxes comprise imperfect pallindromic sequences (see, e.g., Davis, et al. (2002) J. Bacteriol. 184:3287-3295; Movahedzadeh, et al. (1997) Microbiology 143:929-936; Winterling, et al. (1997) J. Bacteriol. 179:1698-1703; Winterling, et al. (1998) J. Bacteriol. 180:2201-2211; Cheo, et al. (1993) J. Bacteriol. 175:5907-5915; Campoy, et al. (2002) Microbiology 148:3583-3597; Lovett, et al. (1993) J. Bacteriol. 175:6842-6849; Loved, et al. (1994) 176:4914-4923; Microbiology 143:885-890; Miller, et al. (1996) J. Biol. Chem. 271:33502-33508). The reagents and methods of the present invention encompass regulatory sequences, e.g., imperfect palindromes, associated with or operably linked with various SOS response genes of B. anthracis, including the B. anthracis recA gene. Provided are methods of engineering an SOS box sequence, such as the following imperfect palindrome (a putative SOS box), or an imperfect palindrome associated with other B. anthracis SOS genes, to be operably linked with the recA gene, and/or with a nucleic acid such as the pagA gene, that encodes an antigen. The present invention further contemplates SOS response elements that are perfect palindromes, that overlap translation start sites, and that do not contain a palindrome. The present invention further contemplates the use of SOS response elements from other bacteria, especially other Bacillus bacteria, for use in the modified Bacillus anthracis bacteria of the present invention.

The present invention comprises nucleic acids, and methods, that utilize an imperfect palindrome that occurs at about 64 base pairs (bp) upstream of the start codon of recA coding sequence in Bacillus anthracis (GenBank Acc. No. NC_{—}007530). This palindrome appears to be an SOS box. This palindrome (26 bp long) resides at nucleotide 3,591,817 to 3,591,843, of the B. anthracis genome (NC_{—}007530). Functioning of this 26 bp imperfect palindrome, and variations of this palindromic sequence, is assessed by measuring expression of a nucleic acid encoding a reporter polypeptide, e.g., beta-galactosidase, where the 26 bp imperfect palindrome resides upstream (5-prime to) the nucleic acid encoding the reporter polypeptide. The regulatory function of the 26 bp imperfect palindrome, or of another B. anthracis-derived palindromic sequence, or of a B. subtilis-derived palindromic sequence, is determined using plasmids, where a B. anthracis strain harbors the plasmid. The ability of the putative regulatory sequence to increase expression of the reporter gene in response to an effector of DNA damage such as treatment with UV-light and/or psoralen that triggers the SOS response can be determined by measuring protein expression levels of the reporter polypeptide in the presence and absence of such an effector of DNA damage and comparing the levels of expression using methods standard in the art.

In some embodiments, UV-light and/or psoralen treatment, or another effector of DNA damage stimulates at least a 10% increase in expression of the reporter polypeptide, at least a 20% increase, at least a 30% increase, at least a 40% increase, at least a 50% increase, most at least a 60% increase, at least a 70% increase, at least an 80% increase, or at least a 90% increase, where expression can be of a nucleic acid encoding, e.g., a reporter polypeptide, or an antigen derived from B. anthracis such as protective antigen (PA). In some embodiments, treatment stimulates at least about a 2-fold increase or treatment stimulates at least about a 10-fold increase. In some embodiments, treatment stimulates at least about a 20-fold increase, at least about a 50-fold increase, at least about a 100-fold increase, at least about a 200-fold increase, or at least about a 1000-fold increase, or more, where expression can be of a nucleic acid encoding, e.g., a reporter polypeptide, or an antigen derived from B. anthracis such as protective antigen (PA).

The present invention further provides an additional sequence appearing to be SOS box residing just upstream of the LexA coding region, at nucleotide 3,453,870 to 3,453,883 of GenBank Acc. No. NC_{—}007530 (Bacillus anthracis). The sequence is: ATGTTTTTTCACAT (SEQ ID NO:46). The LexA coding region in Bacillus anthracis is nucleotides 3,453,933 to 3,454,553. The LexA gene contains an SOS response element (Dullaghan et al. (2002) Microbiology 148:3609-3615). The present invention also provides a consensus sequence ATGTNNNNNNACAT (SEQ ID NO:47) for use in Bacillus anthracis.

Also provided is another sequence which appears to be an SOS box in Bacillus anthracis, located just upstream of the ruvA gene. The palindromic region occurs at nucleotide 4,228,545 to 4,228,559, and has the structure: AAATGTTCTCCATTT (SEQ ID NO:48). The palindromic region occurs just upstream of the coding region of the ruvA gene. The Bacillus anthracis gene coding region is located at nucleotides complement to 4,227,852 to 4,228,469 to GenBank Acc. No. NC_{—}007530. RuvA is an SOS response gene (Brooks et al. (2001) J. Bacteriol. 183:4459-4467). The present invention provides a consensus sequence: AAATGNNNNNCATTT (SEQ ID NO:49) for use in Bacillus anthracis.

The putative SOS boxes provided herein can be operably linked to promoter and a recA gene or an antigen coding sequence to place an antigen coding sequence, such as a nucleic acid encoding protective antigen, or another gene, such as a recA gene, under inducible control.

The present invention further contemplates identifying additional regulatory regions in the B. anthracis genome using, e.g., visual inspection and/or available algorithms. Use of algorithms has revealed regulatory motifs in the B. subtilis genome that are upstream of known regulated E. coli genes, e.g., LexA, Crp, and ArcA in B. subtilis (see, e.g., McGuire, et al. (2000) Genome Res. 10:744-757).

In addition to providing regulatory sequences sensitive to DNA damage, the present invention provides regulatory sequences sensitive to, e.g., temperature, pH, osmotic changes, or alterations in the concentrations of oxygen, ions, or metabolites (see, e.g., Repoila and Gottesman (2003) J. Bacteriol. 185:6609-6614; Schofield, et al. (2003) Appl. Environ. Microbiol. 69:3385-3392; Hanna, et al. (2001) J. Bact. 183:5964-5973; Deuerling, et al. (1995) J. Bacteriol. 177:4105-4112). One or more of these regulatory sequences can be used to reduce or inhibit expression of a B. anthracis antigen or B. anthracis derived antigen during growth of the B. anthracis, but to stimulate or allow expression at a later time, e.g., during treatment with a DNA damaging agent or prior to administration to a human or animal subject.

A “heterologous promoter” which is operably linked to a specific gene or nucleic acid encompasses a promoter that is not normally operably linked in nature with that specific gene or nucleic acid, that is, a promoter that is not operably linked with that specific gene or nucleic acid in a parental bacterial strain or in a wild type bacterial strain. The heterologous promoter can be from the same bacterium, in other words, copied or duplicated from a promoter operably linked to a gene other than the above-identified specific gene or nucleic acid. In another embodiment, the heterologous promoter can be derived from a promoter from another species. In another embodiment, the heterologous promoter can be derived from a promoter from another strain of bacteria, so long as the promoter in the second strain is operably linked to a different gene than in the first strain, or the promoter in the second strain differs in sequence from the corresponding promoter in the first strain. The sequence of the heterologous promoter can be altered from that of a naturally occurring promoter, or it can be substantially or completely different from the sequence of any naturally occurring promoter.

The present invention further encompasses a B. anthracis strain comprising a nucleic acid encoding an antigen, wherein the nucleic acid is operably linked to a heterologous promoter. The antigen is, or is derived from, a polypeptide encoded by, e.g.,pagA gene; lef gene; cya gene; pagA gene and lef gene; pagA gene and cya gene; lef gene and cya gene; or all three of pagA gene, lef gene, and cya gene. In some embodiments, the antigen is protective antigen. Where the heterologous promoter is operably linked a nucleic acid encoding an antigen, the heterologous promoter can be one that is an inducible heterologous promoter. In some embodiments, the antigen encoded by the nucleic acid is full-length and/or the native sequence. In some other embodiments, the antigen is a variant and/or a fragment of the native antigen sequence. Software for determining, e.g., antigenic fragments, are available (see, e.g., Vector NTI® Suite (Informax, Inc., Bethesda, Md.; Welling, et al. (1985) FEBS Lett. 188:215-218; Parker, et al. (1986) Biochemistry 25:5425-5432). In some embodiments the polynucleotide encoding the antigen comprises a mutation (e.g., a mutation which decreases the toxicity of the antigen). In some embodiments, the encoded antigen is mutant lethal factor, mutant edema factor, or mutant protective antigen. In some embodiments, the mutant lethal factor comprises a H686A or Δ686 mutation. In some embodiments, the mutant edema factor comprises a K346Q, K353Q, K346Q/K353Q, Δ346, Δ353, or Δ346/Δ353 mutation. In some embodiments, the mutant protective antigen is a dominant-negative form of protective antigen. In some embodiments, the mutant protective antigen comprises a mutation such as K397D, D425K, F427A, K397D/D425K, or a 2beta2-2beta3 loop deletion (residues 302-325).

In some embodiments, the inducible promoter is inducible by DNA damage. In some embodiments, the inducible promoter is inducible by one or more of the following: ultraviolet light; a nucleic acid cross-linking compound; ultraviolet light and a nucleic acid cross-linking compound; an SOS regulatory pathway; and/or a change or shift in temperature. In some embodiments, the inducible promoter (and the nucleic acid encoding the antigen) is operably linked to an SOS regulatory sequence, such as an SOS box.

In some embodiments, the modified strains of B. anthracis comprise heterologous expression cassettes which encode proteins such as cytolysins. Preferably, expression of the proteins enhances the potency of the immune response to the vaccine containing the bacteria upon administration to an animal. For instance, the modified bacteria of the invention optionally comprise heterologous nucleic acids such as expression cassettes or expression vectors that encode cytolysin. The modified bacteria of the invention optionally comprise heterologous nucleic acids, such as expression cassettes or expression vectors, which encode cytolysins. The heterologous cytolysin expressed by the B. anthracis strain in the vaccine is optionally Listeriolysin O (LLO), Streptolysin, or Perfringolysin, or a mutant version of Listeriolysin O (LLO), Streptolysin, or Perfringolysin.

For instance, the vaccine compositions of the present invention are optionally enhanced by the expression and secretion of Listeriolysin O (LLO), the cholesterol-dependent, pore-forming cytolysin from Listeria monocytogenes, by the B. anthracis strains within the vaccine. LLO is a critical virulence factor from Listeria because its expression in the phagolysosome allows Listeria to escape into the host cell cytosol. Importantly, it has been shown that expression of LLO by other microorganisms, such as Bacillus subtilis (Bielecki et al, Nature. 1990 345:175-6), E. coli (Higgins et al, Mol Microbiol. 1999 31:1631 - 1641), or Mycobacterium bovis BCG (Conradt et al, Microbes Infect. 1999 1:753-764), allows these organisms or their protein antigens to enter the cytosol. This leads to improved antigen presentation via the MHC class I pathway and subsequent generation of CD8+ T cell responses.

In some embodiments, the LLO protein expressed by the bacteria is an LLO fusion protein that comprises a signal sequence, allowing it to be secreted from the intact bacteria. In this mode, the whole bacteria can gain access to the host cell cytosol. In some alternative embodiments, LLO protein that is expressed by the bacteria does not comprise a signal sequence, and the LLO protein is expressed and accumulated inside the bacteria without secretion. In this case, degradation and rupture of the bacteria within the phagolysosome ultimately leads to the release of proteins and/or antigens into the cytosol.

In some embodiments, the heterologous cytolysin that is expressed is a naturally occurring cytolysin. In other embodiments, the cytolysin that is expressed is a mutant form of the naturally occurring cytolysin. In some cases, the mutant cytolysin is more active than the naturally occurring cytolysin.

For instance, mutant forms of LLO that are more active than the native protein at neutral pH have been isolated from Listeria and characterized (Glomski et al., Infect Immun. 2003 71: 6754-6765). These mutant LLO proteins retain activity in the host cell cytosol and are thus cytotoxic to the host cell. The primary stimulus by which wild-type LLO activity is regulated is pH, which differs between the phagolysosome and the cytosol. Normally, LLO is active in the acidic environment of the phagolysosome, but is significantly less active in the cytosol. This enables Listeria to replicate and survive in the infected host cell long enough to infect adjacent cells by direct cell-to-cell spread. The increased activity of mutant LLO proteins in the neutral pH environment of the cytosol leads to premature host cell death and an enhanced immune response, including an anti-listerial response. In the context of the vaccine compositions described here, mutant cytolysins, such as the alternative LLO proteins described here, can be useful in non-Listerial species as a way of augmenting CD8+ T cell activation by promoting MHC class I antigen processing.

Thus, in some embodiments, the modified B. anthracis strains used in the vaccine compositions comprise a heterologous expression cassette which encodes a mutant LLO protein.

In some embodiments, at least one sequence in the expression cassette and/or vector contained within the modified B. anthracis is codon-optimized for expression in B. anthracis, as described, for instance, in the U.S. patent application Ser. No. 10/883,599, filed Jun. 30, 2004, incorporated by reference herein in its entirety. In addition, codon optimization of sequences to be expressed in bacteria and suitable signal sequences are described in U.S. Ser. No. 60/532,598, U.S. Ser. No. 60/556,744, U.S. Ser. No. 60/616,750, and U.S. Ser. No. 11/021,441, each of which is incorporated by reference herein in its entirety.

B. Nucleic Acid Modification to Attenuate Proliferation

The invention further provides B. anthracis strains and/or bacteria, wherein the nucleic acid of the strains or bacteria are modified to attenuate the strains and/or bacteria for proliferation. For instance, in some embodiments, the modified B. anthracis strains of the invention which are attenuated for nucleic acid repair, are asporogenic, and/or express protective antigen under inducible control, are further modified to attenuate proliferation. In some embodiments, the modified B. anthracis strains have been modified by reaction with a nucleic acid targeting compound (also referred to herein as a “nucleic acid targeted compound”) that reacts directly with the nucleic acid of the B. anthracis so that the bacteria are attenuated for proliferation.

Methods of attenuating the proliferation of microbes such as Bacillus anthracis by reaction with nucleic acid targeting compounds are described in U.S. Provisional Application No. 60/446,051, filed Feb. 6, 2003; U.S. Provisional Application No. 60/449,153, filed Feb. 21, 2003; U.S. Provisional Application No. 60/490,089, filed Jul. 24, 2003; U.S. Provisional Application No. 60/511,869, filed Oct. 15, 2003; U.S. Ser. No. 10/773,618, filed Feb. 6, 2004 (U.S. Patent Publication No. 2004/0197343 Al); and in U.S. patent application Ser. No. 10/883,599, filed Jun. 30, 2004, the contents of each of which are incorporated by reference herein in their entirety.

In some embodiments, the nucleic acid of the Bacillus anthracis bacteria described herein has been modified so that the bacteria are attenuated for proliferation. In some embodiments, the nucleic acid of the bacterium has been modified by reaction with a nucleic acid targeted compound that reacts directly with the nucleic acid so that the bacterium is attenuated for proliferation. In some embodiments, the nucleic-acid targeted compound is a nucleic acid alkylator, such as beta-alanine, N-(acridin-9-yl), 2-[bis(2-chloroethyl)amino]ethyl ester. In some embodiments, the nucleic acid targeted compound is activated by irradiation, such as UVA irradiation. In some embodiments, the nucleic acid targeted compound is 4′-(4-amino-2-oxa)butyl-4,5′,8-trimethylpsoralen (“S-59”).

In some embodiments, the nucleic acid of the bacteria and/or strain is modified so that the proliferation of the bacterium is attenuated. In some embodiments, the bacterial gene expression is substantially unaffected. In some embodiments, the bacterial gene expression is substantially unaffected so that an antigen is expressed at a level sufficient to stimulate an immune response to the bacteria upon administration of the bacteria to an individual. In some embodiments, the bacterial nucleic acid is modified by a method selected from the group consisting of exposing the bacteria to radiation and/or reacting the bacteria with a nucleic acid targeted compound that causes the modification of the bacterial nucleic acid. In a preferred embodiment, the bacterial nucleic acid is modified by reacting the bacteria with a nucleic acid targeted compound that reacts directly with the nucleic acid. In one embodiment, the nucleic acid targeted compound is targeted to the nucleic acid by a mode selected from the group consisting of intercalation, minor groove binding, major groove binding, electrostatic binding, and sequence-specific binding. In one embodiment, the nucleic acid targeted compound comprises a nucleic acid alkylator. In a preferred embodiment, the nucleic acid targeted compound is beta-alanine, N-(acridin-9-yl), 2-[bis(2-chloroethyl)amino]ethyl ester. In one embodiment, the nucleic acid targeted compound that reacts directly with the nucleic acid reacts upon activation of the compound by irradiation, preferably by UVA irradiation. In one embodiment, the nucleic acid targeted compound activated by UVA irradiation is a psoralen. In a preferred embodiment, the psoralen is 4′-(4-amino-2-oxa)butyl-4,5′,8-trimethylpsoralen. In one embodiment, the nucleic acid targeted compound indirectly causes the modification of the nucleic acid. In one embodiment, the nucleic acid targeted compound indirectly causes modification upon activation by irradiation, preferably by UVA irradiation.

In some embodiments, the modification of the nucleic acid in the bacterium and/or strain is direct. In other embodiments, the modification is indirect. In some embodiments, the modification is not a genetic modification. In some embodiments, the modification that attenuates the bacteria and/or strain for proliferation comprises breakage of strands of the DNA or cross-linking of the genomic DNA. In some embodiments, the modification constitutes DNA damage.

In some embodiments, the desired extent of modification is such that replication of the bacterium's genome is significantly attenuated while the production of proteins remains sufficiently active (i.e. the bacterium is metabolically active). It is to be understood that whatever the nature of the modification, the level of modification can be represented in terms of the number of modifications on average per base pair of the bacterial genome. For example, if the modification is due to covalent binding of a compound to the nucleic acid (adducts), the modification can be represented in terms of the average number of base pairs between adducts. In some embodiments, the bacteria of the invention can be modified to levels of about 1 modification per 10³-10⁸ base pairs, about 1 modification per 10⁴-10⁸ base pairs, about 1 modification per 10⁴-10⁷, about 1 modification per 10⁵-10⁷, or about 1 modification per 10⁵-10⁶ base pairs. In one embodiment, the level of modification is adjusted to the minimum amount required to block DNA replication in the bacterial population, such that the population shows no observable proliferation, while maintaining sufficient activity of transcription and translation of individual genes (i.e. maintains some metabolic activity) to achieve a safe and effective vaccine.

In some embodiments, the genomic DNA of the modified bacteria comprise at least one covalently linked nucleic acid targeting or cross-linking compound. In some embodiments, the genomic DNA comprises at most ten, often comprises at most about 20, more often comprises at most about 100, most often comprises at most about 200, generally comprises at most about 500, more generally comprises at most about 1000, most generally comprises at most about 2000, normally comprises at most about 5000, more normally comprises at most about 10,000, most normally comprises at most about 20,000, typically comprises at most about 50,000, more typically comprises at most about 100,000, most typically contains at most about 500,000, customarily contains at most about 1,000,000, more customarily contains at most about 2,000,000, or most customarily contains at most about 5,000,000, covalently linked nucleic acid targeting or cross-linking compounds. In one embodiment, the covalently linked nucleic acid targeting or cross-linking compound prevents or inhibits B. anthracis proliferation. In some embodiments, the present invention provides a B. anthracis wherein the genomic DNA is linked to at least one, often at least about ten, more often at least about 20, most often at least about 100, usually at least about 200, more usually at least about 1000, most usually at least about 2000, and conventionally at least about 10,000 covalently linked nucleic acid targeting or cross-linking compounds.

1. Attenuation of B. anthracis Proliferation

In some embodiments, the present invention also involves the modification of bacterial nucleic acid in order to attenuate replication (i.e., proliferation) of the bacteria. (The terms replication and proliferation are used interchangeably herein.) This attenuation in replication can be used to increase the level of safety upon administration of the bacteria to individuals. The ability of a bacterium to proliferate can be measured by culturing a population of bacteria under conditions that provide normal growth. The normal growth of a population of bacteria is considered to be the growth of bacteria having no modifications to the nucleic acid of the bacteria. The modification of the bacterial genome will result in some attenuation so that the bacteria will not undergo normal growth. Attenuation of the replication of the bacteria can be measured as a reduction in the number of colony forming units (CFU). A stock solution of the bacterial colony is serially diluted until the number of colony forming units can be easily measured (e.g. 50-500 CFU, on a 100 mM agar Media containing plate). Typically, dilutions are 10-fold and the number of colonies counted for one or more of the diluted samples is used to estimate the log titer of the sample. For example, an aliquot of diluted bacterial stock is plated on growth media and the resulting colonies are counted. The colony forming units per mL (CFU/mL) of the dilution is calculated, and the colony forming units per mL of the original stock (known as the titer) is calculated from the dilution. The log number is known as the log titer. As an example, 24 colony forming units on plating a 0.2 mL aliquot of a 1×10⁵ dilution gives a 1.2×10⁷ titer, or 7.08 log titer stock. The attenuation can be measured as the comparison of bacterial titer prior to modification of the bacterial nucleic acid to that after modification of the bacterial nucleic acid. The log of the ratio of the titer of unmodified bacteria to the titer of bacteria after modification represents the log attenuation (or simply the difference in log titer of the two). For example, if an unmodified bacterial titer measures 1.2×10⁷ and a modified bacterial titer measures 4.3×10², the resulting level of attenuation is 4.45 log. For embodiments of the invention, the desired amount of attenuation can range from a two-fold reduction to much greater levels of attenuation, including a level where essentially no proliferation is observed, depending on the desired level of safety and the intended application of the bacteria. A two-fold attenuation in replication would be observed if for a given dilution, there are half as many in the population of bacteria where the nucleic acid is modified as there are in an unmodified population of the bacteria (about 0.3 log attenuation). In some embodiments, the attenuation in proliferation is at least about 0.3 log, about 1 log, about 2 log, about 3 log, about 4 log about 5 log, about 6 log, or at least about 8 log. In some embodiments, the attenuation is in the range of about 0.3 to >10 log, about 2 to >10 log, about 4 to >10log, about 6 to >10 log, about 0.3-8 log, about 0.3-7 log, about 0.3-6 log, about 0.3-5 log, about 0.3-4 log, about 0.3-3 log, about 0.3-2 log, about 0.3-1 log, about 1-5 log, or about 2-5 log. In some embodiments, the attenuation is in the range of about 1 to >10 log, 1-8 log, 1-6 log, also about 2-6 log, also about 2-5 log, also about 3-5 log. In one embodiment of the invention, the attenuation results in essentially complete inactivation (e.g. where no colonies are observed to the limit of detection), wherein the bacterial gene expression is sufficiently active. Such a population of bacteria can be achieved by titrating the concentration of the agent used to modify the bacterial nucleic acid to find the lowest concentration at which no colonies or plaques are observed at the limit of detection.

In some embodiments, the modified bacteria are substantially unable to proliferate. In some embodiments, the ability to proliferate may have been reduced by at least about 75%, at least about 90%, at least about 95%, at least about 99%, or 100%, relative to the bacteria without the modification. In some embodiments, the modified bacteria are unable to proliferate.

In some embodiments, the modified bacteria are substantially unable to form colonies. In some embodiments, the ability to form colonies may have been reduced by at least about 75%, at least about 90%, at least about 95%, at least about 99%, or 100%, relative to the bacteria without the modification. In some embodiments, the modified bacteria are unable to form colonies.

It is also possible to assess the attenuation in terms of biological effects of the B. anthracis. For example, the pathogenicity of a strain can be assessed by measurement of the median lethality (LD₅₀) in mice or other vertebrates. The LD₅₀ is the amount (e.g. CFU or number of organisms in the case of non-proliferating bacteria) of bacteria injected into the vertebrate that would result in the death of half of the population of the vertebrate. The LD₅₀ values can be compared for modified and unmodified bacteria as a measure of the amount of attenuation. For example, if an unmodified population of bacteria has an LD₅₀ of 10³ bacteria and the population of bacteria in which the nucleic acid has been modified has an LD₅₀ of 10⁵ bacteria, the bacteria has been attenuated so that its LD₅₀ is increased 100-fold, or by 2 log. In some embodiments, the LD₅₀ is 2-fold to 1000-fold higher. In some embodiments, an attenuated strain is used that already has a relatively high LD₅₀. In such cases, the increase in LD₅₀ of the modified bacteria is limited by how much material can be infused without causing harm. For example, the LD₅₀ of a heat killed organism would not be much higher than about 1-5×10⁹ simply because of the loading of biological material into the mice and/or the inflammatory reaction to the bacterial wall components. The degree of attenuation may also be measured qualitatively by other biological effects, such as the extent of tissue pathology or serum liver enzyme levels. Typically, alanine aminotransferase (ALT), aspartate aminotransferase (AST), albumin, and billirubin levels in the serum are determined at a clinical laboratory for mice injected with bacteria of the present invention. Comparisons of these effects in mice or other vertebrates would be made for unmodified and modified bacteria as a way to assess the attenuation of the bacteria. In addition to measuring the effects of the bacteria on the tissues, the amount of viable bacteria that can be recovered from infected tissues such as liver or spleen as a function of time could also be used as a measure of attenuation by comparing these values in mice injected with unmodified vs. modified strains of Bacillus anthracis.

2. Protein Expression by B. anthracis Attenuated for Proliferation

In some embodiments, the modification of the nucleic acid of the bacteria, in addition to attenuating proliferation of the bacteria, is controlled so that bacterial gene expression is substantially unaffected. To be substantially unaffected, the bacterial gene expression need not be completely active upon modification of the nucleic acid. It is only necessary that in a population of bacteria in which the nucleic acid is modified to attenuate replication, bacterial gene expression is sufficiently active to provide an adequate level of expression of the desired protein by the bacterium. An adequate level of expression depends to some extent on the intended use of the bacteria. For example, if the bacterium contains a particular antigen that is to be used as a vaccine, adequate expression would be determined as the minimum level of expression that provides an effective protective or therapeutic immune response to the vaccine. The bacterial gene expression can also be assessed by both in vitro and in vivo methods in order to assess whether such a vaccine might provide an effective immune response. In general, a population of bacteria in which the nucleic acid has been modified can be compared to an unmodified population of the bacterium with respect to a particular antigen.

One possibility is to measure the presentation of the antigen of interest by an antigen presenting cell that has been mixed with a population of the bacteria. The bacteria may be mixed with a suitable antigen presenting cell or cell line, for example a dendritic cell, and the antigen presentation by the dendritic cell to a T cell that recognizes the antigen can be measured. If the bacteria are expressing the antigen at a sufficient level, it is processed into peptide fragments by the dendritic cells and presented in the context of MHC class I or class II to CD8+ or CD4+ T cells, respectively. For the purpose of detecting the presented antigen, a T cell clone or T cell line responsive to the particular antigen may be used. The T cell may also be a T cell hybridoma, where the T cell is immortalized by fusion with a cancer cell line. Such T cell hybridomas, T cell clones, or T cell lines can comprise either CD8+ or CD4+ T cells. The antigen presenting cell can present to either CD8+ or CD4+ T cells, depending on the pathway by which the antigens are processed. CD8+ T cells recognize antigens in the context of MHC class I while CD4+ T cells recognize antigens in the context of MHC class II. The T cell is stimulated by the presented antigen through specific recognition by its T cell receptor, resulting in the production of certain proteins, such as IL-2 or interferon-gamma (IFN-gamma), that can be quantitatively measured (for example using an ELISA assay). Alternatively, a hybridoma can be designed to include a reporter gene, such as beta-galactosidase, that is activated upon stimulation of the T cell hybridoma by the presented antigens. The increase in the production of beta-galactosidase can be readily measured by its activity on a substrate, such as chlorophenolred-beta-D-galactopyranoside, which results in a color change. The color change can be directly measured as an indicator of specific antigen presentation. It is also possible to directly measure the expression of a particular protein by bacteria of the present invention. For example, a radioactively labeled amino acid can be added to a cell population and the amount of radioactivity incorporated into a particular protein can be determined. The proteins synthesized by the cell population can be isolated, for example by gel electrophoresis or capillary electrophoresis, identified as the protein of interest, e.g. by binding with an antibody-specific for the protein, and the amount of radioactivity can be quantitatively measured to assess the expression level of the particular protein. Alternatively, the proteins can be expressed without radioactivity and detected by various methods, such as an ELISA assay or by gel electrophoresis and Western blot with detection using an enzyme linked antibody or fluorescently labeled antibody.

While it is possible that the modification of the bacterial nucleic acid reduces the level of protein expression as compared to an unmodified bacterium, it is to be understood that this may still provide an effective vaccine. It is the combination of attenuation of proliferation with adequate protein expression that is important in some embodiments of the invention. The efficacy of a vaccine is generally related to the dose of antigen that can be delivered by the bacterium, and in some instances, some level of active gene expression by the bacteria is necessary. The attenuation of replication of the bacteria may be several log while the bacterial gene expression is still sufficiently maintained. If the same dose of an attenuated bacterium is compared to that of an unmodified bacterium, the resulting antigen expression (as assessed by the methods discussed above) in the attenuated bacteria population is at least about 1%, about 5%, about 10%, about 25%, about 50%, about 75% or at least about 90% of the antigen expression in the unmodified bacteria population. Since there may be several log attenuation in replication, in some embodiments the dose of the modified bacteria may be safely increased by up to several log, resulting in an equivalent or greater amount of the antigen presented by the attenuated bacteria relative to unmodified bacteria upon vaccination.

In some embodiments, gene expression (of native and/or foreign genes) by the modified bacteria is sufficient to induce an immune response to a native or foreign antigen expressed by the bacteria. In some embodiments, gene expression by the modified bacteria is sufficient to induce an immune response to the bacteria. In some embodiments, the immune response is a protective immune response (e.g., immunity to B. anthracis or some degree of protection from developing a disease associated with infection by B. anthracis upon exposure to B. anthracis).

3. Nucleic Acid Modification of B. anthracis Genome

The nucleic acid of a population of bacteria can be modified by a variety of methods to attenuate the bacteria for proliferation.

The nucleic acid of the bacteria can be modified by physical means, e.g. irradiation with ultraviolet light or ionizing radiation. Ionizing radiation, such as x-rays or gamma-rays, may be used to cause single-strand or double-strand breaks in the nucleic acid. Ultraviolet radiation may be used to cause pyrimidine dimers in the nucleic acid. The appropriate dose of radiation is determined by assessing the effects of the radiation on replication and protein expression as detailed above. In some embodiments, radiation is used which causes DNA damage.

The nucleic acid of the bacteria can also be modified by chemical means, e.g. by reaction with a nucleic acid targeted compound (also referred to herein as a “nucleic acid targeting compound”). In some embodiments, the bacteria are treated with a nucleic acid targeted compound that can modify the nucleic acid such that the proliferation of the bacteria is attenuated. In some embodiments, the bacteria are treated with a nucleic acid targeted compound that can modify the nucleic acid such that the proliferation of the bacteria is attenuated, wherein the bacterial population is still able to express a desired protein antigen to a degree sufficient to elicit an immune response. The nucleic acid targeted compound is not limited to a particular mechanism of modifying the nucleic acid. Such compounds modify the nucleic acid either by reacting directly with the nucleic acid (i.e. all or some portion of the compound covalently binds to the nucleic acid), or by indirectly causing the modification of the nucleic acid (e.g. by causing oxygen damage via generation of singlet oxygen or oxygen radicals, by generating radicals of the compound that cause damage, or by other mechanisms of reduction or oxidation of the nucleic acid). Enediynes are an example of a class of compounds that form radical species that result in the cleavage of DNA double strands (Nicolaou et al., Proc. Natl. Acad. Sci. USA, 90:5881-5888 (1993)). Compounds that react directly with the nucleic acid may react upon activation of the compound, for example upon radiation of the compound. Compounds that react indirectly to cause modification of the nucleic acid may require similar activation to generate either an activated species of the compound or to generate some other active species. While not being limited to the means for activation of nucleic acid targeted compounds, one embodiment of the invention includes the use of photoactivated compounds that either react directly with the nucleic acid or that generate a reactive species such as a reactive oxygen species (e.g. singlet oxygen) which then reacts with the nucleic acid.

The nucleic acid targeted compounds preferentially modify nucleic acids without significantly modifying other components of a biological sample. Such compounds provide adequate modification of the nucleic acid without significantly altering or damaging cell membranes, proteins, and lipids. Such compounds may modify these other cell components to some degree that is not significant. These cell components such as cell membranes, proteins and lipids are not significantly altered if their biological function is sufficiently maintained. In the case of treating a bacterium with a nucleic acid targeted compound, the nucleic acid modification is such that the replication of the bacteria is attenuated while the cell membranes, proteins and lipids of the bacteria are essentially unaffected such that bacterial gene expression is active (e.g. the enzymes required for this are not significantly affected), and the surface of the bacteria maintains essentially the same antigenicity as a bacterium that has not been treated with the compound. As a result, such compounds are useful in preparing an inactivated bacterium for use as a vaccine since the proliferation of the bacterium is sufficiently attenuated while maintaining sufficient antigenicity or immunogenicity to be useful as a vaccine. Because the compounds specifically modify nucleic acids, the modification can be controlled to a desired level so that replication is attenuated while maintaining a sufficient level of protein expression. The modification can be controlled by varying the parameters of the reaction, such as compound concentration, reaction media, controlling compound activation factors such as light dose or pH, or controlling compounds that cause oxygen damage by controlling the oxygen concentration (either physically, e.g. by degassing, or chemically, by use of oxygen or radical scavengers). A nucleic acid targeted compound is any compound that has a tendency to preferentially bind nucleic acid, i.e. has a measurable affinity for nucleic acid. Such compounds have a stronger affinity for nucleic acids than for most other components of a biological sample,. especially components such as proteins, enzymes, lipids and membranes. The nucleic acid targeting provides specificity for the modification of nucleic acids without significantly affecting other components of the biological sample, such as the machinery for gene transcription and protein translation.

Compounds can be targeted to nucleic acids in a number of modes. Compounds which bind by any of the following modes or combinations of them are considered nucleic acid targeted compounds. Intercalation, minor groove binding, major groove binding, electrostatic binding (e.g. phosphate backbone binding), and sequence-specific binding (via sequence recognition in the major or minor groove) are all non-covalent modes of binding to nucleic acids. Compounds that include one or more of these modes of binding will have a high affinity for nucleic acids. While the invention is not limited to the following compounds, some examples of compounds having these modes of binding to nucleic acid are as follows: intercalators are exemplified by acridines, acridones, proflavin, acriflavine, actinomycins, anthracyclinones, beta-rhodomycin A, daunamycin, thiaxanthenones, miracil D, anthramycin, mitomycin, echinomycin, quinomycin, triostin, diacridines, ellipticene (including dimers, trimers and analogs), norphilin A, fluorenes and flourenones, fluorenodiamines, quinacrine, benzacridines, phenazines, phenanthradines, phenothiazines, chlorpromazine, phenoxazines, benzothiazoles, xanthenes and thio-xanthenes, anthraquinones, anthrapyrazoles, benzothiopyranoindoles, 3,4-benzpyrene, benzopyrene diol epoxidie, 1-pyrenyloxirane, benzanthracene-5,6-oxide, benzodipyrones, benzothiazoles, quinolones, chloroquine, quinine, phenylquinoline carboxamides, furocoumarins (e.g. psoralens, isopsoralens, and sulfur analogs thereof), ethidium salts, propidium, coralyne, ellipticine cation and derivatives, polycyclic hydrocarbons and their oxirane derivatives, and echinimycin; minor groove binders are exemplified by distamycin, mitomycin, netropsin, other lexitropsins, Hoechst 33258 and other Hoechst dyes, DAPI (4′,6′-dianidine-2-phenylindole), berenil, and triarylmethane dyes; major groove binders are exemplified by aflatoxins; electrostatic binders are exemplified by spermine, spermidine, and other polyamines; and sequence-specific binders are exemplified by nucleic acids or analogues which bind by such sequence-specific interactions as triple helix formation, D-loop formation, and direct base pairing to single stranded targets. Other sequence-specific binding compounds include poly pyrrole compounds, poly pyrrrole imidazole compounds, cyclopropylpyrroloindole compounds and related minor groove binding compounds (Wemmer, Nature Structural Biology, 5:169-171 (1998), Wurtz et al., Chemistry & Biology 7(3):153-161 (2000), Anthoney et al., Am. J. Pharmacogenomics 1:67-81 (2001)).

In addition to targeting nucleic acids, some of the compounds are also able to react with the nucleic acid, resulting in covalent binding to the nucleic acid. Nucleic acid alkylators are a class of compounds that can react covalently with nucleic acid and include, but are not limited to, mustards (e.g. mono or bis haloethylamine groups, and mono haloethylsulfide groups), mustard equivalents (e.g. epoxides, alpha-halo ketones) and mustard intermediates (e.g. aziridiries, aziridiniums and their sulfur analogs), methanesulphonate esters, and nitroso ureas. The nucleic acid alkylators typically react with a nucleophilic group on the nucleic acid. It is the combination of the nucleic acid alkylating activity and the nucleic acid targeting ability of these compounds that gives them the ability to covalently react specifically with nucleic acids, providing the desired modification of the nucleic acid of bacteria for use in the present invention. The specificity of these compounds may be further enhanced by the use of a quencher that will not enter the bacteria. Such a quencher will quench reactions with the surface of the bacteria while still allowing the nucleic acid targeted compounds to react with the bacterial nucleic acid. A discussion of such quenching can be found in US Patent number 6,270,952, the disclosure of which is hereby incorporated by reference. The modification of the bacterial nucleic acid can be controlled by adjusting the compound concentration and reaction conditions. The appropriate concentration and reaction conditions are determined by assessing their effects on replication and protein expression as detailed above. The compounds used in the present invention are effective at concentrations of about 10 pM to 10 mM, also about 100 pM to 1 mM, also about 1 nM to 10 μM, also about 1-500 nM, also about 1-200 nM or about 1-100 nM. A discussion of nucleic acid targeted, nucleic acid reactive compounds for specific reaction with nucleic acids, in particular bacterial nucleic acids, can be found in U.S. Pat. Nos. 6,143,490 and 6,093,725, the disclosures of which are hereby incorporated by reference.

The nucleic acid can be modified by using a nucleic acid targeted compound that requires activation with radiation in order to cause the nucleic acid modification. Such compounds are targeted to nucleic acids as discussed above. These compounds include, but are not limited to, acridines, acridones, anthyrl derivatives, alloxazines (e.g. riboflavin), benzotriazole derivatives, planar aromatic diazo derivatives, planar aromatic cyano derivatives, toluidines, flavines, phenothiazines (e.g. methylene blue), furocoumarins, angelicins, psoralens, sulfur analogs of psoralens, quinolones, quinolines, quinoxalines, napthyridines, fluoroquinolones, anthraquinones, and anthracenes. Many of these compounds are used as DNA photocleavage agents (Da Ros et al., Current Pharmaceutical Design 7:1781 (2001)). While the invention is not limited to the method of activation of the nucleic acid targeted compounds, typically, the compounds can be activated with light of particular wavelengths. The effective wavelength of light depends on the nature of the compound and can range anywhere from approximately 200 to 1200 nm. For some of these compounds, activation causes modification of the nucleic acid without direct binding of the compound to the nucleic acid, for example by generating reactive oxygen species in the vicinity of the nucleic acid. For some of these compounds, activation results in binding of the compound directly to the nucleic acid (i.e. the compound binds covalently). Some of these compounds can react with the nucleic acid to form an interstrand crosslink. Psoralens are an example of a class of compounds that crosslink nucleic acids. These compounds are typically activated with UVA light (320-400 nm). Psoralen compounds for use in the present invention are exemplified in U.S. Pat. Nos. 6,133,460 and 5,593,823, the disclosures of which are hereby incorporated by reference. Again, it is the combination of nucleic acid targeting and the ability to modify the nucleic acid upon activation that provide specific reactivity with nucleic acids. The modification of the bacterial nucleic acid can be controlled by adjusting the compound concentration, reaction conditions and light dose. The appropriate concentration and light dose are determined by assessing their effects on replication and protein expression as detailed above. In addition to compound concentration and level of light exposure, the reaction is affected by the conditions under which the sample is dosed with UVA light. For example, the required overall concentration for irradiating a population of bacteria in a buffered media is going to vary from a population that is cultured in a growth media (e.g. BHI, Tripticase Soy Broth). The photoreaction may be affected by the contents of the growth media, which may interact with the psoralen, thereby requiring a higher overall concentration of the psoralen. In addition, the effective dosing of the bacteria may depend on the growth phase of the organism and the presence or absence of compound during the growth phase. In one embodiment, the population of bacteria comprises growth media during the psoralen UVA treatment. In one embodiment, the psoralen is added to the population of bacteria, the population is cultured to grow the bacteria in the presence of psoralen and growth media, and the UVA treatment is performed at some point in the growth phase of the bacteria. In some embodiments, the population is grown to an OD of about 0.5 to about 1 (about 1×10⁷ to about 1×10⁹ CFU/mL) in the presence of the psoralen prior to irradiation with an appropriate dose of UVA light. In some embodiments, the population is grown up to an OD of about 1 (1×10⁹ CFU/mL) or above in the presence of the psoralen prior to irradiation with an appropriate dose of UVA light. Optionally, the population is grown up to a late log phase of an OD of about 1. In some embodiments, the population is grown up to about 1×10¹⁰ CFU/mL in the presence of psoralen, and then treated with UVA light. In some embodiments, the population is grown up to late log phase in the presence of psoralen and then concentrated to a concentration of up to about 1×10¹⁰ CFU/mL, and then treated with UVA light. Psoralen compounds are effective at concentrations of about 10 pM to 10 mM, also about 100 pM to 1 mM, also about 1 nM to 10 μM, also about 1-500 nM, also about 1-200 nM or about 1-100 nM, with the UVA light dose ranging from about 0.1-100 J/cm², also about 0.1-20 J/cm², or about 0.5-10 J/cm², 0.5-6 J/cm² or about 2-6 J/cm2. In one embodiment, the bacteria are treated in the presence of growth media at psoralen concentrations of about 10 pM to 10 mM, also about 1-5000 nM, also about 1-500 nM, also about 5-500 nM, or about 10-400 nM. In one embodiment, the bacteria treated in the presence of growth media is grown to an OD of 0.5-1 in the presence of psoralen at concentrations of about 10 pM to 10 mM, also about 1-5000 nM, also about 1-500 nM, also about 5-500 nM, or about 10-400 nM. Following the growth to an OD of 0.5-1, the bacteria population is irradiated with UVA light at a dose ranging from about 0.1-100 J/cm², also about 0.1-20 J/cm², or about 0.5-10 J/cm², 0.5-6 J/cm² or about 2-6 J/cm².

The modification of the DNA of the repair deficient (e.g. uvrAB or uvrABC deficient) B. anthracis with psoralen can be controlled by adjusting the compound concentration, reaction conditions and light dose. The appropriate concentration, reaction conditions and light dose are determined by assessing their effects on replication and protein expression as detailed above. The use of repair deficient mutants provides an additional level of control of proliferation while maintaining adequate protein expression such that the parameters of concentration, reaction conditions and light dose can be adjusted over a wider range of conditions to provide a suitable population of bacteria. For example, there is a broader range of nucleic acid modification density over which proliferation can be completely inhibited without significantly affecting protein expression. The minimum level of modification required to completely inhibit repair deficient strains is much less than for non-repair deficient strains. As a result, the modification level can be higher than the minimum level required to stop proliferation (ensuring complete inactivation) yet still be below a level that is detrimental to protein expression. Thus, while the invention is effective for non-repair deficient strains, uvrAB or uvrABC deficient strains provide greater flexibility in preparing a desirable population of bacteria that would be effective as a vaccine. Psoralen compounds are effective at concentrations of about 10 pM to 10 mM, also about 100 pM to 1 mM, also about 1 nM to 10 1M, also about 1-500 nM, also about 1-200 nM or about 1-100 nM, with the UVA light dose ranging from about 0.1-100 J/cm², also about 0.1-20 J/cm², also about 0.5-10 J/cm², or about 0.5-6 J/cm² or about 2-6 J/cm². In one embodiment, the bacterium is treated in the presence of growth media at psoralen concentrations of about 10 pM to 10 mM, also about 1-5000 nM, also about 1-500 nM, also about 5-500 nM, or about 10-400 nM. In one embodiment, the bacterium treated in the presence of growth media is grown to an OD of 0.5-1 in the presence of psoralen at concentrations of about 10 pM to 10 mM, also about 1-5000 nM, also about 1-500 nM, also about 5-500 nM, or about 10-400 nM. Following the growth to an OD of 0.5-1, the bacterial population is irradiated with UVA light at a dose ranging from about 0.1-100 J/cm², also about 0.1-20 J/cm², or about 0.5-10 J/cm², 0.5-6 J/cm² or about 2-6 J/cm².

In order to generate primarily psoralen crosslinks in any B. anthracis bacterium, particularly uvrAB or uvrABC deficient mutant B. anthracis, it is possible to dose the psoralen and UVA light initially to form adducts and follow this with a second dose of UVA light alone to convert some or most of the monoadducts to crosslinks. The psoralen photochemistry is such that absorption of a photon of appropriate energy will first form a monoadduct. Absorption of an additional photon will convert this monoadduct to a crosslink when a furan side monoadduct is appropriately situated in the DNA double helix (Tessman et al., Biochemistry 24:1669-1676 (1985)). The sample can be dosed with a lower UVA dose at a desired concentration of psoralen and the unreacted psoralen can be removed, e.g. by washing, dialysis or ultrafiltration of the bacteria. The bacteria containing psoralen adducts (monoadducts and crosslinks) can be further dosed with UVA light to convert some or most of the monoadducts to crosslinks without resulting in significant additional adducts to the bacteria. This allows for the controlled addition of a low number of psoralen adducts with the initial light dose, then converting a substantial number of any monoadducts to crosslink with the second dose. This provides for modification of the bacterial genome at sufficiently low levels wherein a majority of the adducts formed will be crosslinks. This is particularly effective for blocking replication with uvrAB or uvrABC deficient mutants. In such embodiments, psoralen compounds are effective at concentrations of about 10 pM to 10 mM, also about 100 pM to 1 mM, also about 1-500 nM, also about 1-200 nM or about 1-100 nM, with the UVA light dose ranging from about 0.1-10 J/cm², also about 0.1-2 J/cm², or about 0.5-2 J/cm². Following removal of most of the unreacted psoralen by washing, dialysis or ultrafiltration of the bacteria, the bacteria may be dosed with UVA light ranging from 0.1-100 J/cm², also about 0.1-20 J/cm², or about 0.5-10 J/cm² or about 2-6 J/cm².

In some embodiments, the bacteria are exposed to a psoralen, such as S-59, in a psoralen concentration of about 1 to 200 nM psoralen, about 1 to 150 nM psoralen, or about 5 to 100 nM psoralen. In some embodiments, the bacteria are grown to an optical density (OD) of at least about 1, before irradiation with UVA light. In some embodiments, the bacteria are grown to an optical density of at least about 1.5 or at least about 2, before irradiation with UVA light. In some embodiments, the bacteria are dosed with UVA light ranging from 0.5-10 J/cm² or about 2-8 J/cm².

In some preferred embodiments, the nucleic acid targeted compound used to modify the nucleic acid of the Bacillus anthracis strain is an alkylator such as β-alanine, N-(acridin-9-yl), 2-[bis(2-chloroethyl)amino]ethyl ester. In other preferred embodiments, the nucleic acid targeted compound used to modify the nucleic acid of the Bacillus anthracis strain is a psoralen compound (e.g., 4′-(4-amino-2-oxa)butyl-4,5′,8-trimethylpsoralen, also referred to herein as “S-59”) activated by UVA irradiation.

Additional information regarding the modification of microbes such as Bacillus anthracis can be found in U.S. Provisional Application No. 60/446,051, filed Feb. 6, 2003; U.S. Provisional Application No. 60/449,153, filed Feb. 21, 2003; U.S. Provisional Application No. 60/490,089, filed Jul. 24, 2003; U.S. Provisional Application No. 60/511,869, filed Oct. 15, 2003; U.S. Ser. No. (U.S. Patent Publication No. 2004/0197343 A1), filed Feb. 6, 2004; and U.S. patent application Ser. No. 10/883,599, filed Jun. 30, 2004, both of which are incorporated by reference herein in their entirety.

The increased sensitivity of a B. anthracis uvrAB deletion mutant to psoralen/UVA treatment is illustrated in Example 3, 14, and 17 below.

II. Pharmaceutical Compositions, Immunogenic Compositions, Vaccines, and Formulations

Compositions comprising any of the modified B. anthracis strains described herein are also provided. In some embodiments, the compositions are pharmaceutical compositions. In some embodiments, the compositions are immunogenic compositions. The pharmaceutical compositions optionally comprise a pharmaceutically acceptable carrier or adjuvant. In some embodiments, the compositions are vaccine compositions (i.e., vaccines). The vaccine compositions optionally comprise a pharmaceutically acceptable carrier or adjuvant.

For instance, in some embodiments, the invention provides an immunogenic composition comprising a modified B. anthracis bacterium and/or strain described herein.

In some embodiments, the invention provides a pharmaceutical composition comprising a modified B. anthracis bacterium and/or strain described herein and a pharmaceutically acceptable carrier.

In some embodiments, the invention provides a vaccine comprising a modified B. anthracis bacterium and/or strain described herein. In some embodiments, the vaccine further comprises an adjuvant.

The vaccine compositions of the present invention can be used to stimulate an immune response in an individual. The formulations can be administered to an individual by a variety of administration routes. Methods of administration of such a vaccine composition are known in the art, and include oral, nasal, intraveneous, intradermal, intraperitoneal, intramuscular, intralymphatic and subcutaneous routes of administration, as well any other route that is relevant for infectious disease.

The vaccine compositions may further comprise additional components known in the art to improve the immune response to a vaccine, such as adjuvants, T cell co-stimulatory molecules, or antibodies, such as anti-CTLA4. The invention also includes medicaments comprising the pharmaceutical compositions of the invention. An individual to be treated with such vaccines, is any vertebrate, preferably a mammal, including domestic animals, sport animals, and primates, including humans.

The vaccine is typically administered as a prophylactic. In some alternative embodiments, however, the vaccines may be used as therapeutic vaccines.

Vaccine formulations are known in the art. Known vaccine formulations can include one or more possible additives, such as carriers, preservatives, stabilizers, adjuvants, antibiotics, and other substances. Preservatives, such as thimerosal or 2-phenoxy ethanol, can be added to slow or stop the growth of bacteria or fungi resulting from inadvertent contamination, especially as might occur with vaccine vials intended for multiple uses or doses. Stabilizers, such as lactose or monosodium glutamate (MSG), can be added to stabilize the vaccine formulation against a variety of conditions, such as temperature variations or a freeze-drying process. Adjuvants, such as aluminum hydroxide or aluminum phosphate, are optionally added to increase the ability of the vaccine to trigger, enhance, or prolong an immune response. Additional materials, such as cytokines, chemokines, and bacterial nucleic acid sequences, like CpG, are also potential vaccine adjuvants. Antibiotics, such as neomycin and streptomycin, are optionally added to prevent the potentially harmful growth of germs. Vaccines may also include a suspending fluid such as sterile water or saline. Vaccines may also contain small amounts of residual materials from the manufacturing process, such as cell or bacterial proteins, egg proteins (from vaccines that are produced in eggs), DNA or RNA, or formaldehyde from a toxoiding process. Formulations may be resuspended or diluted in a suitable diluent such as sterile water, saline, isotonic buffered saline (e.g. phosphate buffered to physiological pH), or other suitable diluent.

The modified B. anthracis vaccine (or other composition comprising the modified B. anthracis) is optionally administered to a host in a physiologically acceptable carrier. Optionally, the vaccine formulation further comprises an adjuvant. Useful carriers known to those of ordinary skill in the art include, e.g., citrate-bicarbonate buffer, buffered water, 0.4% saline, and the like.

In some embodiments, the vaccine compositions are prepared as liquid suspensions. In other embodiments, the vaccine compositions comprising the B. anthracis strains are lyophilized (i.e., freeze-dried). The lyophilized preparation can then be combined with a sterile solution (e.g., citrate-bicarbonate buffer, buffered water, 0.4% saline, or the like) prior to administration.

III. Immunogenicity and In Vivo Efficacy

The immunogenicity of a modified (e.g., mutant) B. anthracis bacteria described herein or a composition comprising the modified B. anthracis bacteria can be readily evaluated using a variety of assays known to those skilled in the art. For instance, as an initial step in the evaluation of a potential vaccine candidate, expression of the relevant antigens from the mutant can be assessed, typically by Western blot analysis (see e.g., Example 9, below). Mass spectrometry can also be used to assess expression of the desired proteins by theB. anthracis mutant (Lenz et al., Proc. Natl. Acad. Sci. USA, 100:12432-12437 (2003)). Assays well known to those in the art such as the Elispot assay (see, e.g., Example 10, below) and intracellular cytokine staining assay (ICS) can be used to assess the immunogenicity of the candidate vaccines. T-cell proliferation following immunization also provides an indication of immunogenicity (see, e.g., Example 11, below). Alternatively, the cytokine expression of stimulated spleen cells can be evaluated following immunization (e.g., immunization of a model organism, such as a mouse model) to determine the immunogenicity of the strain or composition (see, e.g., Example 11). In addition, the cytotoxic activity of antigen-specific T cells produced using the B. anthracis can also be assessed, either in vivo or in vitro, using methods familiar to those in the art. The levels of antibodies produced in response to immunization of a mouse model or other host with the B. anthracis can also be assessed to evaluate the immunogenicity of the vaccine (see, e.g., Example 10, below). The ability of the generated antibodies to neutralize a Bacillus anthracis toxin can also be tested (see, e.g., Example 10).

The efficacy of the vaccines can be evaluated in an individual, for example in a mouse. A mouse model is recognized as a model for efficacy in humans and is useful in assessing and defining the vaccines of the present invention. The mouse model is used to demonstrate the potential for the effectiveness of the vaccines in any individual. Vaccines can be evaluated for their ability to provide either a prophylactic or therapeutic effect against a particular disease. For example, in the case of infectious diseases, a population of mice can be vaccinated with a desired amount of the B. anthracis vaccine. The mice can be subsequently infected with the infectious B. anthracis agent and assessed for protection against infection. The progression of the infectious disease can be observed relative to a control population (either non vaccinated or vaccinated with vehicle only). For instance, the protection against spore and lethal toxin afforded by the modified (e.g., mutant) B. anthracis bacterium or a vaccine comprising the modified bacterium can be evaluated using a mouse model system (see, e.g., Example 12, below).

In some embodiments, the present invention provides modified Bacillus anthracis bacteria, and compositions thereof (e.g., vaccine compositions), which are capable of generating an immune response upon administration to a host. In some embodiments, the immune response is a response which protects the host from a disease related to infection by Bacillus anthracis, such as anthrax. In some embodiments, the immune response comprises a CD4+ immune response, a CD8+ immune response, or both a CD4+ and CD8+ immune response. In some embodiments, the immune response comprises an immune response specific to lethal factor (LF), edema factor (EF), protective antigen (PA), capsule, and/or whole bacteria. In some embodiments, the bacteria and/or compositions described are capable of protecting a host against spore and/or lethal toxin challenge.

IV. Methods of Use

A variety of methods of using the modified B. anthracis strains, vaccines, and pharmaceutical compositions described herein are provided by the present invention. For instance, methods of using any of the modified B. anthracis strains, vaccines, and pharmaceutical compositions described herein to induce immune responses and/or to prevent disease (i.e., disease due to infection of the host by B. anthracis) are provided. Methods of using the modified B. anthracis strains to prepare vaccines and other compositions are also provided. The prevention of disease (also referred to herein as protection of a host from disease) provided by the vaccine compositions described herein need not necessarily be complete in order for the vaccine compositions to be useful.

In another aspect, the invention provides a method of protecting a host from a disease comprising administering to the host an effective amount of a compostion comprising a modified Bacillus anthracis bacterium and/or a strain described herein. In some embodiments, the disease is a disease associated with infection of a host by Bacillus anthracis. In some embodiments, the disease is anthrax.

As used herein, the terms “preventing” disease or “protecting a host” from disease (used interchangeably herein) encompass, but are not limited to, one or more of the following: stopping, deferring, hindering, slowing, retarding, and/or postponing the infection by Bacillus anthracis of a host; stopping, deferring, hindering, slowing, retarding, and/or postponing progression of an infection by Bacillus anthracis of a host; stopping, deferring, hindering, slowing, retarding, and/or postponing the onset or progression of a disease associated with infection of a host by Bacillus anthracis ; and stabilizing the progression of a disease associated with infection of a host by Bacillus anthracis.

In some embodiments, the invention provides a method of inducing an immune response in a host to Bacillus anthracis comprising administering to the host an effective amount of a composition comprising a modified Bacillus anthracis bacterium and/or strain described herein. In some embodiments, the immune response to Bacillus anthracis comprises an immune response to one or more Bacillus anthracis antigens (e.g., protective antigen, lethal factor and/or edema factor), capsule, and/or whole bacteria.

In some embodiments, the immune response which is induced by the compositions described herein is a humoral response. In other embodiments, the immune response which is induced is a cellular immune response. In some embodiments, the immune response is B-cell response. In some embodiments, the immune response is a T-cell response (either a CD4+ T-cell response, a CD8+ T-cell response, or both). In some embodiments, the immune response comprises both cellular and humoral immune responses. In some embodiments, the immune response comprises an antigen-specific immune response.

In some embodiments, the invention provides a method of inducing an immune response in a host to Bacillus anthracis comprising administering to the host an effective amount of a composition comprising a bacterium of an asporogenic B. anthracis strain that is attenuated for nucleic acid repair.

The invention also provides a method of protecting a host from a disease, comprising administering to the host an effective amount of a composition comprising a bacterium of an asporogenic B. anthracis strain that is attenuated for nucleic acid repair.

In addition, the invention provides a method of inducing an immune response in a host to Bacillus anthracis comprising administering to the host an effective amount of a composition comprising a bacterium of a B. anthracis strain comprising a heterologous expression cassette comprising a sequence encoding protective antigen, wherein the protective antigen coding sequence is operably linked to an inducible promoter.

The invention further provides a method of protecting a host from a disease, comprising administering to the host an effective amount of a composition comprising a bacterium of a B. anthracis strain comprising a heterologous expression cassette comprising a sequence encoding protective antigen, wherein the protective antigen coding sequence is operably linked to an inducible promoter.

In addition, the invention provides a method of inducing an immune response in a host to Bacillus anthracis comprising administering to the host an effective amount of a composition comprising a bacterium of a B. anthracis strain comprising a heterologous expression cassette that expresses protective antigen under the control of an SOS regulatory sequence.

The invention further provides a method of protecting a host from a disease, comprising administering to the host an effective amount of a composition comprising a bacterium of a B. anthracis strain comprising a heterologous expression cassette that expresses protective antigen under the control of an SOS regulatory sequence.

The invention further provides a method of inducing an immune response in a host to Bacillus anthracis comprising administering to the host an effective amount of a composition comprising a bacterium of a B. anthracis strain that is defective with respect to RecA (e.g., a conditional or repressible recA mutant).

The invention also provides a method of protecting a host from a disease, comprising administering to the host an effective amount of a composition comprising a bacterium of a B. anthracis strain that is defective with respect to RecA (e.g., a conditional or repressible recA mutant).

Furthermore, the invention provides a method of inducing an immune response in a host to Bacillus anthracis comprising administering to the host an effective amount of a composition comprising a bacterium of a B. anthracis strain comprising a temperature sensitive recA gene.

Furthermore, the invention provides a method of inducing an immune response in a host to Bacillus anthracis comprising administering to the host an effective amount of a composition comprising a bacterium of an asporogenic B. anthracis strain attenuated for nucleic acid repair, comprising a temperature sensitive recA gene and a mutation in the uvrAB gene.

The invention also provides a method of protecting a host from a disease, comprising administering to the host an effective amount of a composition comprising a bacterium of a B. anthracis strain comprising a temperature sensitive recA gene.

The invention also provides a method of protecting a host from a disease, comprising administering to the host an effective amount of a composition comprising a bacterium of an asporogenic B. anthracis strain attenuated for nucleic acid repair, comprising a temperature sensitive recA gene and a mutation in the uvrAB gene.

The delivery of the recombinant bacteria, or a composition comprising the strain, may be by any suitable method, such as intradermal, subcutaneous, intraperitoneal, intravenous, intramuscular, intralymphatic, oral or intranasal.

The compositions comprising the recombinant bacteria and an immunostimulatory agent may be administered to a host simultaneously, sequentially or separately. Examples of immunostimulatory agents include, but are not limited to IL-2, IL-12, GMCSF, IL-15, B7.1, B7.2, and B7-DC and IL-14.

The host in the methods described herein, is any vertebrate, preferably a mammal, including domestic animals, sport animals, and primates, including humans. The term “host” is used interchangeably herein with the term “subject.”

The dosage of the pharmaceutical compositions, immunogenic compositions, or vaccines described herein that are given to the host will vary depending on the species of the host, the size of the host, and the condition or disease of the host. The dosage of the compositions will also depend on the frequency of administration of the compositions and the route of administration. The exact dosage is chosen by the individual physician in view of the patient to be treated.

In some embodiments, a single dose of the pharmaceutical composition, immunogenic composition, or vaccine comprising a modified (e.g., mutant) Bacillus anthracis described herein comprises from about 1×10² to about 1×10¹² of the Bacillus anthracis organisms. In some embodiments, a single dose of the composition comprises from about 1×10⁵ to about 1×10¹¹ organisms. In another embodiment, a single dose of the composition or vaccine comprises from about 1×10⁶ to about 1×10¹¹ of the Bacillus anthracis organisms. In still another embodiment, a single dose of the composition or vaccine comprises from about 1×10⁶ to about 1×10¹⁰ of the Bacillus anthracis organisms. In still another embodiment, a single dose of the composition or vaccine comprises from about 1×10⁷ to about 1×10¹⁰ of the Bacillus anthracis organisms. In still another embodiment, a single dose of the composition or vaccine comprises from about 1×10⁷ to about 1×10⁹ of the Bacillus anthracis organisms.

In some embodiments, a single dosage comprises at least about 1×10² Bacillus anthracis organisms. In some embodiments, a single dose of the composition comprises at least about 1×10⁵ organisms. In another embodiment, a single dose of the composition or vaccine comprises at least about 1×10⁶ Bacillus anthracis organisms. In still another embodiment, a single dose of the composition or vaccine comprises at least about 1×10⁷ of the Bacillus anthracis organisms.

In some embodiments, a single dose of the pharmaceutical composition, immunogenic composition, or vaccine comprising a modified (e.g., mutant and/or treated with a nucleic acid targeting compound) Bacillus anthracis described herein comprises from about 1 CFU/kg to about 1×10¹⁰ CFU/kg (CFU=colony forming units). In some embodiments, a single dose of the composition comprises from about 10 CFU/kg to about 1×10⁹ CFU/kg. In another embodiment, a single dose of the composition or vaccine comprises from about 1×10² CFU/kg to about 1×10⁸ CFU/kg. In still another embodiment, a single dose of the composition or vaccine comprises from about 1×10³ CFU/kg to about 1×10⁸ CFU/kg. In still another embodiment, a single dose of the composition or vaccine comprises from about 1×10⁴ CFU/kg to about 1×10⁷ CFU/kg. In some embodiments, a single dose of the composition comprises at least about 1 CFU/kg. In some embodiments, a single dose of the composition comprises at least about 10 CFU/kg. In another embodiment, a single dose of the composition or vaccine comprises at least about 1×10² CFU/kg. In still another embodiment, a single dose of the composition or vaccine comprises at least about 1×10³ CFU/kg. In still another embodiment, a single dose of the composition or vaccine comprises from at least about 1×10⁴ CFU/kg. (In some embodiments, for instance, in some embodiments where the modified bacteria are attenuated for proliferation by a nucleic acid targeting compound, the CFU amounts may refer to CFU of the bacteria prior to the modification with the compound that attenuates the bacteria for proliferation.)

In some embodiments, the proper (i.e., effective) dosage amount for one host, such as human, may be extrapolated from the LD₅₀ data for another host, such as a mouse, using methods known to those in the art.

In some embodiments, multiple adminstrations of the dosage unit are preferred, either in a single day or over the course of a week or month or year or years. In some embodiments, the dosage unit is administered every day for multiple days, or once a week for multiple weeks.

In another aspect, the invention provides the use of any modified Bacillus anthracis bacterium and/or strain described herein in the manufacture of a medicament for inducing an immune response in a host to Bacillus anthracis.

In still another aspect, the invention provides the use of any modified Bacillus anthracis bacterium and/or strain described herein in the manufacture of a medicament for protecting a host against a disease. In some embodiments, the disease is a disease associated with Bacillus anthracis infection (e.g., anthrax).

In addition to use directly in vaccines and other immunogenic compositions, the modified B. anthracis bacteria of the present invention provide reagents and methods for the expression, production, or biosynthesis of, e.g., enzymes and proteins, for use in, for example, industrial, veterinary, medical, and/or diagnostic applications. For instance, in some embodiments, the modified bacteria may be used for the production of protective antigen, an antigen currently often used in current anthrax vaccines.

V. Kits

The invention further provides kits (or articles of manufacture) comprising the modified Bacillus anthracis strains and bacteria of the present invention.

In one aspect, the invention provides a kit comprising both (a) a composition comprising a modified B. anthracis strain and/or bacterium described herein, and (b) instructions for the use of the composition in the prevention or treatment of a disease in a host. In some embodiments, the instructions are on a label. In other embodiments, the instructions are on an insert contained within the kit.

In another aspect, the invention provides a kit comprising both (a) a composition comprising a modified B. anthracis strain and/or bacterium described herein; and (b) instructions for the administration of the composition to a host. In some embodiments, the instructions are on a label. In other embodiments, the instructions are on an insert contained within the kit.

In another aspect, the invention provides a kit comprising both (a) a composition comprising a modified B. anthracis strain and/or bacterium described herein; and (b) instructions for selecting a host to which the composition is to be administered. In some embodiments, the instructions are on a label. In other embodiments, the instructions are on an insert contained within the kit.

In some embodiments of each of the aforementioned aspects, the composition is a vaccine. In some embodiments of each of the aforementioned aspects, the B. anthracis is defective with respect to a DNA repair enzyme. In some embodiments of each of the aforementioned aspects, the nucleic acid of the B. anthracis has been modified by reaction with a nucleic acid targeted compound that reacts directly with the nucleic acid. In some embodiments, the B. anthracis has been S-59/UVA treated.

### EXAMPLES

The following examples are provided to illustrate, but not to limit, the invention.

### Example 1

### Bacterial Vaccines Derived from Nucleotide-excision Repair (NER) Mutants

The examples described herein illustrate the efficacy of vaccine compositions utilizing genomic inactivation through photochemical treatment of the recombinant delivery platform encoding antigens related to infectious and malignant disease. According to this composition, while the genomes are inactivated and cannot separate during replication, the transcriptional profile remains largely intact, thus resulting in antigen expression de novo in the vaccinated individual, and optimal induction of pathogen-specific immune responses, including CD8+ cytotoxic T cells (CTL). Furthermore, by utilizing a vaccine platform in this composition in which the DNA nucleotide excision repair (NER) machinery has been inactivated by any number of means, including by engineered genetic deletion, the sensitivity to photochemical inactivation in these mutants is dramatically increased.

As a result of the requirement of significantly fewer DNA cross-links to inactivate the DNA repair mutants, in the context of the population of bacterial genomes comprising a vaccine dose, the expression of any one gene will not be significantly affected, due to the low level of DNA crosslinking resulting in essentially no interruption of expression, at that given gene.

Thus, the overall utility of gene-based vaccines utilizing bacterial platforms derived from pathogens can be increased dramatically by combining photochemical inactivation with a vector defective in NER. While the inactivated vaccine cannot cause disease, it still retains its efficient ability to induce potent immunity, including T-cell mediated cellular immunity, specific for the vector-expressed heterologous antigens. Furthermore, the uvrAB mutation can be combined with any other attenuating mutation(s), in order to derive a safe and efficacious vaccine platform combining both photochemical and genetic attenuation.

Significantly, these compositions can be used as an approach for deriving a safe and efficacious vaccine derived from a selected bacterial pathogen, in order to protect against challenge with the wild-type pathogen in vaccinated individuals. According to this application, it is not feasible in many cases to derive a safe and efficacious vaccine that is derived from an attenuated viable form of the pathogen, as the possibility for reactivity and disease pathogenesis in particular individuals receiving the vaccine remain high. While subunit or inactivated vaccines related to a selected bacterial pathogen might be safe, on the other hand, these vaccines are often not efficacious because they do not efficiently elicit the breadth, depth, and durability of pathogen-specific immune responses that are required to protect the vaccinated individual against challenge with the wild-type form of the said pathogen. Thus, it is well known in the art that there is a clear need for vaccine compositions that combine safety with an efficient ability to elicit the type of immune responses in vaccinated individuals that are protective.

As such, mutants in the nucleotide-excision repair (NER) pathway of B. anthracis provide a composition that can be used for safe and efficacious vaccines that elicit protection against challenge in immunized individuals with amounts of bacteria that are sufficient to cause disease in non-vaccinated individuals. NER is catalyzed by an ATP-dependent nuclease made of three subunits, known as the ABC excinuclease, and encoded by the genes uvrA, uvrB, and uvrC. Mutations in any one or more than one of the three uvr genes results in cells, including microbes of pathogenic organisms like B. anthracis, extremely sensitive to DNA modification, including photochemical inactivation utilizing psoralens and UVA light.

As an example, mutation of the uvr genes of Bacillus anthracis (B. anthracis), the etiological agent of Anthrax, is provided. The current acellular anthrax vaccines that are licensed for human use are based on sterile culture supernatants of attenuated B. anthracis adsorbed on alum hydroxide (U.S. vaccine), or precipitated with alum phosphate (U.K. vaccine). It is well known that these vaccines are rather weak, requiring at least six immunizations for protection as well as annual boosters.

In the composition described herein, the uvrA, uvrB, or uvrC genes, or any B. anthracis gene involved in NER, alone, or in any combination, is mutated such that a functional form of the protein is not expressed.

As an example, mutation in the uvrA, uvrB, or uvrC genes, or any B. anthracis gene involved in NER, can be performed, for example, by allelic exchange using the pKSV7 vector, as described in Camilli et al., Molecular Microbiology, 8:143-147 (1993). B. anthracis genes involved in NER are identified through a homology search with the genomes of related organisms in whose uvr genes are known. For example, the genome ofB. anthracis, that is, the main chromosome and the two virulence plasmids can be compared with Bacillus subtilis (B. subtilis), a related bacterium from the same genera as B. anthracis. The genomic scaffold representing the main chromosome of the Florida B. anthracis isolate (Read et. al. 2002. Science 296, 2028-2033) has a GenBank accession number of AAACO100000001. B. subtilis has a GenBank accession number of NC_{—}000964. The B. subtilis uvrA gene encompasses nts. 3609064 to 3611997, and the B. subtilis uvrB gene encompasses nts. 3612005-3613990. A BLAST search was performed using the B. subtilis uvrA and uvrB coding sequences against the B. anthracis sequence. This analysis identified a region of 72% sequence identity in the genome of B. anthracis that corresponds to the uvrA and uvrB genes of this organism. TheB. anthracis uvrA gene maps from 226021-228783, and bears 72% sequence homology to the B. subtilis uvrA gene (2082/2867 identical sequence homology alignment). The B. anthracis uvrB gene maps from 228864-230771, and bears 72% sequence homology to the B. subtilis uvrB gene (1401/1925 identical sequence homology alignment). Thus, the B. anthracis uvrAB genes include nts. 226021 to 230771 of the main chromosome of B. anthracis.

Deletion of the B. anthracis uvrAB genes, including nts. 22602.1 to 230771 of the main bacterial chromosome is accomplished according to the methods described in Camilli et al., Molecular Microbiology, 8:143-147 (1993) and as described for Listeria monogenes in Example 7 of U.S. Ser. No. 10/773,618, filed Feb. 6, 2004 (U.S. Patent Publication No. 2004/0197343 A1). Briefly, this region and approximately 1000 bps both upstream and downstream of the B. anthracis genome are amplified by PCR, and subsequently cloned into the pKSV7 allelic exchange plasmid vector. As an alternative, a Bacillus genera-specific or B. anthracis-specific temperature-sensitive (ts) replicon may be substituted for the Listeria ts replicon present in the pKSV7 allelic exchange plasmid vector. Using convenient restriction endonuclease recognition sites mapping specifically within the uvrAB region, any part of the uvrA, uvrB, or all of the uvrAB genes sequence are deleted.

Finally, the allelic exchange plasmid is introduced into B. anthracis and NER mutants are selected as described in Camilli et al., Molecular Microbiology, 8:143-147 (1993) and as described for Listeria monogenes in Example 7 of U.S. Patent Publication No. 2004/0197343 Al. Briefly, bacteria electroporated with the pKSV7-heterologous protein expression cassette plasmid are selected by plating on BHI agar media containing chloramphenicol (10 μ/ml), and incubated at the permissive temperature of 30° C. Single cross-over integration into the bacterial chromosome or deletion of the uvrAB genes from the bacterial chromosome is selected by passaging several individual colonies for multiple generations at the non-permissive temperature of 41° C. in media containing chloramphenicol. Finally, plasmid excision and curing (double cross-over) is achieved by passaging several individual colonies for multiple generations at the permissive temperature of 30° C. in BHI media not containing chloramphenicol. Verification of integration of the heterologous protein expression cassette into the bacteria chromosome is verified by PCR, utilizing a primer pair that amplifies a region defined from within the heterologous protein expression cassette to the bacterial chromosome targeting sequence not contained in the pKSV7 plasmid vector construct, or utilizing a primer pair that hybridizes to the bacterial chromosome beyond the deleted region, and thus deletion of the targeted gene is observed by a PCR amplicon of decreased molecular weight, as compared to the parental strain. Any selected B. anthracis strain can be used as a parent strain for derivation of the NER-defective vaccine, including, for example, the following strains: Ames, Vollum, A1.a/10, A1.b/23, A2/29, A3.a/34, A3.b/57, A4/69, B/80, Δsterne, VN41Δ1, Dames, NNR1Δ1, and DNH 1. An exemplary preparation of the uvrAB deletion mutant of the Bacillus anthracis Sterne strain is described below in Example 2.

Additionally, other attenuating mutations can be incorporated into the genome of the selected NER mutant B. anthracis strain, to enable vaccine compositions combining DNA modification, including photochemical inactivation, with genetic inactivation. Such B. anthracis vaccine compositions are able to induce immune responses against known correlates of anthrax immunity and protection, including lethal factor (LF), edema factor (EF), and protective antigen (PA). Additionally, since the expression profile of the NER mutant vaccine composition is not significantly affected, immune responses against other antigens, including those expressed from the two virulence plasmids pXO1 and pXO2 and the main chromosome are also induced.

The compositions described herein in some embodiments, using B. anthracis NER mutants as a component of vaccine, can be used in a prophylactic immunization against disease caused by cutaneous, gastrointestinal or respiratory infections.

### Example 2

### Construction of a Bacillus anthracis Strene ΔuvrAB.

The allelic exchange methods detailed in Camilli et al., Molecular Micro., 8:143-147 (1993) and as described in U.S. Patent Publication No. 2004/0197343 A1, for alteration of Listeria monogenes were used to modify the Bacillus anthracis Strene strain. The virulence of this strain is attenuated (pXO1⁺, pXO2⁻). All of the TOPO vectors used here were derived from pCR®2.1-TOPO® (Invitrogen, Carlsbad, Calif.).

The uvrAB gene from Bacillus anthracis was identified (Genbank accession number AE017040, Bacillus anthracis Ames strain, section 17 of 18 of the complete genome, uvrAB genes coding sequence: nts. 212613-217471) and a plasmid based on pKSV7 with the uvrAB gene deletion was constructed (pKSV7-dl uvrAB) using Splice Overlap Extension (SOE) PCR and the steps described below:

Primary PCR reactions: Approximately 1000 bps of sequence upstream and downstream from the B. anthracis uvrAB genes 5′ and 3′ ends, respectively, were amplified.

Template: B. anthracis Sterne genomic DNA

Primer pair 1: Amplification of region 1000 bp upstream from 5′ end of uvrB. (Amplicon Size (bps): 1029)

Ba-225099F: 5′-CTGTGCTTTGCGAATGGAAAGAAGC (SEQ ID NO:6) (Tₘ: 74° C.)

Ba-(3′ uvrA-R+) 226109R: 5′-GTTTTCATTCATACACTTAGACAAGCGTTGGCTTTTGCACTTC (SEQ ID NO:7) (Tₘ: 120° C.) (Underlined sequence is complementary to region downstream of uvrA carboxy terminus.) or Ba-226109R: 5′-GACAAGCGTTGGCTTTTGCACTTC (SEQ ID NO:8) (Tₘ: 72° C.).

Primer pair 2: Amplification of region downstream from 3′ end of uvrA. (Amplicon size (bps): 990)

Ba-(3′ uvrA-R+) 230779F: 5′-CAAAAGCCAACGCTTGTCTAAGTGTATGAATGAAAACCGAGTGG (SEQ ID NO:9) (T^{m}: 126° C.) (Underlined sequence is complementary to region upstream of uvrB amino terminus.) or Ba-230779F: 5′-AAGTGTATGAATGAAAACCGAGTGG (SEQ ID NO:10) (Tₘ: 70° C.)

Ba-231769R: 5′-CATATAAAGGTTCCACAATTGCCTTTTC (SEQ ID NO:11) (Tₘ: 76° C.)

Secondary PCR reaction: Fusion of primary PCR amplicons through SOE PCR, taking advantage of complementarity between reverse primer of pair 1 and the forward primer of pair 2. Results in precise deletion of uvrAB coding sequence: nts. 226110-230779=4670 bps.

Template: Cleaned primary PCR reactions

Primer pair: (Amplicon size (bps): 1973)

| | | |
|---|---|---|
| Ba-225118F: | | |
| 5′-GAAGCAGAAATGAAGCCAA | (SEQ ID NO:12) | (Tₘ: 78° C.) |
| TACTCAATC | | |
| Ba-231761R: | | |
| 5′-GGTTCCACAATTGCCTTTT | (SEQ ID NO:13) | (Tₘ: 74° C.) |
| CAATAATC | | |

Construction: Primary PCR reactions (3 temperature cycle) were performed using Vent DNA polymerase (NEB) and Strene strain genomic DNA. Four primary PCR reactions were performed both with and without primers used for splice overlap extension (SOE). (If reactions containing Ba-(3 ′ uvrA-R+) 226109R or Ba-(3′ uvrA-R+) 226109R primers did not yield significant amplicon product, then these primers on amplicons from reactions with Ba-225099F/Ba-226109R or Ba-230779F/Ba-231769R primer pairs were used.) The expected size of anthracis primary amplicons by 1% agarose gel (1029 bps and 990 bps) was verified. The reaction was cleaned with S6 columns (BioRad, Hercules, Calif.) or GeneClean (BIO 101, Irvine, Calif.).

The secondary PCR reaction was performed, utilizing approximately equal amounts of each primary reaction as template (ca., 5 μl) were performed. The expected size of the Listeria amplicon from secondary PCR reaction by 1% agarose gel (1973 bps) was verified.

The anthracis dl uvrAB amplicon was inserted into pCR2.1-Blunt II-TOPO vector. The plasmid pCR2.1-TOPO-dl uvrAB plasmid DNA was digested with KpnI and PstI and gel-purify 2033 bp fragment. The KpnI/PstI 2033 bp fragment was inserted into pKSV7 vector, that had been prepared by digestion with KpnI and PstI and treatment with CIAP (pKSV7-dl uvrAB). The fidelity of dl uvrAB sequence in pKSV7-dl uvrAB was verified.

The uvrAB genes were deleted from B. anthracis Strene by allelic exchange with pKSV7-dl uvrAB plasmid. The plasmid pKSV7-dl uvrAB was introduced into theB. anthracis Sterne strain by electroporation selecting for chloramphenicol resistance. The electroporation was done using a freezing step that significantly increased the frequency of electroporation. B. anthracis culture was grown O/N in 3 ml BHI 0.5% glycerol shaking at 37° C. 0.5 ml culture was transferred to 50 ml BHI 0.5% glycerol (OD₆₀₀=0.1) in 500 ml E-flask. The sample was incubated at 200 rpm 37° C. (or 0.1-0.2 ml to 25 ml BHI 0.5% glycerol in 250 ml flask). At OD₆₀₀=0.6-0.8 (approx 1 hour 45 min), bacteria were collected in 500 ml disposable sterile filter apparatus. The bacteria were washed 3×25 ml each with cold electroporation buffer (1 mM HEPES 10% glycerol pH 7.4). The cells were resuspended in 1/20× original volume (2.45 ml of electroporation buffer for 50 ml culture) and kept on ice. The efficiency of electroporation can be enhanced by freezing the electrocompetent B. anthracis at −80° C. A 0.2 ml suspension of ice-cold or thawed electrocompetent B. anthracis cells were mixed with 1 micrograms (1 to 5 microliters of miniprep) of “very clean” unmethylated plasmid DNA to 0.2 ml cells suspension in a 0.2 cm gap electroporation cuvette (control=no DNA). The sample was then kept on ice for 15 min. The cells were then pulsed at 25 μFD, 200Ω, 2.5 kV (or, alternatively, 0.4 ml cells were pulsed in 0.4 cm cuvette at 400Ω). Time constant was approximately 4-5 msec. Immediately after pulse, 1 ml BGGM (BHI containing 10% glycerol, 0.4% glucose and 10 mM MgCl₂) was added. The cells are transfered to a sterile polypropylene tube and incubated 37° C. 1½hour, shaking. The cells are pelleted, resuspended in 200 microliters BGGM and plated on selective media.

The pKSV7-dl uvrAB was integrated into the B. anthracis chromosome at 410° C. pKSV7-dl uvrAB was allowed to excise and cure at the permissive temperature, resulting in chloramphenicol sensitive colonies. PCR primers were designed to detect the deletion on the chromosome. 20% of the chloramphenicol sensitive colonies harbored the deletion in the B anthracis chromosome. PCR analysis of the uvrAB⁻strain indicated retention of the pXO1 virulence plasmid.

### Example 3

### S-59/UVA Treatment of Bacillus anthracis Sterne Strain with and without uvrAB Deletion.

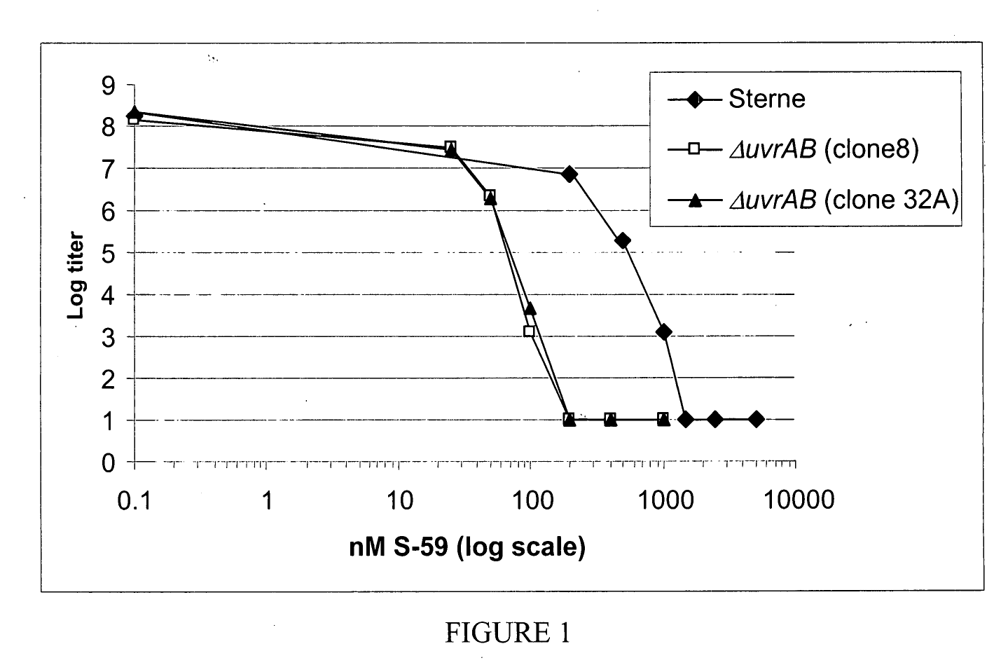

Two uvrAB⁻clones constructed as indicated in Example 2 (clone 8 and clone 32A) were S-59-treated, along with the parent strain, by growing in BHI at 37° C. at 300 rpm to and OD₆₀₀ of 0.3, at which point 50 mL of solution was transferred to a clean flask and S-59 was added to the concentrations indicated in Table 2. These samples were incubated at 37° C. at 300 rpm with vigorous shaking for approximately 1 hour (OD₆₀₀ approximately 1.0, approximately 1×10⁹/mL). A 1 mL aliquot was removed to assess the titer and the remaining was transferred to a 150 mm Petri dish and irradiated at a UVA dose of 6 J/cm² (FX-1019), resulting in a six-log reduction in titer, as compared to the parental strain, as indicated in Table 2, below, and FIG. 1.

TABLE 2
Attenuation of Bacillus anthracis Sterne strain vs. uvrAB⁻ mutant with
psoralen S-59/UVA treatment.

| | Bacterial log titer | | | Log attenuation | | |
|---|---|---|---|---|---|---|
| S-59 | | uvrAB⁻ | | | | uvrAB⁻ |
| nM | Sterne | (1) | uvrAB⁻ (2) | Sterne | uvrAB⁻ (1) | (2) |
| 0 | 8.26 | 8.13 | 8.31 | — | — | — |
| 25 | — | 7.46 | 7.45 | — | 0.67 | 0.86 |
| 50 | — | 6.31 | 6.28 | — | 1.82 | 2.03 |
| 100 | — | 3.11 | 3.68 | — | 5.02 | 4.63 |
| 200 | 6.84 | <1 | <1 | 1.42 | >7.13 | >7.31 |
| 400 | — | <1 | <1 | — | >7.13 | >7.31 |
| 500 | 5.29 | — | — | 2.97 | — | — |
| 1000 | 3.11 | <1 | <1 | 5.15 | >7.13 | >7.31 |
| 1500 | 1 | — | — | >7.26 | — | — |
| 2500 | 1 | — | — | >7.26 | — | — |
| 5000 | 1 | — | — | >7.26 | — | — |

### Example 4

### Asporogenic B. anthracis Vaccine Strains

The spoIIE in-frame deletion. In the following examples, the spoIlE gene mutant is a deletion mutant, while the cya gene mutation is a point mutation and the lef gene mutation is a point mutation.

The SpoIIE region of B. anthracis is identified by homology to the same gene in B. subtilis. In order to isolate an in-frame deletion of B. anthracis spoIIE, the spoIIE gene is first amplified by PCR and cloned it into pCR-Blunt II-TOPO (Invitrogen, Carlsbad, Calif.). Next, most of the spoIIE gene is deleted by using the technique of gene splicing by overlap extension (SOE) (Horton et al., Biotechniques 8:528-35 (1990)). This in-frame deleted spoIIE gene is cloned into the shuttle vector pKSV7, which carries a chloramphenicol-resistance gene and cannot replicate at 42° C. (Smith et al., Biochimie, 74:705-11 (1992)). pKSV7 containing the deleted spoIIE gene is then electroporated into B. anthracis, and cells are grown at 42° C. in the presence of chloramphenicol to select for strains in which the plasmid has integrated by homologous recombination into the spoIIE gene. Further growth at 30° C. without chloramphenicol selection allows excision and loss of the plasmid. Chloramphenicol-sensitive strains should be found at about 1%, and about half of them should contain the deleted spoIIE allele (Camilli et al., (1993)). The presence of the deletion is confirmed by PCR and Southern blot analyses.

An in-frame deletion of the spoIIE gene of B. anthracis was generated by SOE PCR. Briefly, a fragment containing the 5′ region of spoIIE and sequences upstream was amplified with primers Spo2A and Spo2B. Primer Spo2A was: TAACGACCGCGCTCCAAAAG (SEQ ID NO:30). Primer Spo2B was: GGCATATTTTTCTTCACTTTTGCCCACTTTACTCCTCCAAGCTGACC (SEQ ID NO:31). A fragment containing the 3′ region of spoIIE and downstream sequences was amplified with primer spo2C and primer spo2D. Primer spo2C was: GGTCAGCTTGGAGGAGTAAAGTGGGCAAAAGTGAAGAAAAATATGCCG (SEQ ID NO:32). Primer spo2D was: TGCATTCATCCCGTATTGCT (SEQ ID NO:33). The fragments were combined and the overlapping region was annealed, yielding a product that was amplified with primers spo2A and spo2D. This fragment of the spoIIE region contains the putative promoter region of spoIIE and encodes an allele of spoIIE from which amino acids 12-787 are deleted. The construct was cloned into the topo-TA plasmid and the sequence was confirmed. This allele was subcloned into the tempterature sensitive shuttle vector pKSV7, and allelic exchange was performed. The presence of the deletion allele and absence of the wild-type allele was confirmed by PCR and by Southern blotting.

The spoIIE/uvrAB double deletion strain. Starting with the spoIIE deletion strain, an in-frame deletion of the uvrA and uvrB genes is made. Once again, the genes of interest are amplified and and cloned into pCR-Blunt II-TOPO. Then we shall delete most of the uvrA and uvrB genes by the SOE technique. This in-frame deleted uvrAB region is cloned into pKSV7, and the construct is electroporated into the B. anthracis spoIIE deletion strain. Chloramphenicol-resistance is selected at 42° C. in order to select for the integration of the plasmid into the uvrAB region. Growth at 30° C. without drug selection is allowed in order to encourage the growth of segregants that have lost the plasmid. Chloramphenicol-sensitive colonies are picked and tested by PCR for loss of the uvrAB region, and that loss is confirmed by Southern blot analysis.

### Example 5

### A Temperature Sensitive recA Mutant of B. anthracis

To generate a temperature sensitive recA mutant of B. anthracis which grows well at 30° C. and is very sensitive to psoralen at 42° C., a mutation is made in B. anthracis which is analogous to the V246M mutation of the temperature sensitive recA mutant of E. coli, recA44 (Kawashima et al. Mol. Gen. Genet. 193:288-92 (1984)). To make the B. anthracis mutant, the sequence 245KVVKNK250 (SEQ ID NO:14; numbering is according to Kawashima et al. (1984)), which is conserved between E. coli and B. anthracis, is mutated. The V246M mutation of recA44 (corresponding to a V244M mutation in B. anthracis RecA) is introduced into the cloned B. anthracis recA gene by mismatched oligonucleotide mutagenesis, using the Stratagene Quick Change kit (Stratagene, La Jolla, Calif.). The mutations are confirmed by sequence analysis, and the mutated gene is transferred into pKSV7, in order that they can be introduced into the chromosome of B. anthracis spoIIE uvrAB by allelic exchange. Alternatively, the recA gene from the B. anthracis strains is deleted and replaced with the recA44(ts) allele of E. coli. (It is known that B. anthracis recA functions in E. coli (Ko et al., J. Bacteriol 184:3917-22 (2002)).)

In the wild-type E. coli RecA coding sequence SEQ ID NO:50, below, (GenBank Acc. No. V00328), a mutation in the coding sequence that would encode a mutation analogous to that of E. coli recA44 would comprise a mutation of “g” to “a” at nucleotide 739. This would encode a RecA protein comprising a V to M mutation at amino acid 247. Amino acid Val-247 occurs in the context: 245-VKVVKNK-251 (SEQ ID NO:56). The position of the amino acid, indicated by Kawashima, et al., supra, to reside at amino acid-246 in E. coli recA, occurs in the RecA coding sequence of GenBank Acc. No. V00328 at amino acid-247 of E. coli recA. The protein sequence of the E. coli RecA of GenBank Acc. No. V00328 in which the temperature sensitive mutation has been made is shown in SEQ ID NO:52, below.

Valine-244 of the B. anthracis recA wild type sequence corresponds to the valine-247 of the E. coli recA wild type sequence of (GenBank Acc. No. V00328) and valine-246 of the sequence referred to in Kawashima, et al., supra, (the amino acid that is mutated to methionine-247 in the temperature sensitive E. coli mutant). Valine-244 of the B. anthracis recA wild type sequence occurs in the context: 242-VKVVKNK-248 (SEQ ID NO:56). Thus, mutating valine-244 of the B. anthracis recA wild type sequence to a residue encoding methionine-244 is expected to produce a temperature sensitive recA mutant. Such a protein sequence is shown as SEQ ID NO:54, below.

### Example 6

### Introduction of Mutations in the Active Sites of B. anthracis Antigens

As mentioned above, in the following examples, the spoIIE gene mutant is a deletion mutant, while the cya gene mutation is a point mutation and the lef gene mutation is a point mutation. The lethal factor mutation H686A inactivates its protease activity, and the edema factor mutations K346Q and K353Q (together) inactivate its adenyl cyclase activity (Brossier et al., Infect. Immun., 68:1781-1786 (2000)). These mutations are introduced into B. anthracis strains to be used in vaccines, such as the spoIIE uvrAB and spoIIE uvrAB recAts strains. The lef (lethal factor) and cya (edema factor, adenyl cyclase) genes are cloned and mutagenized with the Quick Change kit (Stratagene, La Jolla, Calif.) to create the mutant genes. The mutant genes are then transferred to pKSV7 and finally introduced into the host pXO1 plasmid by allelic exchange.

Mutagenesis of Bacillus anthracis toxin genes was accomplished as follows:

Generation of edema factor mutations K346Q and K353Q (together) that inactivate its adenyl cyclase activity used the following protocol. A fragment of cyaa gene was amplified with primers: cyaF: AGATAAAATACAGCAGACACAAGAC (SEQ ID NO:34) cyaR: TAGTTGAATCCGGTTTCCTC (SEQ ID NO:35), and cloned into the topo TA vector. To create the mutant allele primers were used in the Quick Change® kit (Stratagene, La Jolla, Calif.):

| | |
|---|---|
| cyal1F: | |
| GGTGTGGCTACACAGGGATTGAATGTTCATG | (SEQ ID NO:36) |
| cyal1R: | |
| CATGAACATTCAATCCCTGTGTAGCCACACC. | (SEQ ID NO:37) |

Mutation of the second residue was effected using the following primers:

| | |
|---|---|
| cyal2F: | |
| GATTGAATGTTCATGGACAGAGTTCGGATTGGG | (SEQ ID NO:38) |
| cyal2R: | |
| CCCAATCCGAACTCTGTCCATGAACATTCAATC. | (SEQ ID NO:39) |

The following primers introduced a silent mutation, which was used to check for mutagenesis:

| | |
|---|---|
| cyaMSF: | |
| ACAGGGATTGAATGTCCATGGACAGAGTTCG | (SEQ ID NO:40) |
| cyaMSR: | |
| CGAACTCTGTCCATGGACATTCAATCCCTGT. | (SEQ ID NO:41) |

This allele was transferred to pKSV7 and then introduced into the host pXO1 plasmid by allelic exchange. The mutation was verified by PCR followed by a restriction digest that discerns between the wild type and mutant alleles.

Generation of a mutation in lethal factor H686A that inactivates its protease activity, was accomplished as follows. A fragment of the lefA gene (encoding lethal factor) was amplified with primers CAGGAGGGTTAATTGATAGTCCGTC (SEQ ID NO:42) and TTTGCGTCTTATTTGGCTTTAACG (SEQ ID NO:43), and the fragment was cloned into the topo-TA vector (Invitrogen). The following primers were then used to mutate the residue encoding H686 to A using the Quick Change® ( kit (Stratagene, La Jolla, Calif.): LefMF: AGTGAGGGTTTTATAGCTGAGTTTGGACATGCTGTG (SEQ ID NO:44) and LefMR: CACAGCATGTCCAAACTCAGCTATAAAACCCTCACT (SEQ ID NO:45). This allele has been transferred to pKSV7. Once transferred to pKSV7, the allele is introduced into the host pXO1 plasmid by allelic exchange.

### Example 7

### Inducible Expression of Protective Antigen at High Levels

The use of SOS regulatory sequences for expressing protective antigen at high levels. Cheo et al (Cheo et al., J. Bacteriol., 175:5907-15 (1993)) have shown that the consensus sequence GAACN₄GTTC (SEQ ID NO:15) defines the LexA repressor site for genes in the SOS response of B. subtilis. This sequence was identified within the promoter regions of DNA damage-inducible (din) genes from Bacillus subtilis. This sequence has been proposed to function as an operator site that is required for regulation of the SOS system of B. subtilis, and the consensus sequence was modified to 5′-CGAACRNRYGTTYC-3′ (R=G or A; Y=C or T; N=A, G, C, or T; SEQ ID NO:29) by Winterling et al (Winterling et al., J. Bacteriol., 180:2201-221 (1998)). A similar consensus sequence upstream of the promoters for the B. anthracis recA and uvrAB genes, which are part of the SOS regulon, is identified. To make a B. anthracis strain in which expression of protective antigen is induced in response to photochemical treatment, the protective antigen gene is put under the control of the SOS regulatory sequence and introduced into the B. anthracis spoIIE ΔuvrAB strain, so that treatment with psoralen and UVA light will induce expression of high levels of protective antigen to be made. The gene of interest, in this case, the protective antigen gene, is functionally linked with a B. anthracis promoter that is under the control of the SOS response.

Homologous recombination using the pKSV7 vector as described above in Example 2 is used to insert the heterologous cassette expressing protective antigen at a desired site in the chromosome. This can include replacement of the homologous protective antigen with the modified heterologous sequence.

The use of other inducible promoters. In some embodiments, for instance, when the B. anthracis strain carries both uvrAB and recA mutations, the SOS response will not occur, since this response depends upon RecA protein. In these cases, it is desirable to use a different sort of inducible promoter. Suitable promoters for this use can be determined by first identifying which proteins are expressed at high levels after S-59/UVA treatment of an uvrAB recA double mutant. The mass spectrometry techniqued described in Lenz et al., Proc. Natl. Acad. Sci. USA, 0:12432-12437 (2003), can, for instance, be used for this purpose. Once the proteins expressed at high level under S-59 UVA treatment conditions are determined, the promoters controlling expression of the highly expressed proteins can be identified through techniques known to those of ordinary skill in the art. A promoter identified in this manner can then be fused to the gene expressing the protective antigen. The construct can then be introduced into the chromosome of the mutant Bacillus anthracis using one of the integration vectors described herein or another vector known in the art. The present invention provides a heterologous promoter operably linked with a nucleic acid encoding, e.g., protective antigen.

### Example 8

### Exemplary Mutant B. anthracis Strains

A variety of different mutant B. anthracis strains are prepared using combinations of the methods described in the Examples, above. Exemplary mutant B. anthracis strains to be used in vaccine compositions are listed in Table 3.

TABLE 3
B. anthracis strains and candidate vaccines

| | Relevant Characteristics and | Use and Vaccine Strain |
|---|---|---|
| Strain and/or Genotype | Phenotype | Number |
| Ames pXO1+/pXO2+ | Fully virulent wild-type B. anthracis | Initial host strain for |
| | (Toxigenic and encapsulated) | construction of all vaccine |
| | | candidates |
| | | Production of virulent spores |
| | | for challenge experiments in |
| | | mice and guinea pigs |
| Sterne | Toxigenic, non-encapsulated | Production of virulent spores |
| pXO1+/pXO2− | | for challenge experiments in |
| | | mice and guinea pigs |
| spoIIE pXO1+/pXO2+ | Non-sporogenic | Vaccine strain #1 |
| | Toxigenic, encapsulated | |
| SpoIIE/uvrAB | Non-sporogenic | Vaccine strain #2 |
| pXO1+/pXO2+ | NER-¹ (Increased S-59/UVA | |
| | sensitivity) | |
| | Toxigenic, encapsulated | |
| SpoIIE/uvrAB/recA ts³ | Non-sporogenic | Vaccine strain #3 |
| pXO1+/pXO2+ | NER-(Increased S-59/UVA | |
| | sensitivity) | |
| | Toxigenic, encapsulated | |
| SpoIIE/uvrAB/recA ts | Non-sporogenic | Vaccine strain #4 |
| pXO1+/pXO2+ | NER-/conditional HR-⁴ | |
| | (Increased S-59/UVA sensitivity) | |
| | Toxigenic, encapsulated | |
| spoIIE/uvrAB/pXO1 | Non-sporogenic | Vaccine strain #5 |
| (lef686/cya346/35)/ | NER-(Increased S-59/UVA | |
| pXO2+ | sensitivity) | |
| | Encapsulated | |
| | Non-toxigenic (LF/EF functional | |
| | domains mutated) | |
| spoIIE/uvrAB/recA ts/ | Non-sporogenic | Vaccine strain #6 |
| pXO1(lef686/cya346/35)/ | NER-/conditional HR- | |
| pXO2+ | (Increased S-59/UVA sensitivity) | |
| | Encapsulated | |
| | Non-toxigenic (LF/EF functional | |
| | domains mutated) | |
| spoIIE/uvrAB/ | Non-sporogenic | Vaccine strain #7 |
| pXO1 | NER-(Increased S-59/UVA | |
| (lef686/cya346/35)/ | sensitivity) | |
| pXO2+/ | Encapsulated | |
| Pro_{S-59}-PA | Non-toxigenic (LF/EF functional | |
| | domains mutated) | |
| | S-59 psoralen inducible PA | |
| spoIIE/uvrAB/recA ts/ | Non-sporogenic | Vaccine strain #8 |
| pXO1(lef686/cya346/35)/ | NER-/conditional HR- | |
| pXO2+/ | (Increased S-59/UVA sensitivity) | |
| Pro_{S-59}-PA | Encapsulated | |
| | Non-toxigenic (LF/EF functional | |
| | domains mutated) | |
| | S-59 psoralen inducible PA | |

¹NER, nucleotide excision repair
³Conditional recA strains under the control of a lacI repressible promoter can also be derived
⁴HR, homologous recombination

### Example 9

### Characterization of Protein Expression Levels, Including Protective Antigen and Capsule, in Psoralen-inactivated B. anthracis Strains

To show that inactivated B. anthracis strains can still metabolize, the cells are incubated in minimal medium with bicarbonate (Thome et al., J. Gen. Microbiol.,17:505-516 (1957)). After such incubation the cells are removed by centrifugation and save the supernatant. The supernatant is subjected to SDS-polyacrylamide gel electrophoresis. After staining with Coomassie Blue, protective antigen stands out, and its presence is confirmed by Western blot analysis (Brossier et al., Infect. Immun. 68:5731-5734 (2000)) and by mass spectometry. In addition, mass spectrometry is used to identify the other proteins that are excreted under these conditions, using the methods described in Lenz et al., Proc. Natl. Acad. Sci. U.S.A., 100:12432-12437 (2003). In order to assess whether polyglutamate capsule is made under these conditions, pXO2, which encodes the genes for capsule synthesis, is introduced into the strains by transduction and (Green et al., Infect. Immun., 49:291-297 (1985). Capsule is measured by rocket immunoelectrophoresis (Uchida et al., Mol. Microbiol, 23:1229-1240 (1997)).

### Example 10

### Characterization of the Humoral and Mucosal Responses in Swiss Webster and A/J Mice Immunized with Attenuated B. anthracis Strains

Mouse Immunization. Mice are injected with the S-59/UVA vaccines by the intramuscular (IM) or the subcutaneous (SC) routes to determine which route of immunization results in the best bacterial-specific humoral and cellular responses. Intranasal (IN) immunization of mice is also tested to assess mucosal responses induced by the candidate vaccines. IN immunization with 5 μ of a designated vaccine preparation into each nare of lightly anesthetized mice is performed as described previously (Boyaka, et al., J. Immunol., 170: 5636-5643 (2003)). Mice are immunized with 0.1 LD₅₀ doses of the candidate vaccines. Any of the eight S-59/UVA inactivated vaccine candidates in which a median lethality level is not observed is given at an initial dose of 10⁸ particles. Mice that are immunized by more than one route are not injected with a combined dose that exceeds the 0.1 LD₅₀ dose, or is greater than 10⁸ particles. Mice given multiple immunizations receive consistent vaccine doses with all injections. As immunization on three consecutive days with S-59/UVA inactivated Listeria uvrAB resulted in increased humoral and cellular immunity as compared to a single immunization, the same strategy is used with the B. anthracis strain vaccines. Mice are also given booster immunizations at 14 days and 28 days following the primary immunization.

Quantification of antibodies to PA, LF, EF, capsule, and whole bacteria. The mucosal and antibody responses in mice immunized with the various vaccine candidates are characterized. Serum is taken from the retroorbital plexus prior to immunization as well as 1 week after each immunization. Saliva and nasal washes for measurement of IgA levels are performed at the time of sacrifice one week after the final immunization. The durability of the humoral and mucosal immunity induced by the candidate vaccines at 45 days after the final immunization is also characterized. Humoral and mucosal responses against PA, capsule, and vegetative bacteria (Sterne strain) are determined by enzyme-linked immunosorbent assays (ELISAs), as published previously (Ballard et al., Proc. NatL. Acad. Sci. U.S.A, 93:12531-12534 (1996); Rhie et al., Proc. Natl. Acad. Sci. U.S.A., 100:10925-10930 (2003)). Briefly, Immulon 96-well Maxisorp plates (Nalge Nunc) are first coated by 5 μg purified PA, LF, EF, BSA conjugated with poly-γ-D-glutamic acid (PGA) capsule prepared as described previously (Rhie et al., Proc. Natl. Acad. Sci. U.S.A, 100:10925-10930 (2003)), or with S-59 psoralen/UVA inactivated bacteria ground under liquid nitrogen using a mortar and pestle in 50 mM carbonate buffer (pH 9.6) at 4° C. for 16 h, and blocked with TSTA buffer (50 mMTris (pH 7.6), 142 mM NaCl, 0.05% sodium azide, 0.05% Tween 20, 2% bovine serum albumin). Ser. two-fold dilutions of mouse plasma or mucosal secretions are added to the 96-well plates coated with PA, PGA-BSA, or Sterne respectively. Binding of Abs to the immobilized antigens is determined by incubation with isotype-specific peroxidase goat anti-mouse μ, γ, or α H chain-specific antibodies from Southern Biotechnology Associates (Birmingham, Ala.). Biotinylated rat anti-mouse γ 1 (clone G1-7.3), γ 2b (clone R12-3), or γ 3 (clone R40-82) H chain-specific mAbs (BD PharMingen, San Diego, Calif.) and streptavidin-conjugated peroxidase are used for IgG Ab subclass analysis (Cole, J. Bacteriol., 107:846-852 (1971); Cole et al., Basic Life Sci., 5B:487-495 (1975)). The colorimetric reaction is developed by addition of ABTS substrate (Sigma-Aldrich, St. Louis, Mo.). End-point titers are expressed as the reciprocal log₂ dilution giving OD₄₁₅ greater than two standard deviations above those obtained with control, non-immunized mice.

Enzyme-linked immunospot (ELISPOT) assay for the detection of Ig-secreting cells. The frequency of PA-specific Ig-secreting lymphocytes is determined by ELISPOT analysis (Boyaka et al., J. Immunol., 170:5636-5643 (2003)). Briefly, spleens or cervical lymph nodes of vaccinated and control mice are rapidly dissected out and placed in ice-cooled RPMI 1640 medium and single cell suspensions are prepared. 96-well PVDF-based plates (BD Biosciences, San Jose) are coated overnight with 2.5 μg/ml purified PA (List Biological Laboratories, Campbell, Ca.). The plates are washed, blocked for 2 hrs at 37° C. with 200 μl complete RPMI, and serial dilutions of cell suspensions are added to 96-well plates. Cells are incubated on the plates for 6 hours at 37° C. in 5% CO₂. Antigen-specific Antibody Forming Cells (AFC) are detected with isotype-specific biotin-labeled anti-mouse μ, γ, or α H chain-specific antibodies (Southern Biotechnology Associates). After incubation at RT for 2 h, the plates are washed, and goat anti-biotin: 1 nm Gold conjugate (GAB 1; Ted Pella) is added for 1 hour at RT. After extensive washing, 30 μl of the silver substrate (Silver Enhancing Kit; Ted Pella) is added into each well and the spot development is monitored. Spots in each well are counted using an automated ELISPOT plate reader (CTL, Cleveland). The humoral response is expressed as the number of antibody forming cells per 106 spleen or lymph node cells.

Toxin Neutralization Assays. Neutralizing antibodies induced in mice immunized with the vaccine candidates are evaluated for the ability to protect the J774 macrophage cell line from lethal toxin (PA+LF) (Mock et al., Annu. Rev. Microbiol., 55:647-671 (2001); Boyaka et al. (2003); Rhie et al., Proc. Natl. Acad. Sci. U.S.A, 100:10925-10930 (2003)). Briefly, J774 cells (ATCC, Manassus, Va.) are added to 96-well flat-bottom plates (Nunc) at 5×10⁴ cells/well and incubated for 12 hours at 37° C. in 5% CO₂. Test serum or mucosal secretions are serially diluted two-fold in TSTA buffer. PA and LF (400 ng/ml PA and 40 ng/ml LF) are added to the antiserum dilutions. After incubation for 1 hour the antiserum/lethal toxin complex mixture is added to the cell suspension and incubated for an additional 5 hours. Cell viability is monitored by the MTT assay (absorbance measured at 540 nm). Assays are performed in triplicate with a negative control (normal serum) and a positive control (MAbs, 14B7 and 1G3) (Mikesell et al., Infect Immun., 39:371-376 (1983); Starnbach et al., Nature Medicine, 9:996-997 (2003)) included in each plate. The mean and standard deviation of each triplicate sample dilution is calculated. The endpoint is expressed as the highest serum dilution exhibiting 50% neutralization of the anthrax toxin as compared to normal control serum.

### Example 11

### Characterization of the PA-, LF-, and EF-specific CD4+ T cell-mediated Responses in A/J Mice Vaccinated with ModifiedB. anthracis

T cell Proliferation. CD4+ T cell proliferation are determined from PBMC, spleen and lymph node cells of vaccinated and naïve A/J mice. Spleen and cervical lymph nodes are dispersed to obtain single cell suspensions as previously described (Boyaka et al., J. Immunol., 162: 122-8 (1999); Lillard et al., J. Immunol., 166:162-169 (2001); Little et al., Infect. Immun., 65:5171-5 (1997)). CD4+ T cells are isolated by negative selection using the Mouse CD4+ T cell isolation kit from Miltenyi Biotec (Auburn, Ca.). Purified CD4+ T cells from individual mouse spleens, from pooled lymph nodes or PBMCs are cultured at 4×10⁶ cells/ml and stimulated with varying concentrations of PA, LF or EF in the presence of T-cell-depleted, non-dividing syngeneic naive spleen feeder cells (8×10⁶ cells/ml) in complete RPMI (RPMI supplemented with 10% FBS, 10 mM Hepes, 2 mM L-glutamine, 1 mM sodium pyruvate, non-essential amino acids, 23.8 mM sodium bicarbonate, 5×10⁻⁵ M μ-Mercapthoethanol, 100 U/ml penicillin and 100 μg/ml streptomycin). The replication of splenic feeder cells is arrested by brief photochemical treatment with S-59 psoralen. Cultures are incubated for 4 days at 37° C. and 5% C0₂ prior to addition of 0.5 μCi of tritiated thymidine ([³H]TdR) for the final 18 to 20 hours The cells are harvested onto glass fiber sheets and the amount of incorporated thymidine is determined by measuring the radioactivity on the scintillation counter (Wallac, Turku, Finland).

Analysis of PA-, EF- or LF-induced cytokine responses. CD4+ T cells are isolated by negative selection using the Mouse CD4+ T cell isolation kit from Miltenyi Biotec (Auburn, Ca.). Purified CD4+ T cells from spleens or lymph node of individual mice are cultured in round-bottom 96-well plates at 1×10⁵ cells/well and stimulated with varying concentrations of PA, LF or EF in the presence of T cell-depleted, non-dividing syngeneic naïve spleen feeder cells (1×10⁵ cells/well) in complete RPMI. The T cell-depleted spleen feeder cells are arrested by a brief photochemical treatment with S-59. T cell cultures are incubated for 2 days at 37° C. and 5% CO₂. Expression of T helper-1 and T helper-2 cytokines is determined from supernatants of antigen-stimulated CD4+ T cells using the Th1/Th2 Cytometric Bead Array kit (BD Pharmingen, San Diego, Calif.).

### Example 12

### Characterization of the Extent of Protection Against Spore and Lethal Toxin Challenge in Swiss Webster and A/J Mice at 45 Days Post Last Immunization Dose with Modified B. anthracis Vaccines

Protection of mice against lethal toxin challenge. Mice immunized with selected candidate vaccines are challenged by tail vein injection with lethal toxin, as described previously (Price et al., Infect. Immun., 69:4509-15 (2001); Rhie et al., Proc. Natl. Acad. Sci. U.S.A., 100:10925-10930 (2003)). Lethal toxin is prepared by mixing recombinant PA and LF recombinant proteins (List Biological Laboratories, Campbell, Calif.) as described (Rhie et al. (2003)). The lethal toxin IV LD₅₀ per mouse is approximately 12 μg of PA mixed with 6 μg of LF. The median lethality in mice of freshly prepared lethal toxin is determined by tail vein injection over a 0.1-10 LD₅₀ dose range of the published values. The protection studies include lethal toxin challenge over a range of 5-10 times the LD₅₀ dose. In this model, unprotected mice succumb within 24 h. Initially, death by anthrax is confirmed in selected mice by plating blood on tryptic soy agar and incubating overnight at 37° C. Plates are observed for colonies with 2-3mm typical anthracis-like “ground glass” appearance. All mice treated with lethal toxin are monitored daily, and experiments are terminated after 2 weeks and all protected mice are sacrificed.

Spore preparation. Strene strain spores are prepared as described (Barnard and Friedlander, 1999). Briefly, single colonies are inoculated into 5 ml of FA medium (3.3% tryptone, 2% yeast extract (dialyzed overnight against water), 0.2% L-histidine, 0.8% Na₂HPO₄, 0.4% KH₂PO₄, 0.74% NaCl) contained in a 100-ml bottle and shaken for 5 h at 37° C. One-tenth-millimeter aliquots are spread on L agar plates, and incubated at 37° C. Bacterial lawns are scraped from the plates, washed extensively with sterile water, heat shocked for 30 min at 60° C., washed with water, purified on 58% Renografin-76 (Bristol-Myers Squibb, Princeton, N.J.) in water, as previously described (Palucka et al., Nature Medicine, 5:868-870 (1999)), and washed once more with water. The spores are then sedimented to a pellet at 10,000×g and resuspended in 1% phenol in water. This yield of this process has been published to range from 0.5×10⁹ to 5.0×10⁹ spores per plate.

Protection of mice against lethal spore challenge. The LD₅₀ value of heat-shocked Sterne strain spores given by intramuscular (IM) injection is determined over a dose range of 10³ to 10⁸ spores. To evaluate protection in vaccinated mice against inhalation anthrax, challenge experiments are also performed by intratrachial (IT) spore administration, as described previously (Brook et al., J. Med. Microbiol., 50:702-11 (2001)). Briefly, the tongue of immobilized and anesthetized mice are gently pulled outward and laterally with forceps, and the vaccine is delivered using a syringe fitted with a blunt 1.5 inch 22-gauge needle bent at a gentle angle, approximately 1 inch from the tip. We anticipate that the Sterne strain LD₅₀ value administered by IM or IT routes is approximately 10³ in A/J mice, and up to 10-fold higher in Swiss Webster mice. The protection studies include up to 100 LD₅₀ dose spore challenge. All mice treated with spores are monitored daily, and experiments are terminated after 2 weeks and all protected mice are sacrificed. In all challenge experiments, the mean time to death is determined in non-surviving cohorts. In additional challenge experiments, the protection of mice against a fully virulent Ames strains (pXO1+, pXO2+) is tested.

### Example 13

### Sequences Useful in Production of Recombinant or Mutant Bacillus anthracis Strains

Recombinant and/or mutant Bacillus anthracis strains are described in the Examples above. Information is provided in the Table 4, below regarding some sequences of use in construction of some of the recombinant B. anthracis strains described above. Each of the sequences identified by accession number or by other reference in Table 4 is incorporated by reference herein in its entirety.

TABLE 4
Additional B. anthracis sequence information (including Genbank accession numbers)

| | | | | Coordinates in |
|---|---|---|---|---|
| | | | | the genome or |
| Bacteria | Accession # | Gene | Location | plasmid |
| B. anthracis | NC_003997 | spoIIE | Chromosome | 64936-67314 |
| Ames | | | | |
| B. anthracis | NC_001496 | cya (encodes | pXO1 | 154224-156626 |
| Sterne | | edema factor) | virulence | |
| | | | plasmid | |
| B. anthracis | NC_007322 | pagA (encodes | pXO1 | 143779-146073 |
| Ames | | protective | virulence | |
| | | antigen | plasmid | |
| B. anthracis | NC_007322 | lef (encodes | pXO1 | 149357-151786 |
| Ames | | Lethal Factor) | virulence | |
| | | | plasmid | |
| B. anthracis | NC_003997 | lexA (lex | Chromosome | 3453806-3454426 |
| Ames | | repressor) | | |
| B. anthracis | NC_003997 | recA | Chromosome | 3590268-3591626 |
| Ames | | | | (Intron 3590691-3591017; |
| | | | | Ko M., |
| | | | | et. al., J |
| | | | | Bacteriol. 2002. |
| | | | | 184: 3917-3922 |
| B. anthracis | NC_007530 | uvrC | Chromosome | 4324547-4326331 |
| Ames | | | | |

In addition, the B. subtilis SOS promoter region is identified as 5′-CGAACRNRYGTTYC-3′(R=G or A; Y=C or T; N=A, G, C, or T; SEQ ID NO:29) (Winterling, K. W. et. al., J Bateriol, 1998 180:2201-2211).

The protein and nucleotide sequences corresponding to the genes listed in Table 4, above, are also provided below.

### Example 14

### Psoralen-induced and Heat-induced Elimination of Proliferative Ability in B. anthracis Mutants

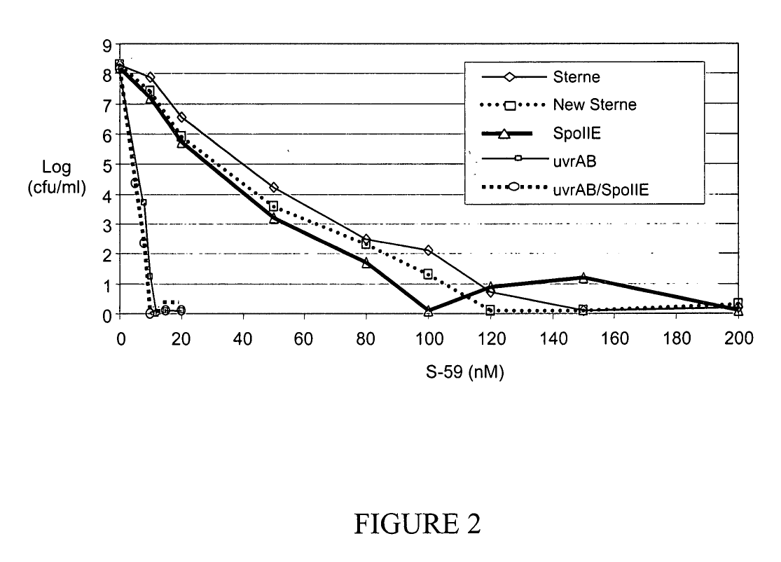

FIG. 2 shows inactivation of various B. anthracis strains versus concentration of psoralen (S-59). Inactivation was measured by colony forming units (CFU). The S-59 concentrations were 0-2000 nM. The indicated B. anthracis strains were exposed to the indicated concentration of S-59, then treated with ultraviolet light (6.5 J/cm²), followed by dilution and plating. At the beginning of the treatment with the psoralen, the concentration of the suspended bacteria was an optical density of about OD=0.6. After addition of the psoralen, the bacteria grew to an OD of about 2.0 and then were treated with the UVA light. The exposure time to the UV light was limited to about one minute. The B. anthracis strains were Strene (- [symbol] -) (open diamond); new Sterne (-□-)(open large square); spoIIE (-Δ-)(open triangle); uvrAB (- [symbol] -) (open small square); and uvrAB/spoIIE (-O-) (open circle) (FIG. 2). The results demonstrated that relatively low concentrations of psoralen, e.g., 100 nM or under 100 nM S-59, were sufficient for effecting nearly a 100 million-fold reduction in viable bacteria, where the B. anthracis was the uvrAB mutant or uvrAB/spoIIE mutant. The efficiency in reducing CFU was somewhat greater with the uvrAB/spoIIE mutant than with the uvrAB mutant. In contrast, much greater concentrations of S-59 were needed to effect similar reductions in CFU with the B. anthracis strains that were not uvrAB or uvrAB/spoIIE mutants. For example, the higher concentration of 1000 to 1200 nM S-59 were needed to produce a 10 million-fold reduction in viable bacteria, where the B. anthracis was Sterne, new Sterne, or the spoIIE mutant. Thus, the excision repair mutants of the present invention enable inactivation of B. anthracis at very low levels of psoralen (FIG. 2).

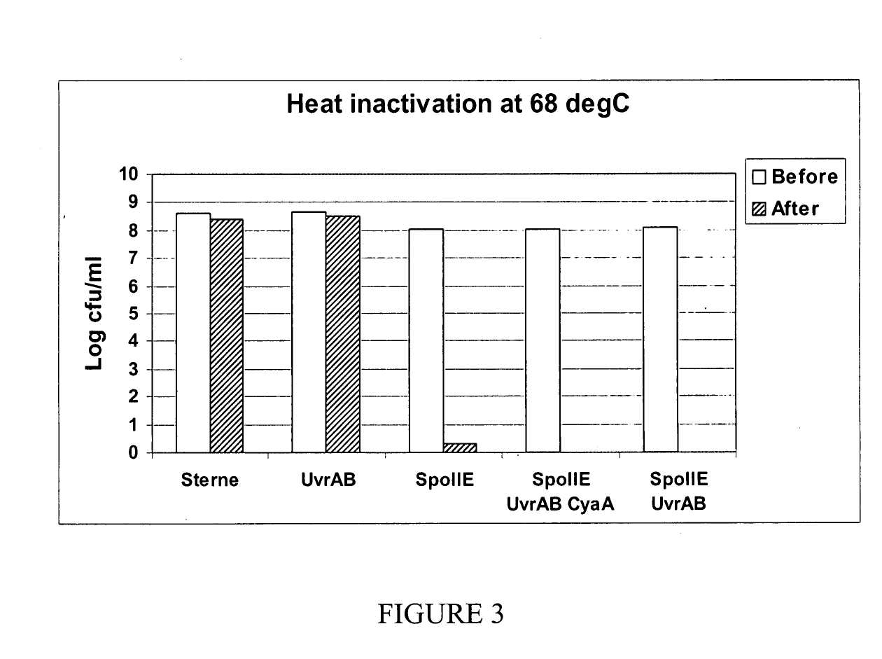

FIG. 3 shows colony forming units of B. anthracis before and after heating. The B. anthracis had been grown under sporulation conditions. The sporulation conditions used were PA medium. Heating was for 30 minutes at 68 degrees in aqueous solution. The following strains of B. anthracis were tested: Sterne; uvrAB mutant; spoIIE mutant, spoIIE/uvrAB/cyaA mutant, and spoIIE/uvrAB mutant. Heat-treatment had no detectable influence on colony forming units of the Sterne strain or uvrAB mutant strain. These two strains did not contain mutations in a spore-forming gene. But heat-treatment of the B. anthracis strains that were spoIIE mutant resulted in a reduction of CFU by over 10-million fold, or in the range of 100-million fold. In short, the SpoIIE mutation prevented spore formation by the heat-treated bacteria.

### Example 15

### Some Exemplary B. anthracis Strains

The present invention includes, but is not limited to, the following strains. The strains are based on B. anthracis Sterne (pXO1+pXO2-) (Institute Pasteur 7702) or on B. anthracis ANR 1 (also known as B. anthracis Ames (pXO1+pXO2−):
(1) B. anthracis Sterne ΔspoIIE;
(2) B. anthracis Sterne ΔuvrAB;
(3) B. anthracis Sterne ΔspoIIE ΔuvrAB;
(4) B. anthracis Strene ΔspoIIE ΔuvrAB cyaK346Q/K353Q (referred to herein as “Strene 3”) (Codons 346 and 353 of cya are mutated as indicated.);
(5) B. anthracis Sterne ΔspoIIE ΔuvrAB cyaK346Q/K353Q lefH686A (referred to herein as “Sterne 4”) (Codons 346 and 353 of cya and codon 686 of lef are mutated, as indicated.);
(6) B. anthracis Sterne ΔspoIIE ΔuvrAB cyaΔ346/Δ353 lefΔ686 (Codons 346 and 353 of the cya gene and codon 686 of the lef gene are deleted.);
(7) B. anthracis Sterne ΔspoIIE ΔuvrAB cyaΔ346/Δ353 lefΔ686 (pXO2) (The strain is modified to contain the plasmid pXO2.);
(8) B. anthracis Sterne ΔspoIIE ΔuvrAB cyaΔ346/Δ353 lefΔ686::pagA (An extra copy of the pagA gene is integrated into the genome, e.g., by site specific integration or by homologous integration, as indicated by the double colon (“::”).);
(9) B. anthracis Sterne ΔspoIIE ΔuvrAB cyaΔ346/Δ353 lefΔ686::spore specific gene. (An extra copy of a spore specific gene is integrated, e.g., by site specific integration or by homologous integration, as indicated by the double colon (“::”).); and

(10) B. anthracis ANR 1 ΔspoIIE ΔuvrAB cyaΔ346/Δ353 lefΔ686.

Modified bacteria comprising the mutations listed above, but derived from B. anthracis strains other than Sterne and/or ANR 1 are also provided.

### Example 16

### Lack of Sporulation by spoIIE Deletion Mutant

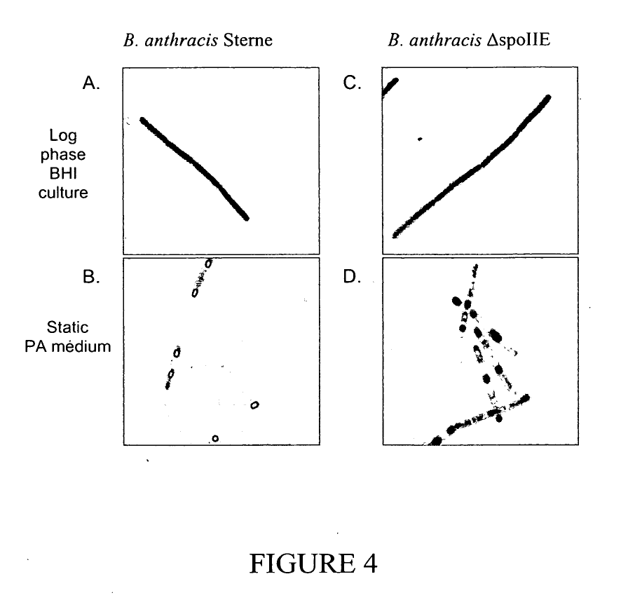

FIG. 4 shows photographs taken of B. anthracis Sterne and B. anthracis Sterne ΔspoIIE, both in log phase when cultured in BHI (Brain Heart Infusion) medium (Difco, Becton Dickinson, Franklin Lakes, N.J.), and in static phase when cultured in PA medium (Thorne (1968) J. Virology.2:657-662). In log phase, neither type of bacterium forms detectable spores, when examined by light microscopy (FIG. 4A and 4C). In the static phase, only theB. anthracis Sterne forms spores (FIG. 4B). The B. anthracis spoIIE mutant cannot sporulate in status phase (FIG. 4D). In static phase, B. anthracis Sterne ΔspoIIE forms chains of bacteria that are unable to septate, and fails to form spores (FIG. 4D).

### Example 17

### Additional Demonstration of Psoralen/UVA-induced Elimination of Proliferative Ability of B. anthracis Mutants

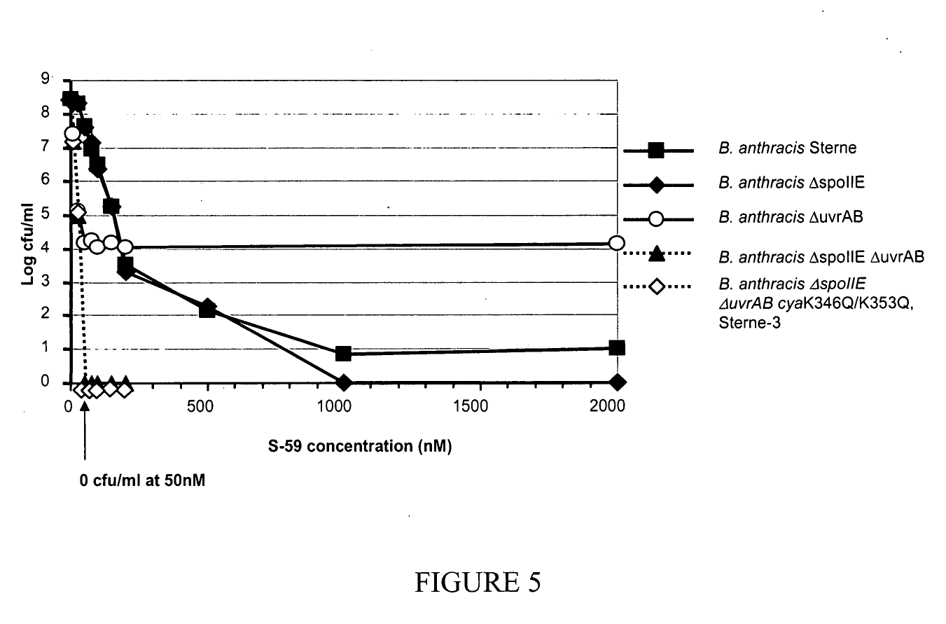

FIG. 5 shows the level of ability of various preparations of B. anthracis to form colonies, where the preparations had been exposed to the indicated concentration of psoralen (S-59) and UVA light. The number of colony forming units (per ml) of the various B. anthracis strains versus the concentration of the psoralen is shown. The bacteria were grown to an OD of about 0.6, followed by addition of psoralen, then followed by growth for an hour to an OD of about 2.0, followed by treatment with UVA light (6.5 Joules per cm 2). All colonies were grown in BHI agar. B. anthracis Strene ΔspoIIE ΔuvrAB (▴; closed triangles) and B. anthracis Strene ΔspoIIE ΔuvrAB cyaK346Q/K353Q (Strene 3) ( [symbol] ; open diamonds) were found to not be able to form colonies after treatment with very low concentrations of psoralen (50 nM psoralen) (FIG. 5). B. anthracis Strene ΔuvrAB prepared at low concentrations of psoralen, e.g., 50 nM psoralen, were also found to be substantially unable to form colonies at low concentrations of psoralen (-∘-; open circles). This particular preparation showed some growth with treatment with higher psoralen levels in some experiments, but no growth with higher psoralen levels in separate experiments. Where resistance of B. anthracis Sterne ΔuvrAB strains to psoralen was found, the appearance of the resistant bacteria occurred because of the stochastic formation of spores, where sporulation had been initiated, where sporulation had occurred, and/or where spores had been formed prior to initiating the psoralen treatment. The dramatic drop in colony forming units found with psoralen-treatment of the B. anthracis Sterne ΔuvrAB ΔspoIIE mutant, demonstrates, in part, the role of the spoIIE deletion mutation in preventing stochastic spore formation (FIG. 5). B. anthracis that are substantially unable to form colonies only at relatively high levels of psoralen are shown by preparations of B. anthracis Sterne (-▪-; filled squares) or B. anthracis Sterne ΔspoIIE (-♦-; filled diamonds), e.g., over 1000 nM psoralen (FIG. 5).

### Example 18

### Metabolic Activity of Psoralen-treated B. anthracis Mutants

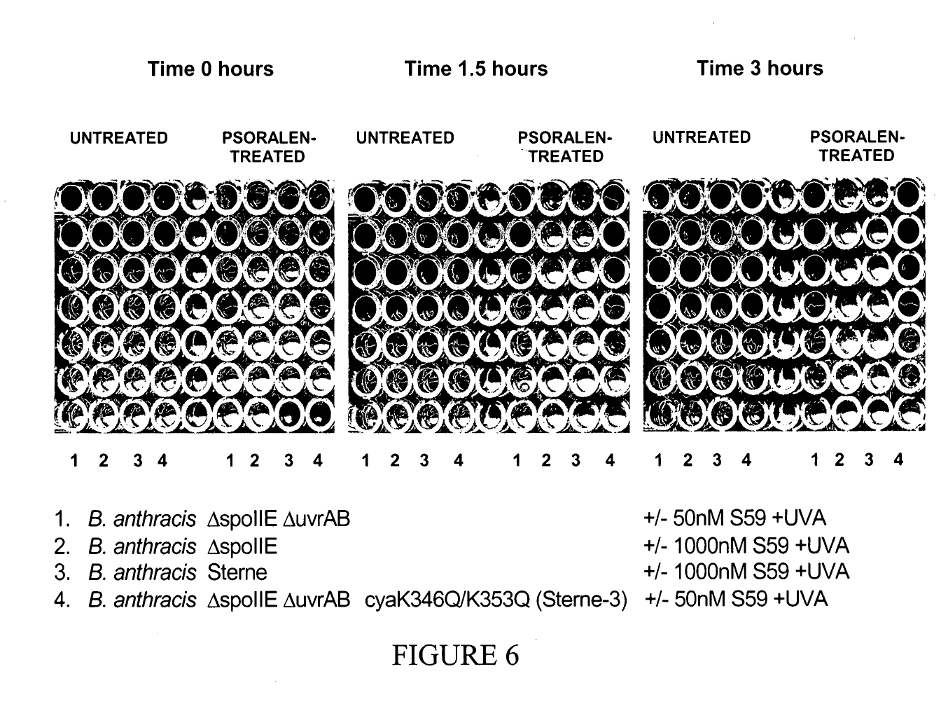

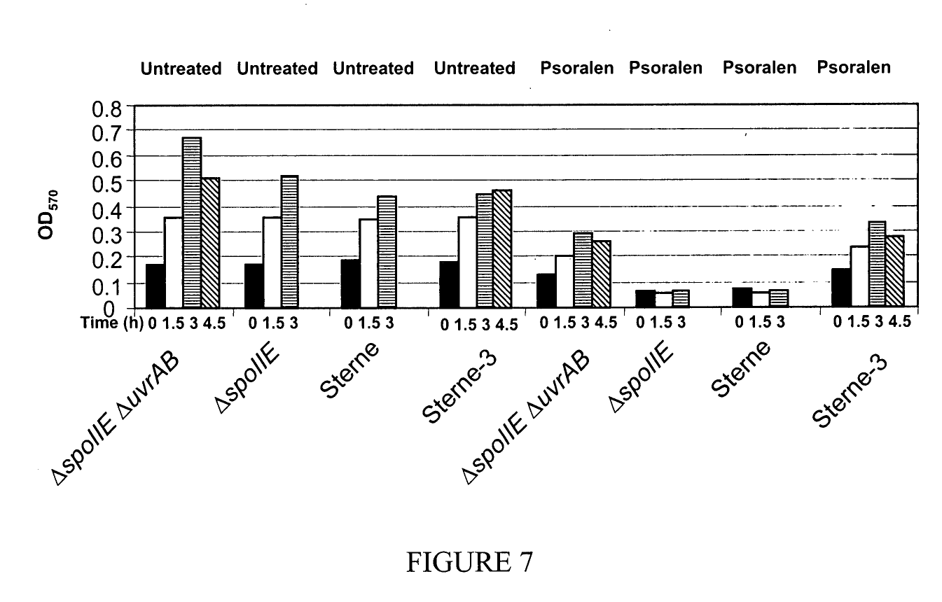

Dye metabolism assay: FIGS. 6 and 7 shows the metabolic activity of psoralen-treated B. anthracis of the present invention, by way of dye metabolism assays.

For production of psoralen-treated bacteria, B. anthracis was prepared in BHI medium, then treated with psoralen and UVA light (“psoralen-treated” bacteria or “psoralen/UVA-treated” bacteria). Other preparations of the bacteria were not treated with psoralen and UVA light (“control” or “untreated” bacteria). Psoralen treatment was at 37 degrees C, followed by cooling on ice, then irradiation with UVA light (6.5 Joules per cm² at room temperature). For the treatment, the bacteria were grown to an OD of about 0.6, followed by addition of psoralen, then followed by growth for an hour to an OD of about 2.0, followed by the treatment with the UVA light. After treating with psoralen S-59 and UVA light, bacteria were washed two times, snap frozen, and stored at minus 80 degrees C. For use in metabolic studies, frozen bacteria were thawed, placed in BHI medium, and dispensed in microtiter plates. Metabolic activity of the bacteria in the microtiter plates was assessed by an MTT assay, an assay that measures conversion of tetrazolium to formazan (Abs. 570 nm). Bacteria were pre-incubated in BHI medium for various periods of time (0, 1.5, 3.0, or 4.5 hours) prior to dispensing in the microtiter plates, assembling the MTT assays in the wells of the plates, incubating the plates for 90 minutes at 37 degrees, and measuring metabolic activity (see, e.g., Hering, et al. (2004) Biologicals 32:17-27; Meletiadis, et al. (2000) J. Clin. Microbiol. 38:2949-2954; Coote and Arain (1996) FEMS Immunol. Med. Microbiol. 13:65-70; Haddad, et al. (1994) Avian Dis. 38:755-761).

FIGS. 6 and 7 show the results of the MTT assays. Bacterial metabolism resulted in a change in the color of the medium from yellow to purple, and an increase in absorbance at 570 nm. In viewing the 3 hour results, the dark spots in the microtiter plates demonstrate that the B. anthracis ΔuvrAB mutants treated with low psoralen concentrations were metabolically active (dark spots), while adjacent light spots from B. anthracis (not deleted in uvrAB) treated with high psoralen levels were not metabolically active (light spots) (FIG. 6).

The wells of microtiter plates in FIG. 6 contained the following: (1) Untreated B. anthracis Strene ΔspoIIE ΔuvrAB; (2) Untreated B. anthracis Sterne ΔspoIIE; (3) Untreated B. anthracis Sterne; (4) Untreated B. anthracis Strene ΔspoIIE ΔuvrAB cyaK346Q/K353Q (Sterne 3); (5) Psoralen-treated (50 nM S-59) B. anthracis Sterne ΔspoIIE ΔuvrAB; (6) Psoralen-treated (1000 nM S-59) B. anthracis Strene ΔspoIIE; (7) Psoralen-treated (1000 nM S-59) B. anthracis Sterne; and (8) Psoralen-treated (50 nM S-59) B. anthracis Strene ΔspoIIE ΔuvrAB cyaK346Q/K353Q (Sterne 3). The quantitative results of the MTT assays are shown in FIG. 7.

The results demonstrate that the preparations of untreated B. anthracis had relatively high levels of metabolism, where pre-incubating for about 1.5 to 3.0 hours resulted in progressively higher levels of metabolism. Regarding the psoralen-treated preparations, the results from B. anthracis Sterne ΔspoIIE ΔuvrAB (psoralen-treated) and B. anthracis Sterne ΔspoIIE ΔuvrAB cyaK346Q/K353Q (Strene 3) (psoralen-treated) also produced relatively high levels of metabolism and, here also, pre-incubation for about 1.5-3 hours resulted in progressively higher levels of metabolism found in the MTT assay. Relatively low levels of metabolism were found in incubations with B. anthracis Strene ΔspoIIE (psoralen-treated) and B. anthracis Strene (psoralen-treated).

Only those B. anthracis mutants with mutations in DNA repair were able to show metabolism after psoralen treatment, because these DNA repair mutations allow low levels of psoralen to be sufficient to impair cell division and prevent colony formation but not high enough to shut down or substantially prevent metabolism.

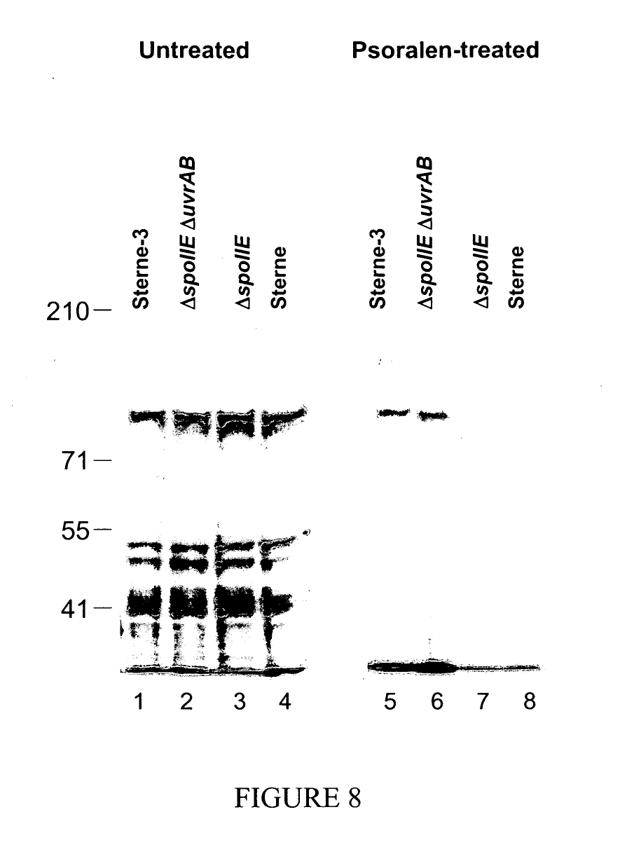

Polypeptide expression assays. FIGS. 8-10 show the metabolic activities of a variety of psoralen-treated B. anthracis strains, by way of polypeptide expression assays.

Polypeptide expression of psoralen/UVA-treated bacteria was assessed by ³⁵S incorporation assays, reflecting biosynthesis of proteins that contain methionine and/or cysteine (FIG. 8). Bacteria were treated with S-59 (50 nM) and UVA light (6.5 Joules per square centimeter) prior to freezing. The psoralen-treated bacteria were grown to an OD of about 0.6, followed by addition of psoralen, then followed by growth for an hour to an OD of about 2.0, followed by treatment with UVA light (6.5 Joules per cm ²). Frozen aliquots of B. anthracis were thawed, and starved for cysteine and methionine by incubating in cysteine free/methionine free medium for 30 minutes, then incubated in medium containing [³⁵S]cysteine and [³⁵S]methionine for 30 minutes. The medium did not contain any added bicarbonate. Bacteria were then collected and separated from the media, and media analyzed for secreted proteins after separation on SDS PAGE and autoradiography (FIG. 8). The assays for metabolic activity shown in FIG. 8 were conducted with the following preparations of bacteria:
Lane 1. Untreated B. anthracis Sterne ΔspoIIE ΔuvrAB cyaK346Q/K353Q (Sterne 3);
Lane 2. Untreated B. anthracis Sterne ΔspoIIE ΔuvrAB;
Lane 3. Untreated B. anthracis Sterne ΔspoIIE;
Lane 4. Untreated B. anthracis Sterne;
Lane 5. Psoralen treated (50 nM S-59) B. anthracis Sterne ΔspoIIE ΔuvrAB cyaK346Q/K353Q (Strene 3);
Lane 6. Psoralen treated (50 nM S-59) B. anthracis Sterne ΔspoIIE ΔuvrAB;
Lane 7. Psoralen treated (1000 riM S-59) B. anthracis Strene ΔspoIIE; and
Lane 8. Psoralen treated (1000 nM S-59) B. anthracis Sterne.

FIG. 8 indicates that all the preparations of untreated bacteria were metabolically active, as revealed by the array of bands, and that psoralen-treated (50 nM S-59) B. anthracis Sterne ΔspoIIE ΔuvrAB cyaK346Q/K353Q (Sterne 3) (Lane 5) and psoralen-treated (50 n/M S-59) B. anthracis Sterne ΔspoIIE ΔuvrAB (Lane 6) were also metabolically active. The preparations of psoralen-treated (1000 nM S-59) B. anthracis Strene ΔspoIIE (Lane 7) and psoralen-treated (1000 riM S-59) B. anthracis Sterne (Lane 8) were not metabolically active (FIG. 8).

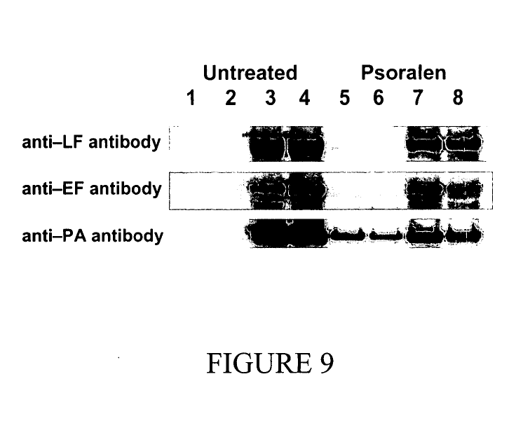

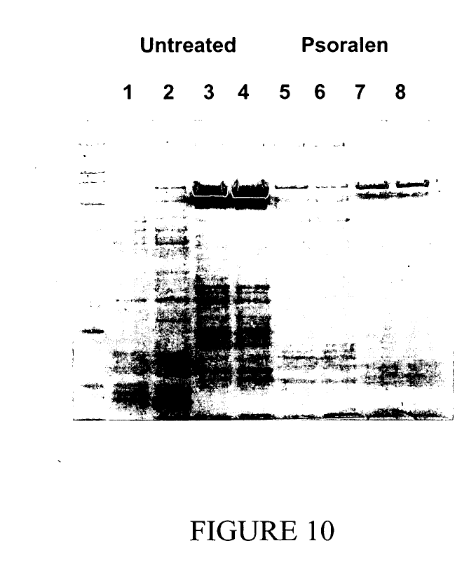

Polypeptide expression was also assessed by western blots, where anti LF antibody, anti EF antibody, or anti PA antibody was used to measure expression of LF (lethal factor), EF (edema factor), or PA (protective antigen), respectively (FIGS. 9 and 10). B. anthracis Sterne ΔuvrAB or B. anthracis Strene ΔspoIIE ΔuvrAB cyaK346Q/K353Q (Strene 3) were used, where indicated, and the bacteria were used untreated or after treating with low dose psoralen (150 nM S-59) and UVA light, where indicated. The psoralen-treated bacteria were grown to an OD of about 0.6, followed by addition of psoralen, then followed by growth for an hour to an OD of about 2.0, followed by treatment with UVA light (6.5 Joules per cm²). Bacteria were then incubated in medium containing added sodium bicarbonate (to induce proteins) or no added sodium bicarbonate, where incubated. The media used were bicarbonate free Ristroph medium, or complete Ristroph medium (0.8% sodium bicarbonate) (Ristroph and Ivins (1983) Infection Immunity 39:483-486).

The samples shown in the lanes of FIGS. 9 and 10 are as follows:
Lane 1. B. anthracis Sterne ΔuvrAB (no bicarb.; no psoralen/UVA);
Lane 2. B. anthracis Sterne ΔspoIIE ΔuvrAB cyaK346Q/K353Q (Sterne 3) (no Bicarb.; no psoralen/UVA);
Lane 3. B. anthracis Sterne ΔuvrAB (+bicarb.; no psoralen/UVA);
Lane 4. B. anthracis Sterne ΔspoIIE ΔuvrAB cyaK346Q/K353Q (Sterne 3) (+Bicarb.; no psoralen/UVA);
Lane 5. B. anthracis Sterne ΔuvrAB (no bicarb.; +psoralen/UVA);
Lane 6. B. anthracis Sterne ΔspoIIE ΔuvrAB cyaK346Q/K353Q (Sterne 3) (no bicarb.; +psoralen/UVA);
Lane 7. B. anthracis Sterne ΔuvrAB (+bicarb. +psoralen/UVA); and

Lane 8. B. anthracis Sterne ΔspoIIE ΔuvrAB cyaK346Q/K353Q (Sterne 3) (+bicarb.; +psoralen/UVA).

The results from western blots (FIG. 9) demonstrated that expression of LF, EF, and PA were all greatly increased with sodium bicarbonate incubation. In short, sodium bicarbonate incubation increased expression of LF, EF, and PA by the untreated bacteria as well as by the psoralen-treated bacteria. Psoralen treatment alone with UVA (without bicarbonate) may increase expression of PA (FIG. 9). The comparable expression of all three antigens in sodium bicarbonate-incubated untreated bacteria and in the psoralen-treated bacteria demonstrates that the psoralen-treated bacteria are, indeed, metabolically active (FIG. 9). Supernatants from the same preparations of bacteria were also separated on SDS PAGE and stained with Coomassie blue stain (FIG. 10), where the results again demonstrated the bicarbonate induction of the secreted proteins (LF, EF, and PA all co-migrating at about 80 kDa), and the result that secretion still occurred in the psoralen-treated bacteria.

The above study compared B. anthracis Sterne ΔuvrAB and B. anthracis Sterne ΔspoIIE ΔuvrAB cyaK346Q/K353Q (FIG. 9), while the following study (FIG. 10) compared B. anthracis Sterne and B. anthracis Sterne ΔspoIIE ΔuvrAB cyaK346Q/K353Q (Sterne 3).

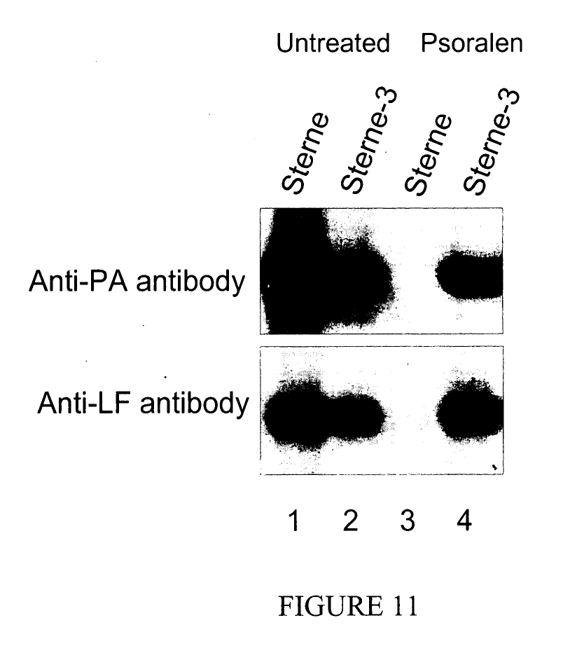

The results indicated that the untreated bacteria are metabolically active (lanes 1 and 2) (FIG. 11), psoralen-treated B. anthracis Sterne (1000 nM S-59) are inactive (lane 3), and psoralen-treated B. anthracis Sterne ΔspoIIE ΔuvrAB cyaK346Q/K353Q (Sterne 3) (50 nM S-59) bacteria are metabolically active (lane 4) (FIG. 11). The psoralen-treated bacteria were grown to an OD of about 0.6, followed by addition of psoralen, then followed by growth for an hour to an OD of about 2.0, followed by treatment with UVA light (6.5 Joules per cm²).

Thus, Bacillus anthracis containing an inactivating mutation in the uvrAB gene that are psoralen/UVA-treated with relatively low levels of psoralen sufficient to render the bacteria unable to form colonies, are metabolically active and therefore able to express substantial amounts of antigens such as protective antigen.

### Example 19

### Toxicity of Psoralen/UVA-treated B. anthracis Mutants

The toxicities of untreated B. anthracis and psoralen-treated B. anthracis were compared, using LD₅₀ assays and mice (Table 5). The mice were DBA/2 (C5 deficient) mice. Vegetative B. anthracis was injected intravenously. The LD₅₀ tests were repeated three times, but with the indicated differences in psoralen concentrations (Table 5). The results demonstrated little or no difference in toxicity of the untreated wild type B. anthracis Sterne strain and the untreated uvrAB mutant (B. anthracis Strene ΔspoIIE ΔuvrAB cyaK346Q/K353Q (Sterne 3)). Regarding the psoralen-treated B. anthracis, the results show that both preparations of bacteria were greatly attenuated. More specifically, regarding the psoralen-treated B. anthracis strains, the toxicity of the metabolically active preparation (B. anthracis Sterne ΔspoIIE ΔuvrAB cyaK346Q/K353Q (Sterne 3)) was about the same or less than that of the B. anthracis Strene that was not metabolically active.

TABLE 5
Toxicity of untreated B. anthracis and of psoralen-treated B. anthracis.

| | Untreated | | Psoralen-treated | |
|---|---|---|---|---|
| | | B. anthracis | | B. anthracis |
| | | Sterne ΔspoIIE | | Sterne ΔspoIIE |
| | | ΔuvrAB | | ΔuvrAB |
| | B. anthracis | cyaK346Q/K353Q | B. anthracis | cyaK346Q/K353Q |
| Experiment | Sterne | (Sterne 3). | Sterne | (Sterne 3). |
| 1 | <1 × 10² | <1 × 10³ | >1 × 10⁸ | >1 × 10⁸ |
| | | | (2000 nM S-59) | (150 nM S-59) |
| 2 | 7.5 × 10 | 7.5 × 10 | 1 × 10⁸ | 5 × 10⁷ |
| | | | (2000 nM S-59) | (150 nM S-59) |
| 3 | 3 × 10² | 5 × 10² | >5 × 10⁸ | >5 × 10⁸ |
| | | | (1000 nM S-59) | (50 nM S-59) |

### Example 20

### Some Exemplary B. anthracis Strains Comprising Mutations in pagA

The invention provides a B. anthracis, including an attenuated B. anthracis, engineered to contain a nucleic acid encoding a mutant protective antigen (PA). Provided are nucleic acids encoding a mutant PA that is a dominant negative inhibitor (DNI). Also provided are B. anthracis strains comprising a nucleic acid encoding a mutant PA that is a dominant negative PA. B. anthracis strains comprising nucleic acids encoding the following PA mutants are provided. These PA mutants include K397D; D425K; F427A; double mutant K397D D425K; 2beta2-2beta3 loop deleted mutant (residues 302-325), and the like (see, e.g., U.S. Publ. Pat. Applic. No. 20020039588 of Collier, et al., Aulinger, et al. (2005) Infection Immunity 73:3408-3414; Mourez, et al. (2001) Nature Biotech. 19:958-961; Sellman, et al. (2001) Science 292:695-697; Sellman, et al. (2001) J. Biol. Chem. 276:8371-8376). The nucleic acid and amino acid sequences of PA are disclosed, for example, below. Other disclosures of PA include SEQ ID NO:21 of U.S. Publ. Pat. Applic. No. 20020039588, as well as GenBank Acc. No. AY997299; AY700758; AF306778; AF268967, and the like.

Where there are discrepancies in the numbering of the above mutants and a disclosed sequence of PA, the skilled artisan will be able to account for discrepancies that amount to minor changes in sequence numbering, shifts in sequence numbering, or slight changes in sequences, in order to provide the mutated PA of the present invention.

B. anthracis encoding at least one of the above nucleic acids can be prepared, for example, by methods involving site-directed mutagenesis to produce a plasmid encoding a mutated PA followed by homologous recombination to integrate the nucleic acid encoding mutated PA in the bacterial genome. Site-directed mutagenesis can be accomplished by, e.g., with a QuikChange® Site Directed Mutagenesis Kit (Stratagene, Inc., La Jolla, Ca.) or chemical polynucleotide synthesis (see, e.g., Zheng, et al. (2004) Nucl. Acids Res. 32:el 15). The nucleic acid encoding mutated PA can be attached to two flanking arms, where the arms flank the region to be mutated, and where each arm has a sequence essentially identical to genomic regions that flank genomic sequence for PA. pKSV7 is a plasmid suitable for mediating homologous recombination into the bacterial genome (see, e.g., Smith and Youngman (1992) Biochimie 74:705-711). The pKSV7 plasmid can be modified to contain the nucleic acid encoding mutated PA flanked by two nucleic acids that are essentially homologous to upstream and downstream regions in the bacterial genome. Bacteria containing mutated genomic pagA can be screened and isolated because pKSV7 encodes and introduces an antibiotic resistance gene. Once isolated, the pagA mutations can be confirmed by sequencing.
The present invention contemplates the following embodiments, and the like:
(1) B. anthracis Sterne ΔuvrAB PA K397D;
(2) B. anthracis Strene ΔuvrAB PA D425K;
(3) B. anthracis Strene ΔuvrAB PA K397D/D425K;
(4) B. anthracis Strene ΔuvrAB PA F427A;
(5) B. anthracis Sterne ΔspoIIE PA K397D;
(6) B. anthracis Sterne ΔspoIIE PA D425K;
(7) B. anthracis Strene ΔspoIIE PA K397D/D425K;
(8) B. anthracis Strene ΔspoIIE PA F427A;
(9) B. anthracis Strene ΔspoIIE ΔuvrAB cyaK346Q/K353Q (Strene 3) PA K397D;
(10) B. anthracis Strene ΔspoIIE ΔuvrAB cyaK346Q/K353Q (Strene 3) PA D425K;
(11) B. anthracis Strene ΔspoIIE ΔuvrAB cyaK346Q/K353Q (Strene 3) PA K397D/D425K;
(12) B. anthracis Strene ΔspoIIE ΔuvrAB cyaK346Q/K353Q (Strene 3) PA F427A;
(13) B. anthracis Strene ΔspoIIE ΔuvrAB cyaK346Q/K353Q lefΔ686 PA K397D;
(14) B. anthracis Strene AspoIIE ΔuvrAB cyaK346Q/K353Q lefΔ686 PA D425K;
(15 ) B. anthracis Strene ΔspoIIE ΔuvrAB cyaK346Q/K353Q lefΔ686 PA K397D/D425K;
(16) B. anthracis Strene ΔspoIIE ΔuvrAB cyaK346Q/K353Q lefΔ686 PA F427A;
(17) B. anthracis Strene ΔspoIIE ΔuvrAB lefΔ686 PA K397D;
(18) B. anthracis Strene ΔspoIIE ΔuvrAB lefΔ686 PA D425K;
(19) B. anthracis Strene ΔspoIIE ΔuvrAB lefΔ686 PA K397D/D425K;
(20) B. anthracis Strene ΔspoIIE ΔuvrAB lefΔ686 PA F427A;
(21) B. anthracis Strene ΔuvrAB PA dominant negative inhibitor (DNI);
(22) B. anthracis Strene ΔspoIIE PA dominant negative inhibitor (DNI);
(23) B. anthracis Strene ΔspoIIE ΔuvrAB cyaK346Q/K353Q (Strene 3) PA dominant negative inhibitor (DNI);
(24) B. anthracis Strene ΔspoIIE ΔuvrAB cyaK346Q/K353Q lefΔ686 PA dominant negative inhibitor (DNI); and
(25) B. anthracis Strene ΔspoIIE ΔuvrAB lefΔ686 PA dominant negative inhibitor (DNI).

Modified bacteria comprising the mutations listed above, but derived from a B. anthracis strain other than Strene are also provided.

### Example 21

### Vaccination with Psoralen-treated Sterne 3 Results in Production of Anti-PA Antibodies

Vaccination of mice with B. anthracis Sterne 3, treated with low concentrations of psoralen (50 nM S-59), resulted in the production of detectable antibodies to protective antigen (PA). The mice used were DBA/2 mice (Charles River Laboratories, Wilmington, Mass.). As mentioned above, B. anthracis Sterne 3 is B. anthracis Sterne ΔspoIIE ΔuvrAB cyaK346Q/K353Q. Psoralen treatment was 50 nM S-59 with UVA light at 6.5 Joules per square cm. The vaccination was with 5×10⁸ infectious units (I.U.) of psoralen (50 nM)-treated B. anthracis Sterne 3. In contrast, mice vaccinated with a control strain, B. anthracis Sterne ΔspoIIE treated with high concentrations of psoralen (1000 nM S-59 with UVA at 6.5 Joules per square cm), did not produce detectable anti-PA antibodies. The level of anti-PA antibodies produced after vaccination with 50 nM S-59-treated B. anthracis Sterne 3 was at least 10 -fold greater than the level of anti-PA antibodies produced after vaccination with 1000 nM S-59-treated B. anthracis Sterne 3. Anti-PA antibodies were measured according to Pombo, et al. (Pombo, et al. (2004) 32 Biologicals 157-163). The presence of anti-PA antibodies correlates with protection from B. anthracis (see, e.g., Karginov, et al. (2004) FEMS Immunol. Med. Microbiol. 40:71-74; Little, et al. (2004) Vaccine 22:422-430).

Many modifications and variations of this invention, as will be apparent to one of ordinary skill in the art, can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example. All publications, patents, patent applications, and accession numbers (including both polynucleotide and polypeptide sequences) cited herein are hereby incorporated by reference herein in their entirety for all purposes to the same extent as if each individual publication, patent, patent application or accession number were specifically and individually indicated to be so incorporated by reference.

### ADDITIONAL SEQUENCE INFORMATION

Coding sequence of the uvrA gene of Bacillus anthracis (SEQ ID NO:1):

ATGGATAAGCCGGATGTAGATACGATTGAAGGCTTATCTCCAGCGATTTC
AATCGATCAAAAAACGACGAGTCGTAATCCGCGTTCAACTGTTGGAACGG
TAACGGAGATTTATGATTACTTACGTTTATTATTTGCGCGAATTGGTACG
CCAATTTGTCCGAATCATGGCATTGAAATTACATCGCAAACAGTAGAGCA
AATGGTAGACCGTGTACTTGAGTACCCTGAACGTACGAAATTACAAGTGT
TAGCTCCTATCGTTTCTGGGCGTAAAGGTGCACATGTAAAAGTACTTGAA
GATATTAAGAAGCAAGGTTATGTTCGTGTACGTGTTGATGGTGAAATGCT
CGATGTGTCTGAAGATATTGCGTTAGATAAAAATAAGAAGCATTCTATTG
AAGTTGTAATTGACCGTATTGTTGTAAAAGAAGGAATCGCAAGCCGTCTT
GCTGATTCTCTTGAAAGTGCATTAAAGCTTGGCGGGGGACGAGTGTTAAT
CGATGTTATGGGAGAAGAGGAGCTTCTATTTAGTGAACATCATGCTTGTC
CGCATTGTGGTTTTTCAATTGGAGAATTAGAGCCGCGTATGTTCTCATTC
AATAGTCCGTTCGGTGCATGTCCTTCTTGTGATGGGCTTGGCTCAAAGTT
AGAGGTAGATTTAGAACTTGTTATTCCGAACTGGGATTTATCATTAAATG
AGCATGCGATTGCGCCTTGGGAACCGACAAGTTCACAATATTACCCACAG
CTTTTACAATCTGTATGTAATCATTATGGCGTGGATATGGATGTGCCTGT
AAAAGATATACCGAAAGATTTATTTGATAAAGTGTTGTACGGAAGCGGTG
AAGAGAAAGTTTATTTCCGCTATGTAAATGAATTTGGTCAAGTAAAGGAA
AATGAGATTTTATTTGAAGGTGTTATTCCAAATATTGAACGTCGTTATCG
TGAGACGAGTTCGGATTACATTCGTGAGCAAATGGAAAAGTATATGGCAG
AACAAGCTTGTCCGAAGTGTAAAGGCGGACGCTTAAAGCCTGAAAGTTTA
GCTGTTTTCGTTGGCGGAAAAACGATTGCTGATGTAACGAAGTATTCTGT
TCAAGAAGTACAGGAATTCTTCTCAAATGTGGAGCTAACAGAGAAACAAC
AAAAAATTGCCCATTTAATTTTAAGAGAAATTCAAGAGCGCGTTGGGTTC
TTAGTAAACGTTGGTTTAGATTATTTAACGTTAAGTCGTGCCGCAGGAAC
TTTATCTGGTGGTGAGGCGCAACGTATTCGTTTAGCAACGCAAATTGGTT
CGCGTCTTACTGGGGTGCTTTACATTCTTGATGAGCCTTCTATCGGTTTG
CATCAGCGCGATAACGATCGTCTTATTCGTACATTGCAAGAAATGCGTGA
TTTAGGTAATACGTTAATTGTTGTTGAGCATGATGAAGATACGATGATGG
CAGCTGATTATTTACTGGATATCGGGCCTGGCGCAGGTATTCACGGTGGA
CAAGTTGTATCAGCGGGTACACCAGCTGAAGTGATGCAAGATGAGAATTC
ACTAACAGGTAAGTATTTAAGCGGTAAAGAGTTTATTCCAGTTCCACTTG
AAAGACGTAAAGGTGATGGACGTAAAGTGGAGATTGTCGGTGCAAAAGAG
AATAACTTAAAGAACGCGAAGATGTCATTCCCGCTTGGTACGTTTGTAGC
GGTAACGGGTGTATCTGGATCAGGTAAAAGTACGATGATTAATGAAGTAC
TATATAAATCGTTAGCGCAAAAGTTATATAAAGCGAAAGCGAAGCCAGGT
ACTCATAAAGAAATTAAAGGTCTTGAGCATTTAGATAAAGTTATCGATAT
TGATCAATCGCCAATCGGTCGTACACCACGTTCTAATCCAGCAACCTATA
CAGGTGTGTTCGATGATATTCGTGATGTGTTTGCGCAAACGAATGAAGCG
AAAGTGCGCGGATATCAAAAAGGGCGTTTCAGCTTTAACGTAAAAGGTGG
ACGTTGTGAAGCGTGCCGTGGTGATGGAATTATTAAAATTGAGATGCACT
TCTTACCAGACGTATACGTTCCGTGTGAAGTTTGTCACGGTAAACGTTAC
AACCGTGAAACGTTAGAAGTGAAATATAAAGATAAAAACATTTCTGAAGT
GTTAGGGATGACGATTGAAGACGGAGTAGAGTTCTTCGCTAATATCCCAA
AAATTAAACGTAAACTTCAAACGCTTGTAGACGTTGGGCTTGGTTATATG
AAATTAGGGCAACCAGCCACGACTTTATCTGGTGGTGAAGCACAGCGTGT
GAAATTAGCTTCTGAATTACACCGTCGTTCTACAGGACGTACACTATACA
TTTTAGACGAGCCAACGACTGGTTTACATGCGCATGATATCGCCCGTCTT
CTAGAAGTGCTGCAACGTCTTGTTGAGAGCGGTGAGACGGTACTTGTCAT
TGAACATAATTTAGATGTAATTAAAACAGCGGATTATATCGTTGACCTTG
GACCAGAAGGCGGAGACAAAGGTGGACAAATCGTTGCTTCCGGAACGCCA
GAGCAAGT.

Coding sequence of the uvrB gene of Bacillus anthracis (SEQ ID NO:2):

ATGGCTCACAATAAAACGTTAGCAGGACAGTTATATAGTGAGTTGAAAGA
CTTTTTCCCGAATAATGCAGTTGAATATTTTGTTAGTTATTACGATTATT
ATCAGCCAGAAGCGTATGTGCCACAAACAGATACGTTTATTGAAAAAGAC
GCGCAGATTAATGATGAAATCGATAAATTGCGTCACTCAGCAACGTCCGC
ATTATTTGAACGGGATGATGTAATTATTGTTGCGAGTGTTTCGTGTATAT
ATGGTTTAGGTTCTCCAGAAGAATACCGCGAGTTAGTTGTTTCACTTCGA
GTTGGTATGGAAAAGGACCGCAATCAATTGCTTCGTGAACTTGTTGATGT
GCAGTATGGACGTAATGATATTGATTTCAAGCGTGGTACATTCCGCGTGC
GCGGAGATGTAGTTGAAATCTTCCCGGCATCACTTGACGAGCATTGCATT
CGAATTGAGTTTTTTGGCGATGAAATTGATCGTATTCGCGAAGTAAATGC
TTTAACGGGAGAAGTATTAGCAGAACGTGATCATGTAGCAATCTTCCCAG
CATCTCACTTCGTTACACGTGAAGAAAAGATGAAGGTCGCTATTGAAAAT
ATCGAAAAAGAATTAGAAGAGCGTTTAAAGGAATTAAATGATAACGGTAA
GTTGTTAGAAGCGCAGCGTATAGAACAGCGTACACGTTATGATTTAGAAA
TGATGCGCGAGATGGGCTTTTGTTCAGGGATTGAAAACTATTCCCGTCAT
TTAACACTTCGTCCAGCGGGTGCAACGCCGTATACGTTATTAGACTATTT
CCCGAAAGATTTCTTAATCGTTATGGATGAGTCCCACGTATCAGTGCCGC
AAGTAAGAGCGATGTATAACGGGGACCAAGCGCGTAAACAAGTGCTTGTG
GATCATGGATTCCGTCTGCCATCAGCTTTAGATAATAGACCGCTCACATT
TGATGAGTTTGAAGAGAAAACGAATCAAGTTATTTACGTTTCAGCAACGC
CAGGACCGTATGAATTAGAGCAGTCGCCAGAAGTAATAGAACAAATTATT
CGTCCAACAGGGCTTTTAGATCCGCCAATTGATATACGACCAATTGAAGG
GCAGATTGACGATCTATTAGGAGAGATTCAAGATCGCATTGCAAAAAATG
AACGTGTATTAATTACAACTTTAACGAAGAAGATGTCAGAGGATTTAACA
GACTACTTAAAAGATGTAGGAATTAAGGTGAATTATCTGCATTCTGAAGT
GAAAACGTTAGAACGTATTGAAATTATACGAGATCTTCGCCTTGGTAAGT
TTGATGTTCTCGTTGGTATTAACTTATTGCGAGAAGGATTAGATATTCCA
GAAGTATCCCTTGTAGCTATTTTAGATGCCGATAAGGAAGGATTCTTGCG
TTCAGAGCGTTCGTTAATTCAAACAATTGGCCGTGCAGCACGTAATGAAA
ACGGTCGCGTTATTATGTACGCAGATCGTATAACGAGATCGATGGGGATT
GCGATTGAAGAGACGAAGCGTCGTCGTAGTATACAAGAAGCTTACAATGA
AGAGCATGGTATTACGCCGAAAACGATTCAAAAAGGTGTGCGTGATGTAA
TCCGTGCAACGACAGCTGCTGAAGAGCCGGAAACATATGAAGCGACGCCA
GCTAAGAAGATGACGAAAAAAGAGCGTGAAAAGACAATTGCGAAGATGGA
AGCAGAAATGAAAGAAGCAGCAAAAGCA.

Protein sequence of Stage II Sporulation Protein E (SpoIIE) from Bacillus anthracis (SEQ ID NO:16):

MKFEQVFFRWGFIIVVIGFLLGRAYILTNILPFALPFFAAVYVMKRDKMP
LAFLALMGGALSVSIDNLFFTFASIFTFFIYNIFFSRFTRKTVGLVPFQV
FISALTAHLVVVYFAQQTVTMYDLLVSTIEAGLSFVLTMIFLQSVPLLVE
RKGKQQALETEEIVCLIILLASVLTGTTDWFVYDASIQHIFTRYLVLVFA
FIAGAATGSTVGVVTGLILSLANVSSLSQLSLLAFSGLLGGLLKEGKRIG
VSLGLLIGTSLITLYVDKQTNIVTTLIESGVAIAFFLLTPKLVMDRIAKF
MPGTQEHSQDQQQYLRRMRDVTANKINQFANVFAALSNSFSVYGYVEEED
KETEADLFLSTITAKTCQTCFKKDQCWVVNFDKTYDYMKQIMSETEEGTL
QHNRKLVREWDKHCVRGKKVTDLVAGELDFYEGQKLRKQMKENRRIVAEQ
LLGVSKVMEDFAKEIQRERENHQVQEEQILQAFRDFGVEIEHVDIYCLDR
GSIDIEMLIPVASNEHGECEKLVAPMLSDILKENIVVKHEEKSSYPNGHS
LISFGSAKTYSLDTGLATAAKGGGFVSGDSYAMMDLSVGKYALAISDGMG
NGQRAHMESKETVKLLQKILQSGIDEEIAKIKSINSILSLRTTEEMFTTL
DLAMVDLRDASAKFLKIGSTPSFVKRANNILKIEASNLPMGIIEDVEVDV
VGEQLKTGDILIMMSDGIFEGAQHVENHELWMKRKIEKELQTEDPQEIAD
IIMEEVIRSGDGYINDDMTIVVAKVKKNMPKWATIPIVQMQAQ.

Coding sequence of spoIIE from Bacillus anthracis (SEQ ID NO:17):

| | |
|---|---|
| 1 | atgaagtttg agcaagtttt ctttagatgg ggatttatta |
| | ttgttgttat tggttttctt |
| 61 | ttaggacgag catatatatt aacaaacatt ttaccgtttg |
| | cactgccgtt ttttgctgct |
| 121 | gtttatgtta tgaagcggga taaaatgccg cttgcattct |
| | tggccctaat ggggggcgca |
| 181 | ctgtcagttt cgatagataa tttattcttt acctttgcat |
| | ctatttttac tttcttcatt |
| 241 | tataatatct tctttagtcg gtttacacgt aaaactgttg |
| | gacttgttcc attccaagta |
| 301 | tttatctccg cattaaccgc acatttagtt gtcgtatatt |
| | ttgcgcaaca aacagtcacc |
| 361 | atgtacgatt tacttgttag tacaattgag gccgggctta |
| | gcttcgtatt aactatgata |
| 421 | tttttacaaa gtgtcccgct tttagtagaa agaaaaggga |
| | aacaacaagc cttagagaca |
| 481 | gaagaaattg tttgtttaat tatattacta gcatctgttt |
| | taacaggtac aacagattgg |
| 541 | ttcgtatatg acgcttctat tcaacatatt tttactaggt |
| | atttagtgct tgtatttgcg |
| 601 | tttatcgcag gagcggctac aggatctaca gtaggggtcg |
| | tcactgggtt aatattaagc |
| 661 | ctggcaaatg tctccagttt gtcccaactt agcctacttg |
| | ctttttctgg attgcttggt |
| 721 | ggtttgttaa aagaagggaa gcgcataggt gttagtttag |
| | gtttattaat tgggacaagc |
| 781 | ctgattacgc tatatgtaga caagcaaaca aacattgtga |
| | caactttaat cgaatctggt |
| 841 | gtggcgattg ctttcttctt attaacaccg aaacttgtta |
| | tggatcgtat tgctaaattt |
| 901 | atgccaggca cacaggagca ttcgcaagat caacaacagt |
| | atttaagaag gatgcgtgat |
| 961 | gttacagcga ataaaattaa tcaatttgct aatgtgtttg |
| | ctgctttatc taatagcttt |
| 1021 | tctgtatatg gatacgtgga ggaagaagat aaagagacgg |
| | aggcagatct gttcttaagt |
| 1081 | acaattactg caaaaacatg tcaaacatgc tttaaaaagg |
| | accaatgctg ggtagttaat |
| 1141 | ttcgataaaa catacgatta tatgaaacaa ataatgagtg |
| | aaacagaaga ggggacgtta |
| 1201 | cagcataatc ggaagttagt tcgtgaatgg gataagcatt |
| | gtgtgagagg aaagaaagtg |
| 1261 | acggatttag tggcaggcga attagatcac ttctatgagg |
| | gacaaaaatt aagaaaacaa |
| 1321 | atgaaagaaa atcgtagaat agtagcggag caactattgg |
| | gtgtatcaaa agttatggag |
| 1381 | gattttgcta aggagataca aagagagcga gaaaaccatc |
| | aagtacagga agaacagatt |
| 1441 | ctgcaagcgt ttcgtgattt tggtgtagaa atagagcatg |
| | ttgatattta ttgtttagat |
| 1501 | agaggaagta ttgatattga aatgttgatt ccagttgcat |
| | ctaatgaaca cggagagtgt |
| 1561 | gaaaaattag ttgcaccgat gctttctgat attctaaagg |
| | aaaatatcgt tgttaagcat |
| 1621 | gaagaaaaat cttcttatcc gaatggccac agcttaatat |
| | catttggttc agcaaaaacg |
| 1681 | tattctcttg atacgggctt agccacagct gcaaaaggcg |
| | gtgggtttgt ttcaggtgat |
| 1741 | tcttatgcga tgatggattt aagtgttggt aaatatgctc |
| | ttgcgattag tgatggtatg |
| 1801 | ggaaatgggc aaagagctca tatggagagt aaagaaacag |
| | tgaaattatt acaaaaaata |
| 1861 | cttcaatcag gcattgatga agaaatagcg attaagtcta |
| | ttaactctat tctttcttta |
| 1921 | agaacaacag aagagatgtt tactacgtta gatttagcta |
| | tggtagattt gcgggatgcg |
| 1981 | agtgcgaagt ttttaaagat tggatcgacg ccgagttttg |
| | ttaaacgcgc aaacaatatt |
| 2041 | ttgaaaattg aagcaagtaa tttgccgatg ggaataattg |
| | aggatgttga agttgatgta |
| 2101 | gtgggtgagc aattaaaaac aggcgatatc cttattatga |
| | tgagcgatgg gatttttgag |
| 2161 | ggagcgcaac atgtggagaa tcatgaatta tggatgaagc |
| | gtaaaattaa agagttgcaa |
| 2221 | actgaagatc cgcaagaaat cgctgatatc atcatggaag |
| | aggtaattcg ctctggtgat |
| 2281 | ggttatataa atgatgatat gactattgta gtggcaaaag |
| | tgaagaaaaa tatgccgaag |
| 2341 | tgggctacca ttccaattgt gggaatgcag gcacaataa. |

Protein sequence of protective antigen from Bacillus anthracis (SEQ ID NO:18):

MKKRKVLIPLMALSTILVSSTGNLEVIQAEVKQENRLLNESESSSQGLLG
YYFSDLNFQAPMVVTSSTTGDLSIPSSELENIPSENQYFQSAIWSGFIKV
KKSDEYTFATSADNHVTMWVDDQEVINKASNSNKIRLEKGRLYQIKIQYQ
RENPTEKGLDFKLYWTDSQNKKEVISSDNLQLPELKQKSSNSRKKRSTSA
GPTVPDRDNDGIPDSLEVEGYTVDVKNKRTFLSPWISHNIHEKKGLTKYK
SSPEKWSTASDPYSDFEDVTGRIDKNVSPEARHPLVAAYPIVHVDMENII
LSKNEDQSTQNTDSQTRTISKNTSTSRTHTSEVHGNAEVHASFFDIGGSV
SAGFSNSNSSTVAIDHSLSLAGERTWAETMGLNTADTARLNANIRYVNTG
TAPIYNVLPTTSLVLGKNQTLATIKAKENQLSQILAPNNYYPSKNLAPIA
LNAQDDFSSTPITMNYNQFLELEKTKQLRLDTDQVYGNIATYNFENGRVR
VDTGSNWSEVLPQIQETTARIIFNGKDLNLVERRIAAVNPSDPLETTKPD
MTLKEALKIAFGFNEPNGNLQYQGKDITEFDFNFDQQTSQNIKNQLAELN
ATNIYTVLDKIKLNAKMNILIRDKRFHYDRNNIAVGADESVVKEAHREVI
NSSTEGLLLNIDKDIRKILSGYIVEIEDTEGLKEVINDRYDMLNISSLRQ
DGKTFIDFKKYNDKLPLYISNPNKKVNVYAVTKENTIINPSENGDTSTNG
IKKILIFSKKGYEIG.

Nucleotide sequence encoding protective antigen (pagA) from Bacillus anthracis (SEQ ID NO:19):

| | |
|---|---|
| 1 | atgaaaaaac gaaaagtgtt aataccatta atggcattgt |
| | ctacgatatt agtttcaagc |
| 61 | acaggtaatt tagaggtgat tcaggcagaa gttaaacagg |
| | agaaccggtt attaaatgaa |
| 121 | tcagaatcaa gttcccaggg gttactagga tactatttta |
| | gtgatttgaa ttttcaagca |
| 181 | cccatggtgg ttacctcttc tactacaggg gatttatcta |
| | ttcctagttc tgagttagaa |
| 241 | aatattccat cggaaaacca atattttcaa tctgctattt |
| | ggtcaggatt tatcaaagtt |
| 301 | aagaagagtg atgaatatac atttgctact tccgctgata |
| | atcatgtaac aatgtgggta |
| 361 | gatgaccaag aagtgattaa taaagcttct aattctaaca |
| | aaatcagatt agaaaaagga |
| 421 | agattatatc aaataaaaat tcaatatcaa cgagaaaatc |
| | ctactgaaaa aggattggat |
| 481 | ttcaagttgt actggaccga ttctcaaaat aaaaaagaag |
| | tgatttctag tgataactta |
| 541 | caattgccag aattaaaaca aaaatcttcg aactcaagaa |
| | aaaagcgaag tacaagtgct |
| 601 | ggacctacgg ttccagaccg tgacaatgat ggaatccctg |
| | attcattaga ggtagaagga |
| 661 | tatacggttg atgtcaaaaa taaaagaact tttctttcac |
| | catggatttc taatattcat |
| 721 | gaaaagaaag gattaaccaa atataaatca tctcctgaaa |
| | aatggagcac ggcttctgat |
| 781 | ccgtacagtg atttcgaaaa ggttacagga cggattgata |
| | agaatgtatc accagaggca |
| 841 | agacaccccc ttgtggcagc ttatccgatt gtacatgtag |
| | atatggagaa tattattctc |
| 901 | tcaaaaaatg aggatcaatc cacacagaat actgatagtc |
| | aaacgagaac aataagtaaa |
| 961 | aatacttcta caagtaggac acatactagt gaagtacatg |
| | gaaatgcaga agtgcatgcg |
| 1021 | tcgttctttg atattggtgg gagtgtatct gcaggattta |
| | gtaattcgaa ttcaagtacg |
| 1081 | gtcgcaattg atcattcact atctctagca ggggaaagaa |
| | cttgggctga aacaatgggt |
| 1141 | ttaaataccg ctgatacagc aagattaaat gccaatatta |
| | gatatgtaaa tactgggacg |
| 1201 | gctccaatct acaacgtgtt accaacgact tcgttagtgt |
| | taggaaaaaa tcaaacactc |
| 1261 | gcgacaatta aagctaagga aaaccaatta agtcaaatac |
| | ttgcacctaa taattattat |
| 1321 | ccttctaaaa acttggcgcc aatcgcatta aatgcacaag |
| | acgatttcag ttctactcca |
| 1381 | attacaatga attacaatca atttcttgag ttagaaaaaa |
| | cgaaacaatt aagattagat |
| 1441 | acggatcaag tatatgggaa tatagcaaca tacaattttg |
| | aaaatggaag agtgagggtg |
| 1501 | gatacaggct cgaactggag tgaagtgtta ccgcaaattc |
| | aagaaacaac tgcacgtatc |
| 1561 | atttttaatg gaaaagattt aaatctggta gaaaggcgga |
| | tagcggcggt taatcctagt |
| 1621 | gatccattag aaacgactaa accggatatg acattaaaag |
| | aagcccttaa aatagcattt |
| 1681 | ggatttaacg aaccgaatgg aaacttacaa tatcaaggga |
| | aagacataac cgaatttgat |
| 1741 | tttaatttcg atcaacaaac atctcaaaat atcaagaatc |
| | agttagcgga attaaacgca |
| 1801 | actaacatat atactgtatt agataaaatc aaattaaatg |
| | caaaaatgaa tattttaata |
| 1861 | agagataaac gttttcatta tgatagaaat aacatagcag |
| | ttggggcgga tgagtcagta |
| 1921 | gttaaggagg ctcatagaga agtaattaat tcgtcaacag |
| | agggattatt gttaaatatt |
| 1981 | gataaggata taagaaaaat attatcaggt tatattgtag |
| | aaattgaaga tactgaaggg |
| 2041 | cttaaagaag ttataaatga cagatatgat atgttgaata |
| | tttctagttt acggcaagat |
| 2101 | ggaaaaacat ttatagattt taaaaaatat aatgataaat |
| | taccgttata tataagtaat |
| 2161 | cccaattata aggtaaatgt atatgctgtt actaaagaaa |
| | acactattat taatcctagt |
| 2221 | gagaatgggg atactagtac caacgggatc aagaaaattt |
| | taatcttttc taaaaaaggc |
| 2281 | tatgagatag gataa. |

Protein sequence of lethal factor from Bacillus anthracis (SEQ ID NO:20):

MNIKKEFIKVISMSCLVTAITLSGPVFIPLVQGAGGHGDVGMHVKEKEKN
KDENKRKDEERNKTQEEHLKEIMKHIVKIEVKGEEAVKKEAAEKLLEKVP
SDVLEMYKAIGGKIYIVDGDITKHISLEALSEDKKKIKDIYGKDALLHEH
YVYAKEGYEPVLVIQSSEDYVENTEKALNVYYEIGKILSRDILSKINQPY
QKFLDVLNTIKNASDSDGQDLLFTNQLKEHPTDFSVEFLEQNSNEVQEVF
AKAFAYYIEPQHRDVLQLYAPEAFNYMDKFNEQEINLSLEELKDQRMLAR
YEKWEKIKQHYQHWSDSLSEEGRGLLKKLQIPIEPKKDDIIHSLSQEEKE
LLKRIQIDSSDFLSTEEKEFLKKLQIDIRDSLSEEEKELLNRIQVDSSNP
LSEKEKEFLKKLKLDIQPYDINQRLQDTGGLIDSPSINLDVRKQYKRDIQ
NIDALLHQSIGSTLYNKIYLYENMNINNLTATLGADLVDSTDNTKINRGI
FNEFKKNFKYSISSNYMIVDINERPALDNERLKWRIQLSPDTRAGYLENG
KLILQRNIGLEIKDVQIIKQSEKEYIRIDAKVVPKSKIDTKIQEAQLNIN
QEWNKALGLPKYTKLITFNVHNRYASNIVESAYLILNEWKNNIQSDLIKK
VTNYLVDGNGRFVFTDITLPNIAEQYTHQDEIYEQVHSKGLYVPESRSIL
LHGPSKGVELRNDSEGFIHEFGHAVDDYAGYLLDKNQSDLVTNSKKFIDI
FKEEGSNLTSYGRTNEAEFFAEAFRLMHSTDHAERLKVQKNAPKTFQFIN
DQIKFIINS.

Nucleotide sequence encoding lethal factor (lef) from Bacillus anthracis (SEQ 21):

| | |
|---|---|
| 1 | atgaatataa aaaaagaatt tataaaagta |
| | attagtatgt catgtttagt aacagcaatt |
| 61 | actttgagtg gtcccgtctt tatccccctt |
| | gtacaggggg cgggcggtca tggtgatgta |
| 121 | ggtatgcacg taaaagagaa agagaaaaat |
| | aaagatgaga ataagagaaa agatgaagaa |
| 181 | cgaaataaaa cacaggaaga gcatttaaag |
| | gaaatcatga aacacattgt aaaaatagaa |
| 241 | gtaaaagggg aggaagctgt taaaaaagag |
| | gcagcagaaa agctacttga gaaagtacca |
| 301 | tctgatgttt tagagatgta taaagcaatt |
| | ggaggaaaga tatatattgt ggatggtgat |
| 361 | attacaaaac atatatcttt agaagcatta |
| | tctgaagata agaaaaaaat aaaagacatt |
| 421 | tatgggaaag atgctttatt acatgaacat |
| | tatgtatatg caaaagaagg atatgaaccc |
| 481 | gtacttgtaa tccaatcttc ggaagattat |
| | gtagaaaata ctgaaaaggc actgaacgtt |
| 541 | tattatgaaa taggtaagat attatcaagg |
| | gatattttaa gtaaaattaa tcaaccatat |
| 601 | cagaaatttt tagatgtatt aaataccatt |
| | aaaaatgcat ctgattcaga tggacaagat |
| 661 | cttttattta ctaatcagct taaggaacat |
| | cccacagact tttctgtaga attcttggaa |
| 721 | caaaatagca atgaggtaca agaagtattt |
| | gcgaaagctt ttgcatatta tatcgagcca |
| 781 | cagcatcgtg atgttttaca gctttatgca |
| | ccggaagctt ttaattacat ggataaattt |
| 841 | aacgaacaag aaataaatct atccttggaa |
| | gaacttaaag atcaacggat gctggcaaga |
| 901 | tatgaaaaat gggaaaagat aaaacagcac |
| | tatcaacact ggagcgattc tttatctgaa |
| 961 | gaaggaagag gacttttaaa aaagctgcag |
| | attcctattg agccaaagaa agatgacata |
| 1021 | attcattctt tatctcaaga agaaaaagag |
| | cttctaaaaa gaatacaaat tgatagtagt |
| 1081 | gattttttat ctactgagga aaaagagttt |
| | ttaaaaaagc tacaaattga tattcgtgat |
| 1141 | tctttatctg aagaagaaaa agagctttta |
| | aatagaatac aggtggatag tagtaatcct |
| 1201 | ttatctgaaa aagaaaaaga gtttttaaaa |
| | aagctgaaac ttgatattca accatatgat |
| 1261 | attaatcaaa ggttgcaaga tacaggaggg |
| | ttaattgata gtccgtcaat taatcttgat |
| 1321 | gtaagaaagc agtataaaag ggatattcaa |
| | aatattgatg ctttattaca tcaatccatt |
| 1381 | ggaagtacct tgtacaataa aatttatttg |
| | tatgaaaata tgaatatcaa taaccttaca |
| 1441 | gcaaccctag gtgcggattt agttgattcc |
| | actgataata ctaaaattaa tagaggtatt |
| 1501 | ttcaatgaat tcaaaaaaaa tttcaaatat |
| | agtatttcta gtaactatat gattgttgat |
| 1561 | ataaatgaaa ggcctgcatt agataatgag |
| | cgtttgaaat ggagaatcca attatcacca |
| 1621 | gatactcgag caggatattt agaaaatgga |
| | aagcttatat tacaaagaaa catcggtctg |
| 1681 | gaaataaagg atgtacaaat aattaagcaa |
| | tccgaaaaag aatatataag gattgatgcg |
| 1741 | aaagtagtgc caaagagtaa aatagataca |
| | aaaattcaag aagcacagtt aaatataaat |
| 1801 | caggaatgga ataaagcatt agggttacca |
| | aaatatacaa agcttattac attcaacgtg |
| 1861 | cataatagat atgcatccaa tattgtagaa |
| | agtgcttatt taatattgaa tgaatggaaa |
| 1921 | aataatattc aaagtgatct tataaaaaag |
| | gtaacaaatt acttagttga tggtaatgga |
| 1981 | agatttgttt ttaccgatat tactctccct |
| | aatatagctg aacaatatac acatcaagat |
| 2041 | gagatatatg agcaagttca ttcaaaaggg |
| | ttatatgttc cagaatcccg ttctatatta |
| 2101 | ctccatggac cttcaaaagg tgtagaatta |
| | aggaatgata gtgagggttt tatacacgaa |
| 2161 | tttggacatg ctgtggatga ttatgctgga |
| | tatctattag ataagaacca atctgattta |
| 2221 | gttacaaatt ctaaaaaatt cattgatatt |
| | tttaaggaag aagggagtaa tttaacttcg |
| 2281 | tatgggagaa caaatgaagc ggaatttttt |
| | gcagaagcct ttaggttaat gcattctacg |
| 2341 | gaccatgctg aacgtttaaa agttcaaaaa |
| | aatgctccga aaactttcca atttattaac |
| 2401 | gatcagatta agttcattat taactcataa. |

Protein sequence of edema factor from Bacillus anthracis (SEQ ID NO:22):

MTRNKFIPNKFSIISFSVLLFAISSSQAIEVNAMNEHYTESDIKRNHKTE
KNKTEKEKFKDSINNLVKTEFTNETLDKIQQTQDLLKKIPKDVLEIYSEL
GGEIYFTDIDLVEHKELQDLSEEEKNSMNSRGEKVPFASRFVFEKKRETP
KLIINIKDYAINSEQSKEVYYEIGKGISLDIISKDKSLDPEFLNLIKSLS
DDSDSSDLLFSQKFKEKLELNNKSIDINFIKENLTEFQHAFSLAFSYYFA
PDHRTVLELYAPDMFEYMNKLEKGGFEKISESLKKEGVEKDRIDVLKGEK
ALKASGLVPEHADAFKKIARELNTYILFRPVNKLATNLIKSGVATKGLNV
HGKSSDWGPVAGYIPFDQDLSKKHGQQLAVEKGNLENKKSITEHEGEIGK
IPLKLDHLRIEELKENGIILKGKKEIDNGKKYYLLESNNQVYEFRISDEN
NEVQYKTKEGKITVLGEKFNWRNIEVMAKNVEGVLKPLTADYDLFALAPS
LTEIKKQIPQKEWDKVVNTPNSLEKQKGVTNLLIKYGIERKPDSTKGTLS
NWQKQMLDRLNEAVKYTGYTGGDVVNHGTEQDNEEFPEKDNEIFIINPEG
EFILTKNWEMTGRFIEKNITGKDYLYYFNRSYNKIAPGNKAYIEWTDPIT
KAKINTIPTSAEFIKNLSSIRRSSNVGVYKDSGDKDEFAKKESVKKIAGY
LSDYYNSANHIFSQEKKRKISIFRGIQAYNEIENVLKSKQIAPEYKNYFQ
YLKERITNQVQLLLTHQKSNIEFKLLYKQLNFTENETDNFEVFQKIIDEK
.

Nucleotide sequence encoding edema factor (cya) from Bacillus anthracis (SEQ ID NO:23):

| | |
|---|---|
| 1 | atgactagaa ataaatttat acctaataag tttagtatta |
| | tatccttttc agtattacta |
| 61 | tttgctatat cctcctcaca ggctatagaa gtaaatgcta |
| | tgaatgaaca ttacactgag |
| 121 | agtgatatta aaagaaacca taaaactgaa aaaaataaaa |
| | ctgaaaaaga aaaatttaaa |
| 181 | gacagtatta ataacttagt taaaacagaa tttaccaatg |
| | aaactttaga taaaatacag |
| 241 | cagacacaag acttattaaa aaagatacct aaggatgtac |
| | ttgaaattta tagtgaatta |
| 301 | ggaggagaaa tctattttac agatatagat ttagtagaac |
| | ataaggagtt acaagattta |
| 361 | agtgaagaag agaaaaatag tatgaatagt agaggtgaaa |
| | aagttccgtt tgcatcccgt |
| 421 | tttgtatttg aaaagaaaag ggaaacacct aaattaatta |
| | taaatatcaa agattatgca |
| 481 | attaatagtg aacaaagtaa agaagtatat tatgaaattg |
| | gaaaggggat ttctcttgat |
| 541 | attataagta aggataaatc tctagatcca gagtttttaa |
| | atttaattaa gagtttaagc |
| 601 | gatgatagtg atagtagcga ccttttattt agtcaaaaat |
| | ttaaagagaa gctagaattg |
| 661 | aataataaaa gtatagatat aaattttata aaagaaaatt |
| | taactgaatt tcagcatgcg |
| 721 | ttttctttag cgttttctta ttattttgca cctgaccata |
| | gaacggtatt agagttatat |
| 781 | gcccccgaca tgtttgagta tatgaataag ttagaaaaag |
| | ggggatttga gaaaataagt |
| 841 | gaaagtttga agaaagaagg tgtggaaaaa gataggattg |
| | atgtgctgaa aggagaaaaa |
| 901 | gcacttaaag cttcaggttt agtaccagaa catgcagatg |
| | cttttaaaaa aattgctaga |
| 961 | gaattaaata catatattct ttttaggcct gttaataagt |
| | tagctacaaa ccttattaaa |
| 1021 | agtggtgtgg ctacaaaggg attgaatgtt catggaaaga |
| | gttcggattg gggccctgta |
| 1081 | gctggataca taccatttga tcaagattta tctaagaagc |
| | atggtcaaca attagctgtc |
| 1141 | gagaaaggaa atttagaaaa taaaaaatca attacagagc |
| | atgaaggtga aataggtaaa |
| 1201 | ataccattaa agttagacca tttaagaata gaagagttaa |
| | aggaaaatgg gataattttg |
| 1261 | aagggtaaaa aagaaattga taatggtaaa aaatattatt |
| | tgttagaatc gaataatcag |
| 1321 | gtatatgaat ttagaattag cgatgaaaac aacgaagtac |
| | aatacaagac aaaagaaggt |
| 1381 | aaaattactg ttttagggga aaaattcaat tggagaaata |
| | tagaagtgat ggctaaaaat |
| 1441 | gtagaagggg tcttgaagcc gttaacagct gactatgatt |
| | tatttgcact tgccccaagt |
| 1501 | ttaacagaaa taaaaaaaca aataccacaa aaagaatggg |
| | ataaagtagt taacacccca |
| 1561 | aattcattag aaaagcaaaa aggtgttact aatttattga |
| | ttaaatatgg aattgagagg |
| 1621 | aaaccggatt caactaaggg aactttatca aattggcaaa |
| | aacaaatgct tgatcgtttg |
| 1681 | aatgaagcag tcaaatatac aggatataca gggggggatg |
| | tggttaacca tggcacagag |
| 1741 | caagataatg aagagtttcc tgaaaaagat aacgaaattt |
| | ttataattaa tccagaaggt |
| 1801 | gaatttatat taactaaaaa ttgggagatg acaggtagat |
| | ttatagaaaa aaacattacg |
| 1861 | ggaaaagatt atttatatta ttttaaccgt tcttataata |
| | aaatagctcc tggtaataaa |
| 1921 | gcttatattg agtggactga tccgattaca aaagccaaaa |
| | taaataccat ccctacgtca |
| 1981 | gcagagttta taaaaaactt atccagtatc agaagatctt |
| | caaatgtagg agtttataaa |
| 2041 | gatagtggcg acaaagacga atttgcaaaa aaagaaagcg |
| | tgaaaaaaat tgcaggatat |
| 2101 | ttgtcagact attacaattc agcaaatcat attttttctc |
| | aggaaaaaaa gcgtaaaata |
| 2161 | tcaatatttc gtggaatcca agcctataat gaaattgaaa |
| | atgttctaaa atctaaacaa |
| 2221 | atagcaccag aatacaaaaa ttattttcaa tatttaaagg |
| | aaaggattac caatcaagtt |
| 2281 | caattgcttc taacacatca aaaatctaat attgaattta |
| | aattattgta taaacaatta |
| 2341 | aactttacag aaaatgaaac ggataatttt gaggtcttcc |
| | aaaaaattat tgatgaaaaa |
| 2401 | taa. |

Protein sequence of Lex Repressor (LexA) from Bacillus anthracis (SEQ ID NO:25):

MEKLTKRQQDILDFIKLKVQEKGYPPSVREIGQAVGLASSSTVHGHLSRL
EEKGYIRRDPTKPRAIEILGEDRMDTETQSVIQVPIVGKVTAGLPITAVE
SVEEHFPLPASIVAGADQVFMLRISGDSMIEAGIFDGDLVVVRQQQSAYN
GEIVVALTEDNEATVKRFYKEKDHFRLQPENSSLEPIILKQVSVIGKVIG
VYRDLH.

Nucleotide sequence encoding Lex Repressor (LexA) from Bacillus anthracis SEQ ID NO:25):

| | |
|---|---|
| 1 | atggaaaagt taacgaaacg ccagcaagac attctcgact |
| | ttattaagct aaaagtacaa |
| 61 | gaaaaaggat atccaccctc cgtacgtgaa atcggtcaag |
| | cagtcggcct cgcttctagt |
| 121 | tctacagtgc acggacattt atcaagatta gaagaaaaag |
| | gatacattcg acgcgatcca |
| 181 | acaaaaccac gtgcaattga aattttaggt gaagaccgaa |
| | tggatacaga aacacaatct |
| 241 | gttattcaag ttccaattgt cggaaaagtt actgccggtt |
| | taccaattac agcggtcgaa |
| 301 | agcgtagagg agcacttccc tcttccagct agcattgtcg |
| | caggagcaga tcaagtgttt |
| 361 | atgttacgta tttccgggga tagtatgatt gaggctggca |
| | ttttcgatgg agatttagtt |
| 421 | gttgttcgcc aacaacagtc tgcatataat ggtgaaattg |
| | tagtcgcttt aacagaagat |
| 481 | aatgaagcaa ctgttaaacg tttctataaa gaaaaagacc |
| | atttccgtct acaaccggaa |
| 541 | aactcttcat tagaacctat cattttaaag caagtgtcag |
| | ttatcggtaa agtaattggc |
| 601 | gtatatcgtg atttacatta a. |

Protein sequence of RecA from Bacillus anthracis (SEQ ID NO:26):

MSDRQAALDMALKQIEKQFGKGSIMKLGEQAERKVSTVSSGSLALDVALG
VGGYPRGRIIEIYGPESSGKTTVSLHAIAEVQRQGGQAAFIDAEHAMDPV
YAQKLGVNIDELLLSQPDTGEQGLEIAEALVRSGAVDIIVIDSVAALVPK
AEIEGDMGDSHVGLQARLMSQALRKLSGAINKSKTIAIFINQIREKVGVM
FGNPETTPGGRALKFYSTVRLEVRRAEQLKQGNDIVGNKTKVKVVKNKVA
PPFRVAEVDIMYGEGISREGEILDMASELDIVQKSGAWYSYNEERLGQGR
ENSKQFLKENTDLREEIAFFIREHHGISEDSGAEGMEDPNLLD.

Coding sequence of the uvrC gene of Bacillus anthracis (SEQ ID NO:28):

ATGAAAGATAGGCAAGGAACGGTTATATATGTCGGAAAGGCAAAAGTGCT
TAAAAATCGTGTGCGCTCGTACTTTACTGGTTCGCATGACGGGAAAACAC
TTCGGTTAGTAGGAGAAATTGTAGATTTTGAATATATTGTAACCTCCTCA
AATCTAGAGGCGCTCATTTTGGAGTTAAACTTAATAAAAAAACATGACCC
AAAATATAATATTCAATTAAAAGATGATAAAACATATCCTTTTATTAAAA
TTACAGCTGAGAAACAACCGCGCTTACTTATTACGCGAAATGTAAAAAAG
GATAAAGGAAAGTATTTTGGCCCTTATCCGAATGCACAATCAGCTCATGA
AACGAAAAAACTGCTGGATCGTATGTATCCGCTTCGTAAGTGCTCAAATA
TGCCGGATAAAGTTTGTTTATATTATCATATGGGTCAATGTTTAGCACCT
TGTGTGAAAGAAGTGACGGAAGAACAAAATAAAGAAATTGTAGATGAGAT
TATTAAGTTTTTAAATGGTGGGCATAAAGAAGTTCGTTCAGAATTAGAAA
CAAAAATGTATGAAGCTTCAGAGAAACTAGAGTTTGAACGTGCAAAAGAG
TTACGTGATCAAATCGCTCATATCGATGCGATTATGGAAAAACAAAAGAT
GATTATGAGTGATTTAGTGGACCGTGATGTGTTTGGCTATGCAGTTGATA
AAGGGTGGATGTGTGTTCAAGTTTTCTTCGTTCGGAAAGGAAAGTTAATT
GAACGTGATGTTTCTATGTTTCCAATATATGATGAACCAGAAGAGGGATT
CTTAACGTTTATCGGTCAATTTTATGAAAACAGCAGTCATTTTAAGCCGA
AAGAAATAGTTTGTTCCAGGAAGTATAGACTCAGAATTAGTAGAACGCTT
TTTAGAAGTGGAAGCGACACAGCCGAAACGCGGTAAGAAAAAAGATCTTG
TAGAACTGGCAAAAAAAATGCGAAGATTGCCCTGGAAGAGAAATTCTATT
TAATTGAACGTGATGAAGAGCGAACGATTAAAGCTGTAGAGAATTTAGGG
AAGCAGCTCGGAATTGAAACGCCTTATCGTATTGAAGCATTTGATAACTC
AAATATTCAAGGGACAAATCCTGTTTCTGCAATGATTGCTTTTATTGATG
GGAAACCAGCTAAGAAAGAATACAGGAAATATAAAATTAAAACAGTTCAA
GGACCAGATGATTATGAGTCTATGAGAGAAGTTGTGAGACGCCGTTATAC
AAGGGCGCTGAAAGAGGGTTTACCTTTACCAGATTTAATCATTATTGATG
GCGGAAAAGGTCATCTGGCGGCTGCAAGTGATGTTCTAGAAAATGAGCTC
GGTTTATATATTCCGATGGCAGGTCTTGTAAAAGATGACAAACATAAAAC
ATCTCATTTAATTATTGGAGATCCACCTGAACCTGTGATGCTGGAGAGAA
ATAGCCAAGAATTTTATTTATTGCAGCGTGTTCAAGATGAAGTGCATCGA
TTTGCAATTACATTTCATCGTCAATTACACGGGAAATCTGTCATTCAATC
AGCACTGGATGATATTCCTGGAATCGGTGATAAACGGAAAAAGGTATTGT
TAAAACATTTTGGTTCATTAAAGAAGATGAAAGAAGCTTCTATAGAGGAA
TTTGTCGAAGCAGGTATGCCGAAAAATGTCGCAGAGACGATTTATACTTA
TTTAACAGATAAGAAGACGTTG.

The following nucleotide sequence is that of the wild type E. coli RecA coding sequence (from GenBank Acc. No. V00328) (SEQ ID NO:50):

| | |
|---|---|
| 1 | atggctatcg acgaaaacaa acagaaagcg ttggcggcag |
| | cactgggcca gattgagaaa |
| 61 | caatttggta aaggctccat catgcgcctg ggtgaagacc |
| | gttccatgga tgtggaaacc |
| 121 | atctctaccg gttcgctttc actggatatc gcgcttgggg |
| | caggtggtct gccgatgggc |
| 181 | cgtatcgtcg aaatctacgg accggaatct tccggtaaaa |
| | ccacgctgac gctgcaggtg |
| 241 | atcgccgcag cgcagcgtga aggtaaaacc tgtgcgttta |
| | tcgatgctga acacgcgctg |
| 301 | gacccaatct acgcacgtaa actgggcgtc gatatcgaca |
| | acctgctgtg ctcccagccg |
| 361 | gacaccggcg agcaggcact ggaaatctgt gacgccctgg |
| | cgcgttctgg cgcagtagac |
| 421 | gttatcgtcg ttgactccgt ggcggcactg acgccgaaag |
| | cggaaatcga aggcgaaatc |
| 481 | ggcgactctc acatgggcct tgcggcacgt atgatgagcc |
| | aggcgatgcg taagctggcg |
| 541 | ggtaacctga agcagtccaa cacgctgctg atcttcatca |
| | accagatccg tatgaaaatt |
| 601 | ggtgtgatgt tcggtaaccc ggaaaccact accggtggta |
| | acgcgctgaa attctacgcc |
| 661 | tctgttcgtc tcgacatccg tcgtatcggc gcggtgaaag |
| | agggcgaaaa cgtggtgggt |
| 721 | agcgaaaccc gcgtgaaagt ggtgaagaac aaaatcgctg |
| | cgccgtttaa acaggctgaa |
| 781 | ttccagatcc tctacggcga aggtatcaac ttctacggcg |
| | aactggttga cctgggcgta |
| 841 | aaagagaagc tgatcgagaa agcaggcgcg tggtacagct |
| | acaaaggtga gaagatcggt |
| 901 | cagggtaaag cgaatgcgac tgcctggctg aaagataacc |
| | cggaaaccgc gaaagagatc |
| 961 | gagaagaaag tacgtgagtt gctgctgagc aacccgaact |
| | caacgccgga tttctctgta |
| 1021 | gatgatagcg aaggcgtagc agaaactaac gaagattttt |
| | aa. |

The wild type E. coli RecA amino acid sequence is as follows (from GenBank Acc. No. V00328) (SEQ ID NO:51):

MAIDENKQKALAAALGQIEKQFGKGSIMRLGEDRSMDVETISTGSLSLDI
ALGAGGLPMGRIVEIYGPESSGKTTLTLQVIAAAQREGKTCAFIDAEHAL
DPIYARKLGVDIDNLLCSQPDTGEQALEICDALARSGAVDVIVVDSVAAL
TPKAEIEGEIGDSHMGLAARMMSQAMRKLAGNLKQSNTLLIFINQIRMKI
GVMFGNPETTTGGNALKFYASVRLDIRRIGAVKEGENVVGSETRVKVVKN
KIAAPFKQAEFQILYGEGINFYGELVDLGVKEKLIEKAGAWYSYKGEKIG
QGKANATAWLKDNPETAKEIEKKVRELLLSNPNSTPDFSVDDSEGVAETN
EDF.

The amino acid sequence of a predicted temperature sensitive E. coli RecA protein is as follows (SEQ ID NO:52) (the mutation relative to the GenBank Acc. No. V00328 wild-type sequence is underlined in the sequence):

MAIDENKQKALAAALGQIEKQFGKGSIMRLGEDRSMDVETISTGSLSLDI
ALGAGGLPMGRIVEIYGPESSGKTTLTLQVIAAAQREGKTCAFIDAEHAL
DPIYARKLGVDIDNLLCSQPDTGEQALEICDALARSGAVDVIVVDSVAAL
TPKAEIEGEIGDSHMGLAARMMSQAMRKLAGNLKQSNTLLIFINQIRMKI
GVMFGNPETTTGGNALKFYASVRLDIRRIGAVKEGENVVGSETRVKMVKN
KIAAPFKQAEFQILYGEGINFYGELVDLGVKEKLIEKAGAWYSYKGEKIG
QGKANATAWLKDNPETAKEIEKKVRELLLSNPNSTPDFSVDDSEGVAET
NEDF.

The wild type B. anthracis RecA (GenBank Acc. No. NC_{—}007530) coding region, which in the B. anthracis genome occurs in two segments, separated by an intron, has the following sequence (after the intron is removed in silico) (SEQ ID NO:53):

| | |
|---|---|
| 1 | atgagtgatc gtcaagcagc gttagatatg gcgttaaaac |
| | aaatagagaa gcaattcggt |
| 61 | aaaggttcaa ttatgaaatt aggagaacaa gcagagcgca |
| | aagtttctac ggtttctagt |
| 121 | ggttctttag cacttgatgt ggcattaggg gtaggcggat |
| | acccacgcgg ccgtattatc |
| 181 | gaaatttacg gacctgaaag ttcaggtaaa acaacagttt |
| | cattacacgc aattgcagaa |
| 241 | gtacagcgtc aaggtggaca agcagcgttc attgatgctg |
| | agcatgcaat ggatcctgta |
| 301 | tatgcacaaa aactaggtgt taacatcgat gaattactat |
| | tatcacaacc tgatacaggg |
| 361 | gagcaaggtt tagaaatcgc agaagcactt gtacgaagtg |
| | gtgcggttga tattatcgta |
| 421 | attgactctg tagcagctct tgtaccgaaa gctgaaattg |
| | aaggagacat gggtgactca |
| 481 | cacgtaggtt tacaagctcg tctaatgtct caagcacttc |
| | gtaaactttc aggtgcaatc |
| 541 | aataaatcaa aaacaatcgc aatctttatt aaccaaattc |
| | gtgaaaaagt tggggttatg |
| 601 | ttcggaaatc cagaaacaac tccaggtggt cgtgcgttga |
| | aattctattc aactgttcgt |
| 661 | cttgaagtgc gtcgtgcgga gcaattaaaa caaggtaacg |
| | acatcgttgg taataaaaca |
| 721 | aaagtaaaag tagttaaaaa taaagtggca ccaccattcc |
| | gtgttgcgga agttgatatt |
| 781 | atgtacggag aaggtatttc aagagaaggt gaaatcttag |
| | atatggcctc tgaacttgat |
| 841 | atcgttcaaa agagcggtgc ttggtactct tataatgaag |
| | aacgcttagg acaaggtcgt |
| 901 | gaaaattcga agcagttctt aaaagaaaat acggatttaa |
| | gagaagaaat tgccttcttt |
| 961 | attcgtgagc atcacggaat tagcgaagat tctggtgctg |
| | aaggtatgga agatccaaat |
| 1021 | cttcttgatt aa. |

The amino acid sequence of a predicted temperature sensitive B. anthracis RecA protein comprising a mutation (underlined) analogous to that of the E. Coli recA44 temperature sensitive mutant is as follows (SEQ ID NO:54):

MSDRQAALDMALKQIEKQFGKGSIMKLGEQAERKVSTVSSGSLALDVALG
VGGYPRGRIIEIYGPESSGKTTVSLHAIAEVQRQGGQAAFIDAEHAMDPV
YAQKLGVNIDELLLSQPDTGEQGLEIAEALVRSGAVDIIVIDSVAALVPK
AEIEGDMGDSHVGLQARLMSQALRKLSGAINKSKTIAIFINQIREKVGVM
FGNPETTPGGRALKFYSTVRLEVRRAEQLKQGNDIVGNKTKVKMVKNKVA
PPFRVAEVDIMYGEGISREGEILDMASELDIVQKSGAWYSYNEERLGQGR
ENSKQFLKENTDLREEIAFFIREHHGISEDSGAEGMEDPNLLD.

The region of the B. anthracis genome that includes the uvrA gene and uvrB gene appears below (SEQ ID NO:55) (coding regions are underlined; sequence from GenBank Acc. No. NC_{—}007530). The UvrA protein and UvrB protein are encoded by the complement of the shown underlined sequence. Complement of the UvrA coding sequence is found at: 4,884,383 to 4,887,259. Complement of the UvrB coding sequence is found at: 4,887,265 to 4,889,241. A region of only five base pairs separates the two coding regions. Also shown are upstream and downstream sequences.

| | |
|---|---|
| 4884001 | tcatccatac ttaataaact cgcatctttt |
| | attagtagta catcatcaca gtgttgatag |
| 4884061 | aagatgtagc aatccttttt cacttcatta |
| | tgaaacgcgt tctccgcttc ttctccctct |
| 4884121 | aaacacttct ttctctcata actaaatatg |
| | tgccatagat acccacatgc atgcttatct |
| 4884181 | ccataaagga aaatatcttc tttttcctca |
| | tcacttacat gatttgcaaa atgatcctcc |
| 4884241 | catcgcttcc gaaaatatac accccacttt |
| | tgaaactctc ttaccttcat atttttcttt |
| 4884301 | cgcaacatat ctaaaaactc catcttctcc |
| | ccctatacaa taaaaaccga gcgtcacttc |
| 4884361 | cactcggttt tcattcatac acttattgtg |
| | ataactctac ttcttttatt ttctctttca |
| 4884421 | ttctcgcttt atcacgattt aaaatctctt |
| | ttaaatactt acctgtatac gagcgctctt |
| 4884481 | ctttcactac ttgctctggc gttccggaag |
| | caacgatttg tccacctttg tctccgcctt |
| 4884541 | ctggtccaag gtcaacgata taatccgctg |
| | ttttaattac atctaaatta tgttcaatga |
| 4884601 | caagtaccgt ctcaccgctc tcaacaagac |
| | gttgcagcac ttctagaaga cgggcgatat |
| 4884661 | catgcgcatg taaaccagtc gttggctcgt |
| | ctaaaatgta tagtgtacgt cctgtagaac |
| 4884721 | gacggtgtaa ttcagaagct aatttcacac |
| | gctgtgcttc accaccagat aaagtcgtgg |
| 4884781 | ctggttgccc taatttcata taaccaagcc |
| | caacgtctac aagcgtttga agtttacgtt |
| 4884841 | taatttttgg gatattagcg aagaactcta |
| | ctccgtcttc aatcgtcatc cctaacactt |
| 4884901 | cagaaatgtt tttatcttta tatttcactt |
| | ctaacgtttc acggttgtaa cgtttaccgt |
| 4884961 | gacaaacttc acacggaacg tatacgtctg |
| | gtaagaagtg catctcaatt ttaataattc |
| 4885021 | catcaccacg gcacgcttca caacgtccac |
| | cttttacgtt aaagctgaaa cgcccttttt |
| 4885081 | gatatccgcg cactttcgct tcattcgttt |
| | gcgcaaacac atcacgaata tcatcgaaca |
| 4885141 | cacctgtata ggttgctgga ttagaacgtg |
| | gtgtacgacc gattggcgat tgatcaatat |
| 4885201 | cgataacttt atctaaatgc tcaagacctt |
| | taatttcttt atgagtacct ggcttcgctt |
| 4885261 | tcgctttata taacttttgc gctaacgatt |
| | tatatagtac ttcattaatc atcgtacttt |
| 4885321 | tacctgatcc agatacaccc gttaccgcta |
| | caaacgtacc aagcgggaat gacatcttcg |
| 4885381 | cgttctttaa gttattctct tttgcaccga |
| | caatctccac tttacgtcca tcacctttac |
| 4885441 | gtctttcaag tggaactgga ataaactctt |
| | taccgcttaa atacttacct gttagtgaat |
| 4885501 | tctcatcttg catcacttca gctggtgtac |
| | ccgctgatac aacttgtcca ccgtgaatac |
| 4885561 | ctgcgccagg cccgatatcc agtaaataat |
| | cagctgccat catcgtatct tcatcatgct |
| 4885621 | caacaacaat taacgtatta cctaaatcac |
| | gcatttcttg caatgtacga ataagacgat |
| 4885681 | cgttatcgcg ctgatgcaaa ccgatagaag |
| | gctcatcaag aatgtaaagc accccagtaa |
| 4885741 | gacgcgaacc aatttgcgtt gctaaacgaa |
| | tacgttgcgc ctcaccacca gataaagttc |
| 4885801 | ctgcggcacg acttaacgtt aaataatcta |
| | aaccaacgtt tactaagaac ccaacgcgct |
| 4885861 | cttgaatttc tcttaaaatt aaatgggcaa |
| | ttttttgttg tttctctgtt agctccacat |
| 4885921 | ttgagaagaa ttcctgtact tcttgaacag |
| | aatacttcgt tacatcagca atcgtttttc |
| 4885981 | cgccaacgaa aacagctaaa ctttcaggct |
| | ttaagcgtcc gcctttacac ttcggacaag |
| 4886041 | cttgttctgc catatacttt tccatttgct |
| | cacgaatgta atccgaactc gtctcacgat |
| 4886101 | aacgacgttc aatatttgga ataacacctt |
| | caaataaaat ctcattttcc tttacttgac |
| 4886161 | caaattcatt tacatagcgg aaataaactt |
| | tctcttcacc gcttccgtac aacactttat |
| 4886221 | caaataaatc tttcggtata tcttttacag |
| | gcacatccat atccacgcca taatgattac |
| 4886281 | atacagattg taaaagctgt gggtaatatt |
| | gtgaacttgt cggttcccaa ggcgcaatcg |
| 4886341 | catgctcatt taatgataaa tcccagttcg |
| | gaataacaag ttctaaatct acctctaact |
| 4886401 | ttgagccaag cccatcacaa gaaggacatg |
| | caccgaacgg actattgaat gagaacatac |
| 4886461 | gcggctctaa ttctccaatt gaaaaaccac |
| | aatgcggaca agcatgatgt tcactaaata |
| 4886521 | gaagctcctc ttctcccata acatcgatta |
| | acactcgtcc cccgccaagc tttaatgcac |
| 4886581 | tttcaagaga atcagcaaga cggcttgcga |
| | ttccttcttt tacaacaata cggtcaatta |
| 4886641 | caacttcaat agaatgcttc ttatttttat |
| | ctaacgcaat atcttcagac acatcgagca |
| 4886701 | tttcaccatc aacacgtaca cgaacataac |
| | cttgcttctt aatatcttca agtactttta |
| 4886761 | catgtgcacc tttacgccca gaaacgatag |
| | gagctaacac ttgtaatttc gtacgttcag |
| 4886821 | ggtactcaag tacacggtct accatttgct |
| | ctactgtttg cgatgtaatt tcaatgccat |
| 4886881 | gattcggaca aattggcgta ccaattcgcg |
| | caaataataa acgtaagtaa tcataaatct |
| 4886941 | ccgttaccgt tccaacagtt gaacgcggat |
| | tacgactcgt cgttttttga tcgattgaaa |
| 4887001 | tcgctggaga taagccttca atcgtatcta |
| | catccggctt atccatttgc cctaaaaact |
| 4887061 | ggcgtgcata cgcagataac gattctacgt |
| | atctgcgctg cccttctgca taaatcgtat |
| 4887121 | caaatgctaa tgaggatttc cctgaaccag |
| | acaatcctgt tacaacgaca agttgatttc |
| 4887181 | tcggaatggt tacatcaata ttttttaagt |
| | tatgtgctct agcacctttt acaacgataa |
| 4887241 | aatccttgct cttactcact cttttcaccc |
| | ttccgctttt aattctaata gtaaatctct |
| 4887301 | taattcagct gcacgctcga agtctaatgc |
| | ttttgctgct tctttcattt ctgcttccat |
| 4887361 | cttcgcaatt gtcttttcac gctctttttt |
| | cgtcatcttc ttagctggcg tcgcttcata |
| 4887421 | tgtttccggc tcttcagcag ctgtcgttgc |
| | acggattaca tcacgcacac ctttttgaat |
| 4887481 | cgttttcggc gtaataccat gctcttcatt |
| | gtaagcttct tgtatactac gacgacgctt |
| 4887541 | cgtctcttca atcgcaatcc ccatcgatct |
| | cgttatacga tctgcgtaca taataacgcg |
| 4887601 | accgttttca ttacgtgctg cacggccaat |
| | tgtttgaatt aacgaacgct ctgaacgcaa |
| 4887661 | gaatccttcc ttatcggcat ctaaaatagc |
| | tacaagggat acttctggaa tatctaatcc |
| 4887721 | ttctcgcaat aagttaatac caacgagaac |
| | atcaaactta ccaaggcgaa gatctcgtat |
| 4887781 | aatttcaata cgttctaacg ttttcacttc |
| | agaatgcaga taattcacct taattcctac |
| 4887841 | atcttttaag tagtctgtta aatcctctga |
| | catcttcttc gttaaagttg taattaatac |
| 4887901 | acgttcattt tttgcaatgc gatcttgaat |
| | ctctcctaat agatcgtcaa tctgcccttc |
| 4887961 | aattggtcgt atatcaattg gcggatctaa |
| | aagccctgtt ggacgaataa tttgttctat |
| 4888021 | tacttctggc gactgctcta attcatacgg |
| | tcctggcgtt gctgaaacgt aaataacttg |
| 4888081 | attcgttttc tcttcaaact catcaaatgt |
| | gagcggtcta ttatctaaag ctgatggcag |
| 4888141 | acggaatcca tgatccacaa gcacttgttt |
| | acgcgcttgg tccccgttat acatcgctct |
| 4888201 | tacttgcggc actgatacgt gggactcatc |
| | cataacgatt aagaaatctt tcgggaaata |
| 4888261 | gtctaataac gtatacggcg ttgcacccgc |
| | tggacgaagt gttaaatgac gggaatagtt |
| 4888321 | ttcaatccct gaacaaaagc ccatctcgcg |
| | catcatttct aaatcataac gtgtacgctg |
| 4888381 | ttctatacgc tgcgcttcta acaacttacc |
| | gttatcattt aattccttta aacgctcttc |
| 4888441 | taattctttt tcgatatttt caatagcgac |
| | cttcatcttt tcttcacgtg taacgaagtg |
| 4888501 | agatgctggg aagattgcta catgatcacg |
| | ttctgctaat acttctcccg ttaaagcatt |
| 4888561 | tacttcgcga atacgatcaa tttcatcgcc |
| | aaaaaactca attcgaatgc aatgctcgtc |
| 4888621 | aagtgatgcc gggaagattt caactacatc |
| | tccgcgcacg cggaatgtac cacgcttgaa |
| 4888681 | atcaatatca ttacgtccat actgcacatc |
| | aacaagttca cgaagcaatt gattgcggtc |
| 4888741 | cttttccata ccaactcgaa gtgaaacaac |
| | taactcgcgg tattcttctg gagaacctaa |
| 4888801 | accatatata cacgaaacac tcgcaacaat |
| | aattacatca tcccgttcaa ataatgcgga |
| 4888861 | cgttgctgag tgacgcaatt tatcgatttc |
| | atcattaatc tgcgcgtctt tttcaataaa |
| 4888921 | cgtatctgtt tgtggcacat acgcttctgg |
| | ctgataataa tcgtaataac taacaaaata |
| 4888981 | ttcaactgca ttattcggga aaaagtcttt |
| | caactcacta tataactgtc ctgctaacgt |
| 4889041 | tttattgtga gccatgacaa gcgttggctt |
| | ttgcacttct ttaatgacat ttgaaatcgt |
| 4889101 | aaatgtctta cccgttcctg tcgccccaag |
| | caacacttgc tttttctttc cactattaat |
| 4889161 | tccctctaca agcttctcta tagctaccgg |
| | ctgatcacct tgcggggaat acgctgagac |
| 4889221 | aatttcaaat tgacgttcca agaaaatccg |
| | acctgctaca aaaaatatag acaaatgtat |
| 4889281 | attcttagag tgttcctgct tctacgtgtc |
| | ctgccttgcc aaccactcgc tactacagtt |
| 4889341 | tgatctaaca ctccacaggc gtaaattccg |
| | atgtagccca ccgtacatat atacaatcta |
| 4889401 | tttacgttag tattgtatat ttttttgctt |
| | gtaaaaggtt tattgacgcg ttcaaatctc |
| 4889461 | tatccattac atttccacag ttacatgtat |
| | atactcggtc ggatagcttt aagtctactt. |

What is claimed is:

1. An isolated, asporogenic Bacillus anthracis strain that is attenuated for nucleotide excision repair (NER).

2. The Bacillus anthracis strain of claim 1, which is defective with respect to SpoIIE.

3. The Bacillus anthracis strain of claim 1, comprising an inhibiting mutation in, or a modification that attenuates expression of, a sporulation gene.

4. The Bacillus anthracis strain of claim 1, which comprises a mutation in the spoIIE gene.

5. The Bacillus anthracis strain of claim 1, which is defective with respect to one or more enzymes selected from the group consisting of UvrA, UvrB, and UvrC.

6. The Bacillus anthracis strain of claim 1, which comprises a mutation in at least one DNA nucleotide excision repair gene.

7. The Bacillus anthracis strain of claim 6, which comprises a mutation in one or more genes selected from the group consisting of uvrA gene, uvrB gene, and uvrC gene.

8. The Bacillus anthracis strain of claim 1, wherein the strain is defective with respect to SpoIIE, UvrA, and UvrB.

9. The Bacillus anthracis strain of claim 1, further comprising an inhibiting mutation in, or a modification that attenuates expression of, a recombinational repair gene.

10. The Bacillus anthracis strain of claim 9, wherein the recombinational repair gene is recA.

11. The Bacillus anthracis strain of claim 9, wherein the mutation comprises a conditional mutation.

12. The Bacillus anthracis strain of claim 9, wherein the modification comprises a nucleic acid molecule encoding a RecA protein, wherein expression of the RecA protein is under the control of a transcriptional or translational activator or repressor.

13. The Bacillus anthracis strain of claim 1, which is defective with respect to RecA.

14. The Bacillus anthracis strain of claim 13, which comprises a mutant recA gene which encodes a temperature-sensitive RecA.

15. The strain of claim 13, which is an inducible or repressible recA mutant.

16. The Bacillus anthracis strain of claim 1, further comprising an inhibiting mutation in, or a modification that attenuates expression of, at least one toxin gene.

17. The Bacillus anthracis strain of claim 16, wherein the at least one toxin gene comprises the:
a) lef gene;
b) cya gene; or c) lef and cya genes.

18. The Bacillus anthracis strain of claim 1, which comprises one or more mutations in the lef gene, the cya gene, or both genes that decreases the toxicity of the strain.

19. The Bacillus anthracis strain of claim 1, comprising a nucleic acid encoding at least one antigen, wherein the nucleic acid encoding the antigen is operably linked to a heterologous promoter.

20. The Bacillus anthracis strain of claim 19, wherein the heterologous promoter is inducible.

21. The Bacillus anthracis strain of claim 20, wherein the heterologous promoter is inducible by:
a) ultraviolet light;
b) a nucleic acid targeted compound;
c) a nucleic acid cross-linking compound; or d) an SOS regulatory pathway.

22. The Bacillus anthracis strain of claim 19, wherein the nucleic acid encoding the antigen is or is derived from the:
a) pagA gene;
b) lef gene;
c) cya gene;
d) pagA and lef genes;
e) pagA and cya genes;
f) cya and lef genes; or
g) pagA, lef, and cya genes.

23. The Bacillus anthracis strain of claim 20, wherein the antigen is protective antigen and the nucleic acid encoding the protective antigen is operably linked to an SOS regulatory sequence.

24. The Bacillus anthracis strain of claim 1 that comprises a poly-D-glutamate capsule.

25. The Bacillus anthracis strain of claim 1 that lacks a poly-D-glutamate capsule.

26. A bacterium of the Bacillus anthracis strain of claim 1, wherein the nucleic acid of the bacterium has been modified so that the bacterium is attenuated for proliferation.

27. The bacterium of claim 26, wherein the bacterium has been modified by a nucleic acid targeted compound that reacts directly with the nucleic acid.

28. The bacterium of claim 27, wherein the bacterium has been treated with a psoralen and UVA radiation.

29. A bacterium of the Bacillus anthracis strain of claim 1, further comprising at least one covalently linked nucleic acid cross-linking compound, linked to the genomic DNA of the bacterium.

30. The bacterium of claim 29, wherein the cross-linking compound comprises a psoralen.

31. A vaccine or composition comprising a bacterium of the Bacillus anthracis strain of claim 1 or the bacterium of claim 26.

32. A method of inducing an immune response in a host comprising administering to the host an effective amount of a composition comprising a bacterium of the strain of claim 1.

33. A Bacillus anthracis strain comprising a heterologous expression cassette comprising a nucleic acid molecule encoding protective antigen (PA), wherein the nucleic acid molecule encoding protective antigen is operably linked to an inducible promoter.

34. The Bacillus anthracis strain of claim 33, further comprising an inhibiting mutation in, or a modification that attenuates expression of, at least one nucleic acid repair gene.

35. The Bacillus anthracis strain of claim 34, wherein the nucleic acid repair gene comprises:
a) a nucleotide excision repair (NER) gene;
b) a recombinational repair gene; or
c) an NER gene and a recombinational repair gene.

36. The Bacillus anthracis strain of claim 35, wherein the nucleic acid repair gene comprises:
a) uvrA;
b) uvrB;
c) uvrC;
d) uvrA and uvrB; or
e) uvrA, uvrB, and uvrC.

37. The Bacillus anthracis strain of claim 35, wherein the nucleic acid repair gene is recA.

38. The Bacillus anthracis strain of claim 37, wherein the mutation comprises a conditional mutation.

39. The Bacillus anthracis strain of claim 34, wherein the modification comprises a nucleic acid molecule encoding a RecA protein, wherein expression of the RecA protein is under the control of a transcriptional or translational activator or repressor.

40. The Bacillus anthracis strain of claim 33, wherein the nucleic acid encoding the protective antigen is operably linked to an SOS regulatory sequence.

41. The Bacillus anthracis strain of claim 33, wherein expression of the protective antigen is induced by treatment of the strain with a psoralen and UVA radiation.

42. The Bacillus anthracis strain of claim 33, further comprising an inhibiting mutation in, or a modification attenuating expression of, at least one:
a) sporulation gene; or
b) toxin gene.

43. The Bacillus anthracis strain of claim 42, wherein the at least one toxin or sporulation gene comprises:
a) spoIIE gene;
b) lef gene;
c) cya gene;
d) SpoIIE and lef genes;
e) spoIIE and cya genes;
f) lef and cya genes;
g) spoIIE, lef, and cya genes.

44. The Bacillus anthracis strain of claim 33 that comprises a poly-D-glutamate capsule.

45. The Bacillus anthracis strain of claim 33 that lacks a poly-D-glutamate capsule.

46. A bacterium of the Bacillus anthracis strain of claim 33, further comprising at least one covalently linked nucleic acid cross-linking compound, linked to the genomic DNA of the bacterium.

47. The bacterium of claim 46, wherein the cross-linking compound comprises a psoralen.

48. A vaccine or composition comprising a bacterium of the Bacillus anthracis strain of claim 33.

49. A method of inducing an immune response in a host to Bacillus anthracis comprising administering to the host an effective amount of a composition comprising a bacterium of the strain of claim 33.

50. An isolated Bacillus anthracis bacterium comprising a mutation in a Bacillus anthracis recA gene, wherein the recA gene encodes a temperature-sensitive RecA protein comprising a V244M mutation.

51. A Bacillus anthracis bacterium comprising a recA gene derived from a foreign bacterium, wherein the recA gene encodes a temperature-sensitive RecA protein.

52. The Bacillus anthracis bacterium of claim 51, wherein the foreign bacterium is E. Coli.

53. An isolated Bacillus anthracis bacterium which is a repressible or inducible recA mutant.

54. The isolated Bacillus anthracis bacterium of claim 53, which is a repressible recA mutant.

55. The isolated Bacillus anthracis bacterium of claim 53, wherein expression of a RecA protein is under the control of a heterologous transcriptional repressor or activator.

56. The isolated Bacillus anthracis bacterium of claim 53, wherein expression of a recA gene is under control of a lac repressor.

57. The isolated Bacillus anthracis bacterium of claim 53, which comprises a nucleic acid encoding a RecA protein, wherein the nucleic acid is operably linked to a heterologous operator which binds a repressor or activator.

58. The isolated Bacillus anthracis bacterium of claim 53, which comprises a nucleic acid that produces an RecA antisense RNA upon transcription, wherein the nucleic acid is operably linked to an operator which binds a repressor or activator.

59. A vaccine or composition comprising the bacterium of claim 50, 51, 53.

60. A method of inducing an immune response in a host to Bacillus anthracis comprising administering to the host an effective amount of a composition comprising a bacterium of claim 50, 51, or 53.

61. A Bacillus anthracis strain comprising a nucleic acid encoding an antigen, wherein the nucleic acid encoding the antigen is operably linked to a heterologous promoter.

62. The Bacillus anthracis strain of claim 61, wherein the nucleic acid encoding the antigen is, or is derived from:
a) pagA gene;
b) lef gene;
c) cya gene;
d) pagA and lef genes;
e) pagA and cya genes;
f) cya and lef genes; or g) pagA, lef, and cya genes.

63. The Bacillus anthracis strain of claim 61, wherein the heterologous promoter is inducible.

64. The Bacillus anthracis strain of claim 61, wherein the heterologous promoter is inducible by:
a) ultraviolet light;
b) a nucleic acid cross-linking compound;
c) ultraviolet light and a nucleic acid cross-linking compound;
d) an SOS regulatory pathway; or
e) a change or shift in temperature.

65. A vaccine or composition comprising a bacterium of the Bacillus anthracis strain of claim 61.

66. A method of inducing an immune response in a host to Bacillus anthracis comprising. administering to the host an effective amount of a composition comprising a bacterium of the strain of claim 61.

67. A Bacillus anthracis strain comprising at least one inhibiting mutation in each of uvrA gene, uvrB gene, lef gene, cya gene, and SpoIIE gene.

68. A bacterium of the Bacillus anthracis strain of claim 67, further comprising at least one covalently linked nucleic acid cross-linking compound linked to the genomic DNA of the bacterium.

69. An isolated, asporogenic Bacillus anthracis bacterium, wherein the nucleic acid of the bacterium has been modified so that the bacterium is attenuated for proliferation.

70. The bacterium of claim 69, wherein the nucleic acid of the bacterium has been modified by a nucleic acid targeted compound that reacts directly with the nucleic acid.

71. The bacterium of claim 70, wherein the nucleic-acid targeted compound is a nucleic acid alkylator.

72. The bacterium of claim 70, wherein the nucleic acid targeted compound is a psoralen compound activated by UVA irradiation.

73. The bacterium of claim 69, comprising at least one covalently linked nucleic acid cross-linking compound, linked to the genomic DNA of the bacterium.

74. The bacterium of claim 73, wherein the cross-linking compound comprises a psoralen.

75. The bacterium of claim 69, which is defective with respect to SpoIIE.

76. The bacterium of claim 69, which comprises a mutation in a sporulation gene.

77. The bacterium of claim 76, which comprises a mutation in the spoIIE gene.

78. The bacterium of claim 69, which is attenuated for nucleic acid repair.

79. The bacterium of claim 69, which is defective with respect to at least one DNA repair enzyme.

80. The bacterium of claim 79, which is defective with respect to one or more enzyme selected from the group consisting of UvrA, UvrB, and UvrC.

81. The bacterium of claim 78, which comprises a mutation in one or more genes selected from the group consisting of uvrA, uvrB, and uvrC.

82. The bacterium of claim 69, which is attenuated for recombinational repair.

83. The bacterium of claim 82, which is defective with respect to RecA.

84. The bacterium of claim 83, which comprises a mutation in the recA gene.

85. The bacterium of claim 84, which comprises a mutant recA gene which encodes a temperature-sensitive RecA.

86. The bacterium of claim 83, which is a repressible or inducible recA mutant.

87. The bacterium of claim 69, wherein the bacterium expresses protective antigen (PA) under the control of an SOS regulatory sequence.

88. The bacterium of claim 69, which comprises one or more mutations in the lef gene, the cya gene, or both genes that decreases the toxicity of the strain.

89. A vaccine or composition comprising a bacterium of claim 69.

90. A method of inducing an immune response in a host to Bacillus anthracis comprising administering to the host an effective amount of a composition comprising a bacterium of claim 69.

91. An isolated, sporulation-deficient Bacillus anthracis strain that is attenuated for nucleotide excision repair (NER).

92. An isolated, sporulation-deficient Bacillus anthracis bacterium, wherein the nucleic acid of the bacterium has been modified so that the bacterium is attenuated for proliferation.

93. The bacterium of claim 92, wherein the bacterium has been modified with a nucleic acid targeted compound that reacts directly with the nucleic acid, so that the bacterium is attenuated for proliferation.

94. An isolated Bacillus anthracis strain which is defective with respect to SpoIIE.

95. The Bacillus anthracis strain of claim 94, which is sporulation-deficient.

96. The Bacillus anthracis strain of claim 94, which is asporogenic.

97. The Bacillus anthracis strain of claim 94, which comprises a mutation in SpoIIE.

98. A vaccine or composition comprising a bacterium of claim 92 or a bacterium of the Bacillus anthracis strain of claim 91 or 94.

99. A method of inducing an immune response in a host to Bacillus anthracis comprising administering to the host an effective amount of a composition comprising a bacterium of claim 92 or a bacterium of the strain of claim 91 or 94.

100. A vaccine composition comprising a bacterium from an asporogenic Bacillus anthracis strain that is attenuated for nucleic acid repair.

101. A method of inducing an immune response in a host to Bacillus anthracis comprising administering to the host an effective amount of a composition comprising a bacterium from an asporogenic Bacillus anthracis strain that is attenuated for nucleic acid repair.

102. A vaccine composition comprising a bacterium from a sporulation-deficient Bacillus anthracis strain that is attenuated for nucleic acid repair.

103. A method of inducing an immune response in a host to Bacillus anthracis comprising administering to the host an effective amount of a composition comprising a bacterium from a sporulation-deficient Bacillus anthracis strain that is attenuated for nucleic acid repair.
